SEQUENCE LISTING (2 sequences)

SEQ ID NO: 1 - protein, 1008 aa, Homo sapiens
MRVNEKYSTLPAEDRSVHIINICAIEDIGYLPSEGTLLNSLSVDPDAECKYGLYFRDGRRKVDYILVYHHKRPSGNRTLVRRVQHSDTPSGARSVKQDHPLPGKGASLDAGSGEPPMDYHEDDKRFRREEYEGNLLEAGLELERDEDTKIHGVGFVKIHAPWNVLCREAEFLKLKMPTKKMYHINETRGLLKKINSVLQKITDPIQPKVAEHRPQTMKRLSYPFSREKQHLFDLSDKDSFFDSKTRSTIVYEILKRTTCTKAKYSMGQGEGRKKDSALLSKRRKCGKYGITSLLANGVYAAAYPLHDGDYNGENVEFNDRKLLYEEWARYGVFYKYQPIDLVRKYFGEKIGLYFAWLGVYTQMLIPASIVGIIVFLYGCATMDENIPSMEMCDQRHNITMCPLCDKTCSYWKMSSACATARASHLFDNPATVFFSVFMALWAATFMEHWKRKQMRLNYRWDLTGFEEEEEAVKDHPRAEYEARVLEKSLKKESRNKEKRRHIPEESTNKWKQRVKTAMAGVKLTDKVKLTWRDRFPAYLTNLVSIIFMIAVTFAIVLGVIIYRISMAAALAMNSSPSVRSNIRVTVTATAVIINLVVIILLDEVYGCIARWLTKIEVPKTEKSFEERLIFKAFLLKFVNSYTPIFYVAFFKGRFVGRPGDYVYIFRSFRMEECAPGGCLMELCIQLSIIMLGKQLIQNNLFEIGIPKMKKLIRYLKLKQQSPPDHEECVKRKQRYEVDYNLEPFAGLTPEYMEMIIQFGFVTLFVASFPLAPLFALLNNIIEIRLDAKKFVTELRRPVAVRAKDIGIWYNILRGIGKLAVIINAFVISFTSDFIPRLVYLYMYSKNGTMHGFVNHTLSSFNVSDFQNGTAPNDPLDLGYEVQICRYKDYREPPWSENKYDISKDFWAVLAARLAFVIVFQNLVMFMSDFVDWVIPDIPKDISQQIHKEKVLMVELFMREEQDKQQLLETWMEKERQKDEPPCNHHNTKACPDSLGSPAPSHAYHGGVL

SEQ ID NO: 2 - DNA, 4876 bp, Homo sapiens
aaaggcgggccggctggcgtccaagttcctgaccaggcgcgggccggcccgcgggaccagcagccgggtggcggcgcgatcggccccgagaggctcaggcgccccccgcatcgagcgcgcgggccgggcgggccagggcggcgggcggagcgggaggcggccacgtccccggcgggcctgggcgcggggaggcccggccccctgcgagcgcgccgcgaacgctgcggtctccgcccgcagaggccgccggggccgtggatggggagggcgcgccgcccggcggtcccagcgcacaggcggccacgatgagggtcaacgagaagtactcgacgctcccggccgaggaccgcagcgtccacatcatcaacatctgcgccatcgaggacatcggctacctgccgtccgagggcacgctgctgaactccttatctgtggaccctgatgccgagtgcaagtatggcctgtacttcagggacggccggcgcaaggtggactacatcctggtgtaccatcacaagaggccctcgggcaaccggaccctggtcaggagggtgcagcacagcgacaccccctctggggctcgcagcgtcaagcaggaccaccccctgccgggcaagggggcgtcgctggatgcaggctcgggggagcccccgatggactaccacgaggatgacaagcgcttccgcagggaggagtacgagggcaacctcctggaggcgggcctggagctggagcgggacgaggacactaaaatccacggagtcgggtttgtgaaaatccatgccccctggaacgtgctgtgcagagaggccgagtttctgaaactgaagatgccgacgaagaagatgtaccacattaatgagacccgtggcctcctgaaaaaaatcaactctgtgctccagaaaatcacagatcccatccagcccaaagtggctgagcacaggccccagaccatgaagagactctcctatcccttctcccgggagaagcagcatctatttgacttgtctgataaggattcctttttcgacagcaaaacccggagcacgattgtctatgagatcttgaagagaacgacgtgtacaaaggccaagtacagcatgggccaaggcgagggaagaaagaaggactccgcccttctaagtaaaaggcggaaatgtgggaagtatggcatcacgagcctgctggccaatggtgtgtacgcggctgcatacccactgcacgatggagactacaacggtgaaaacgtcgagttcaacgacagaaaactcctgtacgaagagtgggcacgctatggagttttctataagtaccagcccatcgacctggtcaggaagtattttggggagaagatcggcctgtacttcgcctggctgggcgtgtacacccagatgctcatccctgcctccatcgtgggaatcattgtcttcctgtacggatgcgccaccatggatgaaaacatccccagcatggagatgtgtgaccagagacacaatatcaccatgtgcccgctttgcgacaagacctgcagctactggaagatgagctcagcctgcgccacggcccgcgccagccacctcttcgacaaccccgccacggtcttcttctctgtcttcatggccctctgggctgccaccttcatggagcactggaagcggaaacagatgcgactcaactaccgctgggacctcacgggctttgaagaggaagaggaggctgtcaaggatcatcctagagctgaatacgaagccagagtcttggagaagtctctgaagaaagagtccagaaacaaagagaagcgccggcatattccagaggagtcaacaaacaaatggaagcagagggttaagacagccatggcgggggtgaaattgactgacaaagtgaagctgacatggagagatcggttcccagcctacctcactaacttggtctccatcatcttcatgattgcagtgacgtttgccatcgtcctcggcgtcatcatctacagaatctccatggccgccgccttggccatgaactcctccccctccgtgcggtccaacatccgggtcacagtcacagccaccgcagtcatcatcaacctagtggtcatcatcctcctggacgaggtgtatggctgcatagcccgatggctcaccaagatcgaggtcccaaagacggagaaaagctttgaggagaggctgatcttcaaggctttcctgctgaagtttgtgaattcctacacccccatcttttacgtggcgttcttcaaaggccggtttgttggacgcccgggcgactacgtgtacattttccgttccttccgaatggaagagtgtgcgccagggggctgcctgatggagctatgcatccagctcagcatcatcatgctggggaaacagctgatccagaacaacctgttcgagatcggcatcccgaagatgaagaagctcatccgctacctgaagctgaagcagcagagcccccctgaccacgaggagtgtgtgaagaggaaacagcggtacgaggtggattacaacctggagcccttcgcgggcctcaccccagagtacatggaaatgatcatccagtttggcttcgtcaccctgtttgtcgcctccttccccctggccccactgtttgcgctgctgaacaacatcatcgagatccgcctggacgccaaaaagtttgtcactgagctccgaaggccggtagctgtcagagccaaagacatcggaatctggtacaatatcctcagaggcattgggaagcttgctgtcatcatcaatgccttcgtgatctccttcacgtctgacttcatcccgcgcctggtgtacctctacatgtacagtaagaacgggaccatgcacggcttcgtcaaccacaccctctcctccttcaacgtcagtgacttccagaacggcacggcccccaatgaccccctggacctgggctacgaggtgcagatctgcaggtataaagactaccgagagccgccgtggtcggaaaacaagtacgacatctccaaggacttctgggccgtcctggcagcccggctggcgtttgtcatcgtcttccagaacctggtcatgttcatgagcgactttgtggactgggtcatcccggacatccccaaggacatcagccagcagatccacaaggagaaggtgctcatggtggagctgttcatgcgggaggagcaagacaagcagcagctgctggaaacctggatggagaaggagcggcagaaggacgagccgccgtgcaaccaccacaacaccaaagcctgcccagacagcctcggcagcccagcccccagccatgcctaccacgggggcgtcctgtagctatgccagcggggctgggcaggccagccgggcatcctgaccgatgggcaccctctcccagggcaggcggcttcccgctcccaccagggcccggtgggtcctgggttttctgcaaacatggaggaccactttctgataggacattttcctttcttctttctgttttctttcccttgtttttgcacaaagccattatgcagggaatattttttaatctgtagtattcaagatgaatcaaaatgatggctggtaatacggcaataaggtagcaaaggcaggtgctttgcagaaagaatgcttggaaacttgagtctccctagaggtgaaaagtgagcagaggcccgtagaaaccctcctctgaatcctcctaattccttaagatagatgcaaaatggtaagccgaggcatcgcgcaaaagctggtgcgatgcttcagggaaaatggaaaacccacgcaagaataatgattgattccggttccaaaaggtgtcacctacctgtttcagaaaagttagactttccatcgccttttccttccatcagttgagtggctgagagagaagtgcctcatccctgagccacacagggggcgtgggagcatcccagttatccctggaaagctagaaggggacagaggtgtccctgattaagcaggaaacagcacccttggcgtccccagcaggctccccactgtcagccacacacctgcccccatcacaccaagccgacctcagagttgttcatcttccttatgggacaaaaccggttgaccagaaaatgggcagagagagatgacctggaagcatttccacagatggtgtcagggtttcaagaagtcttagggcttccaggggtcccctggaagctttagaatatttatgggtttttttttcaaatatcaattatatggtagattgaggattttttttctgtagctcaaaggtggagggagtttattagttaaccaaatatcgttgagaggaatttaaaatactgttactaccaaagatttttattaataaaggcttatattttggtaacacttctctatatttttactcacaggaatgtcactgttggacaattattttaaaagtgtataaaaccaagtctcataaatgatatgagtgatctaaatttgcagcaatgatactaaacaactctctgaaatttctcaagcaccaagagaaacatcattttagcaaaggccaggaggaaaaatagaaataaatttgtcttgaagatctcattgatgtgatgttacattccctttaatctgccaactgtggtcaaagttcataggtgtcgtacatttccattatttgctaaaatcatgcaatctgatgcttctcttttctcttgtacagtaagtagtttgaagtgggttttgtatataaatactgtattaaaaattaggcaattaccaaaaatccttttatggaaaccatttttttaaaaagtgaatgtacacaaatccacagaggactgtggctggacattcatctaaataaatttgaatatacgacacttttctcacttgaaaaa

(19) United States
(12) Patent Application Publication
(10) Pub. No.: US 2022/0306617 A1
(43) Pub. Date: Sep. 29, 2022

(54) SUBSTITUTED 1,3-PHENYL HETEROARYL DERIVATIVES AND THEIR USE IN THE TREATMENT OF DISEASE

(71) Applicant: NOVARTIS AG, Basel (CH)

(72) Inventors: Claire ADCOCK, Pulborough (GB); Jake AXFORD, Waltham, MA (US); Ying HOU, Belmont, MA (US); Hyungchul KIM, Wilmington, DE (US); Yiping SHEN, Cambridge, MA (US); Nichola SMITH, Burlington, MA (US); Catherine Fooks SOLOVAY, Arlington, MA (US); Moo Je SUNG, Belmont, MA (US); Megan LIGHTFOOT, Nottingham (GB); Alessandro MAZZACANI, Hove (GB); Emily STANLEY, Horsham (GB); Lewis WHITEHEAD, Swampscott, MA (US)

(21) Appl. No.: 17/638,106

(22) PCT Filed: Aug. 24, 2020
(86) PCT No.: PCT/IB2020/057905

§ 371 Date: Feb. 24, 2022

Related U.S. Application Data
(60) Provisional application No. 62/892,664, filed on Aug. 28, 2019.

(51) Int. Cl. C07D 413/10; A61K 31/422; A61K 45/06; C07D 413/14; A61K 31/454; A61K 31/5377; A61K 31/675; C07F 9/6558; A61P 11/00

(52) U.S. Cl. CPC C07D 413/10; A61K 31/422; A61K 45/06; C07D 413/14; A61K 31/454; A61K 31/5377; A61K 31/675; C07F 9/65583; A61P 11/00

(57) ABSTRACT

The invention relates to heterocyclic compounds of the formula (I) in which all of the variables are as defined in the specification; capable of modulating the ctivity of TMEM16a. The invention further provides a method for manufacturing compounds of the invention, and its therapeutic uses. The invention further provides methods to their preparation, to their medical use, in particular to their use in the treatment and management of diseases or disorders including COPD, bronchiectasis, asthma, cystic fibrosis, primary ciliary dyskinesia, chronic bronchitis, cystic fibrosis, primary ciliary dyskinesia, respiratory tract infections (acute and chronic; viral and bacterial), lung carcinoma.

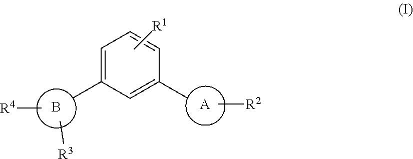

### FIELD OF THE INVENTION

The present invention relates substituted 1,3-Phenyl heteroaryl derivatives and pharmaceutically acceptable salts, hydrates and co-crystals thereof, compositions of these compounds, either alone or in combination with at least one additional therapeutic agent, processes for their preparation, their use in the treatment of diseases, their use, either alone or in combination with at least one additional therapeutic agent and optionally in combination with a pharmaceutically acceptable carrier, for the manufacture of pharmaceutical preparations, use of the pharmaceutical preparations for the treatment of diseases, and a method of treatment of said diseases, comprising administering the substituted 1,3-Phenyl heteroaryl derivatives to a warm-blooded animal, especially a human.

### BACKGROUND

Chronic obstructive pulmonary disease (COPD) is a chronic inflammatory disease of the lung characterized by persistent respiratory symptoms (dyspnea, cough, sputum production) and poorly reversible airflow limitation that is due to airway and/or alveolar abnormalities usually caused by significant exposure to noxious particles/gases, in particular cigarette smoke and biomass smoke exposure. Chronic airflow limitation is caused by a mixture of small airways disease (obstructive bronchiolitis) and parenchymal destruction (emphysema).

COPD is a critically important disease, as the tenth leading cause of death worldwide (GBD 2015 Mortality and Causes of Death 2016). COPD is associated with episodic periods of symptom deterioration termed exacerbations. Exacerbations are amongst the most common causes of medical admission to hospital and are also important events in the natural history of COPD that drive lung function decline (Donaldson et al., 2002).

Current standard of care in the management of COPD consists of short and long acting bronchodilators (LABA/LAMA)+/− inhaled corticosteroids (ICS) indicated in patients experiencing symptoms and exacerbations. Mucolytics have shown small and inconsistent benefits on exacerbation reduction and the efficacy of mucolytics on top of maximal inhaled treatment has yet to be clearly established (Wedzicha et al 2017). Thus despite currently available treatments almost 70% of patients remain significantly limited by breathlessness (mMRC 2) and 40% experience>2 moderate or >1 severe exacerbation per year (Mullerova et al., 2017).

TMEM16A has been identified as a calcium activated chloride channel (see, e.g., Yang et al., Nature, 455:1210-1215 (2008)). It is also known by some other names, such as ANO1, TAOS2, ORAOV2, and DOG-1. TMEM16A belongs to the anoctamin/TMEM16 family of membrane proteins. This family includes other members, such as TMEM16B-K. All TMEM16 proteins have similar putative topology, consisting of ten transmembrane segments and cytosolic N- and C- termini (see, e.g., Galietta, Biophysical J. 97:3047-3053, (2009); Dang et. Al, Nature, v. 552, pp. 426-429, 2017).

Calcium activated chloride channels functions in many physiological processes, including trans-epithelial secretion, cardiac and neuronal excitation, sensory transduction, smooth muscle contraction, and fertilization. TMEM16A is potentially involved in epithelial fluid secretion, olfactory and phototransduction, neuronal and cardiac excitability, and regulation of vascular tone including gut motility (see, e.g., Galietta, 2009).

TMEM16A is a calcium activated chloride channel expressed in the airway epithelium. TMEM16A provides a surrogate pathway for epithelial chloride secretion in the absence of the cystic fibrosis transmembrane conductance regulator (CFTR) such as in the disease cystic fibrosis. TMEM16A potentiator promotes a durable chloride flux from pulmonary epithelia with defective ion transport (COPD/CF) without promoting mucus secretion, enhancing mucociliary clearance (MCC), reducing the incidence of infectious exacerbations and improving the prognosis for patients with Bronchiectasis, COPD, asthma, and cystic fibrosis.

In view of the above, TMEM16A potentiators of formula (I) are considered to be of value in the treatment and/or prevention of chronic bronchitis, COPD, bronchiectasis, asthma, cystic fibrosis, primary ciliary dyskinesia, respiratory tract infections (acute and chronic; viral and bacterial), lung carcinoma and related disorders.

### SUMMARY

A first aspect of the invention relates to a compound of formula (I):

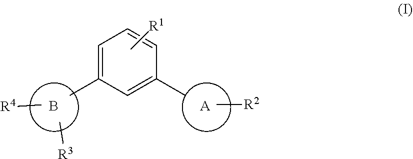

Wherein:
Ring A is a 5 membered heteroaryl containing 2 heteroatoms selected from N and O;
Ring B is a 5 membered heteroaryl containing 2 or 3 heteroatoms each independently selected from N, S and O, wherein at least one of said heteroatoms is N or ring B is a 6 membered heteroaryl containing 1 or 2 heteroatoms selected from N;
R¹ is hydrogen or halogen;
R² is selected from the group consisting of:

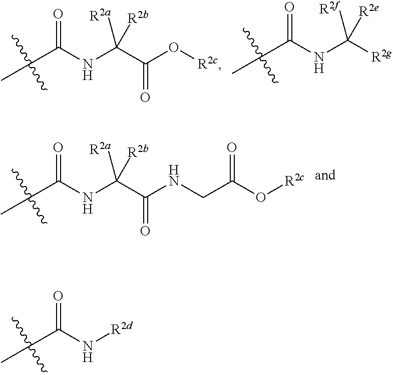

where

R^{2a} is H, (C₁-C₄)alkyl or phenyl, wherein said (C₁-C₄)alkyl is optionally substituted with halogen, (C₃-C₆)cycloalkyl, phenyl, —O—(C₁-C₄)alkyl or —S—(C₁-C₄)alkyl;

R^{2b} is H, (C₁-C₄)alkyl or R^{2b} taken together with R^{2a} forms a (C₃-C₆)cycloalkyl ring;

R^{2c} is (C₁-C₄)alkyl, (C₂-C₄)alkenyl or benzyl;

R^{2d} is (C₁-C₄)alkyl, (C₃-C₆)cycloalkyl, adamantyl, a 5 or 6 membered heteroaryl wherein said heteroaryl contains 1 or 2 heteroatoms independently selected from N and O, or phenyl; wherein said phenyl is optionally substituted with 1 or 2 substituents independently selected from (C₁-C₄)alkyl, halo-(C₁-C₄)alkyl and nitrile;

R^{2c} is H, (C₁-C₄)alkyl or (C₃-C₆)cycloalkyl ring;

R^{2f} is H, (C₁-C₄)alkyl or (C₃-C₆)cycloalkyl ring optionally substituted with (C₁-C₄)alkyl or R^{2c} taken together with R^{2f} forms a (C₃-C₆)cycloalkyl ring;

R^{2g} is H, (C₁-C₄)alkyl, a fused moiety selected from benzo[d][1,3]dioxole and indolin-2-one, where said fused moiety is optionally substituted with halogen or (C₁-C₄)alkyl, (C₃-C₆) heterocycloalkyl containing 1 or 2 heteroatoms selected from N and O, -(C₀-C₂)alkyl-phenyl wherein said phenyl is optionally substituted 1 or 2 groups independently selected from halogen and (C₁-C₄)alkyl;

R³ is H, (C₁-C₅)alkyl or a 4 to 6 membered saturated heterocycle containing O; wherein said (C₁-C₅)alkyl is optionally substituted with 1 to 3 groups independently selected from hydroxyl, (C₁-C₅)alkoxy, halogen, diethyl phosphate, —C(O)O(C₁-C₄)alkyl, NH-benzyl, O-benzyl, benzo[d][1,3]dioxole, isoindolinyl, —O—(C₂-C₄)alkyl-O—(C₁-C₄)alkyl, and a 4 to 6 membered saturated heterocycle containing 1 or 2 heteroatoms selected from N and O wherein said heterocycle is optionally substituted with 1 or 2 groups selected from (C₁-C₄)alkyl and —C(O)NH(CHR⁵)C(O)O—(C₁-C₄)alkyl;

R⁴ is selected from the group consisting of:

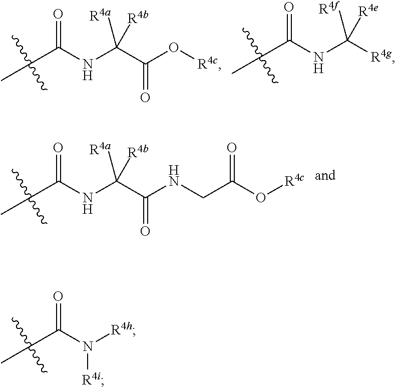

where

R^{4a} is H, (C₁-C₄)alkyl or phenyl, wherein said (C₁-C₄)alkyl is optionally substituted with 1 to 3 halogens, (C₃-C₆)cycloalkyl, phenyl, —O—(C₁-C₄)alkyl or —S—(C₁-C₄)alkyl;

R^{4b} is H or (C₁-C₄)alkyl or R^{4b} taken together with R^{4a} to form a (C₃-C₆)cycloalkyl ring;

R^{4c} is (C₁-C₄)alkyl, (C₂-C₄)alkenyl or benzyl;

R^{4e} e is H, (C₁-C₄)alkyl, (C₁-C₄)alkoxy or (C₃-C₆)cycloalkyl ring;

R^{4f} is H, (C₁-C₄)alkyl or (C₃-C₆)cycloalkyl ring optionally substituted with nitrile or (C₁-C₄)akyl or R^{4e} taken together with R^{4f} to form a (C₃-C₆)cycloalkyl ring;

R^{4g} is H, (C₁-C₄)alkyl, a fused moiety selected from benzo[d][1,3]dioxole and indolin-2-one, where said fused moiety is optionally substituted with halogen or (C₁-C₄)alkyl, (C₃-C₆)heterocycloalkyl containing 1 or 2 heteroatoms selected from N and O, -(C₀-C₂)alkyl-phenyl wherein said phenyl is optionally substituted with 1 or 2 halogens;

R^{4h} is (C₁-C₄)alkyl, (C₃-C₆)cycloalkyl optionally substituted with 1 or 2 halogens, adamantyl, a 5 or 6 membered heteroaryl wherein said heteroaryl contains 1 or 2 heteroatoms independently selected from N and O, or phenyl; wherein said phenyl is optionally substituted with 1 or 2 substituents independently selected from (C₁-C₄)alkyl, (C₁-C₅)alkoxy, halo-(C₁-C₄)alkyl, halo-(C₁-C₄)alkoxy and nitrile;

R⁴ⁱ is H or R⁴ⁱ taken together with R^{4h} forms a (C₃-C₆)heterocycloalkyl ring optionally substituted with 1 or 2 substituents independently selected from (C₁-C₄)alkyl, (C₁-C₄)alkoxy and —C(O)O(C₁-C₄)alkyl; and

R⁵ is H or (C₁-C₄)alkyl, wherein said (C₁-C₄)alkyl is optionally substituted with (C₃-C₆)cycloalkyl, phenyl, —O—(C₁-C₄)alkyl or —S—(C₁-C₄)alkyl; or a pharmaceutically acceptable salt, hydrate or co-crystal thereof.

Another aspect of the invention relates to polymorphs and salts of the compounds of formula (I).

Another aspect of the invention relates to pharmaceutical compositions comprising compounds of the invention or pharmaceutically acceptable salts or co-crystals thereof, and a pharmaceutical carrier. Such compositions can be administered in accordance with a method of the invention, typically as part of a therapeutic regimen for treatment or prevention of conditions and disorders mediated by potentiation of TMEM16A. In a particular aspect, the pharmaceutical compositions may additionally comprise further one or more therapeutically active ingredients suitable for use in combination with the compounds of the invention. In a more particular aspect, the further therapeutically active ingredient is an agent for the treatment of COPD and related disorders.

Another aspect of the invention relates to pharmaceutical combinations comprising compounds of the invention and other therapeutic agents for use as a medicament in the treatment of patients having disorders mediated by the potentiation of TMEM16A. Such combinations can be administered in accordance with a method of the invention, typically as part of a therapeutic regimen for treatment or prevention of COPD and related disorders.

Another aspect of the invention relates to polymorphs, hydrates and solvates of the compound of formula (I).

### BRIEF DESCRIPTION OF THE DRAWINGS

FIG. 1A. XRPD of monohydrate form of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide.

FIG. 1B. DSC thermogram of monohydrate form of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide.

FIG. 1C. DSC thermogram of micronized monohydrate form of the free base of N-(pentan-3-yl)-2-(3-(3-(((S)-1-((S)-tetrahydrofuran-2-yl)ethyl)carbamoyl)-1H-pyrazol-5-yl)phenyl)oxazole-5-carboxamide.

FIG. 2A. XRPD of metastable hydrate form of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide.

FIG. 2B. DSC thermogram of metastable hydrate form of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide.

FIG. 3A. XRPD of anhydrous form A of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide.

FIG. 3B. DSC thermogram of anhydrous form A of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide.

FIG. 4A. XRPD of anhydrous form B of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide.

FIG. 4B. DSC thermogram of anhydrous form B of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide.

FIG. 5A. XRPD of anhydrous form C of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide.

FIG. 5B. DSC thermogram of anhydrous form C of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide.

### DETAILED DESCRIPTION

An aspect of the present invention provides compounds and pharmaceutical formulations thereof that are useful in the treatment or prevention of diseases mediated by the potentiation of TMEM16A, such as chronic bronchitis, chronic obstructive pulmonary disease (COPD), bronchiectasis, asthma, cystic fibrosis, primary ciliary dyskinesia, respiratory tract infections (acute and chronic; viral and bacterial), lung carcinoma and related disorders.

A first embodiment of the invention provides a compound of formula (I):

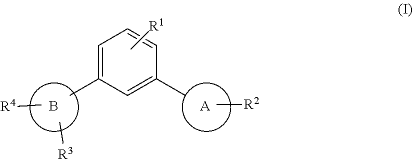

Wherein:
Ring A is a 5 membered heteroaryl containing 2 heteroatoms selected from N and O;
Ring B is a 5 heteroaryl containing 2 or 3 heteroatoms each independently selected from N, S and O, wherein at least one of said heteroatoms is N or ring B is a 6 membered heteroaryl containing 1 or 2 heteroatoms selected from N;
R¹ is hydrogen or halogen;
R² is selected from the group consisting of:

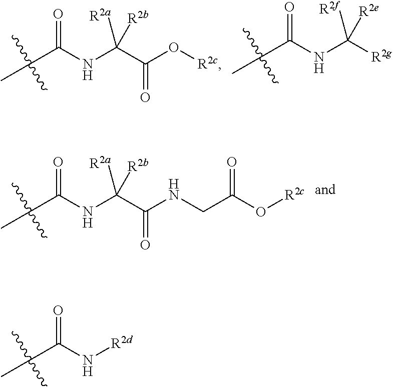

where

R^{2a} is H, (C₁-C₄)alkyl or phenyl, wherein said (C₁-C₄)alkyl is optionally substituted with halogen, (C₃-C₆)cycloalkyl, phenyl, —O—(C₁-C₄)alkyl or —S—(C₁-C₄)alkyl;

R^{2b} is H, (C₁-C₄)alkyl or R^{2b} taken together with R^{2a} forms a (C₃-C₆)cycloalkyl ring;

R^{2c} is (C₁-C₄)alkyl, (C₂-C₄)alkenyl or benzyl;

R^{2d} is (C₁-C₄)alkyl, (C₃-C₆)cycloalkyl, adamantyl, a 5 or 6 membered heteroaryl wherein said heteroaryl contains 1 or 2 heteroatoms independently selected from N and O, or phenyl; wherein said phenyl is optionally substituted with 1 or 2 substituents independently selected from (C₁-C₄)alkyl, halo-(C₁-C₄)alkyl and nitrile;

R^{2c} is H, (C₁-C₄)alkyl or (C₃-C₆)cycloalkyl ring;

R^{2f} is H, (C₁-C₄)alkyl or (C₃-C₆)cycloalkyl ring optionally substituted with (C₁-C₄)alkyl or R^{2e} taken together with R^{2f} forms a (C₃-C₆)cycloalkyl ring;

R^{2g} is H, (C₁-C₄)alkyl, a fused moiety selected from benzo[d][1,3]dioxole and indolin-2-one, where said fused moiety is optionally substituted with halogen or (C₁-C₄)alkyl, (C₃-C₆)heterocycloalkyl containing 1 or 2 heteroatoms selected from N and O, -(C₀-C₂)alkyl-phenyl wherein said phenyl is optionally substituted 1 or 2 groups independently selected from halogen and (C₁-C₄)alkyl;

R³ is H, (C₁-C₅)alkyl or a 4 to 6 membered saturated heterocycle containing O; wherein said (C₁-C₅)alkyl is optionally substituted with 1 to 3 groups independently selected from hydroxyl, (C₁-C₅)alkoxy, halogen, diethyl phosphate, —C(O)O(C₁-C₄)alkyl, NH-benzyl, O-benzyl, benzo[d][1,3]dioxole, isoindolinyl, —O—(C₂-C₄)alkyl-O—(C₁-C₄)alkyl, and a 4 to 6 membered saturated heterocycle containing 1 or 2 heteroatoms selected from N and O wherein said heterocycle is optionally substituted with 1 or 2 groups selected from (C₁-C₄)alkyl, and —C(O)NH(CHR⁵)C(O)O—(C₁-C₄)alkyl;

R⁴ is selected from the group consisting of:

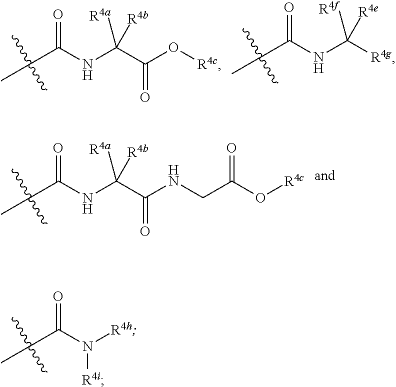

where

R^{4a} is H, (C₁-C₄)alkyl or phenyl, wherein said (C₁-C₄)alkyl is optionally substituted with 1 to 3 halogens, (C₃-C₆)cycloalkyl, phenyl, —O—(C₁-C₄)alkyl or —S—(C₁-C₄)alkyl;

R⁴⁰ is H or (C₁-C₄)alkyl or R⁴⁰ taken together with R^{4a} to form a (C₃-C₆)cycloalkyl ring;

R^{4c} is (C₁-C₄)alkyl, (C₂-C₄)alkenyl or benzyl;

R^{4e} e is H, (C₁-C₄)alkyl, (C₁-C₄)alkoxy or (C₃-C₆)cycloalkyl ring;

R^{4f} is H, (C₁-C₄)alkyl or (C₃-C₆)cycloalkyl ring optionally substituted with nitrile or (C₁-C₄)akyl or R^{4e} taken together with R^{4f} to form a (C₃-C₆)cycloalkyl ring;

R^{4g} is H, (C₁-C₄)alkyl, a fused moiety selected from benzo[d][1,3]dioxole and indolin-2-one, where said fused moiety is optionally substituted with halogen or (C₁-C₄)alkyl, (C₃-C₆)heterocycloalkyl containing 1 or 2 heteroatoms selected from N and O, -(C₀-C₂)alkyl-phenyl wherein said phenyl is optionally substituted with 1 or 2 halogens;

R^{4h} is (C₁-C₄)alkyl, (C₃-C₆)cycloalkyl optionally substituted with 1 or 2 halogens, adamantyl, a 5 or 6 membered heteroaryl wherein said heteroaryl contains 1 or 2 heteroatoms independently selected from N and O, or phenyl; wherein said phenyl is optionally substituted with 1 or 2 substituents independently selected from (C₁-C₄)alkyl, (C₁-C₅)alkoxy, halo-(C₁-C₄)alkyl, halo-(C₁-C₄)alkoxy and nitrile;

R⁴ⁱ is H or R⁴ⁱ taken together with R^{4h} forms a (C₃-C₆)heterocycloalkyl ring optionally substituted with 1 or 2 substituents independently selected from (C₁-C₄)alkyl, (C₁-C₄)alkoxy and —C(O)O(C₁-C₄)alkyl; and

R⁵ is H or (C₁-C₄)alkyl, wherein said (C₁-C₄)alkyl is optionally substituted with (C₃-C₆)cycloalkyl, phenyl, —O—(C₁-C₄)alkyl or —S—(C₁-C₄)alkyl;

or a pharmaceutically acceptable salt, hydrate, or co-crystal thereof.
A second embodiment of the invention provides a compound of formula (Ia):

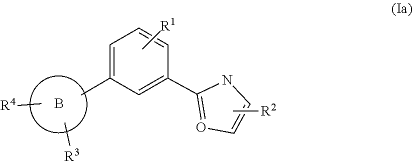

### Wherein:

Ring B is selected from the group consisting of:

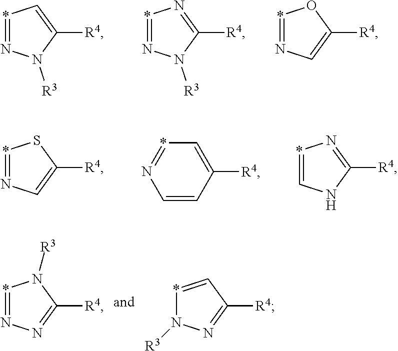

and * indicates the point of attachment;
or a pharmaceutically acceptable salt, hydrate, or co-crystal thereof.
A third embodiment of the invention provides a compound of embodiment 1 or 2 of formula (Ia):

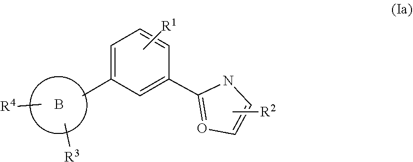

### Wherein:

Ring B is selected from the group consisting of:

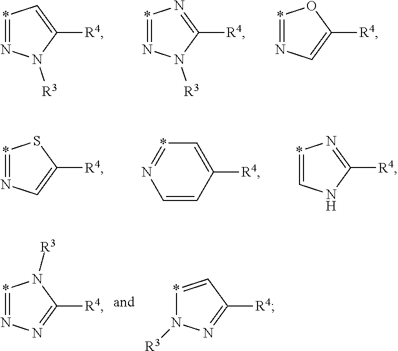

and * indicates the point of attachment;
R³ is selected from the group consisting of H or:

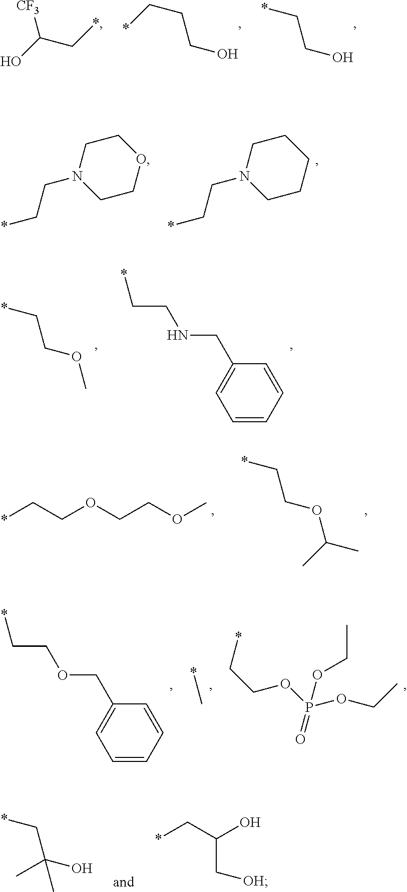

and * indicates the point of attachment;
or a pharmaceutically acceptable salt, hydrate, or co-crystal thereof.
A fourth embodiment of the invention provides a compound of any of the preceding embodiment's wherein:

R¹ is hydrogen;

or a pharmaceutically acceptable salt, hydrate, or co-crystal thereof.
A fifth embodiment of the invention provides a compound of embodiment 1 or 2 of formula (IIa):

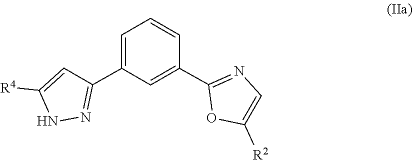

or a pharmaceutically acceptable salt, hydrate, or co-crystal thereof.
A sixth embodiment of the invention provides a compound of embodiment 1 or 2 of formula (IIb):

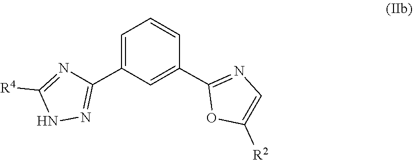

or a pharmaceutically acceptable salt, hydrate, or co-crystal thereof.
A seventh embodiment of the invention provides a compound of embodiment 1 or 2 of formula (IIc):

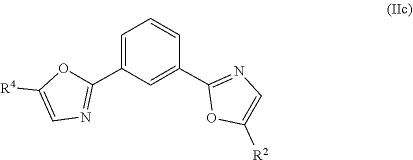

or a pharmaceutically acceptable salt, hydrate, or co-crystal thereof.
An eighth embodiment of the invention provides a compound of embodiment 1 or 2 of formula (IId):

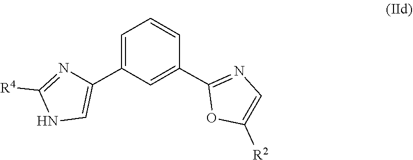

or a pharmaceutically acceptable salt, hydrate, or co-crystal thereof.
A ninth embodiment of the invention provides a compound of any of the preceding embodiments wherein:

R¹ is H;

R² is selected from the group consisting of:

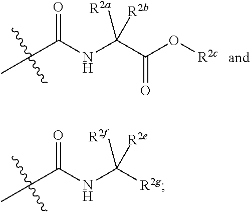

R^{2a} is H, (C₁-C₄)alkyl or phenyl, wherein said (C₁-C₄)alkyl is optionally substituted with halogen, (C₃-C₆)cycloalkyl, phenyl, —O—(C₁-C₄)alkyl or —S—(C₁-C₄)alkyl;

R^{2b} is H, (C₁-C₄)alkyl or R^{2b} taken together with R^{2a} forms a (C₃-C₆)cycloalkyl ring;

R^{2c} is (C₁-C₄)alkyl, (C₂-C₄)alkenyl or benzyl;

R^{2c} is H, (C₁-C₄)alkyl or (C₃-C₆)cycloalkyl ring;

R^{2f} is H, (C₁-C₄)alkyl or (C₃-C₆)cycloalkyl ring optionally substituted with (C₁-C₄)alkyl or

R^{2c} taken together with R^{2f} forms a (C₃-C₆)cycloalkyl ring;

R^{2g} is H, (C₁-C₄)alkyl, (C₃-C₆)heterocycloalkyl containing 1 or 2 heteroatoms selected from N and O, -(C₀-C₂)alkyl-phenyl wherein said phenyl is optionally substituted 1 or 2 groups independently selected from halogen and (C₁-C₄)alkyl;

R³ is H;

R⁴ is selected from the group consisting of:

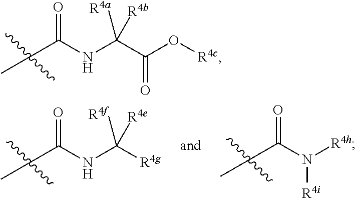

where

R^{4a} is H, (C₁-C₄)alkyl, phenyl, wherein said (C₁-C₄)alkyl is optionally substituted with 1 to 3 halogens, (C₃-C₆)cycloalkyl, phenyl, —O—(C₁-C₄)alkyl or —S—(C₁-C₄)alkyl;

R^{4b} is H or (C₁-C₄)alkyl or R⁴⁰ taken together with R^{4a} to form a (C₃-C₆)cycloalkyl ring;

R^{4c} is (C₁-C₄)alkyl, (C₂-C₄)alkenyl and benzyl;

R^{4e} e is H, (C₁-C₄)alkyl, (C₁-C₄)alkoxy or (C₃-C₆)cycloalkyl ring;

R^{4f} is H, (C₁-C₄)alkyl or (C₃-C₆)cycloalkyl ring optionally substituted with nitrile or (C₁-C₄)akyl or R^{4e} taken together with R^{4f} to form a (C₃-C₆)cycloalkyl ring;

R^{4g} is H, (C₁-C₄)alkyl, (C₃-C₆)heterocycloalkyl containing 1 or 2 heteroatoms selected from N and O, -(C₀-C₂)alkyl-phenyl wherein said phenyl is optionally substituted with 1 or 2 halogens;

R^{4h} is (C₁-C₄)alkyl, (C₃-C₆)cycloalkyl optionally substituted with 1 or 2 halogens, adamantyl, a 5 or 6 membered heteroaryl wherein said heteroaryl contains 1 or 2 heteroatoms independently selected from N and O, or phenyl; wherein said phenyl is optionally substituted with 1 or 2 substituents independently selected from (C₁-C₄)alkyl, (C₁-C₅)alkoxy, halo-(C₁-C₄)alkyl, halo-(C₁-C₄)alkoxy and nitrile; and

R⁴ⁱ is H or R⁴ⁱ taken together with R^{4h} forms a (C₃-C₆)heterocycloalkyl ring optionally substituted with 1 or 2 substituents independently selected from (C₁-C₄)alkyl, (C₁-C₄)alkoxy and —C(O)O(C₁-C₄)alkyl;

or a pharmaceutically acceptable salt, hydrate, or co-crystal thereof.
A tenth embodiment of the invention provides a compound of embodiments 1 or 2 wherein:
R² is selected from the group consisting of:

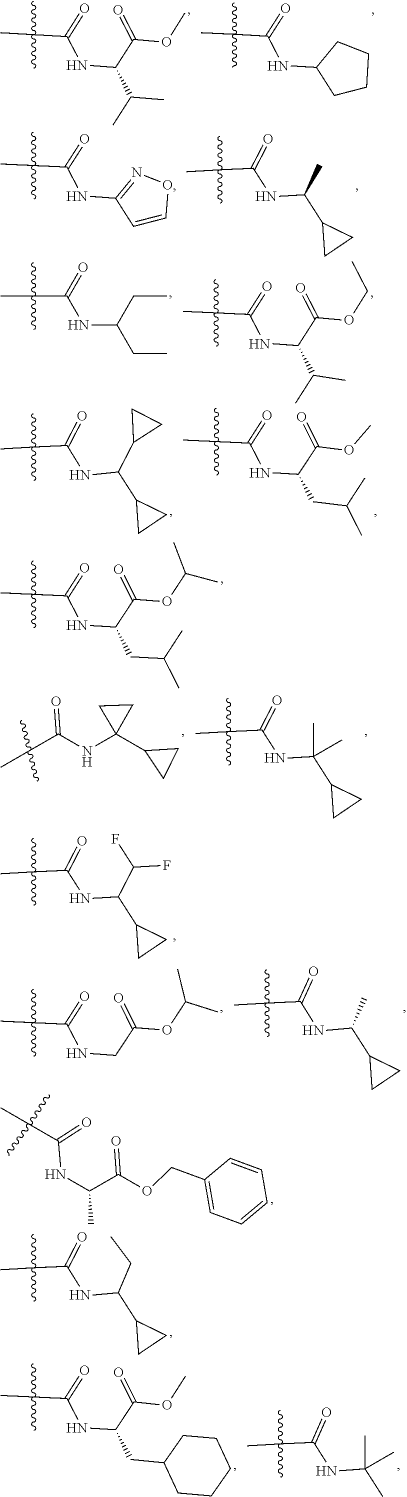

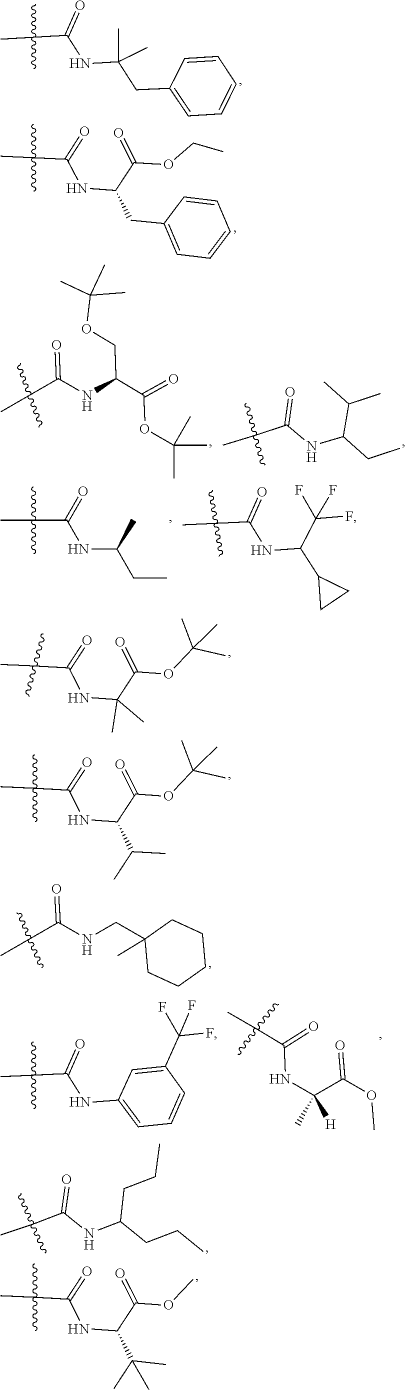

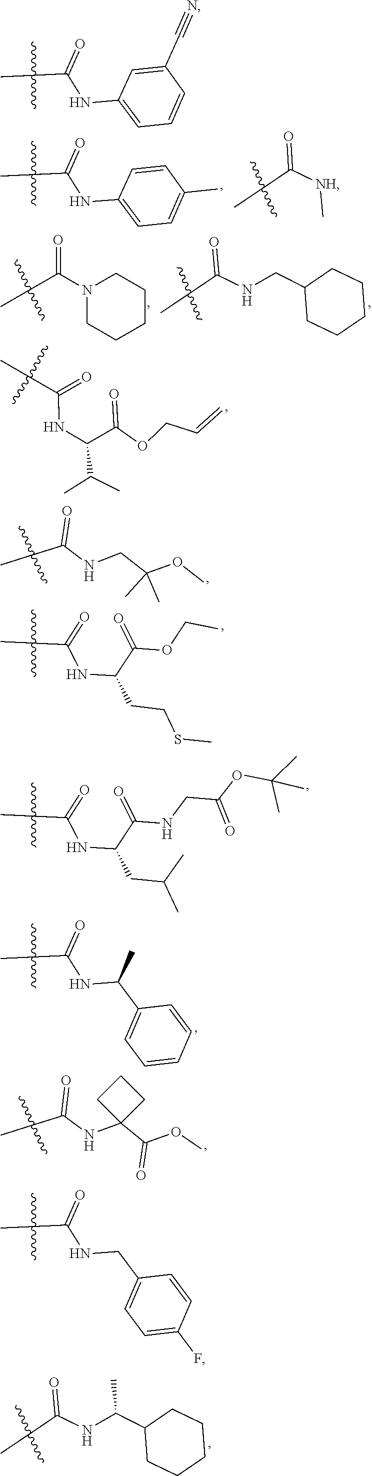

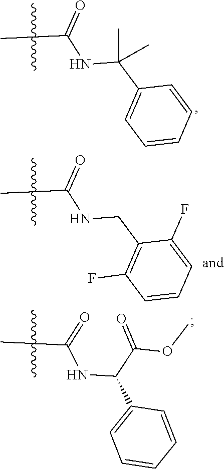

or a pharmaceutically acceptable salt, hydrate, or co-crystal thereof.
An eleventh embodiment of the invention provides a compound of embodiments 1 or 2
wherein:
R⁴ is selected from the group consisting of:

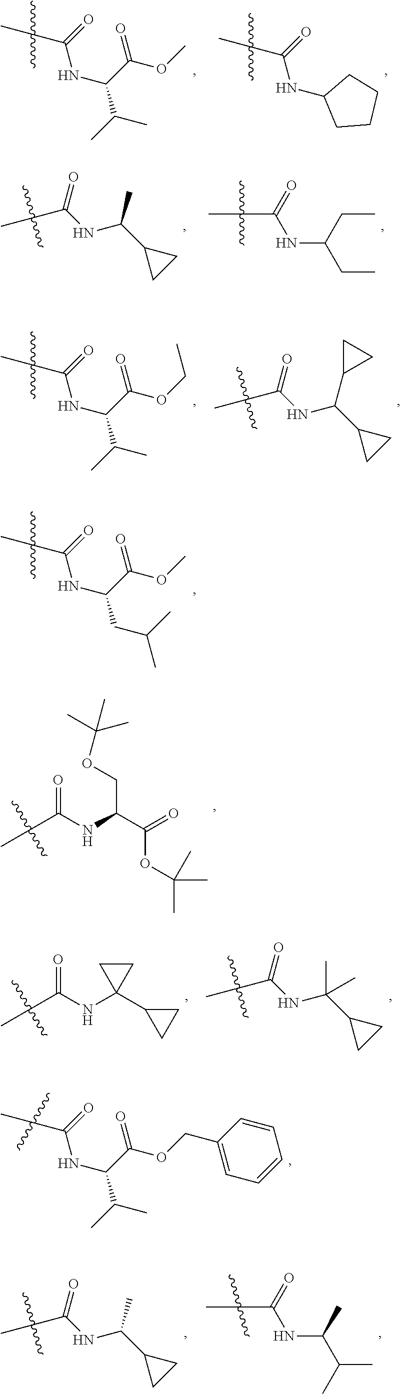

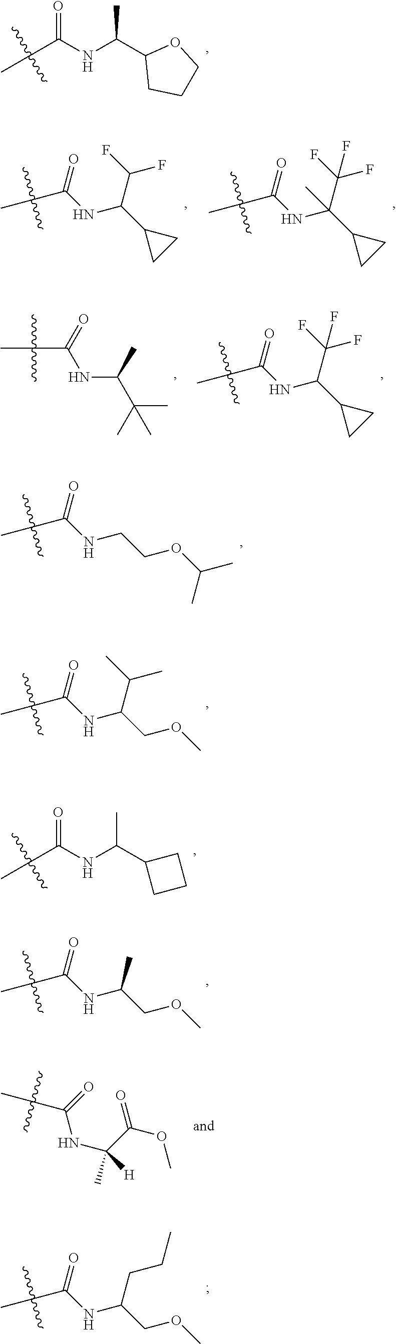

or a pharmaceutically acceptable salt, hydrate, or co-crystal thereof.
A twelve embodiment of the invention provides a compound of embodiments 1, 2 or 5 having the formula:

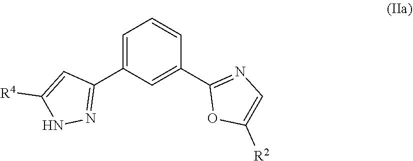

wherein R² is selected from the group consisting of:

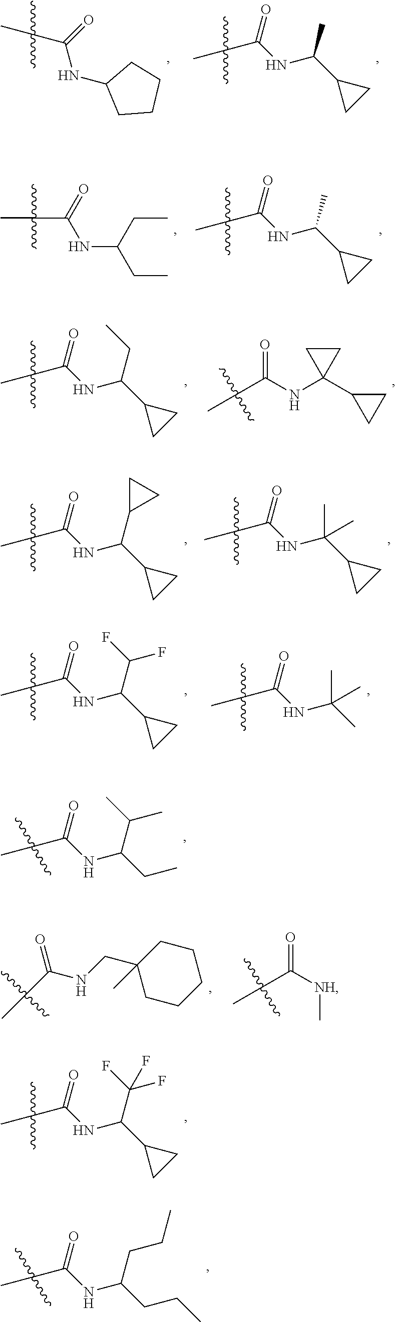

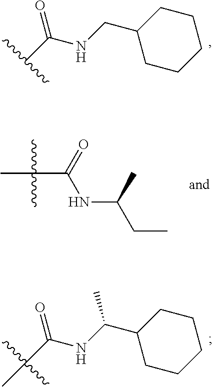

R⁴ is selected from the group consisting of:

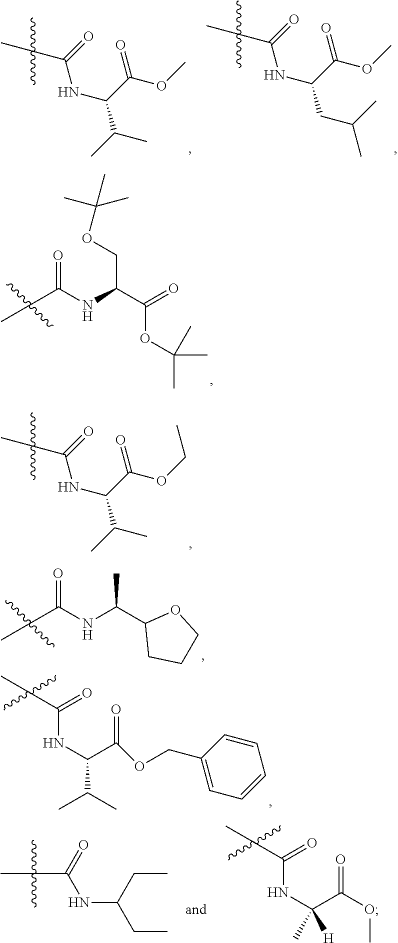

or a pharmaceutically acceptable salt, hydrate, or co-crystal thereof.
A thirteenth embodiment of the invention provides a compound of embodiments 1, 2 or 6 of formula (IIb):

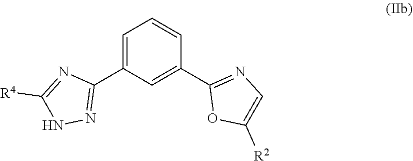

wherein R² is selected from the group consisting of:

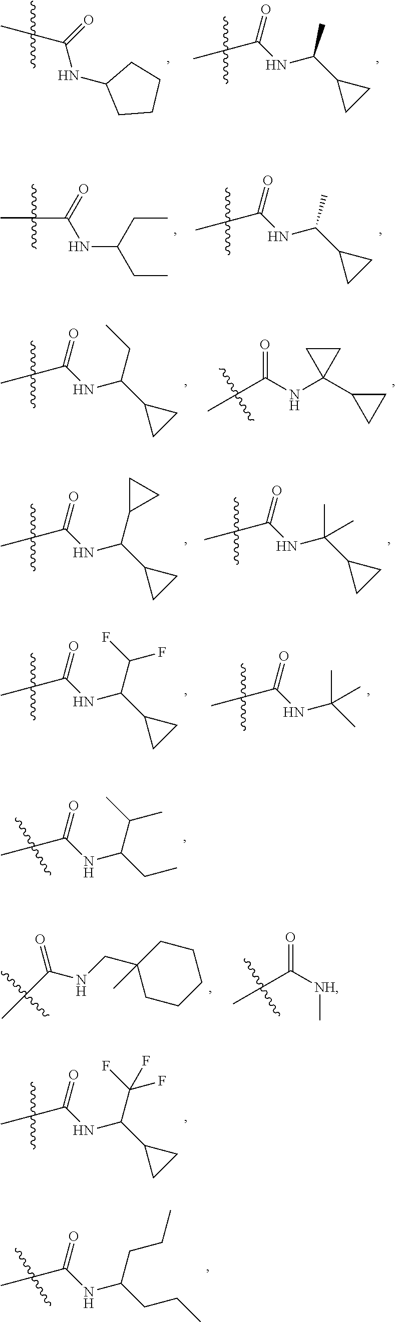

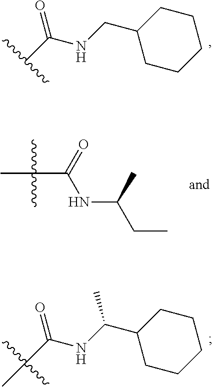

R⁴ is selected from the group consisting of:

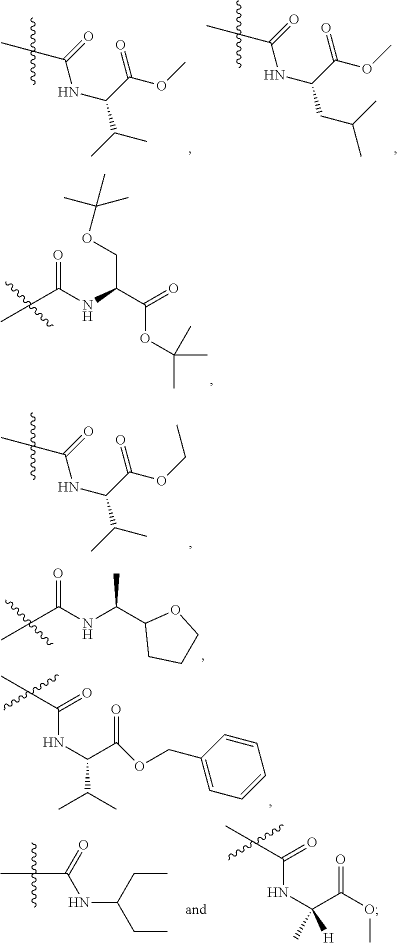

or a pharmaceutically acceptable salt, hydrate, or co-crystal thereof.
A fourteenth embodiment of the invention provides a compound of embodiments 1, 2 or 7 of formula (IIc):

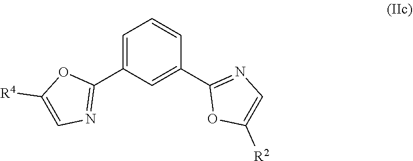

wherein R² is selected from the group consisting of:

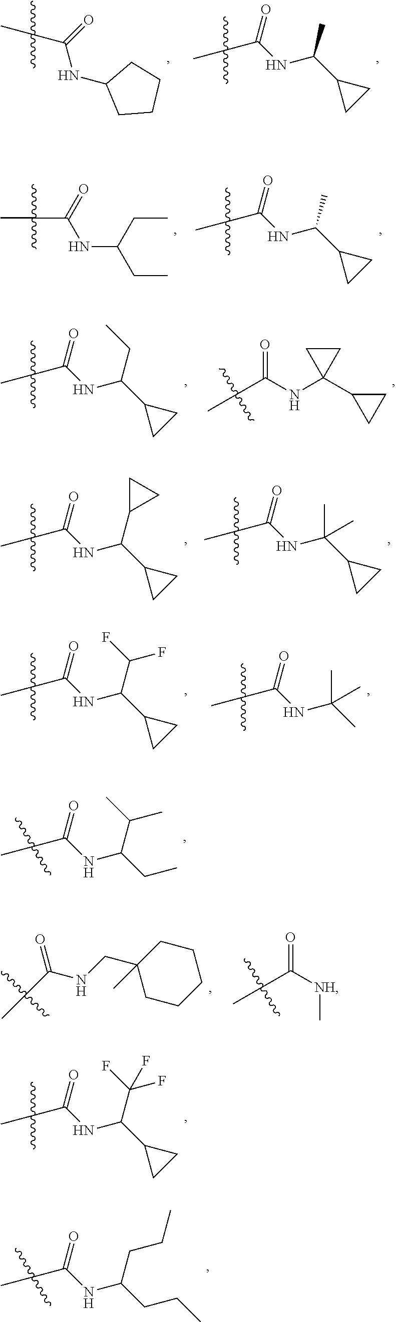

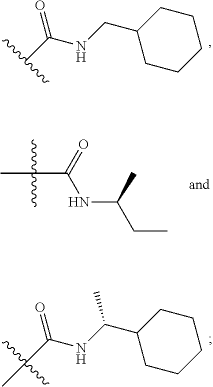

R⁴ is selected from the group consisting of:

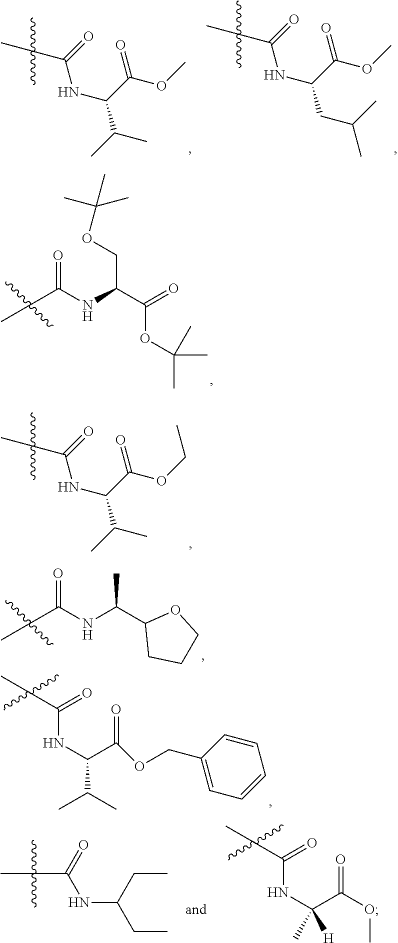

or a pharmaceutically acceptable salt, hydrate, or co-crystal thereof.
A fifteenth embodiment of the invention provides a compound of embodiments 1, 2 or 8 of formula (IId):

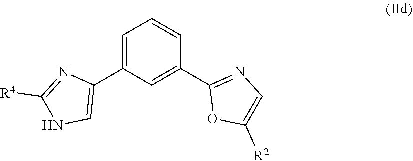

wherein R² is selected from the group consisting of:

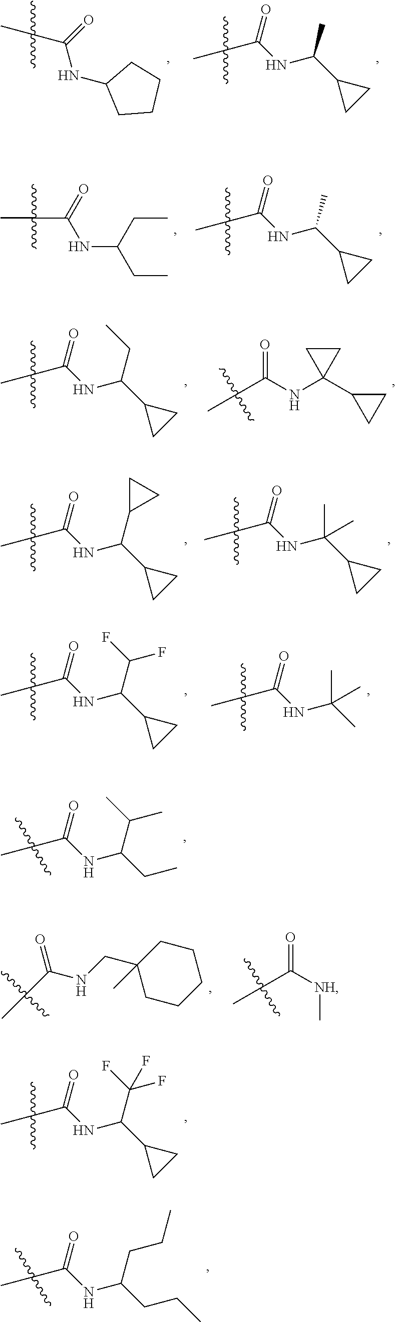

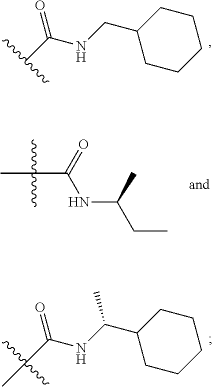

R⁴ is selected from the group consisting of:

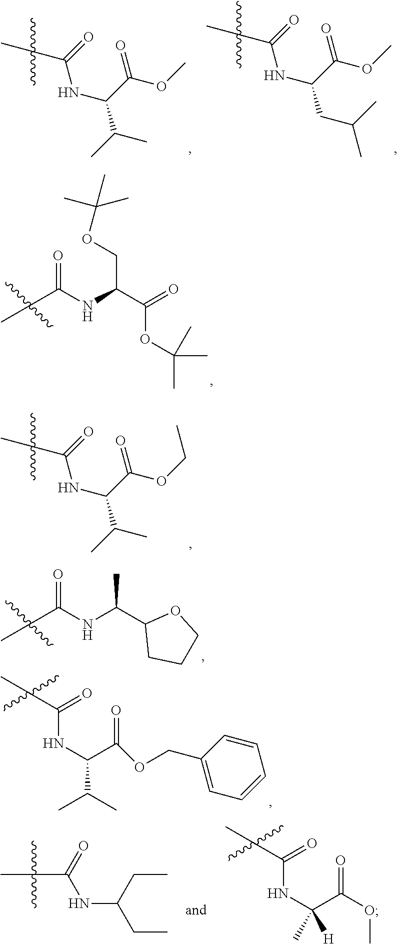

or a pharmaceutically acceptable salt, hydrate, or co-crystal thereof.
A sixteenth embodiment of the invention provides a compound of embodiments 1, 2, 12, 13, 14 or 15 wherein
R² is selected from the group consisting of:

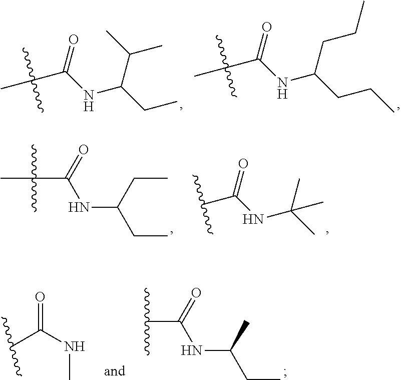

R⁴ is selected from the group consisting of:

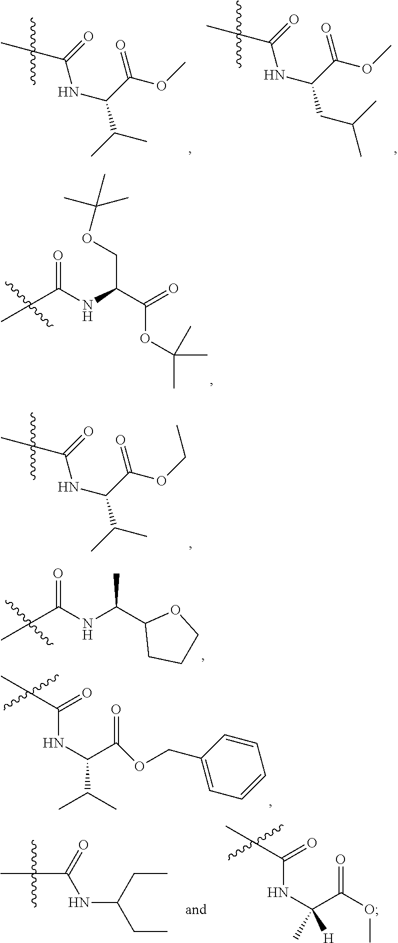

or a pharmaceutically acceptable salt, hydrate, or co-crystal thereof.
A seventeenth embodiment of the invention provides a compound of embodiment 1 selected from the group consisting of:

### methyl (2-(3-(5-((dicyclopropylmethyl)carbamoyl)-1-(3,3,3-trifluoro-2-hydroxypropyl)-1 H-pyrazol-3-yl)phenyl)oxazole-5-carbonyl)-L-valinate

N-cyclopentyl-2-(3-(5-(cyclopentylcarbamoyl)-1-(3-hydroxypropyl)-1 H-pyrazol-3-yl)phenyl)oxazole-5-carboxamide;

### 2-(3-(2-(((S)-1-cyclopropylethyl)carbamoyl)-1-(3,3,3-trifluoro-2-hydroxypropyl)-1 H-imidazol-4-yl)phenyl)-N-(pentan-3-yl)oxazole-5-carboxamide

2-(3-(1-(2-hydroxyethyl)-5-(pentan-3-ylcarbamoyl)-1 H-1,2,4-triazol-3-yl)phenyl)-N-(pentan-3-yl)oxazole-5-carboxamide;

(S)-2-(3-(5-((1-cyclopropylethyl)carbamoyl)-4H-1,2,4-triazol-3-yl)phenyl)-N-(pentan-3-yl)oxazole-5-carboxamide;

### ethyl (1-(2-morpholinoethyl)-3-(3-(5-(pentan-3-ylcarbamoyl)oxazol-2-yl)phenyl)-1 H-pyrazole-5-carbonyl)-L-valinate

ethyl (2-(3-(5-((dicyclopropylmethyl)carbamoyl)-1-(2-hydroxyethyl)-1 H-pyrazol-3-yl)phenyl)oxazole-5-carbonyl)-L-valinate;

### methyl (2-(3-(5-((dicyclopropylmethyl)carbamoyl)-1-(2-hydroxyethyl)-1H-pyrazol-3-yl)phenyl)oxazole-5-carbonyl)-L-valinate

N-(2-methylpentan-3-yl)-2-(3-(3-(pentan-3-ylcarbamoyl)-1 H-pyrazol-5-yl)phenyl)oxazole-5-carboxamide;

### N-(pentan-3-yl)-2-(3-(5-(pentan-3-ylcarbamoyl)-1-(2-(piperidin-1-yl)ethyl)-1H-1,2,4-triazol-3-yl)phenyl)oxazole-5-carboxamide

methyl (2-(3-(5-(((S)-1-cyclopropylethyl)carbamoyl)-1-(3,3,3-trifluoro-2-hydroxypropyl)-1 H-pyrazol-3-yl)phenyl)oxazole-5-carbonyl)-L-valinate;

### 2-(3-(5-(((S)-1-cyclopropylethyl)carbamoyl)-1-(3,3,3-trifluoro-2-hydroxypropyl)-1 H-pyrazol-3-yl)phenyl)-N-(pentan-3-yl)oxazole-5-carboxamide

2-(3-(1-(2-methoxyethyl)-5-(pentan-3-ylcarbamoyl)-1 H-1,2,4-triazol-3-yl)phenyl)-N-(pentan-3-yl)oxazole-5-carboxamide;

### ethyl (2-(3-(5-((dicyclopropylmethyl)carbamoyl)-1-(3-hydroxypropyl)-1H-pyrazol-3-yl)phenyl)oxazole-5-carbonyl)-L-valinate

2-(3-(1-(2-(benzylamino)ethyl)-5-(pentan-3-ylcarbamoyl)-1 H-1,2,4-triazol-3-yl)phenyl)-N-(pentan-3-yl)oxazole-5-carboxamide;

### ethyl (5-(3-(5-(pentan-3-ylcarbamoyl)oxazol-2-yl)phenyl)-1 H-pyrazole-3-carbonyl)-L-leucinate

N-(pentan-3-yl)-2-(3-(3-((1-(tetrahydrofuran-2-yl)ethyl)carbamoyl)-1 H-pyrazol-5-yl)phenyl)oxazole-5-carboxamide;

tert-butyl (2-(3-(3-(pentan-3-ylcarbamoyl)-1 H-pyrazol-5-yl)phenyl)oxazole-5-carbonyl)-L-valinate;

(S)-2-(3-(5-((1-cyclopropylethyl)carbamoyl)-1-(oxetan-3-yl)-1 H-pyrazol-3-yl)phenyl)-N-(pentan-3-yl)oxazole-5-carboxamide;

### ethyl (2-(3-(5-((dicyclopropylmethyl)carbamoyl)-4H-1,2,4-triazol-3-yl)phenyl)oxazole-5-carbonyl)-L-valinate

2-(3-(3-((1-cyanopropyl)carbamoyl)-1 H-pyrazol-5-yl)phenyl)-N-(pentan-3-yl)oxazole-5-carboxamide;

### 2-(3-(3-((1-cyclopropyl-2-methoxyethyl)carbamoyl)-1 H-pyrazol-5-yl)phenyl)-N-(pentan-3-yl)oxazole-5-carboxamide

N-((S)-1-cyclopropylethyl)-2-(3-(5-(((S)-1-cyclopropylethyl)carbamoyl)-1-(3-hydroxypropyl)-1 H-1,2,4-triazol-3-yl)phenyl)oxazole-5-carboxamide;

### 2-(3-(1-(2-(2-methoxyethoxy)ethyl)-5-(pentan-3-ylcarbamoyl)-1 H-1,2,4-triazol-3-yl)phenyl)-N-(pentan-3-yl)oxazole-5-carboxamide

(S)-2-(3-(5-((1-cyclopropylethyl)carbamoyl)-1-(2-isopropoxyethyl)-1H-pyrazol-3-yl)phenyl)-N-(pentan-3-yl)oxazole-5-carboxamide;

### methyl (2-(3-(5-((dicyclopropylmethyl)carbamoyl)-4H-1,2,4-triazol-3-yl)phenyl)oxazole-5-carbonyl)-L-valinate

methyl (2-(3-(5-(((S)-1-cyclopropylethyl)carbamoyl)-1-(3,3,3-trifluoro-2-hydroxypropyl)-1 H-pyrazol-3-yl)phenyl)oxazole-5-carbonyl)-L-valinate; benzyl (5-(3-(5-(pentan-3-ylcarbamoyl)oxazol-2-yl)phenyl)-1 H-pyrazole-3-carbonyl)-L-valinate;

### ethyl (1-(2-(benzyloxy)ethyl)-3-(3-(5-(pentan-3-ylcarbamoyl)oxazol-2-yl)phenyl)-1 H-pyrazole-5-carbonyl)-L-valinate

(S)-2-(3-(5-((1-cyclopropylethyl)carbamoyl)-1-methyl-1 H-pyrazol-3-yl)phenyl)-N-(pentan-3-yl)oxazole-5-carboxamide;

(S)-2-(3-(2-((1-cyclopropylethyl)carbamoyl)-1 H-imidazol-4-yl)phenyl)-N-(pentan-3-yl)oxazole-5-carboxamide;

### ethyl (1-(2-((diethoxyphosphoryl)oxy)ethyl)-3-(3-(5-(pentan-3-ylcarbamoyl)oxazol-2-yl)phenyl)-1 H-pyrazole-5-carbonyl)-L-valinate; (R)-2-(3-(5-((1-cyclopropylethyl)carbamoyl)-1-(2-hydroxyethyl)-1 H-pyrazol-3-yl)phenyl)-N-(pentan-3-yl)oxazole-5-carboxamide

ethyl (2-(3-(5-(((S)-1-cyclopropylethyl)carbamoyl)-1-(2-hydroxyethyl)-1H-pyrazol-3-yl)phenyl)oxazole-5-carbonyl)-L-valinate;

### methyl (2-(3-(5-(((S)-1-cyclopropylethyl)carbamoyl)-1-(3,3,3-trifluoro-2-hydroxypropyl)-1 H-pyrazol-3-yl)phenyl)oxazole-5-carbonyl)-L-valinate; 2-(3-(3-((cyclobutylmethyl)carbamoyl)-1 H-pyrazol-5-yl)phenyl)-N-(pentan-3-yl)oxazole-5-carboxamide

tert-butyl O-(tert-butyl)-N-(5-(3-(5-(pentan-3-ylcarbamoyl)oxazol-2-yl)phenyl)-1 H-pyrazole-3-carbonyl)-L-serinate;

### 2-(3-(3-([1,1′-bi(cyclopropan)]-1-ylcarbamoyl)-1H-pyrazol-5-yl)phenyl)-N-(pentan-3-yl)oxazole-5-carboxamide

methyl (3-(3-(5-((dicyclopropylmethyl)carbamoyl)oxazol-2-yl)phenyl)-1 H-1,2,4-triazole-5-carbonyl)-L-valinate;

### N-(pentan-3-yl)-2-(3-(4-(pentan-3-ylcarbamoyl)pyridin-2-yl)phenyl)oxazole-5-carboxamide

N-(dicyclopropylmethyl)-2-(3-(5-((dicyclopropylmethyl)carbamoyl)-4H-1,2,4-triazol-3-yl)phenyl)oxazole-5-carboxamide;

### N-((R)-1-cyclopropylethyl)-2-(3-(5-(((S)-1-cyclopropylethyl)carbamoyl)-1-(3,3,3-trifluoro-2-hydroxypropyl)-1 H-pyrazol-3-yl)phenyl)oxazole-5-carboxamide

tert-butyl (2-(3-(5-(((S)-1-cyclopropylethyl)carbamoyl)-4H-1,2,4-triazol-3-yl)phenyl)oxazole-5-carbonyl)-L-valinate;

### ethyl (2-(3-(5-((1,1,1-trifluorobutan-2-yl)carbamoyl)-1H-pyrazol-3-yl)phenyl)oxazole-5-carbonyl)-L-valinate

2-(3-(3-((2-cyclopropylpropan-2-yl)carbamoyl)-1 H-pyrazol-5-yl)phenyl)-N-(pentan-3-yl)oxazole-5-carboxamide;

### 2-(3-(3-((1-cyanopropyl)carbamoyl)-1H-pyrazol-5-yl)phenyl)-N-(pentan-3-yl)oxazole-5-carboxamide

ethyl (2-(3-(5-((1-cyclopropyl-2,2,2-trifluoroethyl)carbamoyl)-1H-pyrazol-3-yl)phenyl)oxazole-5-carbonyl)-L-valinate;

### ethyl (3-(3-(5-((dicyclopropylmethyl)carbamoyl)oxazol-2-yl)phenyl)-1-(2-hydroxy-2-methylpropyl)-1 H-pyrazole-5-carbonyl)-L-valinate

2-(3-(3-((cyclopropyl(tetrahydrofuran-2-yl)methyl)carbamoyl)-1 H-pyrazol-5-yl)phenyl)-N-(pentan-3-yl)oxazole-5-carboxamide;

tert-butyl O-(tert-butyl)-N-(2-(3-(3-(pentan-3-ylcarbamoyl)-1 H-pyrazol-5-yl)phenyl)oxazole-5-carbonyl)-L-serinate;

### 2-(3-(5-(((S)-1-cyclopropylethyl)carbamoyl)-1-(3,3,3-trifluoro-2-hydroxypropyl)-1H-pyrazol-3-yl)phenyl)-N-(dicyclopropylmethyl)oxazole-5-carboxamide

methyl (2-(3-(5-((dicyclopropylmethyl)carbamoyl)-1 H-pyrazol-3-yl)phenyl)oxazole-5-carbonyl)-L-valinate;

### ethyl (2-(3-(5-(((S)-1-cyclopropylethyl)carbamoyl)-4H-1,2,4-triazol-3-yl)phenyl)oxazole-5-carbonyl)-L-valinate

N-(pentan-3-yl)-2-(3-(5-(pentan-3-ylcarbamoyl)-1 H-pyrazol-3-yl)phenyl)oxazole-5-carboxamide;

### ethyl (2-(3-(3-(((S)-1-ethoxy-3-methyl-1-oxobutan-2-yl)carbamoyl)-1 H-pyrazol-5-yl)phenyl)oxazole-5-carbonyl)-L-valinate

2-(3-(1-(2-hydroxyethyl)-5-(pentan-3-ylcarbamoyl)-1H-pyrazol-3-yl)phenyl)-N-(2-methylpentan-3-yl)oxazole-5-carboxamide;

### N-(tert-butyl)-2-(3-(3-(pentan-3-ylcarbamoyl)-1 H-pyrazol-5-yl)phenyl)oxazole-5-carboxamide

ethyl (2-(3-(3-(pentan-3-ylcarbamoyl)-1 H-pyrazol-5-yl)phenyl)oxazole-5-carbonyl)-L-methioninate;

tert-butyl (5-(3-(5-(pentan-3-ylcarbamoyl)oxazol-2-yl)phenyl)-1 H-pyrazole-3-carbonyl)-L-leucylglycinate;

### ethyl (1-(2-hydroxyethyl)-3-(3-(5-(pentan-3-ylcarbamoyl)oxazol-2-yl)phenyl)-1 H-pyrazole-5-carbonyl)-L-valinate

(R)-2-(3-(3-((3-methylbutan-2-yl)carbamoyl)-1 H-pyrazol-5-yl)phenyl)-N-(pentan-3-yl)oxazole-5-carboxamide:

### methyl (2-(3-(5-(((S)-1-methoxy-3-methyl-1-oxobutan-2-yl)carbamoyl)-1-(3,3,3-trifluoro-2-hydroxypropyl)-1 H-pyrazol-3-yl)phenyl)oxazole-5-carbonyl)-L-valinate

N-(pentan-3-yl)-2-(3-(3-((2-phenylpropan-2-yl)carbamoyl)-1 H-pyrazol-5-yl)phenyl)oxazole-5-carboxamide;

### 2-(3-(3-((1-cyanopropyl)carbamoyl)-1H-pyrazol-5-yl)phenyl)-N-(pentan-3-yl)oxazole-5-carboxamide

2-(3-(3-(((R)-1-((2R,5R)-5-methyltetrahydrofuran-2-yl)propyl)carbamoyl)-1 H-pyrazol-5-yl)phenyl)-N-(pentan-3-yl)oxazole-5-carboxamide;

### N-((R)-1-cyclopropylethyl)-2-(3-(5-(((S)-1-cyclopropylethyl)carbamoyl)-1-(2-hydroxyethyl)-1 H-pyrazol-3-yl)phenyl)oxazole-5-carboxamide

ethyl (2-(3-(1-(2-(((S)-1-ethoxy-3-methyl-1-oxobutan-2-yl)amino)-2-oxoethyl)-5-(pentan-3-ylcarbamoyl)-1 H-pyrazol-3-yl)phenyl)oxazole-5-carbonyl)-L-valinate;

### 2-(3-(1-(2-hydroxyethyl)-5-(pentan-3-ylcarbamoyl)-1 H-pyrazol-3-yl)phenyl)-N-(p-tolyl)oxazole-5-carboxamide

(S)-N-(1-cyclopropylethyl)-2-(3-(1-(2-hydroxyethyl)-5-(pentan-3-ylcarbamoyl)-1 H-pyrazol-3-yl)phenyl)oxazole-5-carboxamide;

### ethyl (R)-2-(5-(3-(5-(pentan-3-ylcarbamoyl)oxazol-2-yl)phenyl)-1 H-pyrazole-3-carboxamido)-2-phenylacetate

2-(3-(1-(2-(benzyloxy)ethyl)-5-(pentan-3-ylcarbamoyl)-1 H-1,2,4-triazol-3-yl)phenyl)-N-(pentan-3-yl)oxazole-5-carboxamide;

(S)-2-(3-(5-((1-cyclopropylethyl)carbamoyl)-1-(2-hydroxy-2-methylpropyl)-1 H-pyrazol-3-yl)phenyl)-N-(pentan-3-yl)oxazole-5-carboxamide;

### methyl (2-(3-(5-((dicyclopropylmethyl)carbamoyl)-1-(2-hydroxy-2-methylpropyl)-1H-pyrazol-3-yl)phenyl)oxazole-5-carbonyl)-L-valinate

ethyl (2-(3-(1-(4-(tert-butoxy)-4-oxobutyl)-5-(pentan-3-ylcarbamoyl)-1 H-pyrazol-3-yl)phenyl)oxazole-5-carbonyl)-L-valinate;

### diethyl 2,2′-((2,2′-(1,3-phenylene)bis(oxazole-2,5-diyl-5-carbonyl))bis(azanediyl))(2S,2'S)-bis(3-methylbutanoate)

2-(3-(5-(((S)-1-cyclopropylethyl)carbamoyl)-1-(2-hydroxypropyl)-1 H-pyrazol-3-yl)phenyl)-N-(pentan-3-yl)oxazole-5-carboxamide;

### 2-(3-(5-(((S)-1-cyclopropylethyl)carbamoyl)-1-(2,3-dihydroxypropyl)-1 H-pyrazol-3-yl)phenyl)-N-(pentan-3-yl)oxazole-5-carboxamide

2-(3-(1-(2-(isoindolin-2-yl)ethyl)-5-(pentan-3-ylcarbamoyl)-1 H-1,2,4-triazol-3-yl)phenyl)-N-(pentan-3-yl)oxazole-5-carboxamide;

### methyl (S)-3-cyclohexyl-2-(2-(3-(3-((dicyclopropylmethyl)carbamoyl)-1 H-pyrazol-5-yl)phenyl)oxazole-5-carboxamido)propanoate

N-((S)-1-cyclopropylethyl)-2-(3-(5-(((S)-1-cyclopropylethyl)carbamoyl)-1-(2-hydroxyethyl)-1 H-1,2,4-triazol-3-yl)phenyl)oxazole-5-carboxamide;

### N-(pentan-3-yl)-2-(3-(3-(((1 S)-1-(tetrahydrofuran-2-yl)ethyl)carbamoyl)-1 H-pyrazol-5-yl)phenyl)oxazole-5-carboxamide

N-(pentan-3-yl)-2-(3-(3-(((S)-1-((S)-tetrahydrofuran-2-yl)ethyl)carbamoyl)-1H-pyrazol-5-yl)phenyl)oxazole-5-carboxamide;

### N-(pentan-3-yl)-2-(3-(3-(((S)-1-((R)-tetrahydrofuran-2-yl)ethyl)carbamoyl)-1 H-pyrazol-5-yl)phenyl)oxazole-5-carboxamide

ethyl (2-(3-(1-(3-(tert-butoxy)-3-oxopropyl)-5-(pentan-3-ylcarbamoyl)-1 H-pyrazol-3-yl)phenyl)oxazole-5-carbonyl)-L-valinate;

### ethyl (2-(3-(1-(3-(tert-butoxy)-3-oxopropyl)-5-(((S)-1-cyclopropylethyl)carbamoyl)-1H-pyrazol-3-yl)phenyl)oxazole-5-carbonyl)-L-valinate

N-((R)-1-cyclopropylethyl)-2-(3-(5-(((R)-1-cyclopropylethyl)carbamoyl)-1-(2-hydroxyethyl)-1 H-pyrazol-3-yl)phenyl)oxazole-5-carboxamide;

### ethyl (2-(3-(3-(pentan-3-ylcarbamoyl)-1 H-pyrazol-5-yl)phenyl)oxazole-5-carbonyl)-L-valinate

(S)-N-(pentan-3-yl)-2-(3-(3-((1-phenylethyl)carbamoyl)-1 H-pyrazol-5-yl)phenyl)oxazole-5-carboxamide;

### methyl (S)-2-(2-(3-(3-(pentan-3-ylcarbamoyl)-1 H-pyrazol-5-yl)phenyl)oxazole-5-carboxamido)-2-phenylacetate

tert-butyl (2-(3-(3-(pentan-3-ylcarbamoyl)-1 H-pyrazol-5-yl)phenyl)oxazole-5-carbonyl)-L-leucylglycinate;

### ethyl (2-(3-(5-((dicyclopropylmethyl)carbamoyl)-1-(2-hydroxy-2-methylpropyl)-1 H-pyrazol-3-yl)phenyl)oxazole-5-carbonyl)-L-valinate

2,2′-(2-methyl-1,3-phenylene)bis(N-(pentan-3-yl)oxazole-5-carboxamide); methyl (2-(3-(5-(((S)-1-cyclopropylethyl)carbamoyl)-4H-1,2,4-triazol-3-yl)phenyl)oxazole-5-carbonyl)-L-leucinate;

### ethyl (2-(3-(5-((1-cyclopropyl-2,2-difluoroethyl)carbamoyl)-1 H-pyrazol-3-yl)phenyl)oxazole-5-carbonyl)-L-valinate

2-(3-(3-((2-cyclopropyl-1,1,1-trifluoropropan-2-yl)carbamoyl)-1 H-pyrazol-5-yl)phenyl)-N-(pentan-3-yl)oxazole-5-carboxamide

### methyl (2-(3-(3-(((R)-1-cyclopropylethyl)carbamoyl)-1 H-pyrazol-5-yl)phenyl)oxazole-5-carbonyl)-L-valinate

2-(3-(3-((2-methyl-4-phenylbutan-2-yl)carbamoyl)-1 H-pyrazol-5-yl)phenyl)-N-(pentan-3-yl)oxazole-5-carboxamide;

### ethyl (2-(3-(5-((1-cyclopropyl-2,2,2-trifluoroethyl)carbamoyl)-1H-pyrazol-3-yl)phenyl)oxazole-5-carbonyl)-L-valinate

N-(pentan-3-yl)-2-(3-(5-(pentan-3-ylcarbamoyl)-1-(2-(piperidin-1-yl)ethyl)-1 H-pyrazol-3-yl)phenyl)oxazole-5-carboxamide;

### 2,2′-(1,3-phenylene)bis(N-(pentan-3-yl)oxazole-5-carboxamide)

2-(3-(3-((1-methoxy-3-methylbutan-2-yl)carbamoyl)-1 H-pyrazol-5-yl)phenyl)-N-(pentan-3-yl)oxazole-5-carboxamide;

### ethyl (5-(3-(5-(((S)-1-cyclopropylethyl)carbamoyl)oxazol-2-yl)phenyl)-4H-1,2,4-triazole-3-carbonyl)-L-valinate

ethyl (2-(3-(3-((1-cyclopropyl-2,2,2-trifluoroethyl)carbamoyl)-1 H-1,2,4-triazol-5-yl)phenyl)oxazole-5-carbonyl)-L-valinate;

### 2-(3-(3-((2-isopropoxyethyl)carbamoyl)-1 H-pyrazol-5-yl)phenyl)-N-(pentan-3-yl)oxazole-5-carboxamide

2-(3-(3-(cyclohexylcarbamoyl)-1 H-pyrazol-5-yl)phenyl)-N-(pentan-3-yl)oxazole-5-carboxamide;

### N-(pentan-3-yl)-2-(3-(5-(pentan-3-ylcarbamoyl)-4H-1,2,4-triazol-3-yl)phenyl)oxazole-5-carboxamide

ethyl 4-(5-(pentan-3-ylcarbamoyl)-3-(3-(5-(pentan-3-ylcarbamoyl)oxazol-2-yl)phenyl)-1 H-1,2,4-triazol-1-yl)butanoate;

### 2-(3-(3-((1-cyclobutylethyl)carbamoyl)-1 H-pyrazol-5-yl)phenyl)-N-(pentan-3-yl)oxazole-5-carboxamide

ethyl (5-(3-(5-(pentan-3-ylcarbamoyl)oxazol-2-yl)phenyl)-1 H-pyrazole-3-carbonyl)-L-valinate;

### N-(4-fluorobenzyl)-2-(3-(3-(pentan-3-ylcarbamoyl)-1 H-pyrazol-5-yl)phenyl)oxazole-5-carboxamide

2-(3-(3-((2-methylpentan-3-yl)carbamoyl)-1 H-pyrazol-5-yl)phenyl)-N-(pentan-3-yl)oxazole-5-carboxamide;

(R)-N-(1-cyclopropylethyl)-2-(3-(5-(pentan-3-ylcarbamoyl)-1 H-pyrazol-3-yl)phenyl)oxazole-5-carboxamide;

### N-(pentan-3-yl)-2-(3-(3-(((1 S)-1-(tetrahydrofuran-2-yl)ethyl)carbamoyl)-1 H-pyrazol-5-yl)phenyl)oxazole-5-carboxamide

N-(pentan-3-yl)-2-(3-(3-(((S)-1-((S)-tetrahydrofuran-2-yl)ethyl)carbamoyl)-1H-pyrazol-5-yl)phenyl)oxazole-5-carboxamide;

### N-(pentan-3-yl)-2-(3-(3-(((S)-1-((R)-tetrahydrofuran-2-yl)ethyl)carbamoyl)-1 H-pyrazol-5-yl)phenyl)oxazole-5-carboxamide

methyl (S)-2-(2-(3-(5-(((S)-1-cyclopropylethyl)carbamoyl)-4H-1,2,4-triazol-3-yl)phenyl)oxazole-5-carboxamido)-3,3-dimethylbutanoate;

(S)-2-(3-(5-((1-cyclopropylethyl)carbamoyl)-4H-1,2,4-triazol-3-yl)phenyl)-N-(dicyclopropylmethyl)oxazole-5-carboxamide:

### methyl (2-(3-(5-(((S)-1-cyclopropylethyl)carbamoyl)-4H-1,2,4-triazol-3-yl)phenyl)oxazole-5-carbonyl)-L-valinate

(S)-2-(3-(3-(pentan-3-ylcarbamoyl)-1 H-pyrazol-5-yl)phenyl)-N-(1-phenylethyl)oxazole-5-carboxamide;

isopropyl (2-(3-(5-(((S)-1-cyclopropylethyl)carbamoyl)-4H-1,2,4-triazol-3-yl)phenyl)oxazole-5-carbonyl)-L-valinate;

(S)-2-(3-(3-((1-methoxypropan-2-yl)carbamoyl)-1 H-pyrazol-5-yl)phenyl)-N-(pentan-3-yl)oxazole-5-carboxamide;

### methyl (2-(3-(3-(pentan-3-ylcarbamoyl)-1 H-pyrazol-5-yl)phenyl)oxazole-5-carbonyl)-L-leucinate

(S)-N-(1-cyclopropylethyl)-2-(3-(5-((dicyclopropylmethyl)carbamoyl)-4H-1,2,4-triazol-3-yl)phenyl)oxazole-5-carboxamide;

### N-(1-cyclopropylethyl)-2-(3-(5-((1-cyclopropylethyl)carbamoyl)-1-(2-hydroxyethyl)-1 H-pyrazol-3-yl)phenyl)oxazole-5-carboxamide

ethyl (2-(3-(1-(2-morpholinoethyl)-5-(pentan-3-ylcarbamoyl)-1H-pyrazol-3-yl)phenyl)oxazole-5-carbonyl)-L-valinate;

(S)-2-(3-(3-((1-cyclopropylethyl)carbamoyl)-1 H-pyrazol-5-yl)phenyl)-N-(pentan-3-yl)oxazole-5-carboxamide;

(S)-2-(3-(5-((1-cyclopropylethyl)carbamoyl)-1 H-pyrazol-3-yl)phenyl)-N-(dicyclopropylmethyl)oxazole-5-carboxamide;

### methyl 1-(5-(3-(5-(pentan-3-ylcarbamoyl)oxazol-2-yl)phenyl)-1 H-pyrazole-3-carbonyl)pyrrolidine-3-carboxylate

N-(heptan-4-yl)-2-(3-(3-(pentan-3-ylcarbamoyl)-1 H-pyrazol-5-yl)phenyl)oxazole-5-carboxamide;

### 2-(3-(3-(heptan-4-ylcarbamoyl)-1 H-pyrazol-5-yl)phenyl)-N-(pentan-3-yl)oxazole-5-carboxamide

2-(3-(3-((cyclopropyl(tetrahydrofuran-2-yl)methyl)carbamoyl)-1 H-pyrazol-5-yl)phenyl)-N-(pentan-3-yl)oxazole-5-carboxamide;

### N-((S)-1-cyclopropylethyl)-2-(3-(5-(((S)-1-cyclopropylethyl)carbamoyl)-1-(2-hydroxyethyl)-1 H-pyrazol-3-yl)phenyl)oxazole-5-carboxamide

methyl (5-(3-(5-(pentan-3-ylcarbamoyl)oxazol-2-yl)phenyl)-1 H-pyrazole-3-carbonyl)-L-valinate;

(S)-2-(3-(4-((1-cyclopropylethyl)carbamoyl)thiazol-2-yl)phenyl)-N-(pentan-3-yl)oxazole-5-carboxamide;

### 2-(3-(5-((cyclohexylmethyl)carbamoyl)-4H-1,2,4-triazol-3-yl)phenyl)-N-(pentan-3-yl)oxazole-5-carboxamide

N-(2-methyl-4-phenylbutan-2-yl)-2-(3-(3-(pentan-3-ylcarbamoyl)-1 H-pyrazol-5-yl)phenyl)oxazole-5-carboxamide;

### ethyl 6-(5-(pentan-3-ylcarbamoyl)-3-(3-(5-(pentan-3-ylcarbamoyl)oxazol-2-yl)phenyl)-1 H-1,2,4-triazol-1-yl)hexanoate

(S)-2-(3-(5-((1-cyclopropylethyl)carbamoyl)-1-(2-hydroxyethyl)-1H-pyrazol-3-yl)phenyl)-N-(pentan-3-yl)oxazole-5-carboxamide;

(R)-2-(3-(5-((1-cyclopropylethyl)carbamoyl)-4H-1,2,4-triazol-3-yl)phenyl)-N-(dicyclopropylmethyl)oxazole-5-carboxamide;

### ethyl (2-(3-(5-(((R)-1-methoxy-3-methyl-1-oxobutan-2-yl)carbamoyl)-1H-pyrazol-3-yl)phenyl)oxazole-5-carbonyl)-L-valinate

methyl (5-(3-(5-(pentan-3-ylcarbamoyl)oxazol-2-yl)phenyl)-1 H-pyrazole-3-carbonyl)-L-leucinate;

### 2-(3-(3-(2-isopropylpyrrolidine-1-carbonyl)-1 H-pyrazol-5-yl)phenyl)-N-(pentan-3-yl)oxazole-5-carboxamide

ethyl (2-(3-(5-((dicyclopropylmethyl)carbamoyl)-1 H-pyrazol-3-yl)phenyl)oxazole-5-carbonyl)-L-valinate;

### 2-(3-(3-(pentan-3-ylcarbamoyl)-1 H-pyrazol-5-yl)phenyl)-N-(3-(trifluoromethyl)phenyl)oxazole-5-carboxamide

ethyl (2-(3-(3-(pentan-3-ylcarbamoyl)-1 H-pyrazol-5-yl)phenyl)oxazole-5-carbonyl)-L-leucinate;

### N-(3-cyanophenyl)-2-(3-(3-(pentan-3-ylcarbamoyl)-1 H-pyrazol-5-yl)phenyl)oxazole-5-carboxamide

2-(3-(1-(2-(benzyloxy)ethyl)-5-(pentan-3-ylcarbamoyl)-1 H-pyrazol-3-yl)phenyl)-N-(pentan-3-yl)oxazole-5-carboxamide;

### ethyl (2-(3-(5-((1-cyclopropyl-2,2,2-trifluoroethyl)carbamoyl)-1H-pyrazol-3-yl)phenyl)oxazole-5-carbonyl)-L-valinate

methyl (5-(3-(5-(pentan-3-ylcarbamoyl)oxazol-2-yl)phenyl)-1 H-pyrazole-3-carbonyl)-L-alaninate;

### 2-(3-(1-(2-hydroxyethyl)-5-(pentan-3-ylcarbamoyl)-1 H-pyrazol-3-yl)phenyl)-N-(pentan-3-yl)oxazole-5-carboxamide

ethyl (2-(3-(1-(4-(tert-butoxy)-4-oxobutyl)-5-(((S)-1-cyclopropylethyl)carbamoyl)-1H-pyrazol-3-yl)phenyl)oxazole-5-carbonyl)-L-valinate;

(S)-N-(adamantan-1-yl)-2-(3-(5-((1-cyclopropylethyl)carbamoyl)-4H-1,2,4-triazol-3-yl)phenyl)oxazole-5-carboxamide;

### ethyl (R)-2-(2-(3-(3-(pentan-3-ylcarbamoyl)-1 H-pyrazol-5-yl)phenyl)oxazole-5-carboxamido)-2-phenylacetate

methyl (5-(3-(5-(pentan-3-ylcarbamoyl)oxazol-2-yl)phenyl)-1 H-pyrazole-3-carbonyl)phenylalaninate;

### 2-(3-(3-(tert-butylcarbamoyl)-1 H-pyrazol-5-yl)phenyl)-N-(pentan-3-yl)oxazole-5-carboxamide

2-(3-(5-(((S)-1-cyclopropylethyl)carbamoyl)-1-(3,3,3-trifluoro-2-hydroxypropyl)-1H-pyrazol-3-yl)phenyl)-N-(dicyclopropylmethyl)oxazole-5-carboxamide;

(S)-N-(1-cyclohexylethyl)-2-(3-(3-(pentan-3-ylcarbamoyl)-1 H-pyrazol-5-yl)phenyl)oxazole-5-carboxamide;

### methyl N-(2-(3-(3-(((S)-1-cyclopropylethyl)carbamoyl)-1H-pyrazol-5-yl)phenyl)oxazole-5-carbonyl)-S-methyl-D-cysteinate

2-(3-(4-(2-methoxyethyl)-5-(pentan-3-ylcarbamoyl)-4H-1,2,4-triazol-3-yl)phenyl)-N-(pentan-3-yl)oxazole-5-carboxamide;

### N-cyclopentyl-2-(3-(3-(pentan-3-ylcarbamoyl)-1 H-pyrazol-5-yl)phenyl)oxazole-5-carboxamide

methyl (5-(3-(5-(((S)-1-ethoxy-3-methyl-1-oxobutan-2-yl)carbamoyl)oxazol-2-yl)phenyl)-1 H-pyrazole-3-carbonyl)-L-leucinate;

(R)-2-(3-(3-((1-cyclohexylethyl)carbamoyl)-1 H-pyrazol-5-yl)phenyl)-N-(pentan-3-yl)oxazole-5-carboxamide;

### N-(3,5-dimethylphenyl)-2-(3-(3-(pentan-3-ylcarbamoyl)-1 H-pyrazol-5-yl)phenyl)oxazole-5-carboxamide

(S)-2-(3-(4-((1-cyclopropylethyl)carbamoyl)-1 H-imidazol-2-yl)phenyl)-N-(pentan-3-yl)oxazole-5-carboxamide;

### ethyl 3-methyl-1-(5-(3-(5-(pentan-3-ylcarbamoyl)oxazol-2-yl)phenyl)-1 H-pyrazole-3-carbonyl)pyrrolidine-3-carboxylate

ethyl (2-(3-(3-(((R)-1-cyclopropylethyl)carbamoyl)-1H-pyrazol-5-yl)phenyl)oxazole-5-carbonyl)-L-valinate;

tert-butyl (S)-2-(2-(3-(3-(pentan-3-ylcarbamoyl)-1 H-pyrazol-5-yl)phenyl)oxazole-5-carboxamido)-2-phenylacetate;

### ethyl (2-(3-(1-(2-hydroxyethyl)-5-(pentan-3-ylcarbamoyl)-1 H-pyrazol-3-yl)phenyl)oxazole-5-carbonyl)-L-valinate

2-(3-(3-((4-fluorobenzyl)carbamoyl)-1 H-pyrazol-5-yl)phenyl)-N-(pentan-3-yl)oxazole-5-carboxamide;

### 2-(3-(5-(((S)-1-cyclopropylethyl)carbamoyl)-1-((2,2-dimethyl-1,3-dioxolan-4-yl)methyl)-1 H-pyrazol-3-yl)phenyl)-N-(pentan-3-yl)oxazole-5-carboxamide

### ethyl 2-(5-(pentan-3-ylcarbamoyl)-3-(3-(5-(pentan-3-ylcarbamoyl)oxazol-2-yl)phenyl)-1 H-1,2,4-triazol-1-yl)acetate

2-(3-(5-(((S)-1-cyclopropylethyl)carbamoyl)-4H-1,2,4-triazol-3-yl)phenyl)-N-((S)-3-methylbutan-2-yl)oxazole-5-carboxamide;

### methyl (5-(3-(5-(pentan-3-ylcarbamoyl)oxazol-2-yl)phenyl)-1 H-pyrazole-3-carbonyl)-D-methioninate

N-((R)-1-cyclopropylethyl)-2-(3-(5-(((S)-1-cyclopropylethyl)carbamoyl)-4H-1,2,4-triazol-3-yl)phenyl)oxazole-5-carboxamide;

(S)-N-(1-cyclopropylethyl)-2-(3-(5-((4,4-difluorocyclohexyl)carbamoyl)-4H-1,2,4-triazol-3-yl)phenyl)oxazole-5-carboxamide;

### ethyl (5-(3-(5-(pentan-3-ylcarbamoyl)oxazol-2-yl)phenyl)-1 H-pyrazole-3-carbonyl)-L-phenylalaninate

ethyl (2-(3-(3-(((S)-1-cyclopropylethyl)carbamoyl)-1H-pyrazol-5-yl)phenyl)oxazole-5-carbonyl)-L-valinate;

### N-(pentan-3-yl)-2-(3-(3-(((1 S)-1-(tetrahydrofuran-2-yl)ethyl)carbamoyl)-1 H-pyrazol-5-yl)phenyl)oxazole-5-carboxamide

N-(pentan-3-yl)-2-(3-(3-(((S)-1-((S)-tetrahydrofuran-2-yl)ethyl)carbamoyl)-1H-pyrazol-5-yl)phenyl)oxazole-5-carboxamide;

### N-(pentan-3-yl)-2-(3-(3-(((S)-1-((R)-tetrahydrofuran-2-yl)ethyl)carbamoyl)-1 H-pyrazol-5-yl)phenyl)oxazole-5-carboxamide

diallyl 2,2′-((2,2′-(1,3-phenylene)bis(oxazole-2,5-diyl-5-carbonyl))bis(azanediyl))(2S,2'S)-bis(3-methylbutanoate);

### 2-(3-(3-((2-(tert-butylthio)ethyl)carbamoyl)-1 H-pyrazol-5-yl)phenyl)-N-(pentan-3-yl)oxazole-5-carboxamide

(R)-2-(3-(5-((1-cyclopropylethyl)carbamoyl)-1 H-pyrazol-3-yl)phenyl)-N-(pentan-3-yl)oxazole-5-carboxamide;

### 2-(3-(3-(pentan-3-ylcarbamoyl)-1 H-pyrazol-5-yl)phenyl)-N-((tetrahydro-2H-pyran-2-yl)methyl)oxazole-5-carboxamide

N-(pentan-3-yl)-2-(3-(4-(pentan-3-ylcarbamoyl)-1 H-imidazol-2-yl)phenyl)oxazole-5-carboxamide;

### N-((R)-1-cyclopropylethyl)-2-(3-(3-(((R)-1-cyclopropylethyl)carbamoyl)-1 H-1,2,4-triazol-5-yl)phenyl)oxazole-5-carboxamide

isopropyl (2-(3-(3-(pentan-3-ylcarbamoyl)-1 H-pyrazol-5-yl)phenyl)oxazole-5-carbonyl)glycinate;

### methyl (S)-3-cyclohexyl-2-(2-(3-(5-(((S)-1-cyclopropylethyl)carbamoyl)-4H-1,2,4-triazol-3-yl)phenyl)oxazole-5-carboxamido)propanoate

methyl (2-(3-(3-(((S)-1-methoxy-4-methyl-1-oxopentan-2-yl)carbamoyl)-1H-pyrazol-5-yl)phenyl)oxazole-5-carbonyl)-L-leucinate;

### 2-(3-(3-((2,6-difluorobenzyl)carbamoyl)-1 H-pyrazol-5-yl)phenyl)-N-(pentan-3-yl)oxazole-5-carboxamide

2-(3-(3-(4-methoxy-4-methylpiperidine-1-carbonyl)-1 H-pyrazol-5-yl)phenyl)-N-(pentan-3-yl)oxazole-5-carboxamide;

(S)-2-(3-(3-(sec-butylcarbamoyl)-1 H-pyrazol-5-yl)phenyl)-N-(pentan-3-yl)oxazole-5-carboxamide;

### 2-(3-(3-((2-methoxy-2-methylpropyl)carbamoyl)-1 H-pyrazol-5-yl)phenyl)-N-(pentan-3-yl)oxazole-5-carboxamide

tert-butyl 2-methyl-2-(2-(3-(3-(pentan-3-ylcarbamoyl)-1 H-pyrazol-5-yl)phenyl)oxazole-5-carboxamido)propanoate;

### methyl (2-(3-(3-(pentan-3-ylcarbamoyl)-1 H-pyrazol-5-yl)phenyl)oxazole-5-carbonyl)-L-valinate

benzyl (2-(3-(3-(pentan-3-ylcarbamoyl)-1 H-pyrazol-5-yl)phenyl)oxazole-5-carbonyl)-L-alaninate;

tert-butyl (5-(3-(5-(pentan-3-ylcarbamoyl)oxazol-2-yl)phenyl)-1 H-pyrazole-3-carbonyl)-L-valinate;

### methyl (R)-2-(5-(3-(5-(pentan-3-ylcarbamoyl)oxazol-2-yl)phenyl)-1 H-pyrazole-3-carboxamido)-2-phenylacetate

(S)-N-(sec-butyl)-2-(3-(3-(pentan-3-ylcarbamoyl)-1 H-pyrazol-5-yl)phenyl)oxazole-5-carboxamide;

### 2-(3-(3-((3-isopropoxyphenyl)carbamoyl)-1 H-pyrazol-5-yl)phenyl)-N-(pentan-3-yl)oxazole-5-carboxamide

N-((S)-1-cyclopropylethyl)-2-(3-(5-(((R)-1-cyclopropylethyl)carbamoyl)-4H-1,2,4-triazol-3-yl)phenyl)oxazole-5-carboxamide;

### 2-(3-(3-(cyclopentylcarbamoyl)-1 H-pyrazol-5-yl)phenyl)-N-(pentan-3-yl)oxazole-5-carboxamide

N-((S)-1-cyclopropylethyl)-2-(3-(5-(((S)-1-cyclopropylethyl)carbamoyl)-1-(2-hydroxy-2-methylpropyl)-1 H-pyrazol-3-yl)phenyl)oxazole-5-carboxamide;

### 2-(3-(3-((cyclopropyl(tetrahydrofuran-2-yl)methyl)carbamoyl)-1 H-pyrazol-5-yl)phenyl)-N-(pentan-3-yl)oxazole-5-carboxamide

tert-butyl 2-(5-(pentan-3-ylcarbamoyl)-3-(3-(5-(pentan-3-ylcarbamoyl)oxazol-2-yl)phenyl)-1 H-1,2,4-triazol-1-yl)acetate;

### ethyl (5-(3-(5-(pentan-3-ylcarbamoyl)oxazol-2-yl)phenyl)-1 H-pyrazole-3-carbonyl)-L-alaninate

2-(3-(3-(3,3-dimethylpiperidine-1-carbonyl)-1 H-pyrazol-5-yl)phenyl)-N-(pentan-3-yl)oxazole-5-carboxamide:

(S)-N-([1,1′-bi(cyclopropan)]-1-yl)-2-(3-(5-((1-cyclopropylethyl)carbamoyl)-4H-1,2,4-triazol-3-yl)phenyl)oxazole-5-carboxamide;

benzyl (5-(3-(5-(pentan-3-ylcarbamoyl)oxazol-2-yl)phenyl)-1 H-pyrazole-3-carbonyl)-L-alaninate;

(S)-2-(3-(3-((1-cyclohexylethyl)carbamoyl)-1H-pyrazol-5-yl)phenyl)-N-(pentan-3-yl)oxazole-5-carboxamide;

### N-((1-methylcyclohexyl)methyl)-2-(3-(3-(pentan-3-ylcarbamoyl)-1 H-pyrazol-5-yl)phenyl)oxazole-5-carboxamide

(R)-N-(1-cyclohexylethyl)-2-(3-(3-(pentan-3-ylcarbamoyl)-1 H-pyrazol-5-yl)phenyl)oxazole-5-carboxamide;

### methyl (5-(3-(5-(pentan-3-ylcarbamoyl)oxazol-2-yl)phenyl)-1 H-pyrazole-3-carbonyl)-L-phenylalaninate

tert-butyl 1-(5-(3-(5-(pentan-3-ylcarbamoyl)oxazol-2-yl)phenyl)-1 H-pyrazole-3-carbonyl)pyrrolidine-3-carboxylate;

### methyl (S)-1-(2-(3-(5-((1-cyclopropylethyl)carbamoyl)-1 H-pyrazol-3-yl)phenyl)oxazole-5-carboxamido)cyclobutane-1-carboxylate

N-(2,6-difluorobenzyl)-2-(3-(3-(pentan-3-ylcarbamoyl)-1 H-pyrazol-5-yl)phenyl)oxazole-5-carboxamide;

### 2-(3-(3-(((1-methylcyclopropyl)methyl)carbamoyl)-1 H-pyrazol-5-yl)phenyl)-N-(pentan-3-yl)oxazole-5-carboxamide

ethyl (2-(3-(5-(pentan-3-ylcarbamoyl)-1 H-pyrazol-3-yl)phenyl)oxazole-5-carbonyl)-D-valinate;

### N-benzyl-2-(3-(3-(pentan-3-ylcarbamoyl)-1 H-pyrazol-5-yl)phenyl)oxazole-5-carboxamide; methyl (2-(3-(3-(((S)-1-cyclopropylethyl)carbamoyl)-1H-pyrazol-5-yl)phenyl)oxazole-5-carbonyl)-L-valinate

(S)-2-(3-(3-((3,3-dimethylbutan-2-yl)carbamoyl)-1 H-pyrazol-5-yl)phenyl)-N-(pentan-3-yl)oxazole-5-carboxamide; ethyl (2-(3-(1-(2-(tert-butoxy)-2-oxoethyl)-5-(pentan-3-ylcarbamoyl)-1 H-pyrazol-3-yl)phenyl)oxazole-5-carbonyl)-L-valinate;

### ethyl (2-(3-(5-((1,1,1-trifluoropropan-2-yl)carbamoyl)-1H-pyrazol-3-yl)phenyl)oxazole-5-carbonyl)-L-valinate

(R)-N-(3-methylbutan-2-yl)-2-(3-(3-(pentan-3-ylcarbamoyl)-1 H-pyrazol-5-yl)phenyl)oxazole-5-carboxamide;

### 2-(3-(3-(((1-morpholinocyclohexyl)methyl)carbamoyl)-1 H-pyrazol-5-yl)phenyl)-N-(pentan-3-yl)oxazole-5-carboxamide

(R)-N-(pentan-3-yl)-2-(3-(3-((1-phenylethyl)carbamoyl)-1 H-pyrazol-5-yl)phenyl)oxazole-5-carboxamide:

### N-(pentan-3-yl)-2-(3-(3-((3-(trifluoromethoxy)phenyl)carbamoyl)-1 H-pyrazol-5-yl)phenyl)oxazole-5-carboxamide

2-(3-(3-(benzylcarbamoyl)-1 H-pyrazol-5-yl)phenyl)-N-(pentan-3-yl)oxazole-5-carboxamide;

### 2-(3-(5-(((S)-1-cyclopropylethyl)carbamoyl)-1 H-pyrazol-3-yl)phenyl)-N-(1-cyclopropylpropyl)oxazole-5-carboxamide

ethyl (S)-3-cyclohexyl-2-(5-(3-(5-(pentan-3-ylcarbamoyl)oxazol-2-yl)phenyl)-1 H-pyrazole-3-carboxamido)propanoate;

### 2-(3-(3-((cyclohexylmethyl)carbamoyl)-1 H-pyrazol-5-yl)phenyl)-N-(pentan-3-yl)oxazole-5-carboxamide

N-(3-chlorophenyl)-2-(3-(3-(pentan-3-ylcarbamoyl)-1 H-pyrazol-5-yl)phenyl)oxazole-5-carboxamide;

### methyl (R)-2-(2-(3-(3-(pentan-3-ylcarbamoyl)-1 H-pyrazol-5-yl)phenyl)oxazole-5-carboxamido)-2-phenylacetate

2,2′-(4-fluoro-1,3-phenylene)bis(N-(pentan-3-yl)oxazole-5-carboxamide); N-(benzo[d][1,3]dioxol-5-ylmethyl)-2-(3-(3-(pentan-3-ylcarbamoyl)-1 H-pyrazol-5-yl)phenyl)oxazole-5-carboxamide;

### ethyl 2-methyl-2-(2-(3-(3-(pentan-3-ylcarbamoyl)-1 H-pyrazol-5-yl)phenyl)oxazole-5-carboxamido)propanoate

tert-butyl 2-(5-(pentan-3-ylcarbamoyl)-3-(3-(5-(pentan-3-ylcarbamoyl)oxazol-2-yl)phenyl)-1 H-1,2,4-triazol-1-yl)acetate;

### N-((S)-1-cyclopropylethyl)-2-(3-(5-(((S)-1-cyclopropylethyl)carbamoyl)-4H-1,2,4-triazol-3-yl)phenyl)oxazole-5-carboxamide

ethyl (S)-2-(2-(3-(3-(pentan-3-ylcarbamoyl)-1 H-pyrazol-5-yl)phenyl)oxazole-5-carboxamido)-2-phenylacetate;

### N-(isoxazol-3-yl)-2-(3-(3-(pentan-3-ylcarbamoyl)-1 H-pyrazol-5-yl)phenyl)oxazole-5-carboxamide

N-(1-cyclopropylethyl)-2-(3-(5-((1-cyclopropylethyl)carbamoyl)-1 H-pyrazol-3-yl)phenyl)oxazole-5-carboxamide;

(S)-N-(1-cyclopropylethyl)-2-(3-(3-(pentan-3-ylcarbamoyl)-1 H-pyrazol-5-yl)phenyl)oxazole-5-carboxamide;

### N-(pentan-3-yl)-2-(3-(3-(piperidine-1-carbonyl)-1 H-pyrazol-5-yl)phenyl)oxazole-5-carboxamide

ethyl (2-(3-(3-(pentan-3-ylcarbamoyl)-1 H-pyrazol-5-yl)phenyl)oxazole-5-carbonyl)-L-phenylalaninate; and

### 2-(3-(3-((benzo[d][1,3]dioxol-5-ylmethyl)carbamoyl)-1 H-pyrazol-5-yl)phenyl)-N-(pentan-3-yl)oxazole-5-carboxamide

or a pharmaceutically acceptable salt, hydrate, or co-crystal thereof.

An eighteenth embodiment of the invention provides a pharmaceutical composition comprising a compound of any one of embodiments 1-17 or a pharmaceutically acceptable salt, hydrate, or co-crystal thereof, and a pharmaceutically acceptable carrier, or diluent.

A nineteenth embodiment of the invention provides a pharmaceutical composition of embodiment 18 further comprising one or more additional pharmaceutical agent(s).

A twentieth embodiment of the invention provides a pharmaceutical composition of embodiment 19 wherein the additional pharmaceutical agent(s) is selected from a mucolytic agent(s), nebulized hypertonic saline, bronchodilator(s), an antibiotic(s), an anti-infective agent(s), a CFTR modulator(s), and an anti-inflammatory agent(s).

A twenty-first embodiment of the invention provides a pharmaceutical composition of embodiment 19, wherein the additional pharmaceutical agent(s) is a CFTR modulator(s).

A twenty-second embodiment of the invention provides a pharmaceutical composition of embodiment 19, wherein the additional pharmaceutical agent(s) is a CFTR corrector(s).

A twenty-third embodiment of the invention provides a pharmaceutical composition of embodiment 19, wherein the additional pharmaceutical agent(s) is a CFTR potentiator(s).

A twenty-fourth embodiment of the invention provides a pharmaceutical composition of embodiment 19, wherein the additional pharmaceutical agent(s) comprise a CFTR amplifier(s).

A twenty-fifth embodiment of the invention provides a method for treating a disease associated with impaired mucociliary clearance in a subject comprising administering to the subject a compound or a pharmaceutically acceptable salt, hydrate, or co-crystal thereof of any one of embodiments 1 to 17 or the pharmaceutical composition of any one of embodiments 18 to 24.

A twenty-sixth embodiment of the invention provides a method of embodiment twenty-five, wherein the disease associated with impaired mucociliary clearance is selected from cystic fibrosis, asthma, bronchiectasis, COPD, and chronic bronchitis.

A twenty-seventh embodiment of the invention provides a method of embodiments twenty-five or twenty-six, wherein the disease associated with impaired mucociliary clearance is cystic fibrosis or COPD.

A twenty-eighth embodiment of the invention provides a method of embodiments twenty-five to twenty-seven, wherein the disease associated with impaired mucociliary clearance is cystic fibrosis.

A twenty-ninth embodiment of the invention provides a method of embodiment twenty-five wherein said method further comprises administering to the subject one or more additional pharmaceutical agent(s) prior to, concurrent with, or subsequent to the compound of any one of embodiments 1 to 17 or the pharmaceutical composition of any one of embodiments 18 to 24.

A thirtieth embodiment of the invention provides a method of embodiment twenty-nine, wherein the additional pharmaceutical agent(s) is selected from a mucolytic agent(s), nebulized hypertonic saline, bronchodilator(s), an antibiotic(s), an anti-infective agent(s), a CFTR modulator(s), and an anti-inflammatory agent(s).

A thirty-first embodiment of the invention provides a method of embodiment twenty-nine, wherein the additional pharmaceutical agent(s) is a CFTR modulator(s).

A thirty-second embodiment of the invention provides a method of embodiment twenty-nine, wherein the additional pharmaceutical agent(s) is a CFTR potentiator(s).

A thirty-third embodiment of the invention provides a method of embodiment twenty-nine, wherein the additional pharmaceutical agent(s) comprise a CFTR amplifier(s).

A thirty-fourth embodiment of the invention provides a monohydrate form of the free base of N-(pentan-3-yl)-2-(3-(3-(((S)-1-((S)-tetrahydrofuran-2-yl)ethyl)carbamoyl)-1H-pyrazol-5-yl)phenyl)oxazole-5-carboxamide wherein the monohydrate form has an X-ray powder diffraction pattern comprising a characteristic peak, in terms of 2θ, at about 24.6°.

A thirty-fifth embodiment of the invention provides a monohydrate form of embodiment thirty-four, wherein the X-ray powder diffraction pattern further comprises one or more characteristic peaks, in terms of 2θ, selected from peaks at about 7.6°, about 12.0°, about 15.6°, about 16.6°, about 18.6°, about 18.9°, about 21.5°, and about 23.1°.

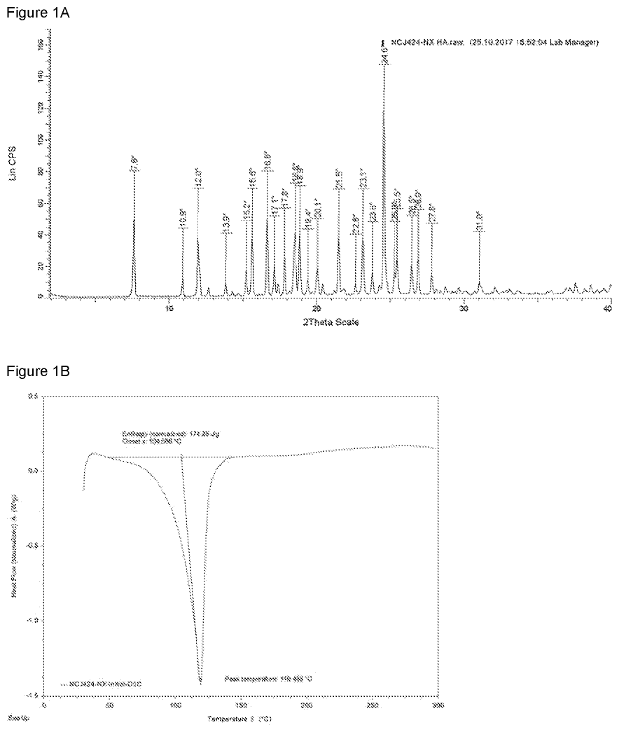

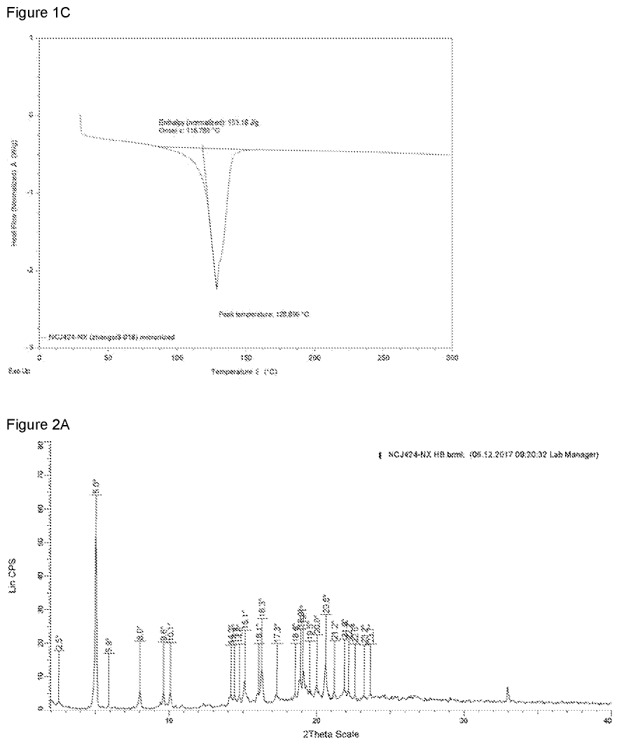

A thirty-sixth embodiment of the invention provides a monohydrate form of embodiment thirty-four having an X-ray powder diffraction pattern substantially as shown in FIG. 1A.

A thirty-seventh embodiment of the invention provides a monohydrate form of embodiment thirty-four having a differential scanning calorimetry thermogram showing an onset of an endotherm at about 104.6° C.

A thirty-eighth embodiment of the invention provides a monohydrate form of embodiment thirty-four having a differential scanning calorimetry thermogram substantially as shown in FIG. 1B.

A thirty-ninth embodiment of the invention provides a monohydrate form of embodiment thirty-four having a differential scanning calorimetry thermogram substantially as shown in FIG. 1C.

A fortieth embodiment of the invention provides a metastable hydrate form of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide wherein the metastable hydrate form has an X-ray powder diffraction pattern comprising a characteristic peak, in terms of 2θ, at about 5.0°.

A forty-first embodiment of the invention provides a metastable hydrate form of embodiment forty, wherein the X-ray powder diffraction pattern further comprises one or more characteristic peaks, in terms of 2θ, selected from peaks at about 15.1°, about 16.3°, about 18.9°, about 19.1°, and about 20.6°.

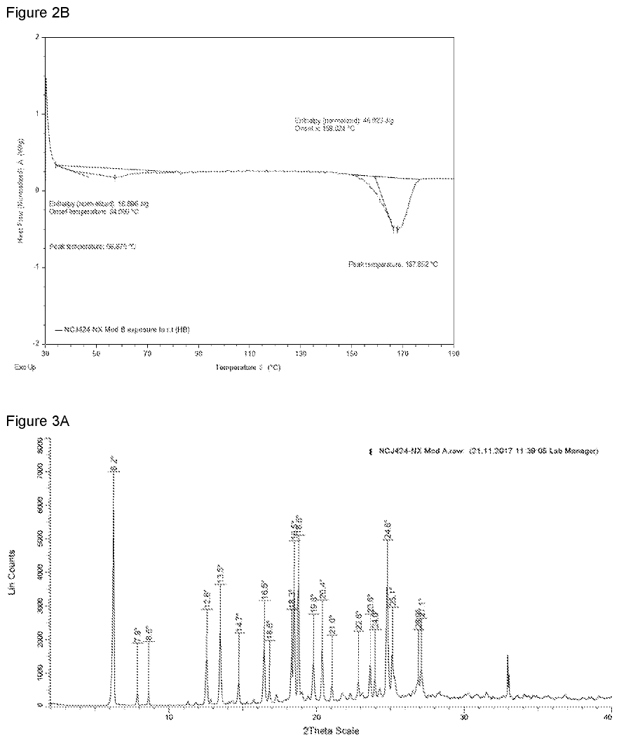

A forty-second embodiment of the invention provides a metastable hydrart form of embodiment forty having an X-ray powder diffraction pattern substantially as shown in FIG. 2A.

A forty-third embodiment of the invention provides a metastable hydrate form of embodiment forty having a differential scanning calorimetry thermogram showing an onset of an endotherm at about 34.0° C. and a second onset of an endotherm at 159.0° C.

A forty-fourth embodiment of the invention provides a metastable hydrate form of embodiment forty having a differential scanning calorimetry thermogram substantially as shown in FIG. 2B.

A forty-fifth embodiment of the invention provides an anhydrous form A of the free base of N-(pentan-3-yl)-2-(3-(3-(((S)-1-((S)-tetrahydrofuran-2-yl)ethyl)carbamoyl)-1H-pyrazol-5-yl)phenyl)oxazole-5-carboxamide wherein the monohydrate form has an X-ray powder diffraction pattern comprising a characteristic peak, in terms of 2θ, at about 6.2°.

A forty-sixth embodiment of the invention provides an anhydrous form A of embodiment forty-five, where in the X-ray powder diffraction pattern further comprises one or more characteristic peaks, in terms of 2θ, selected from peaks at about 13.5°, about 16.5°, about 18.5°, about 18.9°, about 20.4°, and about 24.8°.

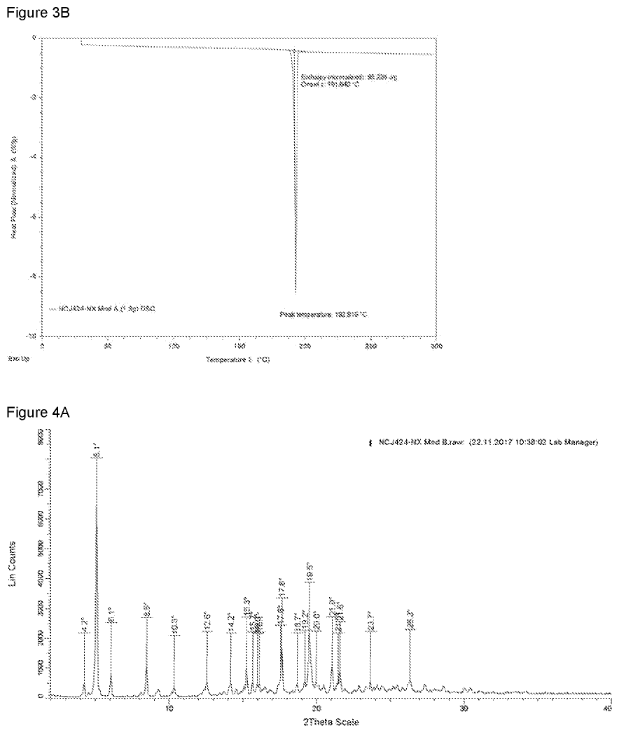

A forty-seventh embodiment of the invention provides an anhydrous form A of embodiment forty-five having an X-ray powder diffraction pattern substantially as shown in FIG. 3A.

A forty-eight embodiment of the invention provides an anhydrous form A of embodiment forty-five having a differential scanning calorimetry thermogram showing an onset of an endotherm at about 191.6° C.

A forty-ninth embodiment of the invention provides an anhydrous form A of embodiment forty-five having a differential scanning calorimetry thermogram substantially as shown in FIG. 3B.

A fiftieth embodiment of the invention provides an anhydrous form B of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide wherein the monohydrate form has an X-ray powder diffraction pattern comprising a characteristic peak, in terms of 2θ, at about 5.1°.

A fifty-first embodiment of the invention provides an anhydrous form B of embodiment fifty, wherein the X-ray powder diffraction pattern further comprises one or more characteristic peaks, in terms of 2θ, selected from peaks at about 8.5°, about 15.3°, about 17.6°, about 19.5°, and about 21.0°.

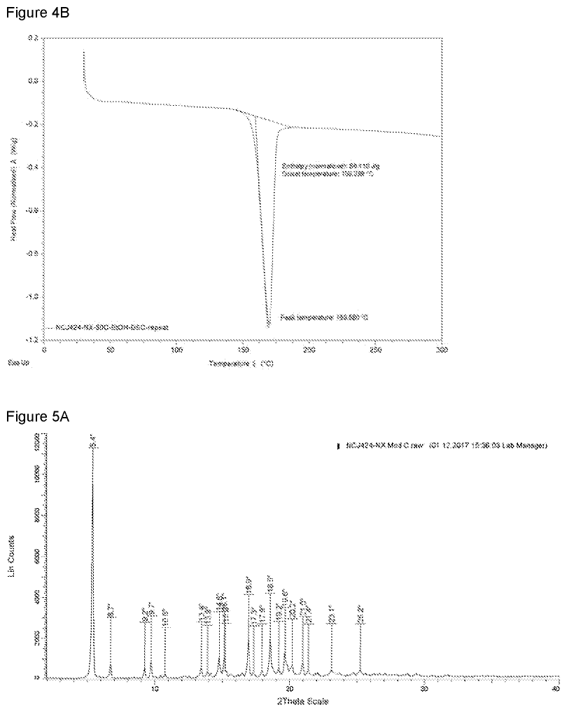

A fifty-second embodiment of the invention provides an anhydrous form B of embodiment fifty having an X-ray powder diffraction pattern substantially as shown in FIG. 4A.

A fifty-third embodiment of the invention provides an anhydrous form B of embodiment fifty having a differential scanning calorimetry thermogram showing an onset of an endotherm at about 159.2C.

A fifty-fourth embodiment of the invention provides an anhydrous form B of embodiment fifty having a differential scanning calorimetry thermogram substantially as shown in FIG. 4B.

A fifty-fifth embodiment of the invention provides an anhydrous form C of the free base of N-(pentan-3-yl)-2-(3-(3-(((S)-1-((S)-tetrahydrofuran-2-yl)ethyl)carbamoyl)-1H-pyrazol-5-yl)phenyl)oxazole-5-carboxamide wherein the monohydrate form has an X-ray powder diffraction pattern comprising a characteristic peak, in terms of 2θ, at about 5.4°.

A fifty-sixth embodiment of the invention provides an anhydrous form C of embodiment fifty-five, wherein the X-ray powder diffraction pattern further comprises one or more characteristic peaks, in terms of 2θ, selected from peaks at about 14.8°, about 15.1°, about 16.9°, about 18.5°, and about 19.6°.

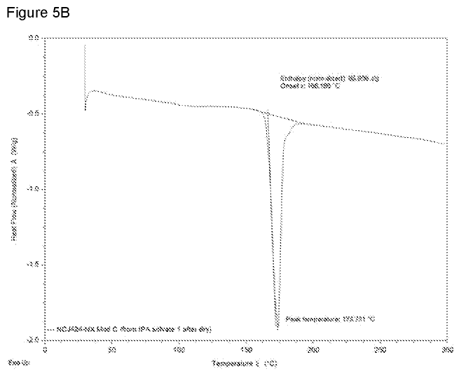

A fifty-seventh embodiment of the invention provides an anhydrous form C of embodiment fifty-five having an X-ray powder diffraction pattern substantially as shown in FIG. 5A.

A fifty-eighth embodiment of the invention provides an anhydrous form C of embodiment fifty-five having a differential scanning calorimetry thermogram showing an onset of an endotherm at about 166.2C.

A fifty-ninth embodiment of the invention provides an anhydrous form C of embodiment fifty-five having a differential scanning calorimetry thermogram substantially as shown in FIG. 5B.

A sixtieth embodiment of the invention provides a solid form of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide wherein the solid form has an X-ray powder diffraction pattern comprising a characteristic peak, in terms of 2θ, at about 24.6°.

A sixty first embodiment of the invention provides a compound of formula Ill

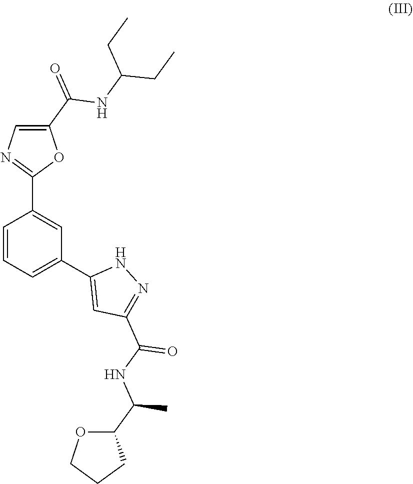 [SMILES: CCC(CC)NC(=O)c1cnc(-c2cccc(-c3cc(C(=O)N[C@@H](C)[C@@H]4CCCO4)n[nH]3)c2)o1]

In certain embodiments, the present invention relates to the aforementioned methods, wherein said compound is administered parenterally.

In certain embodiments, the present invention relates to the aforementioned methods, wherein said compound is administered intramuscularly, intravenously, subcutaneously, orally, pulmonary, intrathecally, topically or intranasally.

In certain embodiments, the present invention relates to the aforementioned methods, wherein said compound is administered systemically.

In certain embodiments, the present invention relates to the aforementioned methods, wherein said subject is a mammal.

In certain embodiments, the present invention relates to the aforementioned methods, wherein said subject is a primate.

In certain embodiments, the present invention relates to the aforementioned methods, wherein said subject is a human.

The compounds and intermediates described herein may be isolated and used as the compound per se. Alternatively, when a moiety is present that is capable of forming a salt, the compound or intermediate may be isolated and used as its corresponding salt. As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholanate, diethanolamine, diethylamine, lysine, meglumine, piperazine and tromethamine.

The salts can be synthesized by conventional chemical methods from a compound containing a basic or acidic moiety. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two.

Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

It will be recognized by those skilled in the art that the compounds of the present invention may contain chiral centers and as such may exist in different stereoisomeric forms.

As used herein, the term “an optical isomer” or “a stereoisomer” refers to any of the various stereo isomeric configurations which may exist for a given compound of the present invention. It is understood that a substituent may be attached at a chiral center of a carbon atom. Therefore, the invention includes enantiomers, diastereomers or racemates of the compound.

“Enantiomers” are a pair of stereoisomers that are non- superimposable mirror images of each other. A 1:1 mixture of a pair of enantiomers is a “racemic” mixture. The term is used to designate a racemic mixture where appropriate. When designating the stereochemistry for the compounds of the present invention, a single stereoisomer with known relative and absolute configuration of the two chiral centers is designated using the conventional RS system (e.g., (1S,2S)); a single stereoisomer with known relative configuration but unknown absolute configuration is designated with stars (e.g., (1R*,2R*)); and a racemate with two letters (e.g, (1 RS,2RS) as a racemic mixture of (1R,2R) and (1S,2S); (1 RS,2SR) as a racemic mixture of (1R,2S) and (1 S,2R)). “Diastereoisomers” are stereoisomers that have at least two asymmetric atoms, but which are not mirror-images of each other. The absolute stereochemistry is specified according to the Cahn- Ingold- Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)- or (S)-.

Unless specified otherwise, the compounds of the present invention are meant to include all such possible stereoisomers, including racemic mixtures, optically pure forms and intermediate mixtures. Optically active (F)- and (S)- stereoisomers may be prepared using chiral synthons or chiral reagents, or resolved using conventional techniques (e.g., separated on chiral SFC or HPLC chromatography columns, such as CHIRALPAK® and CHIRALCEL® available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

### Pharmacology and Utility

The agents of the invention act to potentiate the TMEM16A chloride channel and are useful in the treatment of conditions, which respond to the potentiation of the TMEM16A, particularly conditions benefiting from mucosal hydration.

Transmembrane member 16A (TMEM16A, also known as Anoctamin-1 (ANO1)) is a calcium activated chloride channel expressed in the airway epithelium. Diseases mediated by potentiation of TMEM16A, include diseases associated with the regulation of fluid volumes across epithelial membranes. For example, the volume of airway surface liquid is a key regulator of mucociliary clearance and the maintenance of lung health. The potentiation of TMEM16A will promote a durable chloride flux from pulmonary epithelia leading to fluid accumulation and mucus hydration on the mucosal side of the airway epithelium thereby promoting mucus clearance and preventing the accumulation of mucus and sputum in respiratory tissues (including lung airways). Such diseases include respiratory diseases, such as chronic bronchitis, chronic obstructive pulmonary disease (COPD), bronchiectasis, asthma, cystic fibrosis, primary ciliary dyskinesia, respiratory tract infections (acute and chronic; viral and bacterial) and lung carcinoma. Diseases mediated by potentiation of TMEM16A also include diseases other than respiratory diseases that are associated with abnormal fluid regulation across an epithelium, perhaps involving abnormal physiology of the protective surface liquids on their surface, e.g., xerostomia (dry mouth) or keratoconjunctivitis sire (dry eye). Furthermore, potentiation of TMEM16A in the kidney could be used to promote diuresis and thereby induce a hypotensive effect.

Bronchiectasis is the dilation and damage of the large airways of the lungs (bronchi) with loss of the smooth muscle and loss of elasticity of segments of the bronchi. The resultant airway distortion prevents secretions from being adequately cleared from the lung, allowing bacteria to grow and cause recurrent lung infections. The disease may be localized to one area of a lung, or generalized throughout both lungs. Bronchiectasis represents the final common pathway of a number of infectious, genetic, autoimmune, developmental and allergic disorders and is highly heterogeneous in its etiology, impact and prognosis (Chalmers JD et al, Eur Respir J 2015). The disease is a chronic respiratory disorder characterized by a clinical syndrome of cough, sputum production and bronchial infection, and it is associated with poor quality of life and frequent exacerbations in many patients.

Bronchiectasis patients are typically given prolonged courses of antibiotics for infective exacerbations. Despite antibiotic treatment patients still suffer from frequent exacerbations. Long-term macrolide antibiotics and other antibiotics are complicated by microbial resistance (Pomares et al 2018). Increased secretion of anions via potentiation of TMEM16A in lung epithelia will lead to improved hydration of pathologic mucus, resolving the dysregulation of mucociliary clearance by enhancing the clearance and therefore preventing progressive chronic remodeling driven by recurrent exacerbations, chronic infection and mucus dysregulation.

Chronic obstructive pulmonary disease (COPD) is a chronic inflammatory disease of the lung characterized by persistent respiratory symptoms (dyspnea, cough, sputum production) and poorly reversible airflow limitation that is due to airway and/or alveolar abnormalities. Chronic airflow limitation is caused by a mixture of small airways disease (obstructive bronchiolitis) and parenchymal destruction (emphysema). COPD is associated with episodic periods of symptom deterioration termed exacerbations. Exacerbations are important events in the natural history of COPD that drive lung function decline (Donaldson et al., 2002). COPD exacerbations are associated with systemic and pulmonary inflammation and increased levels of inflammatory mediators and cells have been measured in airway tissues e.g. TNF-α, IL-8, IL-6, leukotriene B4, neutrophils, lymphocytes and eosinophils (Beasley V. et al. COPD, Int J of COPD 2012).

COPD encompasses a spectrum of diseases, with chronic bronchitis at one end and emphysema at the other, with most individuals having some characteristics of both Chronic bronchitis, due to mucous hypersecretion and mucociliary dysfunction characterized by chronic cough and sputum, is a key phenotype in COPD subjects with numerous clinical consequences, including an increased exacerbation rate, accelerated decline in lung function, worse health-related quality of life, and possibly increased mortality. (Kim et al., 2012). COPD patients have decreased mucociliary clearance and increased mucus solids consistent with airway dehydration. Potentiation of TMEM16A will improve airway hydration and potentially act as a surrogate for CFTR-mediated chloride secretion and therefore alter mucus viscosity and enhance mucociliary clearance in COPD.

Asthma is a chronic disease in which inflammation causes the bronchial tubes to narrow and swell, creating breathing difficulties that may range from mild to life-threatening. Asthma includes both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g., of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”).

Prophylactic efficacy in the treatment of asthma will be evidenced by reduced frequency or severity of symptomatic attack, e.g., of acute asthmatic or bronchoconstrictor attack, improvement in lung function or improved airways hyperreactivity. It may further be evidenced by reduced requirement for other, symptomatic therapy, i.e., therapy for or intended to restrict or abort symptomatic attack when it occurs, e.g., anti-inflammatory (e.g., cortico-steroid) or bronchodilatory. Prophylactic benefit in asthma may, in particular, be apparent in subjects prone to “morning dipping”. “Morning dipping” is a recognized asthmatic syndrome, common to a substantial percentage of asthmatics and characterized by asthma attack, e.g., between the hours of about 4-6 am, i.e., at a time normally substantially distant from any previously administered symptomatic asthma therapy.

In certain embodiments, the present invention provides a method of treating a condition, disease, or disorder associated with the regulation of fluid volumes across epithelial membranes, the method comprising administering a composition comprising a compound of formula (I) to a subject, preferably a mammal, in need of treatment thereof.

According to the invention an “effective dose” or an “effective amount” of the compound or pharmaceutical composition is that amount effective for treating or lessening the severity of one or more of the diseases, disorders or conditions as recited above.

The compounds and compositions, according to the methods of the present invention, may be administered using any amount and any route of administration effective for treating or lessening the severity of one or more of the diseases, disorders or conditions recited above.

The compounds of the present invention are typically used as a pharmaceutical composition (e.g., a compound of the present invention and at least one pharmaceutically acceptable carrier). As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this invention, solvates and hydrates are considered pharmaceutical compositions comprising a compound of the present invention and a solvent (i.e., solvate) or water (i.e., hydrate).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present invention is generally formulated for use as a parenteral or oral administration.

For example, the pharmaceutical oral compositions of the present invention can be made up in a solid form (including without limitation capsules, tablets, pills, granules, powders or suppositories), or in a liquid form (including without limitation solutions, suspensions or emulsions). Oral compositions can also include inhaled forms such as dry powders, aerosols, or other atomizable formulation, The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Pharmaceutical compositions in the form of a dry powder for inhalation can be contained in gelatin or plastic capsules or in plastic and/or foil containment blisters, which contain the active ingredient together with
a) Carrier particles, e.g. sugars such as lactose, mannitol, and sorbitol b) Lubricants, e.g. metal stearates such as magnesium stearate;
c) Agglomerates, e.g. lactose anhydrous and glucose anhydrous;
d) Hydrophobic shell formers, e.g. leucine, tri-leucine, glycine;
e) Blowing agents, e.g. ammonium carbonate, PFOB;
f) Stabilizing agents, e.g. sodium chloride, calcium chloride;
g) Controlled releasers, e.g. chitosan and by-products thereof, hyaluronic acid;
h) Absorption enhancers, e.g. citric acid, Hydroxypropyl-beta-cyclodextrin;
i) Stabilizers, e.g. SLS;
j) Buffers, e.g. L-histidine, sodium citrate;
k) Force control agents, e.g. magnesium stearate, sodium stearate, sucrose stearate;
l) pH control agents, e.g. HCl, Sulfuric acid, NaOH;
m) Matrix formers, e.g. raffinose, trehalose, mannitol, FDKP, DSPC, DPPC; and/or
n) Antioxidants, e.g. methionin, glutathion, arginine

Suitable compositions for oral administration include a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present invention or pharmaceutical composition thereof for use in a subject (e.g., human) is typically administered orally or parenterally at a therapeutic dose of less than or equal to about 100 mg/kg, 75 mg/kg, 50 mg/kg, 25 mg/kg, 10 mg/kg, 7.5 mg/kg, 5.0 mg/kg, 3.0 mg/kg, 1.0 mg/kg, 0.5 mg/kg, 0.05 mg/kg or 0.01 mg/kg, but preferably not less than about 0.0001 mg/kg. When administered intravenously via infusion, the dosage may depend upon the infusion rate at which an iv formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either entirely, parenterally, advantageously intravenously, e.g., as a suspension or in aqueous solution. The dosage in vitro may range between about 10-3 molar and 10-9 molar concentrations.

### Polymorphs

In one aspect, compounds of formula one can take the form of polymorphs, hydrates and solvates. In one particular embodiment, the invention provides a monohydrate of the free base of N-(pentan-3-yl)-2-(3-(3-(((S)-1-((S)-tetrahydrofuran-2-yl)ethyl)carbamoyl)-1H-pyrazol-5-yl)phenyl)oxazole-5-carboxamide having an X-ray powder diffraction pattern comprising a characteristic peak, in terms of 2θ, at about 24.6°. In another embodiment, the X-ray powder diffraction pattern further comprises one or more characteristic peaks, in terms of 2θ, selected from peaks at about 7.6°, about 12.0°, about 15.6°, about 16.6°, about 18.6°, about 18.9°, about 21.5°, and about 23.1°. Thus, the X-ray powder diffraction pattern for a monohydrate form of the free base may comprise one, two, three, four, five, six, seven, eight or nine characteristic peaks, in terms of 2θ, selected from peaks at about 7.6°, about 12.0°, about 15.6°, about 16.6°, about 18.6°, about 18.9°, about 21.5°, about 23.1° and 24.6°. The X-ray powder diffraction pattern may further include between one and fifteen additional characteristic peaks, in terms of 2θ, selected from peaks at about 10.9°, about 13.9°, about 15.2°, about 17.1°, about 17.8°, about 19.4°, about 20.1°, about 22.6°, about 23.8°, about 25.3°, about 25.5°, about 26.5°, about 26.9°, about 27.8°, and about 31.0°. In another embodiment, the monohydrate crystalline form of the free base has an X-ray powder diffraction pattern substantially as shown in FIG. 1A. As used herein, the terms “about” and “substantially” indicate, with respect to values of 26, that such values for individual peaks can vary by ±0.4°. In some embodiments, the values of 26 for individual peaks can vary by +0.2°.

The monohydrate crystalline form of the free base of of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide may be characterized thermally. In one embodiment, the monohydrate crystalline form of the free base has a differential scanning calorimetry (DSC) thermogram showing an onset of an endotherm at about 104.6° C. In another embodiment, the monohydrate crystalline form of the free base has a differential scanning calorimetry thermogram substantially as shown in FIG. 1B. In a further embodiment, the monohydrate crystalline form of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide exhibits a slight loss of crystallinity upon micronization resulting in a modified DSC demonstrating an endotherm at 118.8° C. In another embodiment, the micronized monohydrate crystalline form of the free base has a differential scanning calorimetry thermogram substantially as shown in FIG. 1C. As used herein, the terms “about” and “substantially” indicate with respect to features such as endotherms, exotherms, baseline shifts, etc., that their values can vary ±2° C. For DSC, variation in the temperatures observed will depend upon the rate of temperature change as well as sample preparation technique and the particular instrument employed. Thus, the values reported herein relating to DSC thermograms can vary ±4° C.

In another particular embodiment, the invention provides a metastable hydrate of the free base of N-(pentan-3-yl)-2-(3-(3-(((S)-1-((S)-tetrahydrofuran-2-yl)ethyl)carbamoyl)-1H-pyrazol-5-yl)phenyl)oxazole-5-carboxamide having an X-ray powder diffraction pattern comprising a characteristic peak, in terms of 2θ, at about 5.0°. In another embodiment, the X-ray powder diffraction pattern further comprises one or more characteristic peaks, in terms of 2θ, selected from peaks at about 15.1°, about 16.3°, about 18.9°, about 19.1°, and about 20.6°. Thus, the X-ray powder diffraction pattern for an metastable hydrate form of the free base may comprise one, two, three, four, five, or six characteristic peaks, in terms of 2θ, selected from peaks at about 5.0°, about 15.1°, about 16.3°, about 18.9°, about 19.1°, and about 20.6°. The X-ray powder diffraction pattern may further include between one and nineteen additional characteristic peaks, in terms of 2θ, selected from peaks at about 2.5°, about 5.9°, about 8.0°, about 9.6°, about 10.1°, about 14.2°, about 14.4°, about 14.8°, about 16.1°, about 17.3°, about 18.6°, about 19.5°, about 20.0°, about 21.2°, about 21.9°, about 22.2°, about 22.6°, about 23.2°, and about 23.7°. In another embodiment, the metastable hydrate crystalline form of the free base has an X-ray powder diffraction pattern substantially as shown in FIG. 2A.

The metastable hydrate crystalline form of the free base of of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide may be characterized thermally. In one embodiment, the metastable hydrate crystalline form of the free base has a differential scanning calorimetry (DSC) thermogram showing an onset of an endotherm at about 34.0° C. and a secondary onset of an endotherm at 159.0° C. In another embodiment, the metastable hydrate crystalline form of the free base has a differential scanning calorimetry thermogram substantially as shown in FIG. 2B.

In another embodiment, the invention provides an anhydrous form A of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide having an x-ray powder diffraction pattern comprising a characteristic peak, in terms of 2θ, at about 6.2°. In another embodiment, the X-ray powder diffraction pattern further comprises one or more characteristic peaks, in terms of 2θ, selected from peaks at about 13.5°, about 16.5°, about 18.5°, about 18.8°, about 20.4°, and about 24.8°. Thus, the X-ray powder diffraction pattern for an anhydrous A form of the free base may comprise one, two, three, four, five, six, or seven characteristic peaks, in terms of 26, selected from peaks at about 6.2°, about 13.5°, about 16.5°, about 18.5°, about 18.8°, about 20.4°, and about 24.8°. The X-ray powder diffraction pattern may further include between one and fourteen additional characteristic peaks, in terms of 2θ, selected from peaks at about 7.9°, about 8.6°, about 12.6°, about 14.7°, about 16.8°, about 18.3°, about 19.8°, about 21.0°, about 22.8°, about 23.6°, about 24.0°, about 25.1°, about 26.9°, and about 27.1°. In another embodiment, the anhydrous form A of the free base has an X-ray powder diffraction pattern substantially as shown in FIG. 3A.

The anhydrous form A of the free base of of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide may be characterized thermally. In one embodiment, the anhydrous form A of the free base has a differential scanning calorimetry (DSC) thermogram showing an onset of an endotherm at about 191.6° C. In another embodiment, the anhydrous form A of the free base has a differential scanning calorimetry thermogram substantially as shown in FIG. 3B.

In another embodiment, the invention provides an anhydrous form B of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide having an x-ray powder diffraction pattern comprising a characteristic peak, in terms of 2θ, at about 5.1°. In another embodiment, the X-ray powder diffraction pattern further comprises one or more characteristic peaks, in terms of 2θ, selected from peaks at about 8.5°, about 15.3°, about 17.6°, about 19.5°, and about 21.0°.

Thus, the X-ray powder diffraction pattern for an anhydrous B form of the free base may comprise one, two, three, four, five, or six, characteristic peaks, in terms of 2θ, selected from peaks at about 5.1°, about 8.5°, about 15.3°, about 17.6°, about 19.5°, and about 21.0°. The X-ray powder diffraction pattern may further include between one and fifteen additional characteristic peaks, in terms of 2θ, selected from peaks at about 4.2°, about 6.1°, about 10.3°, about 12.6°, about 14.2°, about 15.7°, about 16.0°, about 16.1°, about 18.7°, about 19.2°, about 20.0°, about 21.5°, about 21.6°, about 23.7°, and about 26.3°. In another embodiment, the anhydrous form B of the free base has an X-ray powder diffraction pattern substantially as shown in FIG. 4A.

The anhydrous form B of the free base of of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide may be characterized thermally. In one embodiment, the anhydrous form B of the free base has a differential scanning calorimetry (DSC) thermogram showing an onset of an endotherm at about 159.2° C. In another embodiment, the anhydrous form B of the free base has a differential scanning calorimetry thermogram substantially as shown in FIG. 4B.

In another embodiment, the invention provides an anhydrous form C of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide having an x-ray powder diffraction pattern comprising a characteristic peak, in terms of 2θ, at about 5.4°. In another embodiment, the X-ray powder diffraction pattern further comprises one or more characteristic peaks, in terms of 2θ, selected from peaks at about 14.8°, about 15.1°, about 16.9°, about 18.5°, and about 19.6°.

Thus, the X-ray powder diffraction pattern for an anhydrous form of the free base may comprise one, two, three, four, five, or six, characteristic peaks, in terms of 2θ, selected from peaks at about 5.4°, about 14.8°, about 15.1°, about 16.9°, about 18.5°, and about 19.6°.

The X-ray powder diffraction pattern may further include between one and fifteen additional characteristic peaks, in terms of 2θ, selected from peaks at about 6.7°, about 9.2°, about 9.7°, about 10.8°, about 13.4°, about 13.9°, about 15.2°, about 17.3°, about 17.9°, about 19.2°, about 20.2°, about 21.0°, about 21.4°, about 23.1°, and about 25.2°. In another embodiment, the anhydrous form C of the free base has an X-ray powder diffraction pattern substantially as shown in FIG. 5A.

The anhydrous form C of the free base of of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide may be characterized thermally. In one embodiment, the anhydrous form C of the free base has a differential scanning calorimetry (DSC) thermogram showing an onset of an endotherm at about 166.2° C. In another embodiment, the anhydrous form C of the free base has a differential scanning calorimetry thermogram substantially as shown in FIG. 5B.

In another aspect, the present technology provides a method for making the monohydrate crystalline form of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide, comprising dissolution of 800 g of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide in 3.5 L methanol followed by precipitation via stewpise water addition (total amount of added water: 5.25 L). Yield was 84%.

In another aspect, a method is provided for making the anhydrous form A of the free base of N-(pentan-3-yl)-2-(3-(3-(((S)-1-((S)-tetrahydrofuran-2-yl)ethyl)carbamoyl)-1H-pyrazol-5-yl)phenyl)oxazole-5-carboxamide comprising equilibrating 1.5 g of the monohydrate crystalline form of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide in 10 mL ethyl acetate at 50° C. for 24 hours, separating by filtration at ambient conditions and drying at 50° C. for 2 hours. Yield was 87%.

In another aspect, a method is provided for making the anhydrous form B of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide comprising equilibrating 30 mg of the monohydrate crystalline form of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide with 0.3 mL ethanol to achieve a suspension, slurrying the mixture at 50° C. for 3 week and isolating the solid via filter centrifugation.

In another aspect, a method is provided for making the anhydrous form C of the free base of N-(pentan-3-yl)-2-(3-(3-(((S)-1-((S)-tetrahydrofuran-2-yl)ethyl)carbamoyl)-1H-pyrazol-5-yl)phenyl)oxazole-5-carboxamide comprising equilibrating 30 mg of the monohydrate crystalline form of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide with 0.3 ml isopropanol to achieve a suspension, slurrying the mixture at 50° C. for 3 week, isolating the solid via filter centrifugation, and drying the solid at 50° C.

In another aspect, a method is provided for making the metastable hydrate of the free base of N-(pentan-3-yl)-2-(3-(3-(((S)-1-((S)-tetrahydrofuran-2-yl)ethyl)carbamoyl)-1H-pyrazol-5-yl)phenyl)oxazole-5-carboxamide comprising exposing 40 mg of the anhydrous form B of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide to ambient conditions for a period of two weeks.

Combination Therapy TMEM16A potentiators, including the compounds of formula (I), are also useful as co-therapeutic agents for use in combination with other drug substances, such as anti-inflammatory, bronchodilatory, antihistamine or anti-tussive drug substances, particularly in the treatment of cystic fibrosis, asthma, or obstructive or inflammatory airways diseases such as those mentioned hereinbefore, e.g., as potentiators of therapeutic activity of such drugs or as a means of reducing required dosage or potential side effects of such drugs.

TMEM16A potentiator may be mixed with the other drug substance in a fixed pharmaceutical composition or it may be administered separately, before, simultaneously with or after the other drug substance.

Accordingly, the invention includes a combination of a TMEM16A potentiator with an anti-inflammatory, ENaC blockers, bronchodilatory, antihistamine, anti-tussive, antibiotic, epithelial sodium channel blocker or DNase drug substance, said drug substance being in the same or different pharmaceutical composition.

Suitable antibiotics include macrolide antibiotics, e.g., tobramycin (TOBI™) Suitable DNase drug substances include dornase alfa (Pulmozyme™), a highly-purified solution of recombinant human deoxyribonuclease I (rhDNase), which selectively cleaves DNA. Dornase alfa is used to treat cystic fibrosis.

Other useful combinations of epithelial sodium channel blockers with anti-inflammatory drugs are those with antagonists of chemokine receptors, e.g., CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists, such as Schering-Plough antagonists SC-351 125, SCH-55700 and SCH-D; Takeda antagonists, such as N/-[[4-[[[6,7-dihydro-2-(4-methyl-phenyl)-5H-benzo-cyclohepten-8-yl]carbonyl]amino]phenyl]-methyl]tetrahydro- N/, N/-dimethyl-2/-/-pyran-4-amin-ium chloride (TAK-770); and CCR-5 antagonists described in U.S. Pat. No. 6,166,037 (particularly claims 18 and 19), WO 00/66558 (particularly claim 8), WO 00/66559 (particularly claim 9), WO 04/018425 and WO 04/026873.

Suitable anti-inflammatory drugs include steroids, in particular, glucocorticosteroids, such as budesonide, beclamethasone dipropionate, fluticasone propionate, ciclesonide or mometasone furoate, or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/35668, WO 03/48181, WO 03/62259, WO 03/64445, WO 03/72592, WO 04/39827 and WO 04/66920; non-steroidal glucocorticoid receptor agonists, such as those described in DE 10261874, WO 00/00531, WO 02/10143, WO 03/82280, WO 03/82787, WO 03/86294, WO 03/104195, WO 03/101932, WO 04/05229, WO 04/18429, WO 04/19935 and WO 04/26248; LTD4 antagonists, such as montelukast and zafirlukast; PDE4 inhibitors, such as cilomilast (Ariflo® GlaxoSmithKline), Roflumilast (Byk Gulden),V-1 1294A (Napp), BAY19-8004 (Bayer), SCH-351591 (Schering-Plough), Arofylline (Almirall Prodesfarma), PD1 89659/PD1 68787 (Parke-Davis), AWD-1 2-281 (Asta Medica), CDC-801 (Celgene), SeICID™ CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805; adenosine A2B receptor antagonists such as those described in WO 02/42298; and beta-2 adrenoceptor agonists, such as albuterol (salbutamol), metaproterenol, terbutaline, salmeterol fenoterol, procaterol, and especially, formoterol, carmoterol and pharmaceutically acceptable salts or co-crystals thereof, and compounds (in free or salt or solvate form) of formula (1) of WO 00751 14, which document is incorporated herein by reference, preferably compounds of the Examples thereof, especially a compound of formula:

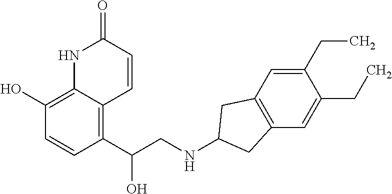 [SMILES: CCc1cc2c(cc1CC)CC(NCC(O)c1ccc(O)c3[nH]c(=O)ccc13)C2]

corresponding to indacaterol and pharmaceutically acceptable salts or co-crystals thereof, as well as compounds (in free or salt or solvate form) of formula (I) of WO 04/16601, and also compounds of EP 1440966, JP 05025045, WO 93/18007, WO 99/64035,
USP 2002/0055651, WO 01/42193, WO 01/83462, WO 02/66422, WO 02/70490, WO 02/76933, WO 03/24439, WO 03/42160, WO 03/42164, WO 03/72539, WO 03/91204, WO 03/99764, WO 04/16578, WO 04/22547, WO 04/32921, WO 04/33412, WO 04/37768, WO 04/37773, WO 04/37807, WO 04/39762, WO 04/39766, WO 04/45618, WO 04/46083, WO 04/80964, WO 04/108765 and WO 04/108676.
Suitable bronchodilatory drugs include anticholinergic or antimuscarinic agents, in particular, ipratropium bromide, oxitropium bromide, tiotropium salts and CHF 4226 (Chiesi), and glycopyrrolate, but also those described in EP 424021, U.S. Pat. No. 3,714,357, USP 5,171,744, WO 01/041 18, WO 02/00652, WO 02/51841, WO 02/53564, WO 03/00840, WO 03/33495, WO 03/53966, WO 03/87094, WO 04/018422 and WO 04/05285.
Suitable dual anti-inflammatory and bronchodilatory drugs include dual beta-2 adrenoceptor agonist/muscarinic antagonists such as those disclosed in USP 2004/0167167, WO 04/74246 and WO 04/74812.
Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine, as well as those disclosed in JP 2004107299, WO 03/099807 and WO 04/026841.
In accordance with the foregoing, the invention also provides a method for the treatment of diseases associated with the regulation of fluid volumes across epithelial membranes, particularly an obstructive airways disease, which comprises administering to a subject, particularly a human subject, in need thereof a compound of formula (I), in free form or in the form of a pharmaceutically acceptable salt, hydrate, or co-crystal. In another aspect, the invention provides a compound of formula (I), in free form or in the form of a pharmaceutically acceptable salt, hydrate, or co-crystal, for use in the manufacture of a medicament for the treatment of a condition responsive to potentiation of TMEM16A, particularly an obstructive airways disease, e.g., chronic bronchitis, COPD and bronchiectasis.

### Definitions

As used herein, the term “TMEM16A” refers to a calcium activated chloride channel belonging to the anoctamin/TMEM16 family of membrane proteins. The TMEM16 family has ten currently known members. TMEM16A and TEMEM16B are the most homologous. TMEM16A pore forming region is highly conserved across the family. TMEM16A is expressed at high levels on certain cancer cells, such as gastrointestinal tract and head and neck cancers. The TMEM16A has four known splice variants named a, b, c. and d (see Table 1). Functional TMEM16A can be one of the following combinations of the splice variants: ac, abc, acd, or the abcd isoform. There is not a known isoform lacking all splice variants that is a functional chloride channel. The nucleic acid and amino acid sequences of human TMEM16A are known, and have been published in, e.g., Caputo A. et al., Science, 24:322(5901)590-594 (2008). One of the isoforms (the full length amino acid sequence) corresponds to NP_060513.5 plus a 22 amino acid in-frame insert variant b (ANO1-007 ENSP00000433445) from the Ensembl database (see the website at http://uswest.ensembl.org/index.html). TMEM16A sequences in some other species are also known. For example, mouse TMEM16A (NM_178642, NP_848757, Gene ID 101772) and rat TMEM16A (NM_001107564, NP_848757, Gene ID 309135) have been published.

Structurally, a TMEM16A protein has eight transmembrane segments and cytosolic amino—and carboxy termini. TMEM16A also encompasses proteins that are a calcium activated chloride channel and have over its full length at least about 90%, 91%, 92%, 93%, 94%, 95%, 96%, 97%, 98%, 99% or 100% sequence identity with the amino acid sequence of SEQ ID NO:1 describe in Table 1 below. A TMEM16A nucleic acid sequence has over its full length at least about 90%, 91%, 92%, 93%, 94%, 95%, 96%, 97%, 98%, 99% or 100% sequence identity with the nucleic acid sequence of SEQ ID NO: 2 described in Table 1 below.

TABLE 1
Human TMEM16A Amino Acid and Nucleic Acid Sequences

```
Amino acid sequence of human TMEM16A(abcd).
(SEQ ID NO: 1, 1008 amino acids)
MRVNEKYSTLPAEDRSVHIINICAIEDIGYLPSEGTLLNSLSVDPDAECKYGLYFRDGRRKVDYILVYHHKRPSG
NRTLVRRVQHSDTPSGARSVKQDHPLPGKGASLDAGSGEPPMDYHEDDKRFRREEYEGNLLEAGLELERDEDTKI
HGVGFVKIHAPWNVLCREAEFLKLKMPTKKMYHINETRGLLKKINSVLQKITDPIQPKVAEHRPQTMKRLSYPFS
REKQHLFDLSDKDSFFDSKIRSTIVYEILKRTTCTKAKYSMGQGEGRKKDSALLSKRRKCGKYGITSLLANGVYA
AAYPLHDGDYNGENVEFNDRKLLYEEWARYGVFYKYQPIDLVRKYFGEKIGLYFAWLGVYTQMLIPASIVGIIVF
LYGCATMDENIPSMEMCDQRHNITMCPLCDKICSYWKMSSACATARASHLFDNPATVFFSVFMALWAATFMEHWK
RKQMRLNYRWDLTGFEEEEEAVKDHPRAEYEARVLEKSLKKESRNKEKRRHIPEESTNKWKQRVKTAMAGVKLTD
KVKLTWRDRFPAYLTNLVSIIFMIAVTFAIVLGVIIYRISMAAALAMNSSPSVRSNIRVTVTATAVIINLVVIIL
LDEVYGCIARWLTKIEVPKTEKSFEERLIFKAFLLKFVNSYTPIFYVAFFKGRFVGRPGDYVYIFRSFRMEECAP
GGCLMELCIQLSIIMLGKQLIQNNLFEIGIPKMKKLIRYLKLKQQSPPDHEECVKRKQRYEVDYNLEPFAGLTPE
YMEMIIQFGFVTLFVASFPLAPLFALLNNIIEIRLDAKKFVTELRRPVAVRAKDIGIWYNILRGIGKLAVIINAF
VISFTSDFIPRLVYLYMYSKNGTMHGFVNHTLSSFNVSDFQNGTAPNDPLDLGYEVQICRYKDYREPPWSENKYD
ISKDFWAVLAARLAFVIVFQNLVMFMSDFVDWVIPDIPKDISQQIHKEKVLMVELFMREEQDKQQLLETWMEKER
QKDEPPCNHHNTKACPDSLGSPAPSHAYHGGVL
Nucleic Acid Sequence of human TMEM16A
(SEQ ID NO: 2)
      1 aaaggcgggc cggctggcgt ccaagttcct gaccaggcgc gggccggccc gcgggaccag
    61 cagccgggtg gcggcgcgat cggccccgag aggctcaggc gccccccgca tcgagcgcgc
  121 gggccgggcg ggccagggcg gcgggcggag cgggaggcgg ccacgtcccc ggcgggcctg
  181 ggcgcgggga ggcccggccc cctgcgagcg cgccgcgaac gctgcggtct ccgcccgcag
  241 aggccgccgg ggccgtggat ggggagggcg cgccgcccgg cggtcccagc gcacaggcgg
  301 ccacgatgag ggtcaacgag aagtactcga cgctcccggc cgaggaccgc agcgtccaca
  361 tcatcaacat ctgcgccatc gaggacatcg gctacctgcc gtccgagggc acgctgctga
  421 actccttatc tgtggaccct gatgccgagt gcaagtatgg cctgtacttc agggacggcc
  481 ggcgcaaggt ggactacatc ctggtgtacc atcacaagag gccctcgggc aaccggaccc
  541 tggtcaggag ggtgcagcac agcgacaccc cctctggggc tcgcagcgtc aagcaggacc
  601 accccctgcc gggcaagggg gcgtcgctgg atgcaggctc gggggagccc ccgatggact
  661 accacgagga tgacaagcgc ttccgcaggg aggagtacga gggcaacctc ctggaggcgg
  721 gcctggagct ggagcgggac gaggacacta aaatccacgg agtcgggttt gtgaaaatcc
  781 atgccccctg gaacgtgctg tgcagagagg ccgagtttct gaaactgaag atgccgacga
  841 agaagatgta ccacattaat gagacccgtg gcctcctgaa aaaaatcaac tctgtgctcc
  901 agaaaatcac agatcccatc cagcccaaag tggctgagca caggccccag accatgaaga
  961 gactctccta tcccttctcc cgggagaagc agcatctatt tgacttgtct gataaggatt
1021 cctttttcga cagcaaaacc cggagcacga ttgtctatga gatcttgaag agaacgacgt
1081 gtacaaaggc caagtacagc atgggccaag gcgagggaag aaagaaggac tccgcccttc
1141 taagtaaaag gcggaaatgt gggaagtatg gcatcacgag cctgctggcc aatggtgtgt
1201 acgcggctgc atacccactg cacgatggag actacaacgg tgaaaacgtc gagttcaacg
1261 acagaaaact cctgtacgaa gagtgggcac gctatggagt tttctataag taccagccca
1321 tcgacctggt caggaagtat tttggggaga agatcggcct gtacttcgcc tggctgggcg
1381 tgtacaccca gatgctcatc cctgcctcca tcgtgggaat cattgtcttc ctgtacggat
1441 gcgccaccat ggatgaaaac atccccagca tggagatgtg tgaccagaga cacaatatca
1501 ccatgtgccc gctttgcgac aagacctgca gctactggaa gatgagctca gcctgcgcca
1561 cggcccgcgc cagccacctc ttcgacaacc ccgccacggt cttcttctct gtcttcatgg
1621 ccctctgggc tgccaccttc atggagcact ggaagcggaa acagatgcga ctcaactacc
1681 gctgggacct cacgggcttt gaagaggaag aggaggctgt caaggatcat cctagagctg
1741 aatacgaagc cagagtcttg gagaagtctc tgaagaaaga gtccagaaac aaagagaagc
1801 gccggcatat tccagaggag tcaacaaaca aatggaagca gagggttaag acagccatgg
1861 cgggggtgaa attgactgac aaagtgaagc tgacatggag agatcggttc ccagcctacc
1921 tcactaactt ggtctccatc atcttcatga ttgcagtgac gtttgccatc gtcctcggcg
1981 tcatcatcta cagaatctcc atggccgccg ccttggccat gaactcctcc ccctccgtgc
2041 ggtccaacat ccgggtcaca gtcacagcca ccgcagtcat catcaaccta gtggtcatca
2101 tcctcctgga cgaggtgtat ggctgcatag cccgatggct caccaagatc gaggtcccaa
2161 agacggagaa aagctttgag gagaggctga tcttcaaggc tttcctgctg aagtttgtga
2221 attcctacac ccccatcttt tacgtggcgt tcttcaaagg ccggtttgtt ggacgcccgg
2281 gcgactacgt gtacattttc cgttccttcc gaatggaaga gtgtgcgcca gggggctgcc
2341 tgatggagct atgcatccag ctcagcatca tcatgctggg gaaacagctg atccagaaca
2401 acctgttcga gatcggcatc ccgaagatga agaagctcat ccgctacctg aagctgaagc
2461 agcagagccc ccctgaccac gaggagtgtg tgaagaggaa acagcggtac gaggtggatt
2521 acaacctgga gcccttcgcg ggcctcaccc cagagtacat ggaaatgatc atccagtttg
2581 gcttcgtcac cctgtttgtc gcctccttcc ccctggcccc actgtttgcg ctgctgaaca
2641 acatcatcga gatccgcctg gacgccaaaa agtttgtcac tgagctccga aggccggtag
2701 ctgtcagagc caaagacatc ggaatctggt acaatatcct cagaggcatt gggaagcttg
2761 ctgtcatcat caatgccttc gtgatctcct tcacgtctga cttcatcccg cgcctggtgt
2821 acctctacat gtacagtaag aacgggacca tgcacggctt cgtcaaccac accctctcct
2881 ccttcaacgt cagtgacttc cagaacggca cggcccccaa tgaccccctg gacctgggct
2941 acgaggtgca gatctgcagg tataaagact accgagagcc gccgtggtcg gaaaacaagt
3001 acgacatctc caaggacttc tgggccgtcc tggcagcccg gctggcgttt gtcatcgtct
3061 tccagaacct ggtcatgttc atgagcgact ttgtggactg ggtcatcccg gacatcccca
3121 aggacatcag ccagcagatc cacaaggaga aggtgctcat ggtggagctg ttcatgcggg
3181 aggagcaaga caagcagcag ctgctggaaa cctggatgga gaaggagcgg cagaaggacg
3241 agccgccgtg caaccaccac aacaccaaag cctgcccaga cagcctcggc agcccagccc
3301 ccagccatgc ctaccacggg ggcgtcctgt agctatgcca gcggggctgg gcaggccagc
3361 cgggcatcct gaccgatggg caccctctcc cagggcaggc ggcttcccgc tcccaccagg
3421 gcccggtggg tcctgggttt tctgcaaaca tggaggacca ctttctgata ggacattttc
3481 ctttcttctt tctgttttct ttcccttgtt tttgcacaaa gccattatgc agggaatatt
3541 ttttaatctg tagtattcaa gatgaatcaa aatgatggct ggtaatacgg caataaggta
3601 gcaaaggcag gtgctttgca gaaagaatgc ttggaaactt gagtctccct agaggtgaaa
3661 agtgagcaga ggcccgtaga aaccctcctc tgaatcctcc taattcctta agatagatgc
3721 aaaatggtaa gccgaggcat cgcgcaaaag ctggtgcgat gcttcaggga aaatggaaaa
3781 cccacgcaag aataatgatt gattccggtt ccaaaaggtg tcacctacct gtttcagaaa
3841 agttagactt tccatcgcct tttccttcca tcagttgagt ggctgagaga gaagtgcctc
3901 atccctgagc cacacagggg gcgtgggagc atcccagtta tccctggaaa gctagaaggg
3961 gacagaggtg tccctgatta agcaggaaac agcacccttg gcgtccccag caggctcccc
4021 actgtcagcc acacacctgc ccccatcaca ccaagccgac ctcagagttg ttcatcttcc
4081 ttatgggaca aaaccggttg accagaaaat gggcagagag agatgacctg gaagcatttc
4141 cacagatggt gtcagggttt caagaagtct tagggcttcc aggggtcccc tggaagcttt
4201 agaatattta tgggtttttt tttcaaatat caattatatg gtagattgag gatttttttt
4261 ctgtagctca aaggtggagg gagtttatta gttaaccaaa tatcgttgag aggaatttaa
4321 aatactgtta ctaccaaaga tttttattaa taaaggctta tattttggta acacttctct
4381 atatttttac tcacaggaat gtcactgttg gacaattatt ttaaaagtgt ataaaaccaa
4441 gtctcataaa tgatatgagt gatctaaatt tgcagcaatg atactaaaca actctctgaa
4501 atttctcaag caccaagaga aacatcattt tagcaaaggc caggaggaaa aatagaaata
4561 aatttgtctt gaagatctca ttgatgtgat gttacattcc ctttaatctg ccaactgtgg
4621 tcaaagttca taggtgtcgt acatttccat tatttgctaa aatcatgcaa tctgatgctt
4681 ctcttttctc ttgtacagta agtagtttga agtgggtttt gtatataaat actgtattaa
4741 aaattaggca attaccaaaa atccttttat ggaaaccatt tttttaaaaa gtgaatgtac
4801 acaaatccac agaggactgt ggctggacat tcatctaaat aaatttgaat atacgacact
4861 tttctcactt gaaaaa
```

As used herein, “mutations” can refer to mutations in the CFTR gene or the CFTR protein. A “CFTR mutation” refers to a mutation in the CFTR gene, and a “CFTR mutation” refers to a mutation in the CFTR protein. A genetic defect or mutation, or a change in the nucleotides in a gene in general results in a mutation in the CFTR protein translated from that gene.

As used herein, co-crystal refers to crystalline materials composed of two or more different molecules, typically active pharmaceutical ingredient (API) and co-crystal formers (“coformers”), in the same crystal lattice.

As used herein, a “F508del mutation” or “F508del” is a specific mutation within the CFTR protein. The mutation is a deletion of the three nucleotides that comprise the codon for amino acid phenylalanine at position 508, resulting in CFTR protein that lacks this phenylalanine residue.

The term “CFTR gating mutation” as used herein means a CFTR mutation that results in the production of a CFTR protein for which the predominant defect is a low channel open probability compared to normal CFTR (Van Goor, F., Hadida S. and Grootenhuis P., “Pharmacological Rescue of Mutant CFTR function for the Treatment of Cystic Fibrosis”, Top. Med. Chem. 3: 91-120 (2008)). Gating mutations include, but are not limited to, G551D, G178R, S549N, S549R, G551S, G970R, G1244E, S1251N, S1255P, and G1349D.

As used herein, a patient who is “homozygous” for a particular mutation, e.g. F508del, has the same mutation on each allele.

As used herein, a patient who is “heterozygous” for a particular mutation, e.g. F508del, has this mutation on one allele, and a different mutation on the other allele.

As used herein, the term “modulator” refers to a compound that increases the activity or amount of a biological compound such as a protein. For example, a CFTR modulator is a compound that increases the activity or amount of CFTR. The increase in activity resulting from a CFTR modulator may be through a corrector mechanism or a potentiator mechanism as described below.

As used herein, the term “CFTR corrector” refers to a compound that increases the amount of functional CFTR protein at the cell surface, resulting in enhanced ion transport.

As used herein, the term “CFTR potentiator” refers to a compound that increases the channel activity of CFTR protein located at the cell surface, resulting in enhanced ion transport.

As used herein, the term “CFTR amplifier” refers to a compound that increases the amount of CFTR protein that the cell makes.

As used herein the term “CFTR” refers to Cystic Fibrosis transmembrane conductance regulator which is a protein kinase A (PKA)-activated epithelial anion channel involved in salt and fluid transport in multiple organs, including the lung.

As used herein the term “CFTR mediated disease” refers to a disease associated with either the reduction of the number of CFTR channels at the cell surface (e.g., synthesis or processing mutations) or impaired CFTR channel function (e.g., gating or conductance mutations) or both.

As used herein, the term “ENaC Inhibitor” refers to an inhibitor of the Epithelial Sodium Channel.

As used herein, the term “modulating” as used herein means increasing or decreasing by a measurable amount.

As used herein, the term “inducing,” as in inducing CFTR activity, refers to increasing CFTR activity, whether by the corrector, potentiator, or other mechanism.

As used herein, the term “mucociliary clearance (MCC)” refers to the primary innate defense mechanism of the lung. The functional components are the protective mucous layer, the airway surface liquid layer, and the cilia on the surface of ciliated cells.

As used herein, the term “onset of an endotherm” refers to the designed intersection point of the extrapolated baseline and the inflectional tangent at the beginning of the melting or crystallization peak. The baseline and the inflectional tangent are determined from the temperature-dependent heat flow signal. In the case of pure and homogeneous materials, the onset-temperature can be indicated as melting temperature.

As used herein, the term “metastable” refers to a crystalline form of a chemical system (i.e. anhydrate, hydrate, or solvate), in which at given environmental conditions (i.e. temperature, pressure, water or solvent activity) there exists at least one additional crystalline form which is thermodynamically more stable than the metastable form. A crystalline form is considered metastable if it can exist or be crystallized at the same environmental conditions but its transition into the most stable form is kinetically hindered (i.e. some activation energy is necessary for transformation into the thermodynamically more stable crystalline form).

As used herein “Asthma” includes both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g., of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.) Prophylactic efficacy in the treatment of asthma will be evidenced by reduced frequency or severity of symptomatic attack, e.g., of acute asthmatic or bronchoconstrictor attack, improvement in lung function or improved airways hyperreactivity. It may further be evidenced by reduced requirement for other, symptomatic therapy, i.e., therapy for or intended to restrict or abort symptomatic attack when it occurs, e.g., anti-inflammatory (e.g., cortico-steroid) or bronchodilatory. Prophylactic benefit in asthma may, in particular, be apparent in subjects prone to “morning dipping”. “Morning dipping” is a recognized asthmatic syndrome, common to a substantial percentage of asthmatics and characterized by asthma attack, e.g., between the hours of about 4-6 am, i.e., at a time normally substantially distant from any previously administered symptomatic asthma therapy.

A “patient,” “subject” or “individual” are used interchangeably and refer to either a human or non-human animal. The term includes mammals such as humans. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. Preferably, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder, refers to the management and care of a patient for the purpose of combating the disease, condition, or disorder and includes the administration of a compound of the present invention to prevent the onset of the symptoms or complications, alleviating the symptoms or complications, or eliminating the disease, condition or disorder.

As used herein, the terms “treatment,” “treating,” and the like generally mean the improvement of CF or its symptoms or lessening the severity of CF or its symptoms in a subject. “Treatment,” as used herein, includes, but is not limited to, the following: (i) to ameliorating the disease or disorder (i.e., slowing or arresting or reducing the development of the disease or at least one of the clinical symptoms thereof); (ii) to alleviating or ameliorating at least one physical parameter including those which may not be discernible by the patient; or (iii) to preventing or delaying the onset or development or progression of the disease or disorder. (iiii) increased growth of the subject, increased weight gain, reduction of mucus in the lungs, improved pancreatic and/or liver function, reduced cases of chest infections, and/or reduced instances of coughing or shortness of breath. Improvements in or lessening the severity of any of these conditions can be readily assessed according to standard methods and techniques known in the art.

As used herein, a subject is “in need of” a treatment if such subject would benefit biologically, medically or in quality of life from such treatment (preferably, a human).

As used herein the term “co-administer” refers to the presence of two active agents in the blood of an individual. Active agents that are co-administered can be concurrently or sequentially delivered.

The term “combination therapy” or “in combination with” or “pharmaceutical combination” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent being administered prior to, concurrent with, or sequentially to each other with no specific time limits. In each case, the treatment regimen will provide beneficial effects of the drug combination in treating the conditions or disorders described herein.

As used herein, the phrase “optionally substituted” is used interchangeably with the phrase “substituted or unsubstituted.” In general the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position.

As used herein, the term “C₁₋₆alkyl” refers to a fully saturated branched or unbranched hydrocarbon moiety having 1 to 6 carbon atoms. The terms “C₁₋₆alkyl”, “C₁₋₄alkyl” and “C₁₋₂ alkyl” are to be construed accordingly. Representative examples of C₁₋₆alkyl include, but are not limited to, methyl, ethyl, n-propyl, iso-propyl, n-butyl, sec-butyl, iso-butyl, tert-butyl, n-pentyl, isopentyl, neopentyl and n-hexyl. Similarly, the alkyl portion (i.e., alkyl moiety) of an alkoxy have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls). “Halo-substituted alkyl” refers to an alkyl group having at least one halogen substitution.

As used herein, the term “C₁₋₄ alkoxy” refers to an alkyl moiety attached through an oxygen bridge (i.e. a -O—C₁₋₄ alkyl group wherein C₁₋₄ alkyl is as defined herein).

Representative examples of alkoxy include, but are not limited to, methoxy, ethoxy, propoxy, 2-propoxy, butoxy, tert-butoxy and the like. Preferably, alkoxy groups have about 1-4 carbons, more preferably about 1-2 carbons.

As used herein, the term “C₁₋₄ alkoxy” refers to a fully saturated branched or unbranched hydrocarbon moiety having 1 to 4 carbon atoms. The term “C₁₋₂alkoxy” is to be construed accordingly.

“Halogen” or “halo” may be fluorine, chlorine, bromine or iodine (preferred halogens as substituents are fluorine and chlorine).

As used herein, the term “halo-substituted-C₁₋₄alkyl” or “halo-C₁₋₄alkyl” refers to a C₁₋₄alkyl group as defined herein, wherein at least one of the hydrogen atoms is replaced by a halo atom. The halo-C₁₋₄alkyl group can be monohalo-C₁₋₄alkyl, dihalo-C₁₋₄alkyl or polyhalo-C₁₋₄ alkyl including perhalo-C₁₋₄alkyl. A monohalo-C₁₋₄alkyl can have one iodo, bromo, chloro or fluoro within the alkyl group. Dihalo-C₁₋₄alkyl and polyhalo-C₁₋₄alkyl groups can have two or more of the same halo atoms or a combination of different halo groups within the alkyl.

Typically the polyhalo-C₁₋₄alkyl group contains up to 9, or 8, or 7, or 6, or 5, or 4, or 3, or 2 halo groups. Non-limiting examples of halo-C₁₋₄alkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-C₁₋₄alkyl group refers to a C₁₋₄alkyl group having all hydrogen atoms replaced with halo atoms.

As used herein, the term “halo-substituted-C₁₋₄alkoxy” or “halo-C₁₋₄alkoxy” refers to C₁₋₄ alkoxy group as defined herein above wherein at least one of the hydrogen atoms is replaced by a halo atom. Non-limiting examples of halo-substituted-C₁₋₄alkoxy include fluoromethoxy, difluoromethoxy, trifluoromethoxy, chloromethoxy, dichloromethoxy, trichloromethoxy, difluorochloromethoxy, dichlorofluoromethoxy, difluoroethoxy, difluoropropoxy, dichloroethoxy and dichloropropoxy and the like.

As used herein, the term “hydroxy-substituted-C₁₋₄alkyl” refers to a C₁₋₄alkyl group as defined herein, wherein at least one of the hydrogen atoms is replaced by a hydroxyl group.

The hydroxy-substituted-C₁₋₄alkyl group can be monohydroxy-C₁₋₄alkyl, dihydroxy-C₁₋₄alkyl or polyhydroxy-C₁₋₄alkyl including perhydroxy-C₁₋₄alkyl. A monohydroxy-C₁₋₄alkyl can have one hydroxyl group within the alkyl group. Dihydroxy-C₁₋₄alkyl and polyhydroxy-C₁₋₄alkyl groups can have two or more of the same hydroxyl groups or a combination of different hydroxyl groups within the alkyl. Typically the polyhydroxy-C₁₋₄alkyl group contains up to 9, or 8, or 7, or 6, or 5, or 4, or 3, or 2 hydroxy groups. Non-limiting examples of hydroxy substituted —C₁₋₄ alkyl include hydroxy-methyl, dihydroxy-methyl, pentahydroxy-ethyl, dihydroxyethyl, and dihydroxypropyl. A perhydroxy-C₁₋₄alkyl group refers to a C₁₋₄alkyl group having all hydrogen atoms replaced with hydroxy atoms.

The term “oxo” (═O) refers to an oxygen atom connected to a carbon or sulfur atom by a double bond. Examples include carbonyl, sulfinyl, or sulfonyl groups (—C(O)-, —S(O)— or -S(O)-) such as, a ketone, aldehyde, or part of an acid, ester, amide, lactone, or lactam group and the like.

The term “aryl or C₆₋₁₀ oaryl” refers to 6- to 10-membered aromatic carbocyclic moieties having a single (e.g., phenyl) or a fused ring system (e.g., naphthalene.). A typical aryl group is phenyl group.

The term “C₃₋₆ cycloalkyl” refers to a carbocyclic ring which is fully saturated (e.g., cyclopropyl, cyclobutyl, cyclopentyl, and cyclohexyl).

The term “C₄₋₆ heterocycle” refers to a monocyclic ring which is fully saturated which has 4 to 6 ring atoms which contains 1 or 2 heteroatoms, independently selected from sulfur, oxygen and/or nitrogen. A typical “C₄₋₆ heterocycle” group includes oxtanyl, tetrahydrofuranyl, dihydrofuranyl, 1,4-dioxanyl, morpholinyl, 1,4-dithianyl, piperazinyl, piperidinyl, 1,3-dioxolanyl, pyrrolinyl, pyrrolidinyl, tetrahydropyranyl, oxathiolanyl, dithiolanyl, 1,3-dioxanyl, 1,3-dithianyl, oxathianyl, thiomorpholinyl, thiomorpholinyl 1,1 dioxide, tetrahydro-thiopyran1,1-dioxide, 1,4-diazepanyl.

The term “fully or partially saturated heterocycle” refers to a nonaromatic ring that is either partially or fully saturated and may exist as a single ring, bicyclic ring (including fused heterocyclic rings) or a spiral ring. Unless specified otherwise, the heterocyclic ring is generally a 4- to 10-membered ring containing 1 to 4 heteroatoms (preferably 1, 2 or 3 heteroatoms) independently selected from sulfur, oxygen and/or nitrogen. A partially saturated heterocyclic ring also includes groups wherein the heterocyclic ring is fused to an aryl or heteroaryl ring (e.g., 2,3-dihydrobenzofuranyl, indolinyl (or 2,3-dihydroindolyl), 2,3-dihydrobenzothiophenyl, 2,3-dihydrobenzothiazolyl, 1,2,3,4-tetrahydroquinolinyl, 1,2,3,4-tetrahydroisoquinolinyl, 5,6,7,8-tetrahydropyrido[3,4-b]pyrazinyl). As used herein the term “spiral” or “spiro” means a two ring system wherein both rings share one common atom.

Examples of spiral rings include 2,6-diazaspiro[3.3]heptanyl, -oxa-6-azaspiro[3.3]heptane, 2 2,6-diazaspiro[3.3]heptane, 3-azaspiro[5.5]undecanyl, 3,9-diazaspiro[5.5]undecanyl, 7-azaspiro[3.5]nonane, 2,6-diazaspiro[3.4]octane, 8-azaspiro[4.5]decane, 1,6-diazaspiro[3.3]heptane, 5-azaspiro[2.5]octane, 4,7-diazaspiro[2.5]octane, 5-oxa-2-azaspiro[3.4]octane, 6-oxa-1-azaspiro[3.3]heptane, 3-azaspiro[5.5]undecanyl, 3,9-diazaspiro[5.5]undecanyl, and the like.

Partially saturated or fully saturated heterocyclic rings include groups such as epoxy, aziridinyl, azetidinyl, tetrahydrofuranyl, dihydrofuranyl, dihydropyridinyl, pyrrolidinyl, imidazolidinyl, imidazolinyl, 1 H-dihydroimidazolyl, hexahydropyrimidinyl, piperidinyl, piperazinyl, pyrazolidinyl, 2H-pyranyl, 4H-pyranyl, oxazinyl, morpholino, thiomorpholino, tetrahydrothienyl, tetrahydrothienyl 1,1-dioxide, oxazolidinyl, thiazolidinyl, 7-oxabicyclo[2.2.1]heptane, and the like.

The term “Fused heterocycle or 8 to 10 membered fused heterocycle” rings include fully or partially saturated groups such as 4,5,6,7-tetrahydro-3H-imidazo[4,5-c]pyridine, 8-azabicyclo[3.2.1]octan-3-ol, octahydropyrrolo[1,2-a]pyrazine, 5,6,7,8-tetrahydroimidazo[1,2-a]pyrazine, 3,8 diazabicyclo[3.2.1]octane, 8-oxa-3-azabicyclo[3.2.1]octane, 7-oxabicyclo[2.2.1]heptane, 1 H-pyrazole, 2,5-diazabicyclo[2.2.1]heptane, 5,6,7,8-tetrahydro-[1,2,4]triazolo[4,3-a]pyrazine or 3-azabicyclo[3.1.0]hexane. A partially saturated heterocyclic ring also includes groups wherein the heterocyclic ring is fused to an aryl or heteroaryl ring (e.g., 2,3-dihydrobenzofuranyl, indolinyl (or 2,3-dihydroindolyl), 2,3-dihydrobenzothiophenyl, 2,3-dihydrobenzothiazolyl, 1,2,3,4-tetrahydroquinolinyl, 1,2,3,4-tetrahydroisoquinolinyl, 5,6,7,8-tetrahydropyrido[3,4-b]pyrazinyl, and the like).

Unless otherwise stated, the term “heteroaryl” refers to aromatic moieties containing at least one heteroatom (e.g., oxygen, sulfur, nitrogen or combinations thereof) within a 5- to 6-membered aromatic ring system (e.g., pyrrolyl, pyridyl, pyrazolyl, indolyl, indazolyl, thienyl, furanyl, benzofuranyl, oxazolyl, imidazolyl, tetrazolyl, triazinyl, pyrimidyl, pyrazinyl, thiazolyl, and the like.) The phrase “pharmaceutically acceptable” indicates that the substance, composition or dosage form must be compatible chemically and/or toxicologically, with the other ingredients comprising a formulation, and/or the mammal being treated therewith.

Unless specified otherwise, the term “compounds of the present invention” refers to compounds of formula (I), as well as all stereoisomers (including diastereoisomers and enantiomers), rotamers, tautomers, isotopically labeled compounds (including deuterium substitutions), and inherently formed moieties (e.g., polymorphs, co-crystals, solvates and/or hydrates). When a moiety is present that is capable of forming a salt, then salts are included as well, in particular pharmaceutically acceptable salts.

As used herein, the term “a,” “an,” “the” and similar terms used in the context of the present invention (especially in the context of the claims) are to be construed to cover both the singular and plural unless otherwise indicated herein or clearly contradicted by the context. The use of any and all examples, or exemplary language (e.g. “such as”) provided herein is intended merely to better illuminate the invention and does not pose a limitation on the scope of the invention otherwise claimed.

In one Embodiment, there is provided a compound of the Examples as an isolated stereoisomer wherein the compound has one stereocenter and the stereoisomer is in the R configuration.

In one Embodiment, there is provided a compound of the Examples as an isolated stereoisomer wherein the compound has one stereocenter and the stereoisomer is in the S configuration.

In one Embodiment, there is provided a compound of the Examples as an isolated stereoisomer wherein the compound has two stereocenters and the stereoisomer is in the R R configuration.

In one Embodiment, there is provided a compound of the Examples as an isolated stereoisomer wherein the compound has two stereocenters and the stereoisomer is in the R S configuration.

In one Embodiment, there is provided a compound of the Examples as an isolated stereoisomer wherein the compound has two stereocenters and the stereoisomer is in the S R configuration.

In one Embodiment, there is provided a compound of the Examples as an isolated stereoisomer wherein the compound has two stereocenters and the stereoisomer is in the S S configuration.

In one Embodiment, there is provided a compound of the Examples, wherein the compound has 1 or 2 stereocenters, as a racemic mixture.

It is also possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

In one Embodiment, the invention relates to a compound of the formula (I) as defined herein, in free form. In another Embodiment, the invention relates to a compound of the formula (I) as defined herein, in salt form. In another Embodiment, the invention relates to a compound of the formula (I) as defined herein, in acid addition salt form. In a further Embodiment, the invention relates to a compound of the formula (I) as defined herein, in pharmaceutically acceptable salt form. In yet a further Embodiment, the invention relates to a compound of the formula (I) as defined herein, in pharmaceutically acceptable acid addition salt form. In yet a further Embodiment, the invention relates to any one of the compounds of the Examples in free form. In yet a further Embodiment, the invention relates to any one of the compounds of the Examples in salt form. In yet a further Embodiment, the invention relates to any one of the compounds of the Examples in acid addition salt form. In yet a further Embodiment, the invention relates to any one of the compounds of the Examples in pharmaceutically acceptable salt form. In still another Embodiment, the invention relates to any one of the compounds of the Examples in pharmaceutically acceptable acid addition salt form.

Furthermore, the compounds of the present invention, including their salts, may also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

Compounds of the invention, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

The compounds of the present invention, including salts, hydrates and solvates thereof, may inherently or by design form polymorphs.

Compounds of the present invention may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

The further optional reduction, oxidation or other functionalization of compounds of formula (I) may be carried out according to methods well known to those skilled in the art. Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T.

W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, and in H.-D. Jakubke and H. Jeschkeit, “Aminosauren, Peptide, Proteine” (Amino acids, Peptides, Proteins), Verlag Chemie, Weinheim, Deerfield Beach, and Basel 1982. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Salts of compounds of the present invention having at least one salt-forming group may be prepared in a manner known to those skilled in the art. For example, acid addition salts of compounds of the present invention are obtained in customary manner, e.g. by treating the compounds with an acid or a suitable anion exchange reagent. Salts can be converted into the free compounds in accordance with methods known to those skilled in the art. Acid addition salts can be converted, for example, by treatment with a suitable basic agent.

Any resulting mixtures of isomers can be separated on the basis of the physicochemical differences of the constituents, into the pure or substantially pure geometric or optical isomers, diastereomers, racemates, for example, by chromatography and/or fractional crystallization.

For those compounds containing an asymmetric carbon atom, the compounds exist in individual optically active isomeric forms or as mixtures thereof, e.g. as racemic or diastereomeric mixtures. Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a commercially available chiral HPLC column.

The invention further includes any variant of the present processes, in which the reaction components are used in the form of their salts or optically pure material. Compounds of the invention and intermediates can also be converted into each other according to methods generally known to those skilled in the art.

For illustrative purposes, the reaction schemes depicted below provide potential routes for synthesizing the compounds of the present invention as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### General Synthetic Methods

The following examples of compounds of the present invention illustrate the invention. Methods for preparing such compounds are described hereinafter.

### Abbreviations:

Abbreviations used are those conventional in the art or the following:

| | |
|---|---|
| Ac: Acetyl | Min(s): minute(s) |
| AcOH, HOAc: acetic acid | Me: methyl |
| ATP: adenosine 5′-triphosphate | DIEA: diethylisopropylamine |
| aq.: aqueous | DME: 1,4-dimethoxyethane |
| app. q: apparent quartet | EDTA: ethylenediamine tetraacetic acid |
| Ar: aromatic | HOBt: 1-hydroxy-7-azabenzotriazole |
| ADME: absorption, distribution, metabolism | m/z: mass to charge ratio |
| and excretion | |
| Mmol: millimol | HBTU 2-(1H-benzotriazol-1-yl)-1,1,3,3- |
| | tetramethyluronium hexafluorophosphate |
| BINAP: racemic 2,2′-bis(diphenylphosphino)- | Alloc: allyloxycarbonyl protecting group |
| 1,1′-binaphthyl | |
| BPR: backpressure regulator | M and mM: molar and millimolar |
| br: broad | mg: milligram |
| BSA: bovine serum albumin | EDCI: 1-ethyl-3-(3-dimethylaminopropyl)carbodiimide |
| BOP: (Benzotriazol-1- | PdCl₂(dppf)-CH₂Cl₂: 1,1′- |
| yloxy)tris(dimethylamino)phosphonium | Bis(diphenylphosphino)ferrocene- |
| hexaflurorophosphate | palladium(II)dichloride dichloromethane |
| | complex |
| DCC: dicyclohexylcarbodiimide | μL, mL and L: microliter, milliliter and liter |
| PyBOP: (Benzotriazol-1- | N: equivalent per liter |
| yloxy)tripyrrolidinophosphonium | |
| hexaflurorophosphate | |
| calc: calculated | n-BuLi: n-butyllitium |
| d: doublet; dd: doublet of doublets | NMR: nuclear magnetic resonance |
| DCM: dichloromethane | o/n: over night |
| Diox: Dioxane | PFA: perfluoroalkoxy (fluoropolymer) |
| DMF: N,N-dimethylformamide | ppm: parts per million |
| DMSO: dimethylsulfoxide | Ph: phenyl |
| DIPEA: N,N-diisopropylethylamine | q: quartet |
| dppp: 1,3-bis(diphenylphosphino)propane | rt: room temperature |
| ESI-MS: electrospray ionization mass | rpm: revolutions per minute |
| spectrometry | |
| Et and EtOAc: ethyl and ethyl acetate | s: singlet |
| FCC: flash column chromatography | SFC: supercritical fluid chromatography |
| HATU: O-(7-azobenzotriazol-1-yl)-1,1,3,3- | t: triplet |
| tetramethyluroniumhexafluorophosphate | |
| HOAt: 1-hydroxy-7-azabenzotriazole | TEA: triethylamine |
| HPLC: high pressure liquid chromatography | THF: tetrahydrofuran |
| h, hr: hour(s) | 2-MeTHF: 2-methyltetrahydrofuran |
| HRMS: high resolution mass spectrometry | TFA: trifluoroacetic acid |
| LC and LCMS: liquid chromatography and | HEK293: Human Embryonic Kidney 293 |
| liquid chromatography-mass spectrometry | cells |
| NMU: N-nitroso-N-methylurea | DMEM: Dulbecco's modified eagle |
| | medium |
| MeOH: methanol | wt: weight |
| HEPES: 4-(2-hydroxyethyl)-1- | TBME: tert-butyl methyl ether |
| piperazineethanesulfonic acid | |
| EGTA: ethylene glycol tetraacetic acid | TFAA: Trifluoroacetic acid |
| PBS: Phosphate Buffered Saline, pH 7.4 | UHP: urea-hydrogen peroxide |
| MS: mass | rac: racemic |
| m: multiplet | TLC: thin layer chromatography |
| Rt: retention time | |
| RM: reaction mixture | |

### Analytical Methods

Mass spectra were acquired on LC-MS, SEC-MS, or GC-MS systems using electrospray, chemical and electron impact ionization methods from a range of instruments of the following configurations: Agilent 1100 HPLC systems with an Agilent 6110 Mass Spectrometer [M+H]+ refers to protonated molecular ion of the chemical species.
NMR spectra were run on Bruker AVANCE 400 MHz or 500 MHz NMR spectrometers using ICON-NMR, under TopSpin program control. Spectra were measured at 298K, unless indicated otherwise, and were referenced relative to the solvent resonance.

### LC/MS:

The sample is dissolved in suitable solvent such as MeCN, DMSO or MeOH and is injected directly into the column using an automated sample handler. The analysis is performed using one of the following methods:

### HPLC Conditions:

MS Methods: Using Agilent 1100 HPLC systems with an Agilent 6110 Mass Spectrometer

Method LowpH v002

| | |
|---|---|
| Column | Phenomenex Gemini C18 50 × 4.6 mm, 3.0 μm |
| Column Temperature | 50° C. |
| Eluents | A: H₂O, B: methanol, both containing 0.1% TFA |
| Flow Rate | 1.0 mL/min |
| Gradient | 5% to 95% B in 2.0 min, 0.2 min 95% B |

Method 2 min low pH LC v003

| | |
|---|---|
| Column | Waters BEH C18 50 × 2.1 mm, 1.7 μm |
| Column Temperature | 50° C. |
| Eluents | A: H₂O, B: acetonitrile, both containing 0.1% |
| | TFA |
| Flow Rate | 0.8 mL/min |
| Gradient | 0.20 min 5% B; 5% to 95% B in 1.30 min, 0.25 |
| | min 95% B |

Method RXNMON Acidic

| | |
|---|---|
| Column | Sunfire C18 3.5 μm 3.0 × 30 mm |
| Column Temperature | 40° C. |
| Eluents | A: Water + 0.05% Trifluoroacetic Acid, B: ACN |
| Flow Rate | 2.0 mL/min |
| Gradient | 5% to 95% B in 2.0 min |

Method RXNMON Basic

| | |
|---|---|
| Column | XBridge C18 3.5 μm 3.0 × 30 mm |
| Column Temperature | 40° C. |
| Eluents | A: Water + 5 mM Ammonium Hydroxide, |
| | B: ACN |
| Flow Rate | 2.0 mL/min |
| Gradient | 5% to 95% B in 2.0 min |

Method RXNMON Acidic NonPolar

| | |
|---|---|
| Column | Sunfire C18 3.5 μm 3.0 × 30 mm |
| Column Temperature | 40° C. |
| Eluents | A: Water + 0.05% Trifluoroacetic Acid, |
| | B: ACN |
| Flow Rate | |
| Gradient | 40% to 98% B in 2.0 min |

Method 8 minLowpHv01

| | |
|---|---|
| Column | Acquity CSH C18 100 × 2.1 mm |
| Column Temperature | 50° C. |
| Eluents | A: H₂O, B: acetonitrile, both containing 0.1% |
| | formic acid |
| Flow Rate | 0.7 mL/min |
| Gradient | 0.0 min 2% B; 2% to 98% B in 6.20 min, 1.0 |
| | min 98% B |

Method Product Analysis Acidic

| | |
|---|---|
| Column | ACQUITY UPLC BEH C18, 130 Å, 1.7 μm, |
| | 2.1 mm × 50 mm |
| Column Temperature | 50° C. |
| Eluents | A: Water + 0.1% Formic Acid, B: ACN |
| Flow Rate | 2.0 mL/min |
| Gradient | 2% to 98% B in 5.0 min |

Method Product Analysis Basic

| | |
|---|---|
| Column | ACQUITY UPLC BEH C18, 130 Å, 1.7 μm, |
| | 2.1 mm × 50 mm |
| Column Temperature | 50° C. |
| Eluents | A: Water + 5 mM Ammonium Hydroxide, |
| | B: ACN |
| Flow Rate | 2.0 mL/min |
| Gradient | 2% to 98% B in 5.0 min |

### SFC Method 1

### Cosolvent: 40% EtOH

### Column: Lux Cellulose-4 30×250 mm

### Detection: UV @ 260 nm

Flow rate: 80 g/minute

### BPR Set Point: 125 bar

### Injection Size: 50 mg

### SFC Method 2

### Cosolvent: 40% MeOH 10 mM NH₄OH

### Column: IC 21×250 mm

### Detection: UV @ 205 nm

Flow rate: 80 g/minute

### BPR Set Point: 100 bar

### SFC Method 3

### Cosolvent: 40% EtOH

### Column: IA 21×250 mm 5 um

### Detection: UV @ 270 nm

Flow rate: 80 g/minute

### BPR Set Point: 125 bar

### Injection Size: 50 mg

### SFC Method 4

Cosolvent: 5-55% MeOH with 10 mM MH40H

### Column: Lux Cellulose-2 4.6×100 mm 5 μm

### Detection: UV @ 250 nm

Flow rate: 5 mL/minute

### SFC Method 5

### Column: IB 21×250 mm

Flow rate: 80 g per minute

### Cosolvent: 15% MeOH 10 mM NH40H

### Detection: 260 nm

### BPR Set Point: 125 bar

### Injection Size: 12 mg

### SFC Method 6

### Column: Chiralpak IB 21×250 mm

Flow rate: 80 g per minute

### Cosolvent: 20% MeOH

### Detection: 254 nm

### BPR Set Point: 125 bar

### Injection Size: 11 mg

Prep HPLC Method 1

| | |
|---|---|
| Column | X-Bridge 30 × 50 mm 5 um column |
| Eluent | A: Aqueous 5 mM NH4OH, B: ACN |
| Flow Rate | 75 ml/min |
| Injection Size: | 1.5 ml injection) |
| Prep HPLC Method 2: | (Low pH 20-50% B formic acid) |

### EXPERIMENTAL

### Preparation of Intermediates

Intermediate 1 of the present invention may be prepared according to Scheme 1.

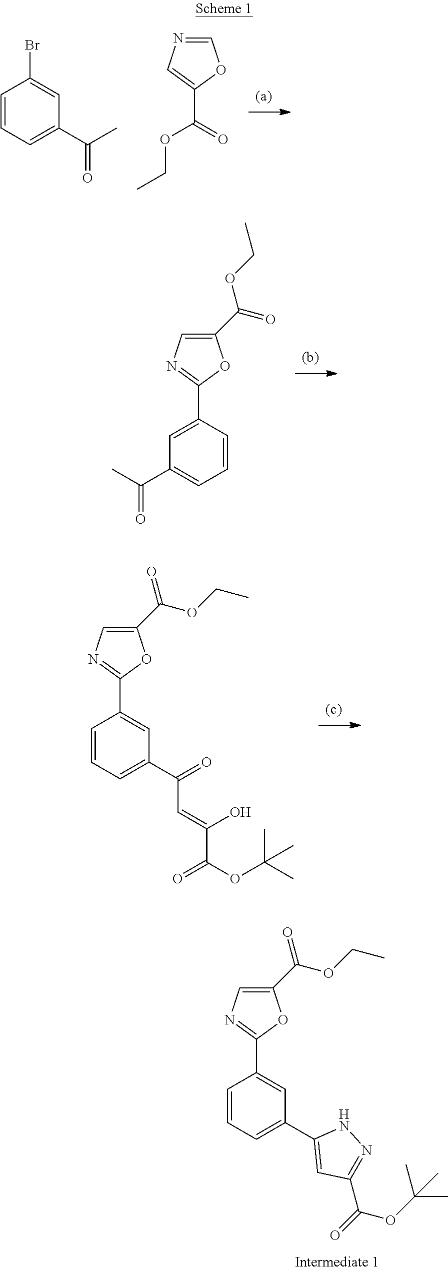

Step (a) involves C-H insertion reaction of oxazole to haloaromatic in a suitable solvent such as DME, DMA, DMF, THE or toluene in the presence of a suitable palladium catalyst such as Pd(OAc)2 or Pd2(dba)3 and ligand such as Xphos, Sphos, cy-JohnPhos or RuPhos or by using commercially available pre-formed palladium ligand adduct catalysts such as Xphos-Pd-G1, G2 or G3, RuPhos-Pd -G1,G2, G3 in the presence of pivalic acid and suitable base such as Cs2CO3 with heating under inert atmosphere.
Step (b) involves deprotonation with strong base such as LiHMDS or LDA in THE at low temperature followed by addition of di-tert-butyl oxalate to give the tert-butyl enoyl acetate which is used crude for the following step.
Step (c) involves formation of the pyrazole ring by treatment of the tert-butyl enoyl acetate intermediate with hydrazine hydrate followed by heating with acetic acid.

### Intermediate 1:

Ethyl 2-(3-(3-(tert-butoxycarbonyl)-1H-pyrazol-5-yl)phenyl)oxazole-5-carboxylate

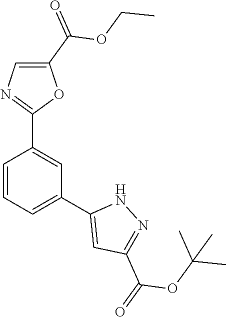 [SMILES: CCOC(=O)c1cnc(-c2cccc(-c3cc(C(=O)OC(C)(C)C)n[nH]3)c2)o1]

Step 1: Ethyl 2-(3-acetylphenyl)oxazole-5-carboxylate

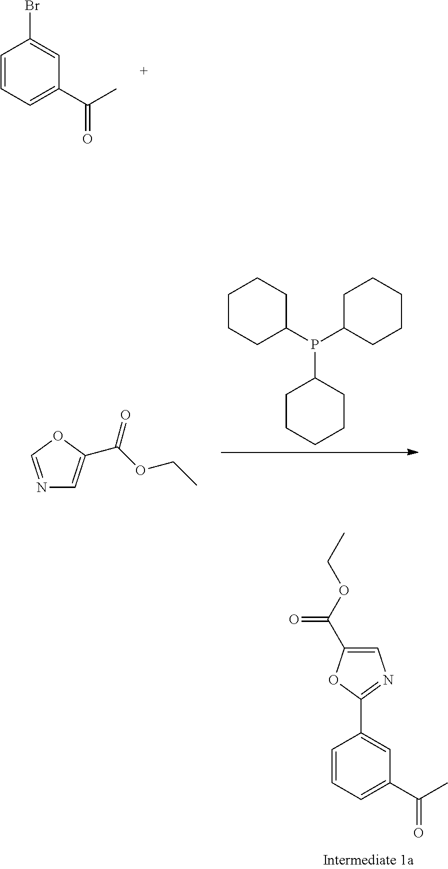

Pivalic acid (24.8 mL, 221 mmol) was added to a solution of ethyl oxazole-5-carboxylate [CAS118994-89-1](78.0 g, 553 mmol) and 1-(3-bromophenyl)ethanone [CAS 2142-63-4](110 g, 553 mmol) in Dioxane (1.4 L) under nitrogen. To this solution K₂CO₃ (229 g, 1659 mmol) was added followed by Tricyclohexylphosphine (10.8 g, 38.71 mmol) and Pd(OAc)₂ (4.7 g, 6.98 mmol). The RM was warmed to 110° C. and stirred for 16 h. The progress of the reaction was monitored by TLC (30% ethyl acetate in pet ether) which indicated complete consumption of the starting materials. The RM was filtered through celite and diluted with water and EtOAc (2×200 mL). The organic layers were dried over Na₂SO₄, filtered, and concentrated to afford 136.0 g (67%) of ethyl 2-(3-acetylphenyl)oxazole-5-carboxylate Intermediate 1a: as an off-white solid.
LCMS Rt: 1.19 mins MS m/z; 260.3 [M+H]+ 2minLowpH_v3
Step 2: (Z)-ethyl 2-(3-(4-(tert-butoxy)-3-hydroxy-4-oxobut-2-enoyl)phenyl)oxazole-5-carboxylate

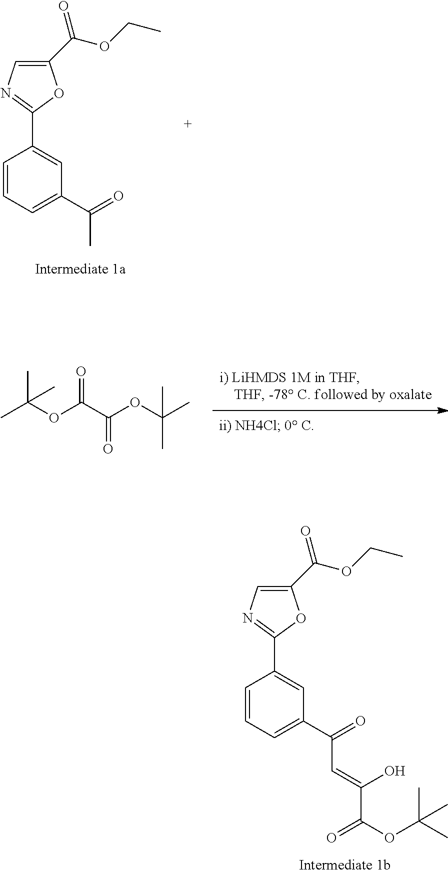 [SMILES: CCOC(=O)c1cnc(-c2cccc(C(C)=O)c2)o1 ; CC(C)(C)OC(=O)C(=O)OC(C)(C)C ; CCOC(=O)c1cnc(-c2cccc(C(=O)/C=C(\O)C(=O)OC(C)(C)C)c2)o1]

To a stirred solution of ethyl 2-(3-acetylphenyl)oxazole-5-carboxylate Intermediate 1a (40 g, 154.4 mmol) in THF (320 mL), LiHMDS (1M in THF) (183.7 mL, 183.7 mmol) was added at -78° C. over 1 h. The RM was kept at −78° C. for 30 min. Di-tert-butyl oxalate [CAS 691-64-5](40.24 g, 199.1 mmol) in THE (100 mL) was added over 30 min while maintaining the internal temperature below −70° C. The resulting solution was stirred at 10° C. for 1 h. The progress of the reaction was monitored by TLC (20% ethyl acetate in petroleum ether) which indicated complete consumption of the ethyl 2-(3-acetylphenyl)oxazole-5-carboxylate. The reaction mixture was quenched with sat. NH₄Cl (300 ml) and extracted with ethyl acetate (300 mL×3). The combined organic layers were dried over Na₂SO₄ and concentrated to afford 215 g (87%) of (Z)-ethyl 2-(3-(4-(tert-butoxy)-3-hydroxy-4-oxobut-2-enoyl)phenyl)oxazole-5-carboxylate Intermediate 1 b; as a brown oil which was used crude for the next step. LCMS Rt: 1.57 mins MS m/z; 388.4 [M+H]+ 2minLowpH_v3
Step 3: Ethyl 2-(3-(3-(tert-butoxycarbonyl)-1 H-pyrazol-5-yl)phenyl)oxazole-5-carboxylate

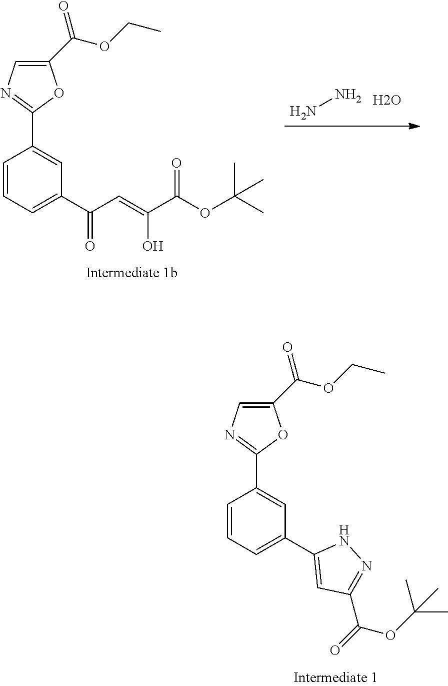

Hydrazine hydrate (9.4 mL, 168.2 mmol) was added to a stirred solution of (Z)-ethyl 2-(3-(4-(tert-butoxy)-3-hydroxy-4-oxobut-2-enoyl)phenyl)oxazole-5-carboxylate (60.0 g, 155.0 mmol) in ethanol (500 mL) and the RM was cooled to 10° C. Acetic acid (23.16 mL, 386 mmol) was added drop wise over 30 min then the temperature was raised to 70° C. and the RM stirred for 1 h. The progress of the reaction was monitored by TLC (20% ethyl acetate in pet ether) which indicated complete consumption of (Z)-ethyl 2-(3-(4-(tert-butoxy)-3-hydroxy-4-oxobut-2-enoyl)phenyl)oxazole-5-carboxylate. The RM was concentrated to give crude material which was added to sat. NaHCO₃ and extracted with ethyl acetate (300 mL×3). The organic layers were sequentially washed with water and brine, dried over Na₂SO₄, filtered, and concentrated. The crude material was purified by FCC (0-30% ethyl acetate/petroleum ether) to afford 25.1 g (47%) of ethyl 2-(3-(3-(tert-butoxycarbonyl)-1 H-pyrazol-5-yl)phenyl)oxazole-5-carboxylate Intermediate 1; as an off-white solid.
LCMS Rt: 1.53 mins MS m/z; 384.2 [M+H]+ 2minLowpH_v3 ¹H NMR (400 MHz, DMSO-d₆) δ ppm 8.52 (s, 1H) 8.17 (s, 1H) 8.09 (br d, J=7.82 Hz, 1H) 8.03 (br d, J=7.82 Hz, 1H) 7.67 (br t, J=7.83 Hz, 1H) 7.30 (s, 1H) 4.39 (q, J=7.01 Hz, 2H) 1.57 (s, 9H) 1.35 (t, J=7.09 Hz, 3H)
Intermediate 2 of the present invention may be prepared according to Scheme 2.

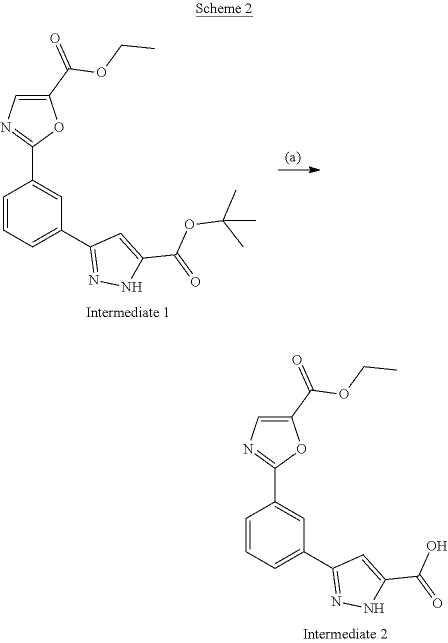 [SMILES: CCOC(=O)c1cnc(-c2cccc(-c3cc(C(=O)OC(C)(C)C)[nH]n3)c2)o1 ; CCOC(=O)c1cnc(-c2cccc(-c3cc(C(=O)O)[nH]n3)c2)o1]

Step (a) of Scheme 2 involves removal of the tert-butyl ester to give a carboxylic acid using a suitable acid such as HCl or TFA in a solvent such as DCM or dioxane.

### Intermediate 2: 3-(3-(5-(ethoxycarbonyl)oxazol-2-yl)phenyl)-1H-pyrazole-5-carboxylic acid

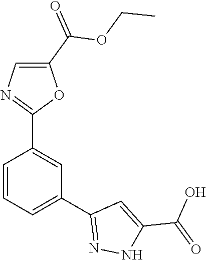 [SMILES: CCOC(=O)c1cnc(-c2cccc(-c3cc(C(=O)O)[nH]n3)c2)o1]

TFA (4.02 mL, 52.2 mmol) was added slowly to a stirred solution of ethyl 2-(3-(5-(tert-butoxycarbonyl)-1 H-pyrazol-3-yl)phenyl)oxazole-5-carboxylate Intermediate 1, 1 g, 2.61 mmol) in DCM (10 mL) and the RM was monitored by LCMS. After 3.5 hours the RM was concentrated to afford 3-(3-(5-(ethoxycarbonyl)oxazol-2-yl)phenyl)-1 H-pyrazole-5-carboxylic acid Intermediate 2 in quantitative yield.

LCMS Rt: 0.87 mins MS m/z; 328.3 [M+H]+ RXNMON-Acidic

Intermediate 3 of the present invention may be prepared according to Scheme 3.

¹H NMR (400 MHz, DMSO-d₆) δ ppm 8.52 (s, 1H) 8.06-8.16 (m, 1H) 8.03 (br d, J=7.58 Hz, 1H) 7.67 (s, 1H) 7.29 (s, 1H) 1.57 (s, 9H)

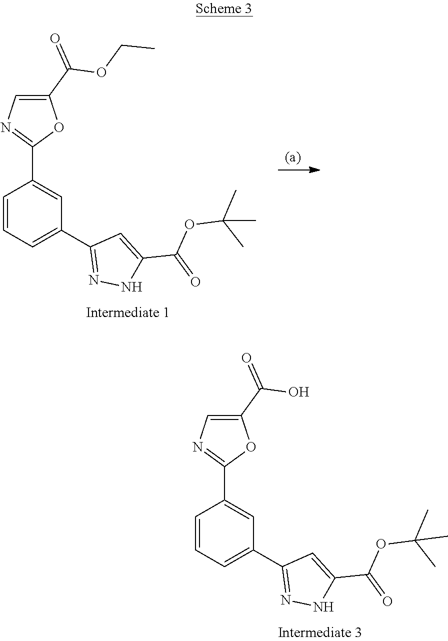 [SMILES: CCOC(=O)c1cnc(-c2cccc(-c3cc(C(=O)OC(C)(C)C)[nH]n3)c2)o1 ; CC(C)(C)OC(=O)c1cc(-c2cccc(-c3ncc(C(=O)O)o3)c2)n[nH]1]

Step (a) of Scheme 3 involves conversion of the ethyl ester of Intermediate 1 to a carboxylic acid using a suitable base such as NaOH, KOH or KOTMS in THF, methanol or water.

Intermediate 3: 2-(3-(3-(tert-butoxycarbonyl)-1 H-pyrazol-5-yl)phenyl)oxazole-5-carboxylic acid

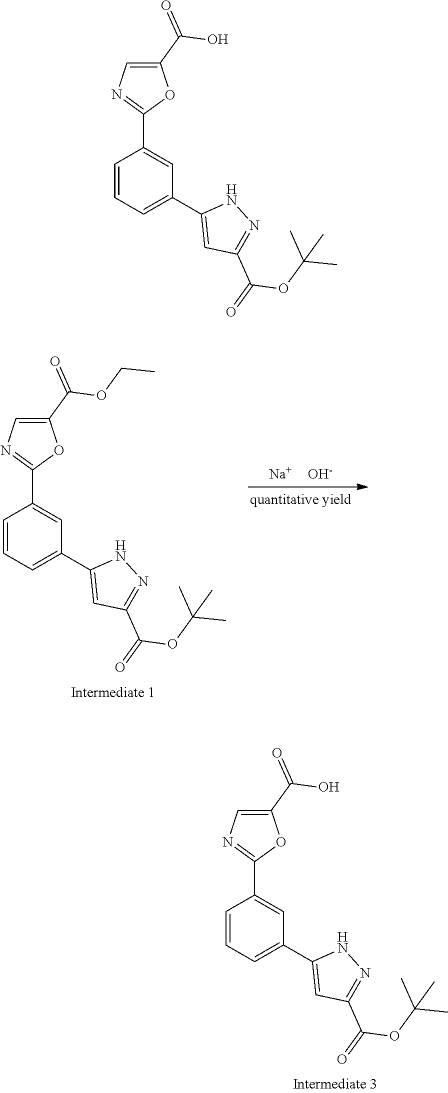

To a suspension of ethyl 2-(3-(3-(tert-butoxycarbonyl)-1 H-pyrazol-5-yl)phenyl)oxazole-5-carboxylate Intermediate 1 (15.38 g, 40.1 mmol) in ethanol (100 mL) was added a solution of NaOH (3.21 g, 80 mmol) in water (40 mL) at room temperature. The RM rapidly became a clear yellow-orange solution. After 45 min added 150 mL of 10% aqueous citric acid to bring to pH 2. The resulting precipitate was filtered washing with water and dried to afford a quantitative yield of 2-(3-(3-(tert-butoxycarbonyl)-1 H-pyrazol-5-yl)phenyl)oxazole-5-carboxylic acid Intermediate 3.

LCMS Rt: 0.90 mins MS m/z; 356.3 [M+H]+ RXNMON-Acidic

¹H NMR (400 MHz, DMSO-d₆) δ ppm 8.53 (t, J=1.52 Hz, 1H) 8.16 (s, 1H) 8.08 (dt, J=7.83, 1.39 Hz, 1H) 8.03 (dt, J=8.08, 1.26 Hz, 1H) 7.66 (t, J=7.83 Hz, 1H) 7.34 (s, 1H) 4.38 (q, J=7.07 Hz, 2H) 1.35 (t, J=7.07 Hz, 3H)

Intermediate 4 of the present invention may be prepared according to Scheme 4.

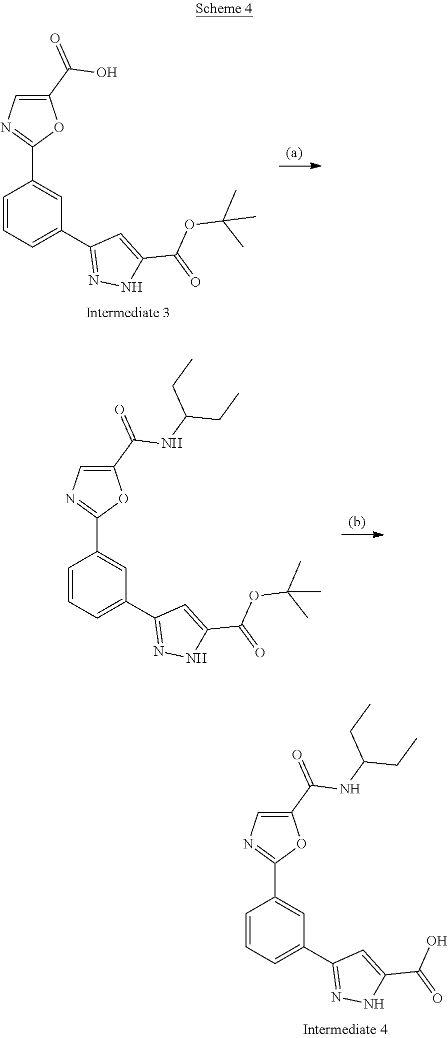 [SMILES: CC(C)(C)OC(=O)c1cc(-c2cccc(-c3ncc(C(=O)O)o3)c2)n[nH]1 ; CCC(CC)NC(=O)c1cnc(-c2cccc(-c3cc(C(=O)OC(C)(C)C)[nH]n3)c2)o1 ; CCC(CC)NC(=O)c1cnc(-c2cccc(-c3cc(C(=O)O)[nH]n3)c2)o1]

Step (a) involves reaction of an amine with Intermediate 3 in a suitable solvent such as DMF or ethyl acetate with a suitable base such as diisopropylethylamine or triethylamine and an amide coupling reagent such as T3P, HCTU, or pyBOP.
Step (b) of Scheme 4 involves removal of the tert-butyl ester to give a carboxylic acid using a suitable acid such as HCl or TFA in a solvent such as DCM or dioxane.

### Intermediate 4: 5-(3-(5-(pentan-3-ylcarbamoyl)oxazol-2-yl)phenyl)-1H-pyrazole-3-carboxylic acid

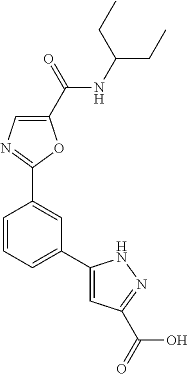 [SMILES: CCC(CC)NC(=O)c1cnc(-c2cccc(-c3cc(C(=O)O)n[nH]3)c2)o1]

Step 1: Tert-butyl 5-(3-(5-(pentan-3-ylcarbamoyl)oxazol-2-yl)phenyl)-1 H-pyrazole-3-carboxylate

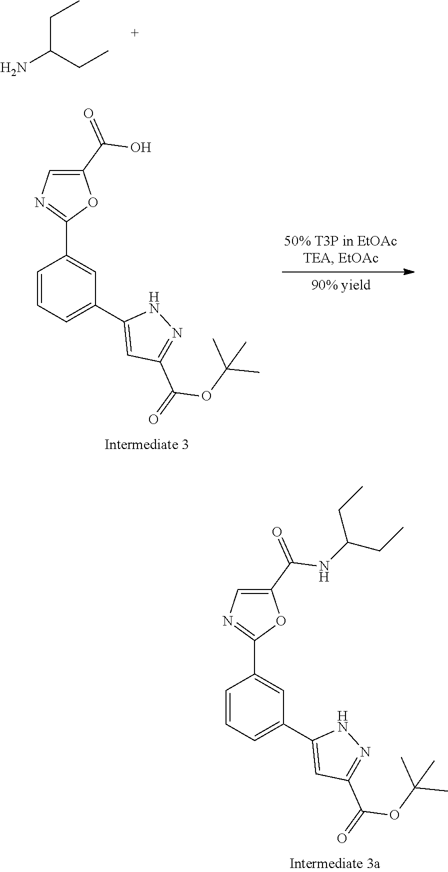

2-(3-(3-(tert-butoxycarbonyl)-1 H-pyrazol-5-yl)phenyl)oxazole-5-carboxylic acid Intermediate 3 (20 g, 56.3 mmol) was stirred in EtOAc (200 mL) to give a fine suspension. TEA (23.53 mL, 169 mmol) and 3-pentylamine (14.43 mL, 124 mmol) were added. T3P (50% in EtOAc) (49.7 mL, 84 mmol) was added drop wise and the RM was stirred overnight at room temp. The reaction mixture was quenched by addition of 5% citric acid (300 mL) and stirred for 20 min at room temp. The aqueous layer was washed with EtOAc. The combined organic layers were washed in sequence with water, 1 N NaOH, water again, and then with brine. The EtOAc was dried over Na2SO4 and concentrated to afford 21.6 g (86%) of tert-butyl 5-(3-(5-(pentan-3-ylcarbamoyl)oxazol-2-yl)phenyl)-1 H-pyrazole-3-carboxylate Intermediate 3a.
LCMS Rt: 1.12 mins MS m/z; 425.2 [M+H]+ RXNMON-Acidic
Step 2: 5-(3-(5-(pentan-3-ylcarbamoyl)oxazol-2-yl)phenyl)-1 H-pyrazole-3-carboxylic acid

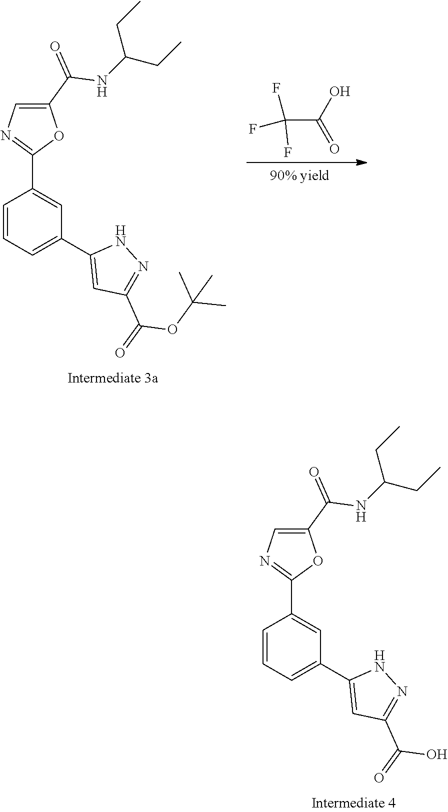 [SMILES: CCC(CC)NC(=O)c1cnc(-c2cccc(-c3cc(C(=O)OC(C)(C)C)n[nH]3)c2)o1 ; O=C(O)C(F)(F)F ; CCC(CC)NC(=O)c1cnc(-c2cccc(-c3cc(C(=O)O)n[nH]3)c2)o1]

To a stirred suspension of tert-butyl 5-(3-(5-(pentan-3-ylcarbamoyl)oxazol-2-yl)phenyl)-1 H-pyrazole-3-carboxylate Intermediate 3a (21.6 g, 50.9 mmol) in dichloromethane (150 mL) was added TFA (40 mL, 519 mmol). The RM was stirred 18 h and monitored by LCMS. The RM was concentrated to give a yellow solid. The solid was suspended in a 50/50 EtOAc/water mixture. A solution of 1 ON NaOH (55 mL) was slowly stirred in to dissolve the crude product in the aqueous layer (pH 10). The EtOAc layer was removed and 25 mL of 6 N HCl was added to the aqueous with good stirring to obtain a white precipitate which was filtered washing with water and dried to afford 16.89 g (90% yield) of 5-(3-(5-(pentan-3-ylcarbamoyl)oxazol-2-yl)phenyl)-1 H-pyrazole-3-carboxylic acid Intermediate 4 as a white solid.
LCMS Rt: 0.84 mins MS m/z; 369.5 [M+H]+ RXNMON-Acidic
1H NMR (400 MHz, DMSO-d₆) δ ppm 14.04 (br s, 1H) 13.46 (br s, 1H) 8.56 (br s, 1H) 8.29 (br d, J=8.56 Hz, 1H) 8.11 (br d, J=7.58 Hz, 1H) 8.05 (br d, J=7.09 Hz, 1H) 7.91 (s, 1H) 7.65 (br t, J=7.34 Hz, 1H) 7.33 (br s, 1H) 3.72-3.85 (m, 1H) 1.55-1.66 (m, 2H) 1.44-1.54 (m, 2H) 0.89 (t, J=7.34 Hz, 6H).
Intermediate 5 of the present invention may be prepared according to Scheme 5.

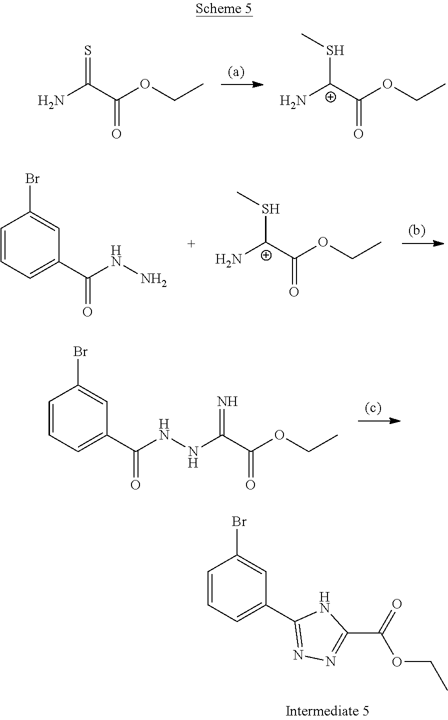

Step (a) involves alkylation of a commercially available thioamide with a reagent such as trimethyloxonium tetrafluoroborate as a suitable temperature such as 0° C.
Step (b) of involves reaction of the alkylated material with 3-bromobenzohydrazide in a suitable solvent such as DCM.
Step (c) of involves heating of the intermediate iminoacetate in a solvent such as NMP or EtOH to a suitable temperature such as 120° C. or 180° C. to provide triazole Intermediate 5.

### Intermediate 5: Ethyl 5-(3-bromophenyl)-4H-1,2,4-triazole-3-carboxylate

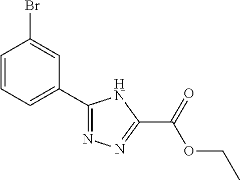 [SMILES: CCOC(=O)c1nnc(-c2cccc(Br)c2)[nH]1]

Step 1: Ethyl 2-(2-(3-bromobenzoyl)hydrazinyl)-2-iminoacetate

To a solution of ethyl carbamothioylformate (250 g, 1.88 mol) in dichloromethane (6.25 L) was added trimethyloxonium tetrafluoroborate (306 g, 2.07 mol) in several batches at 0° C. The resulting solution was stirred for 48 h at room temperature. 3-Bromobenzohydrazide (213 g, 990.48 mmol) was added to the RM followed by dropwise addition of TEA (247 g, 2.44 mol) dropwise with stirring at 0° C. The RM was stirred for 4 h at 40° C. and then cooled to room temperature. The resulting solids were collected by filtration and washed with 2 L of DCM to afford 235 g (40%) of ethyl 2-[(3-bromophenyl)formohydrazido]-2-iminoacetate as a white solid. LCMS Rt: 0.86 mins MS m/z; 316.2 [M+H]+ RXNMON-Acidic

¹H NMR (400 MHz DMSO-d₆, ppm): δ10.11 (s, 1H), 8.02 (s, 1H), 7.89-7.79 (m, 1H), 7.78-7.69 (m, 1H), 7.50-7.40 (m, 1H), 6.83 (br. s., 2H), 4.26 (q, J=7.1 Hz, 2H), 1.29 (t, J=7.1 Hz, 3H)

Step 2: Ethyl 5-(3-bromophenyl)-4H-1,2,4-triazole-3-carboxylate
In a 5-L pressure tank reactor under nitrogen, was placed ethyl 2-[(3-bromophenyl)formohydrazido]-2-iminoacetate (235 g, 748.09 mmol) in NMP (2.35 L). The resulting solution was stirred for 2 h at 180° C. and then cooled to room temperature. The solution was diluted with 6 L of EtOAc and washed with 4×2 L of brine. The mixture was dried over sodium sulfate and concentrated. The crude material was purified by FCC (1:3 ethyl acetate:petroleum ether) to afford 50.99 g (23%) of ethyl 5-(3-bromophenyl)-4H-1,2,4-triazole-3-carboxylate Intermediate 5; as a white solid.
LCMS Rt: 1.22 mins MS m/z; 297.8 [M+H]+ RXNMON-Acidic

¹H NMR (400 MHz DMSO-d₆, ppm): δ15.28-15.11 (s, 1H), 8.20 (s, 1H), 8.05-8.03 (m, 1H), 7.73-7.71 (d, J=6 Hz, 1H), 7.54-7.49 (m, 1H), 4.41-4.34 (m, 2H), 1.36-1.31 (m, 3H).

Intermediate 6 of the present invention may be prepared according to Scheme 6.

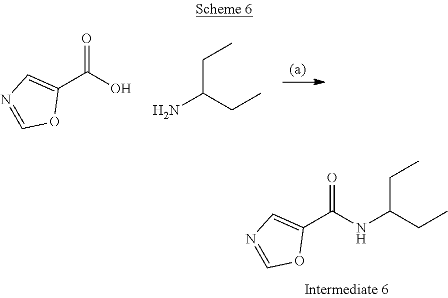

Step (a) involves amide formation in a suitable solvent such as DMF or ethyl acetate with a suitable base such as diisopropylethylamine or triethylamine and an amide coupling reagent such as T3P, pyBOP, or HATU to give Intermediate 6.
Intermediate 6:_N-(pentan-3-yl)oxazole-5-carboxamide

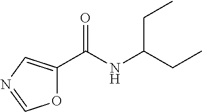

A solution of oxazole-5-carboxylic acid (3 g, 26.5 mmol) in dry DMF (30 ml), was treated with triethylamine (8.88 mL, 63.7 mmol), HATU (12.11 g, 31.8 mmol) and then pentan-3-amine (6.18 mL, 53.1 mmol). The reaction was diluted with water and EtOAc and the aqueous was extracted twice with 4:1 EtOAc:heptane. The organics were combined, washed with water (3×) and brine (1×) and then dried over Na2SO4. The crude material was purified by FCC (0-100% EtOAc in heptane) to give 0.8 g of N-(pentan-3-yl)oxazole-5-carboxamide as a yellow crystalline solid.

¹H NMR (400 MHz, CHLOROFORM-d) d=7.91 (s, 1H), 7.73 (s, 1H), 5.99-5.90 (m, 1H), 4.05-3.94 (m, 1H), 1.75-1.62 (m, 2H), 1.54-1.44 (m, 2H), 0.97 (t, J=7.5 Hz, 6H).

### PREPARATION OF EXAMPLES

### Example 1 of the Present Invention May be Prepared According to Scheme 7

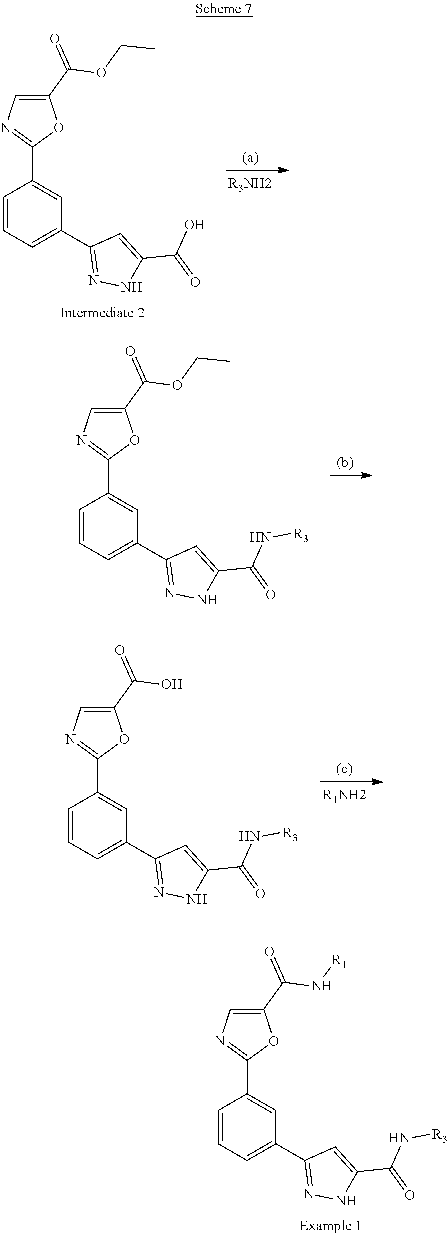

Step (a) involves reaction of an amine(R₃NH2) with Intermediate 2 in a suitable solvent such as DMF or ethyl acetate with a suitable base such as diisopropylethylamine or triethylamine and an amide coupling reagent such as T3P or pyBOP.
Step (b) of Scheme 6 involves conversion of the ethyl ester to a carboxylic acid using a suitable base such as NaOH, KOH or KOTMS in a solvent such as THF, methanol or water.
Step (c) involves reaction of an amine(R₁NH2) with the free acid in a suitable solvent such as DMF or ethyl acetate with a suitable base such as diisopropylethylamine or triethylamine and an amide coupling reagent such as T3P, HOPO/DIC, or pyBOP.

### Example 1.0: (S)-Ethyl 2-(2-(3-(3-(((S)-1-Cyclopropylethyl)Carbamoyl)-1H-Pyrazol-5-Yl)Phenyl)Oxazole-5-Carboxamido)-3-Methylbutanoate

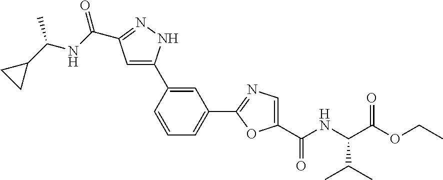 [SMILES: CCOC(=O)[C@@H](NC(=O)c1cnc(-c2cccc(-c3cc(C(=O)N[C@@H](C)C4CC4)n[nH]3)c2)o1)C(C)C]

Step 1: (S)-ethyl 2-(3-(5-((1-cyclopropylethyl)carbamoyl)-1H-pyrazol-3-yl)phenyl)oxazole-5-carboxylate: A solution of T3P (50% solution in EtOAc, 3.11 mL, 5.22 mmol) was added dropwise to a solution of 3-(3-(5-(ethoxycarbonyl)oxazol-2-yl)phenyl)-1 H-pyrazole-5-carboxylic acid Intermediate 2, 0.854 g, 2.61 mmol), TEA (2.18 mL, 15.66 mmol) and (S)-1-cyclopropylethanamine (0.333 g, 3.92 mmol) in EtOAc (13 mL). After 2.5 hours the RM was diluted with EtOAc (˜150 mL) and washed with 50% saturated NaHCO₃ (100 mL). The organic phase was separated, dried over MgSO₄, and filtered. The filtrate was concentrated and purified by FCC (20-60% EtOAc/heptane) to afford 939 mg (91%) of (S)-ethyl 2-(3-(5-((1-cyclopropylethyl)carbamoyl)-1 H-pyrazol-3-yl)phenyl)oxazole-5-carboxylate as a white solid.
LCMS Rt: 1.08 mins MS m/z; 395.1 [M+H]+ RXNMON-Acidic ¹H NMR (400 MHz, Chloroform-d) δ 8.46 (t, J=1.5 Hz, 1H), 8.18 (dt, J=7.8, 1.2 Hz, 1H), 7.93-7.87 (m, 2H), 7.61 (t, J=7.8 Hz, 1H), 7.13 (s, 1H), 6.67 (s, 1H), 4.47 (q, J=7.1 Hz, 2H), 3.71-3.60 (m, 1H), 1.45 (t, J=7.1 Hz, 3H), 1.37 (d, J=6.6 Hz, 3H), 1.03-0.93 (m, 1H), 0.64-0.51 (m, 2H), 0.51-0.44 (m, 1H), 0.38-0.32 (m, 1H).
Step 2: (S)-2-(3-(3-((1-cyclopropylethyl)carbamoyl)-1H-pyrazol-5-yl)phenyl)oxazole-5-carboxylic acid: (S)-ethyl 2-(3-(3-((1-cyclopropylethyl)carbamoyl)-1 H-pyrazol-5-yl)phenyl)oxazole-5-carboxylate (0.60 g, 1.521 mmol) was dissolved in ethanol (10 mL). A solution of 1 M NaOH aq (3.04 mL, 3.04 mmol) was added and the RM was stirred 1 at room temperature. Citric acid (10%, aqueous) was added to bring the RM to pH4. The resulting precipitate was filtered washing with water and dried to afford a quantitative yield of (S)-2-(3-(3-((1-cyclopropylethyl)carbamoyl)-1 H-pyrazol-5-yl)phenyl)oxazole-5-carboxylic acid.
LCMS Rt: 1.13 mins MS m/z; 367.1 [M+H]+ RXNMON-Acidic. ¹H NMR (400 MHz, DMSO-d₆) δ 13.77 (d, J=1.0 Hz, 1H), 8.57-8.13 (m, 2H), 8.06-7.96 (m, 2H), 7.89 (s, 1H), 7.66 (t, J=7.8 Hz, 1H), 7.37 (s, 1H), 3.52-3.41 (m, 1H), 1.24 (d, J=6.7 Hz, 3H), 1.01 (s, 1H), 0.51-0.43 (m, 1H), 0.42-0.36 (m, 1H), 0.35-0.27 (m, 1H), 0.26-0.17 (m, 1H).
Step 3: (S)-ethyl 2-(2-(3-(3-(((S)-1-cyclopropylethyl)carbamoyl)-1 H-pyrazol-5-yl)phenyl)oxazole-5-carboxamido)-3-methylbutanoate: To a solution of (S)-2-(3-(3-((1-cyclopropylethyl)carbamoyl)-1 H-pyrazol-5-yl)phenyl)oxazole-5-carboxylic acid (160 mg, 0.437 mmol) in DMF (Volume: 2.5 mL) was added TEA (0.183 mL, 1.310 mmol), and L-valine ethyl ester (83 mg, 0.459 mmol) to give a colorless solution. T3P (50% EtOAc) (0.338 mL, 0.568 mmol) was added slowly and the reaction allowed to stir at room temperature. The reaction was monitored by LCMS adding additional aliquots of T3P over 24-48 h as needed. The RM was diluted with EtOAc and water with 1 N HCl. The organic phase was separated and washed with brine The EtOAc phase was dried over Na2SO4 and concentrated. The crude material was purified by FCC (0-10% MeOH in DCM) to afford 0.12 g (55.1%) of (S)-ethyl 2-(2-(3-(3-(((S)-1-cyclopropylethyl)carbamoyl)-1H-pyrazol-5-yl)phenyl)oxazole-5-carboxamido)-3-methylbutanoate. LCMS Rt: 1.42 mins MS m/z; 494.2 [M+H]+ RXNMON-Acidic
1H NMR (400 MHz, METHANOL-d₄) δ ppm 8.64 (br. s., 0.3 H) 8.56 (s, 0.7 H) 8.13-8.23 (m, 1H) 8.00-8.06 (m, 0.3 H) 7.89-7.95 (m, 1.7 H) 7.59-7.69 (m, 1H) 7.29 (br. s., 0.3 H) 7.16 (s, 0.7 H) 4.51 (d, J=7.09 Hz, 1H) 4.23 (m, J=3.79 Hz, 2H) 3.49-3.55 (m, 1H) 2.24-2.37 (m, 1H) 1.33 (d, J=6.72 Hz, 3H) 1.30 (t, J=7.15 Hz, 3H) 1.06 (dd, J=8.19, 6.85 Hz, 6H) 0.99-1.03 (m, 1H) 0.53-0.61 (m, 1H) 0.46-0.53 (m, 1H) 0.37-0.43 (m, 1H) 0.25-0.32 (m, 1H).
Examples 1.1 to 1.53 were prepared by a similar method to that of Example 1.0 by replacing the amines in Step 1 and Step 3 with appropriate commercially available amines.

### Example 1.1: (S)-Ethyl 3-Methyl-2-(2-(3-(3-(Pentan-3-Ylcarbamoyl)-1 H-Pyrazol-5-Yl)Phenyl)Oxazole-5-Carboxamido)Butanoate

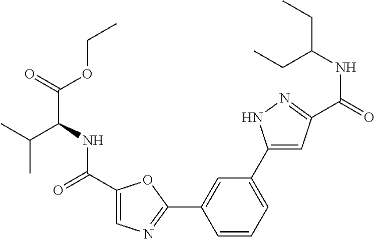 [SMILES: CCOC(=O)[C@@H](NC(=O)c1cnc(-c2cccc(-c3cc(C(=O)NC(CC)CC)n[nH]3)c2)o1)C(C)C]

LCMS Rt: 1.38 mins MS m/z; 496.6 [M+H]+ 2minLowpH_v3

¹H NMR (400 MHz, CHLOROFORM-d) δ ppm 8.38 (s, 1H) 8.09 (d, J=7.83 Hz, 1H) 7.87 (d, J=7.83 Hz, 1H) 7.83 (s, 1H) 7.59 (t, J=7.83 Hz, 1H) 7.13 (s, 1H) 7.07 (d, J=8.59 Hz, 1H) 6.55 (br d, J=9.09 Hz, 1H) 4.77 (dd, J=8.59, 5.31 Hz, 1H) 4.15-4.35 (m, 2H) 4.01-4.12 (m, 1H) 2.24-2.41 (m, 1H) 1.64-1.79 (m, 2H) 1.55 (dt, J=14.21, 7.42 Hz, 2H) 1.33 (t, J=7.07 Hz, 3H) 1.06 (dd, J=6.69, 4.42 Hz, 6H) 0.94-1.01 (m, 6H)

### Example 1.2:(N-Cyclopentyl-2-(3-(3-(Pentan-3-Ylcarbamoyl)-1 H-Pyrazol-5-Yl)Phenyl)Oxazole-5-Carboxamide

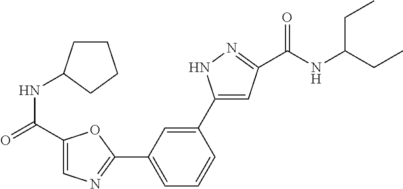 [SMILES: CCC(CC)NC(=O)c1cc(-c2cccc(-c3ncc(C(=O)NC4CCCC4)o3)c2)[nH]n1]

LCMS Rt: 1.19 mins MS m/z; 436.4 [M+H]+ 2minLowpH_v2

### Example 1.3:(N-(3,5-Dimethylphenyl)-2-(3-(3-(Pentan-3-Ylcarbamoyl)-1H-Pyrazol-5-Yl)Phenyl)Oxazole-5-Carboxamide

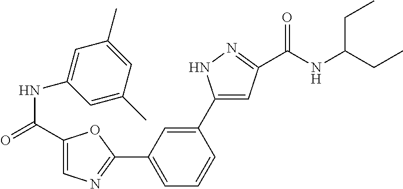 [SMILES: CCC(CC)NC(=O)c1cc(-c2cccc(-c3ncc(C(=O)Nc4cc(C)cc(C)c4)o3)c2)[nH]n1]

LCMS Rt: 1.32 mins MS m/z; 472.4 [M+H]+ 2minLowpH_v2

### Example 1.4: (S)-Methyl 3-Cyclohexyl-2-(2-(3-(3-((Dicyclopropylmethyl)Carbamoyl)-1H-Pyrazol-5-Yl)Phenyl)Oxazole-5-Carboxamido)Propanoate

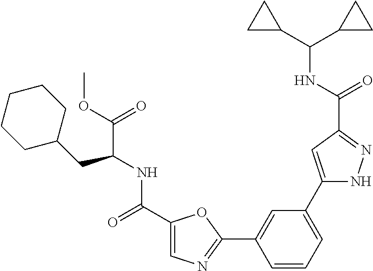 [SMILES: COC(=O)[C@H](CC1CCCCC1)NC(=O)c1cnc(-c2cccc(-c3cc(C(=O)NC(C4CC4)C4CC4)n[nH]3)c2)o1]

LCMS Rt: 1.63 mins MS m/z; 560.3 [M+H]+ RXNMON-Acidic_NonPolar
1H NMR (400 MHz, Methanol-d4) δ 8.57 (brs, 1H), 8.18 (d, J=7.6 Hz, 1H), 7.93 (brs, 1H), 7.91 (s, 1H), 7.69-7.63 (m, 1H), 7.18 (brs, 1H), 4.80-4.72 (m, 1H), 3.75 (s, 3H), 3.09 (t, J=8.1 Hz, 1H), 1.91-1.83 (m, 1H), 1.83-1.78 (m, 2H), 1.77-1.71 (m, 2H), 1.69-1.63 (m, 1H), 1.50-1.38 (m, 1H), 1.36-1.17 (m, 4H), 1.17-1.08 (m, 2H), 1.07-0.93 (m, 2H), 0.63-0.54 (m, 2H), 0.49-0.43 (m, 2H), 0.42-0.35 (m, 4H).

### Example 1.5: (S)-Methyl 2-(2-(3-(3-(((R)-1-Cyclopropylethyl)Carbamoyl)-1H-Pyrazol-5-Yl)Phenyl)Oxazole-5-Carboxamido)-3-Methylbutanoate

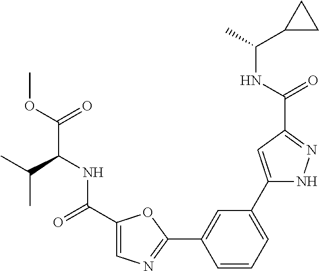 [SMILES: COC(=O)[C@@H](NC(=O)c1cnc(-c2cccc(-c3cc(C(=O)N[C@H](C)C4CC4)n[nH]3)c2)o1)C(C)C]

LCMS Rt: 1.35 min MS m/z; 480.2 [M+H]+ RXNMON-Acidic_NonPolar
1H NMR (400 MHz, Methanol-d4) δ 8.57 (s, 1H), 8.18 (d, J=7.8 Hz, 1H), 7.94 (s, 2H), 7.65 (t, J=7.6 Hz, 1H), 7.18 (s, 1H), 4.53 (d, J=7.1 Hz, 1H), 3.77 (s, 3H), 3.54-3.44 (m, 1H), 2.30 (h, J=6.8 Hz, 1H), 1.33 (d, J=6.7 Hz, 3H), 1.06 (d, J=6.8 Hz, 3H), 1.04 (d, J=6.8 Hz, 3H), 1.03-0.99 (m, 1H), 0.57 (tt, J=8.6, 4.8 Hz, 1H), 0.53-0.46 (m, 1H), 0.40 (dq, J=9.7, 5.0 Hz, 1H), 0.28 (dq, J=9.3, 5.0 Hz, 1H).

### Example 1.6: (S)-N-(1-Cyclopropylethyl)-2-(3-(3-(Pentan-3-Ylcarbamoyl)-1H-Pyrazol-5-Yl)Phenyl)Oxazole-5-Carboxamide

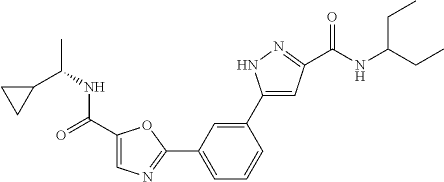 [SMILES: CCC(CC)NC(=O)c1cc(-c2cccc(-c3ncc(C(=O)N[C@@H](C)C4CC4)o3)c2)[nH]n1]

LCMS Rt: 1.18 mins MS m/z; 436.4 [M+H]+ 2minLowpH_v2

¹H NMR (400 MHz, CHLOROFORM-d) δ ppm 8.41 (s, 1H) 8.04 (br d, J=7.82 Hz, 1H) 7.81-7.87 (m, 2H) 7.52 (t, J=7.70 Hz, 1H) 7.11 (s, 1H) 6.78 (br d, J=8.31 Hz, 2H) 4.03-4.14 (m, 1H) 3.55-3.67 (m, 1H) 1.64-1.77 (m, 2H) 1.55 (dquin, J=14.40, 7.40, 7.40, 7.40, 7.40 Hz, 2H) 1.37 (d, J=6.60 Hz, 3H) 0.99 (br t, J=7.34 Hz, 7H) 0.56-0.64 (m, 1H) 0.52 (br dd, J=7.58, 4.89 Hz, 1H) 0.46 (br dd, J=9.41, 4.77 Hz, 1H) 0.27-0.36 (m, 1H)

### Example 1.7:(S)-Methyl 2-(2-(3-(3-(((S)-1-Cyclopropylethyl)Carbamoyl)-1H-Pyrazol-5-yl)Phenyl)Oxazole-5-Carboxamido)-3-Methylbutanoate

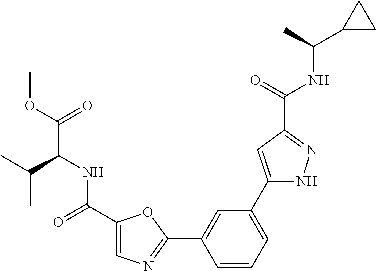 [SMILES: COC(=O)[C@@H](NC(=O)c1cnc(-c2cccc(-c3cc(C(=O)N[C@@H](C)C4CC4)n[nH]3)c2)o1)C(C)C]

LCMS Rt: 1.35 mins MS m/z; 480.3 [M+H]+ RXNMON-Acidic_NonPolar
1H NMR (400 MHz, Methanol-d4) δ 8.57 (br s, 1H), 8.18 (d, J=6.5 Hz, 1H), 7.94 (s, 2H), 7.65 (br s, 1H), 7.18 (br s, 1H), 4.53 (d, J=7.1 Hz, 1H), 3.77 (s, 3H), 3.49 (q, J=6.9 Hz, 1H), 2.37-2.23 (m, 1H), 1.33 (d, J=6.7 Hz, 3H), 1.06 (d, J=6.8 Hz, 3H), 1.04 (d, J=6.8 Hz, 3H), 1.03-0.97 (m, 1H), 0.57 (tt, J=8.6, 4.8 Hz, 1H), 0.53-0.45 (m, 1H), 0.40 (dq, J=9.7, 5.1 Hz, 1H), 0.28 (dq, J=9.3, 4.9 Hz, 1H).

### Example 1.8: N-((R)-1-Cyclopropylethyl)-2-(3-(3-(((S)-1-Cyclopropylethyl)Carbamoyl)-1H-Pyrazol-5-Yl)Phenyl)Oxazole-5-Carboxamide

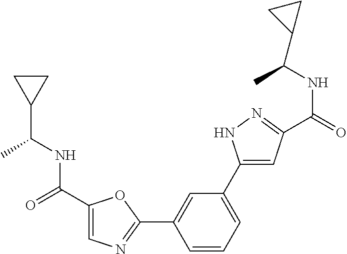 [SMILES: C[C@H](NC(=O)c1cc(-c2cccc(-c3ncc(C(=O)N[C@H](C)C4CC4)o3)c2)[nH]n1)C1CC1]

LCMS Rt: 1.06 mins MS m/z; 434.2[M+H]+ RXNMON-Acidic

¹H NMR (400 MHz, ACETONITRILE-d₃) δ ppm 8.46 (s, 1H) 8.07 (dt, J=7.82, 1.28 Hz, 1H) 7.84 (br d, J=7.58 Hz, 1H) 7.64 (s, 1H) 7.56 (t, J=7.82 Hz, 1H) 7.20 (br d, J=8.07 Hz, 1H) 7.07 (s, 1H) 7.02 (br d, J=7.46 Hz, 1H) 3.30-3.51 (m, 2H) 1.24 (d, J=6.72 Hz, 3H) 1.21 (d, J=6.60 Hz, 3H) 0.94 (s, 2H) 0.42-0.51 (m, 2H) 0.33-0.40 (m, 2H) 0.27 (br dd, J=9.90, 4.89 Hz, 3H) 0.12-0.23 (m, 2H)

### Example 1.9: (S)-Tert-Butyl 3-Methyl-2-(2-(3-(3-(Pentan-3-Ylcarbamoyl)-1H-Pyrazol-5-yl)Phenyl)Oxazole-5-Carboxamido)Butanoate

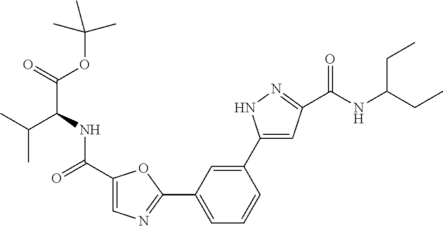 [SMILES: CCC(CC)NC(=O)c1cc(-c2cccc(-c3ncc(C(=O)N[C@H](C(=O)OC(C)(C)C)C(C)C)o3)c2)[nH]n1]

LCMS Rt: 1.25 mins MS m/z; 452.5 [M+H]+ 2minLowpH_v2

### Example 1.10: (S)-2-(3-(5-((1-Cyclopropylethyl)Carbamoyl)-1H-Pyrazol-3-Yl)Phenyl)-N-(Dicyclopropylmethyl)Oxazole-5-Carboxamide

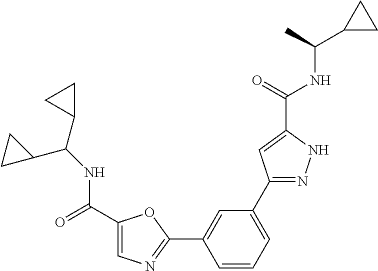 [SMILES: C[C@H](NC(=O)c1cc(-c2cccc(-c3ncc(C(=O)NC(C4CC4)C4CC4)o3)c2)n[nH]1)C1CC1]

LCMS Rt: 1.42 mins MS m/z; 460.2 [M+H]+ RXNMON-Acidic
1H NMR (400 MHz, DMSO-d₆) δ ppm 8.72 (d, J=8.8 Hz, 1H) 8.53 (t, J=1.5 Hz, 1H) 8.25 (d, J=8.4 Hz, 1H) 8.13 (dt, J=7.8, 1.3 Hz, 1H) 8.00 (dt, J=8.0, 1.3 Hz, 1H) 7.93 (s, 1H) 7.61-7.68 (m, 1H) 7.35 (s, 1H) 3.38-3.57 (m, 1H) 2.93 (q, J=8.6 Hz, 1H) 1.25 (d, J=6.7 Hz, 3 H) 1.09-1.20 (m, 2H) 0.98-1.08 (m, 1H) 0.50-0.58 (m, 2H) 0.44-0.50 (m, 1H) 0.35-0.44 (m, 5H) 0.19-0.35 (m, 4H).

### Example 1.11: N-(2-Methylpentan-3-Yl)-2-(3-(3-(Pentan-3-Ylcarbamoyl)-1H-Pyrazol-5-yl)Phenyl)Oxazole-5-Carboxamide

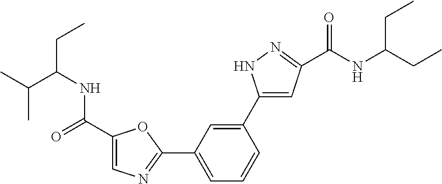 [SMILES: CCC(CC)NC(=O)c1cc(-c2cccc(-c3ncc(C(=O)NC(CC)C(C)C)o3)c2)[nH]n1]

LCMS Rt: 1.33 mins MS m/z; 524.5 [M+H]+ 2minLowpH_v2

### Example 1.12: (S)-Ethyl 2-(2-(3-(5-((Dicyclopropylmethyl)Carbamoyl)-1H-Pyrazol-3-yl)Phenyl)Oxazole-5-Carboxamido)-3-Methylbutanoate

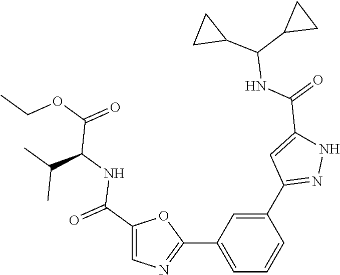 [SMILES: CCOC(=O)[C@@H](NC(=O)c1cnc(-c2cccc(-c3cc(C(=O)NC(C4CC4)C4CC4)[nH]n3)c2)o1)C(C)C]

LCMS Rt: 1.38 mins MS m/z; 520 [M+H]+ RXNMON-Basic

¹H NMR (400 MHz, Methanol-d4) δ 8.58 (s, 1H), 8.18 (d, J=7.8 Hz, 1H), 7.94 (s, 2H), 7.64 (t, J=7.8 Hz, 1H), 7.21 (s, 1H), 4.50 (d, J=7.0 Hz, 1H), 4.24 (qq, J=7.3, 3.7 Hz, 2H), 3.09 (t, J=8.2 Hz, 1H), 2.37-2.25 (m, 1H), 1.30 (t, J=7.1 Hz, 3H), 1.18-1.10 (m, 2H), 1.06 (dd, J=8.4, 6.9 Hz, 6H), 0.59 (td, J=8.3, 2.0 Hz, 2H), 0.46 (ddd, J=10.2, 6.0, 1.6 Hz, 2H), 0.38 (dd, J=4.6, 2.9 Hz, 4H)

### Example 1.13: N-(Tert-Butyl)-2-(3-(3-(Pentan-3-Ylcarbamoyl)-1H-Pyrazol-5-yl)Phenyl)Oxazole-5-Carboxamide

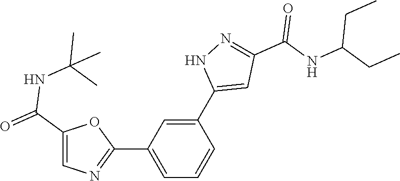 [SMILES: CCC(CC)NC(=O)c1cc(-c2cccc(-c3ncc(C(=O)NC(C)(C)C)o3)c2)[nH]n1]

LCMS Rt: 1.19 mins MS m/z; 424.4 [M+H]+ 2minLowpH_v2

### Example 1.14: (S)-Ethyl 4-(Methylthio)-2-(2-(3-(3-(Pentan-3-Ylcarbamoyl)-1H-Pyrazol-5-yl)Phenyl)Oxazole-5-Carboxamido)Butanoate

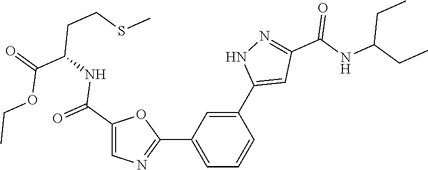 [SMILES: CCOC(=O)[C@H](CCSC)NC(=O)c1cnc(-c2cccc(-c3cc(C(=O)NC(CC)CC)n[nH]3)c2)o1]

LCMS Rt: 1.21 mins MS m/z; 528.4 [M+H]+ 2minLowpH_v2

### Example 1.15: (S)-Methyl 2-(2-(3-(3-(Pentan-3-Ylcarbamoyl)-1H-Pyrazol-5-yl)Phenyl)Oxazole-5-Carboxamido)-2-Phenylacetate

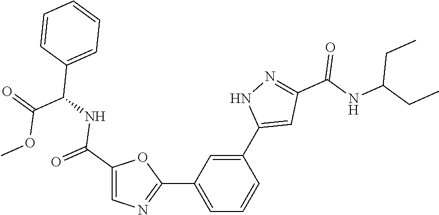 [SMILES: CCC(CC)NC(=O)c1cc(-c2cccc(-c3ncc(C(=O)N[C@H](C(=O)OC)c4ccccc4)o3)c2)[nH]n1]

LCMS Rt: 1.25 mins MS m/z; 539.4 [M+H]+ 2minLowpH_v2

### Example 1.16: (S)-Tert-Butyl 2-(4-Methyl-2-(2-(3-(3-(Pentan-3-Ylcarbamoyl)-1 H-Pyrazol-5-yl)Phenyl)Oxazole-5-Carboxamido)Pentanamido)Acetate

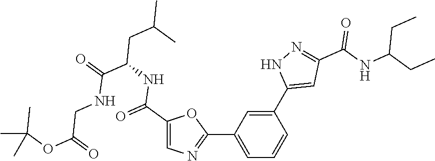 [SMILES: CCC(CC)NC(=O)c1cc(-c2cccc(-c3ncc(C(=O)N[C@@H](CC(C)C)C(=O)NCC(=O)OC(C)(C)C)o3)c2)[nH]n1]

LCMS Rt: 1.33 mins MS m/z; 524.5 [M+H]+ 2minLowpH_v2

### Example 1.17: N-(4-Fluorobenzyl)-2-(3-(3-(Pentan-3-Ylcarbamoyl)-1H-Pyrazol-5-yl)Phenyl)Oxazole-5-Carboxamide

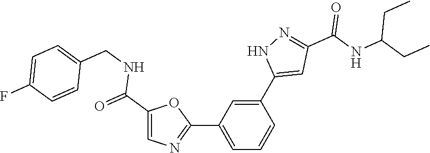 [SMILES: CCC(CC)NC(=O)c1cc(-c2cccc(-c3ncc(C(=O)NCc4ccc(F)cc4)o3)c2)[nH]n1]

LCMS Rt: 1.21 mins MS m/z; 476.4 [M+H]+ 2minLowpH_v2

### Example 1.18: 2-(3-(3-(Pentan-3-Ylcarbamoyl)-1H-Pyrazol-5-Yl)Phenyl)-N-((Tetrahydro-2H-Pyran-2-Yl)Methyl)Oxazole-5-Carboxamide

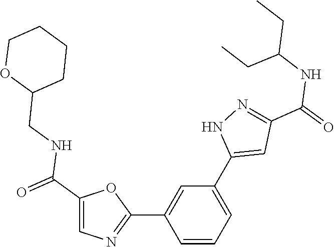 [SMILES: CCC(CC)NC(=O)c1cc(-c2cccc(-c3ncc(C(=O)NCC4CCCCO4)o3)c2)[nH]n1]

LCMS Rt: 1.24 mins MS m/z; 466.3 [M+H]+ 2minLowpH_v3

### Example 1.19: N-Benzyl-2-(3-(3-(Pentan-3-Ylcarbamoyl)-1H-Pyrazol-5-Yl)Phenyl)Oxazole-5-Carboxamide

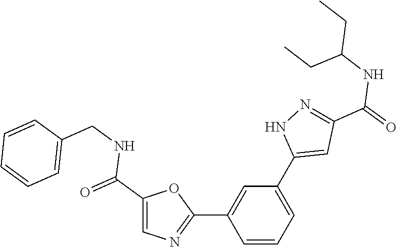 [SMILES: CCC(CC)NC(=O)c1cc(-c2cccc(-c3ncc(C(=O)NCc4ccccc4)o3)c2)[nH]n1]

LCMS Rt: 1.32 mins MS m/z; 458.4 [M+H]+ 2minLowpH_v3

### Example 1.20: N-(Pentan-3-Yl)-2-(3-(5-(Pentan-3-Ylcarbamoyl)-1H-Pyrazol-3-yl)Phenyl)Oxazole-5-Carboxamide

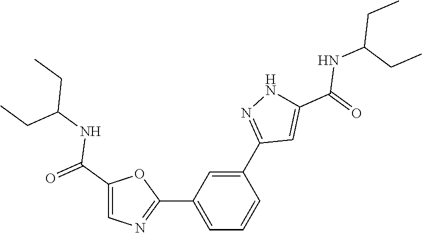 [SMILES: CCC(CC)NC(=O)c1cc(-c2cccc(-c3ncc(C(=O)NC(CC)CC)o3)c2)n[nH]1]

LCMS Rt: 1.33 mins MS m/z; 438.5 [M+H]+ 2minLowpHv03

¹H NMR (400 MHz, CHLOROFORM-d) δ ppm 8.40 (s, 1H) 8.11 (br d, J=8.07 Hz, 1H) 7.82-7.87 (m, 2H) 7.59 (br t, J=7.82 Hz, 1H) 7.14 (s, 1H) 6.54-6.63 (m, 1H) 6.13 (br d, J=8.56 Hz, 1H) 3.99-4.12 (m, 2H) 1.66-1.80 (m, 4H) 1.48-1.64 (m, 4H) 0.96-1.06 (m, 12H)

### Example 1.21: (S)-2-(3-(3-(Pentan-3-Ylcarbamoyl)-1H-Pyrazol-5-Yl)Phenyl)-N-(1-Phenylethyl)Oxazole-5-Carboxamide

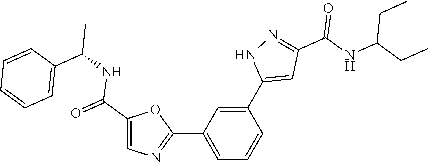 [SMILES: CCC(CC)NC(=O)c1cc(-c2cccc(-c3ncc(C(=O)N[C@@H](C)c4ccccc4)o3)c2)[nH]n1]

LCMS Rt: 1.23 mins MS m/z; 472.4 [M+H]+ 2minLowpH_v2

### Example 1.22: (2S)-Ethyl 3-Methyl-2-(2-(3-(5-((1,1,1-Trifluorobutan-2-Yl)Carbamoyl)-1H-Pyrazol-3-Yl)Phenyl)Oxazole-5-Carboxamido)Butanoate

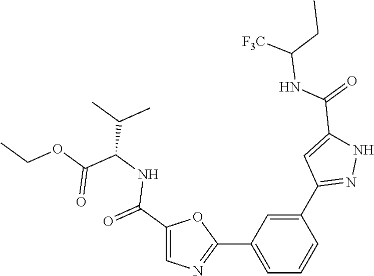 [SMILES: CCOC(=O)[C@@H](NC(=O)c1cnc(-c2cccc(-c3cc(C(=O)NC(CC)C(F)(F)F)[nH]n3)c2)o1)C(C)C]

LCMS Rt: 1.39 mins MS m/z; 536.3 [M+H]+ RXNMON-Basic

¹H NMR (400 MHz, Methanol-d4) δ 8.49 (s, 1H), 8.10 (d, J=7.9 Hz, 1H), 7.85 (s, 1H), 7.56 (t, J=7.8 Hz, 1H), 7.18 (s, OH), 4.57 (ddd, J=11.3, 7.6, 3.7 Hz, 1H), 4.40 (d, J=7.0 Hz, 1H), 4.14 (tq, J=7.1, 3.4 Hz, 1H), 2.21 (h, J=6.8 Hz, 1H), 1.85 (ddd, J=14.0, 7.4, 3.8 Hz, 1H), 1.70 (ddd, J=14.0, 11.1, 7.3 Hz, 1H), 1.25-1.12 (m, 3H), 0.96 (dd, J=8.0, 6.9 Hz, 5H).

### Example 1.24: (S)-N-(1-Cyclohexylethyl)-2-(3-(3-(Pentan-3-Ylcarbamoyl)-1H-Pyrazol-5-yl)Phenyl)Oxazole-5-Carboxamide

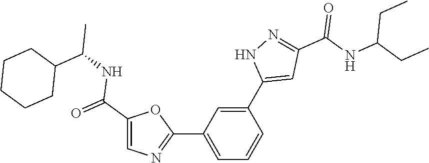 [SMILES: CCC(CC)NC(=O)c1cc(-c2cccc(-c3ncc(C(=O)N[C@@H](C)C4CCCCC4)o3)c2)[nH]n1]

LCMS Rt: 1.33 mins MS m/z; 478.5 [M+H]+ 2minLowpH_v2

### Example 1.25: (S)-Methyl 2-(2-(3-(3-(((S)-1-Cyclopropylethyl)Carbamoyl)-1H-Pyrazol-5-yl)Phenyl)Oxazole-5-Carboxamido)-3-(Methylthio)Propanoate

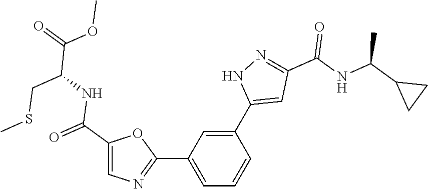 [SMILES: COC(=O)[C@@H](CSC)NC(=O)c1cnc(-c2cccc(-c3cc(C(=O)N[C@@H](C)C4CC4)n[nH]3)c2)o1]

LCMS Rt: 1.30 mins MS m/z; 498.0 [M+H]+ RXNMON-Acidic

### Example 1.26: (S)-Methyl 4-Methyl-2-(2-(3-(3-(Pentan-3-Ylcarbamoyl)-1H-Pyrazol-5-yl)Phenyl)Oxazole-5-Carboxamido)Pentanoate

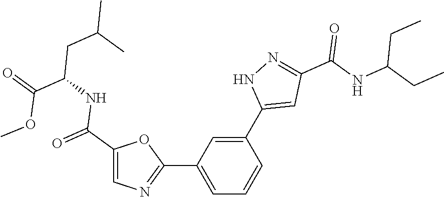 [SMILES: CCC(CC)NC(=O)c1cc(-c2cccc(-c3ncc(C(=O)N[C@@H](CC(C)C)C(=O)OC)o3)c2)[nH]n1]

LCMS Rt: 1.31 mins MS m/z; 496.6 [M+H]+ 2minLowpH_v2

### Example 1.27: N-(3-Cyanophenyl)-2-(3-(3-(Pentan-3-Ylcarbamoyl)-1 H-Pyrazol-5-yl)Phenyl)Oxazole-5-Carboxamide

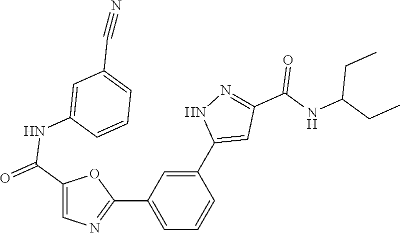 [SMILES: CCC(CC)NC(=O)c1cc(-c2cccc(-c3ncc(C(=O)Nc4cccc(C#N)c4)o3)c2)[nH]n1]

LCMS Rt: 1.21 mins MS m/z; 469.5 [M+H]+ 2minLowpH_v2

### Example 1.28: (R)-Ethyl 2-(2-(3-(3-(Pentan-3-Ylcarbamoyl)-1 H-Pyrazol-5-yl)Phenyl)Oxazole-5-Carboxamido)-2-Phenylacetate

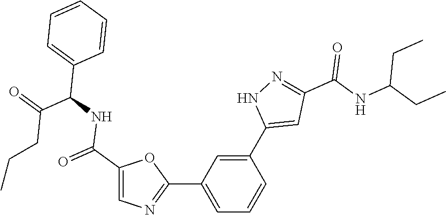 [SMILES: CCCC(=O)[C@H](NC(=O)c1cnc(-c2cccc(-c3cc(C(=O)NC(CC)CC)n[nH]3)c2)o1)c1ccccc1]

LCMS Rt: 1.25 mins MS m/z; 530.4 [M+H]+ 2minLowpH_v2

### Example 1.29: (S)-Methyl 2-(2-(3-(5-((Dicyclopropylmethyl)Carbamoyl)-1 H-Pyrazol-3-yl)Phenyl)Oxazole-5-Carboxamido)-3-Methylbutanoate

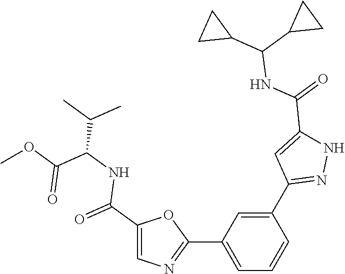 [SMILES: COC(=O)[C@@H](NC(=O)c1cnc(-c2cccc(-c3cc(C(=O)NC(C4CC4)C4CC4)[nH]n3)c2)o1)C(C)C]

LCMS Rt: 1.34 mins MS m/z; 506.5 [M+H]+ RXNMON_Basic.

¹H NMR (400 MHz, Methanol-d4) δ 8.56 (s, 1H), 8.16 (d, J=7.9 Hz, 1H), 7.93 (s, 2H), 7.63 (t, J=7.8 Hz, 1H), 7.21 (s, 1H), 4.53 (d, J=7.0 Hz, 1H), 3.77 (s, 3H), 3.15-3.04 (m, 1H), 2.38-2.22 (m, J=6.8 Hz, 1H), 1.21-1.00 (m, 9H), 0.66-0.53 (m, 2H), 0.53-0.44 (m, 2H), 0.44-0.32 (m, 5H).

### Example 1.30: N-(Isoxazol-3-Yl)-2-(3-(3-(Pentan-3-Ylcarbamoyl)-1 H-Pyrazol-5-yl)Phenyl)Oxazole-5-Carboxamide

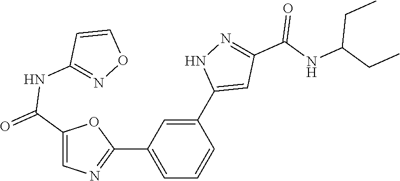 [SMILES: CCC(CC)NC(=O)c1cc(-c2cccc(-c3ncc(C(=O)Nc4ccon4)o3)c2)[nH]n1]

LCMS Rt: 1.10 mins MS m/z; 435.3 [M+H]+ 2minLowpH_v2

### Example 1.31: N-(Benzo[d][1,3]Dioxol-5-Ylmethyl)-2-(3-(3-(Pentan-3-Ylcarbamoyl)-1 H-Pyrazol-5-yl)Phenyl)Oxazole-5-Carboxamide

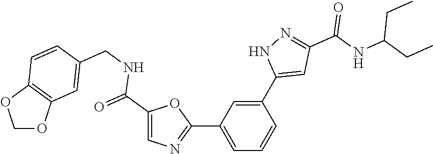 [SMILES: CCC(CC)NC(=O)c1cc(-c2cccc(-c3ncc(C(=O)NCc4ccc5c(c4)OCO5)o3)c2)[nH]n1]

LCMS Rt: 1.17 mins MS m/z; 502.4 [M+H]+ 2minLowpH_v2

### Example 1.32: Isopropyl 2-(2-(3-(3-(Pentan-3-Ylcarbamoyl)-1 H-Pyrazol-5-yl)Phenyl)Oxazole-5-Carboxamido)Acetate

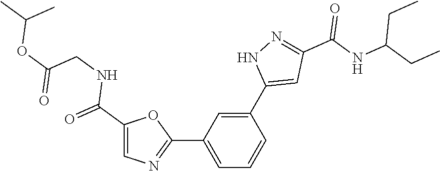 [SMILES: CCC(CC)NC(=O)c1cc(-c2cccc(-c3ncc(C(=O)NCC(=O)OC(C)C)o3)c2)[nH]n1]

LCMS Rt: 1.14 mins MS m/z; 468.4 [M+H]+ 2minLowpH_v2

### Example 1.33: N-(3-Chlorophenyl)-2-(3-(3-(Pentan-3-Ylcarbamoyl)-1H-Pyrazol-5-yl)Phenyl)Oxazole-5-Carboxamide

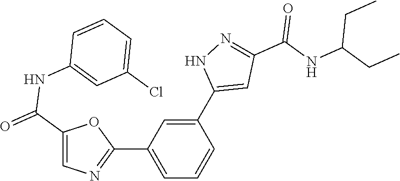 [SMILES: CCC(CC)NC(=O)c1cc(-c2cccc(-c3ncc(C(=O)Nc4cccc(Cl)c4)o3)c2)[nH]n1]

LCMS Rt: 1.31 mins MS m/z; 478.3 [M+H]+ 2minLowpH_v2

### Example 1.34: N-((1-Methylcyclohexyl)Methyl)-2-(3-(3-(Pentan-3-Ylcarbamoyl)-1 H-Pyrazol-5-Yl)Phenyl)Oxazole-5-Carboxamide

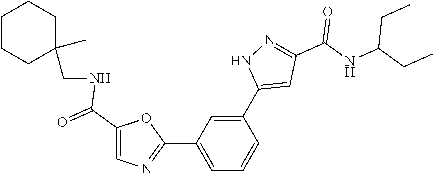 [SMILES: CCC(CC)NC(=O)c1cc(-c2cccc(-c3ncc(C(=O)NCC4(C)CCCCC4)o3)c2)[nH]n1]

LCMS Rt: 1.34 mins MS m/z; 478.5 [M+H]+ 2minLowpH_v2

### Example 1.35: N-(Heptan-4-Yl)-2-(3-(3-(Pentan-3-Ylcarbamoyl)-1H-Pyrazol-5-yl)Phenyl)Oxazole-5-Carboxamide

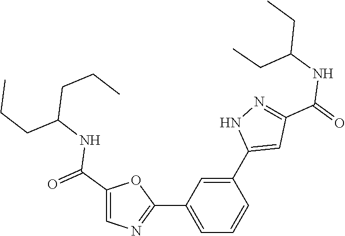 [SMILES: CCCC(CCC)NC(=O)c1cnc(-c2cccc(-c3cc(C(=O)NC(CC)CC)n[nH]3)c2)o1]

LCMS Rt: 1.47 mins MS m/z; 466.4 [M+H]+ 2minLowpH_v3

### Example 1.36: (S)-Ethyl 2-(2-(3-(3-(Pentan-3-Ylcarbamoyl)-1 H-Pyrazol-5-Yl)Phenyl)Oxazole-5-Carboxamido)-2-Phenylacetate

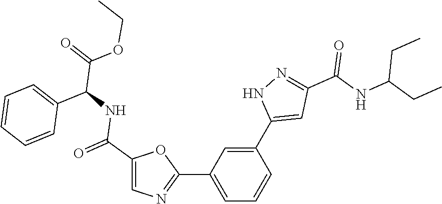 [SMILES: CCOC(=O)[C@@H](NC(=O)c1cnc(-c2cccc(-c3cc(C(=O)NC(CC)CC)n[nH]3)c2)o1)c1ccccc1]

LCMS Rt: 1.25 mins MS m/z; 530.4 [M+H]+ 2minLowpH_v2

### Example 1.37: (S)-Tert-Butyl 2-(2-(3-(3-(Pentan-3-Ylcarbamoyl)-1H-Pyrazol-5-yl)Phenyl)Oxazole-5-Carboxamido)-2-Phenylacetate

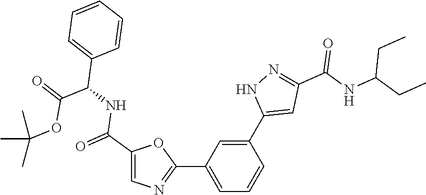 [SMILES: CCC(CC)NC(=O)c1cc(-c2cccc(-c3ncc(C(=O)N[C@H](C(=O)OC(C)(C)C)c4ccccc4)o3)c2)[nH]n1]

LCMS Rt: 1.34 mins MS m/z; 558.4 [M+H]+ 2minLowpH_v2

### Example 1.38: (S)-Ethyl 2-(2-(3-(3-(Pentan-3-Ylcarbamoyl)-1 H-Pyrazol-5-yl)Phenyl)Oxazole-5-Carboxamido)-3-Phenylpropanoate

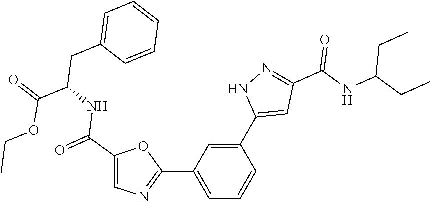 [SMILES: CCOC(=O)[C@H](Cc1ccccc1)NC(=O)c1cnc(-c2cccc(-c3cc(C(=O)NC(CC)CC)n[nH]3)c2)o1]

LCMS Rt: 1.27 mins MS m/z; 544.4 [M+H]+ 2minLowpH_v2

### Example 1.39: (R)-Methyl 2-(2-(3-(3-(Pentan-3-Ylcarbamoyl)-1H-Pyrazol-5-yl)Phenyl)Oxazole-5-Carboxamido)-2-Phenylacetate

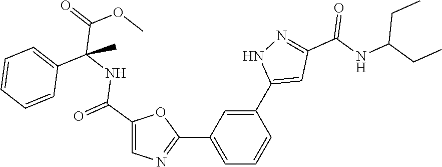 [SMILES: CCC(CC)NC(=O)c1cc(-c2cccc(-c3ncc(C(=O)N[C@@](C)(C(=O)OC)c4ccccc4)o3)c2)[nH]n1]

LCMS Rt: 1.21 mins MS m/z; 516.4 [M+H]+ 2minLowpH_v2

### Example 1.40: (R)-N-(1-Cyclopropylethyl)-2-(3-(5-(Pentan-3-Ylcarbamoyl)-1H-Pyrazol-3-yl)Phenyl)Oxazole-5-Carboxamide

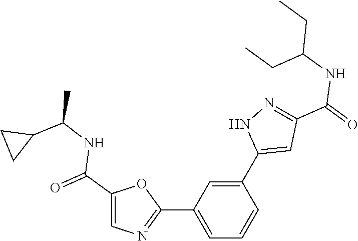 [SMILES: CCC(CC)NC(=O)c1cc(-c2cccc(-c3ncc(C(=O)N[C@H](C)C4CC4)o3)c2)[nH]n1]

LCMS Rt: 1.30 mins MS m/z; 436.4 [M+H]+ 2minLowpH_v2

### Example 1.41: (S)-Benzyl 2-(2-(3-(3-(Pentan-3-Ylcarbamoyl)-1H-Pyrazol-5-yl)Phenyl)Oxazole-5-Carboxamido)Propanoate

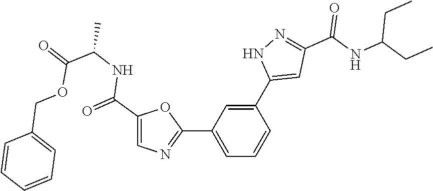 [SMILES: CCC(CC)NC(=O)c1cc(-c2cccc(-c3ncc(C(=O)N[C@@H](C)C(=O)OCc4ccccc4)o3)c2)[nH]n1]

LCMS Rt: 1.25 mins MS m/z; 530.4 [M+H]+ 2minLowpH_v2

### Example 1.42: (R)-N-(1-Cyclohexylethyl)-2-(3-(3-(Pentan-3-Ylcarbamoyl)-1 H-Pyrazol-5-yl)Phenyl)Oxazole-5-Carboxamide

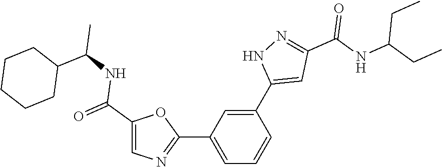 [SMILES: CCC(CC)NC(=O)c1cc(-c2cccc(-c3ncc(C(=O)N[C@H](C)C4CCCCC4)o3)c2)[nH]n1]

LCMS Rt: 1.33 mins MS m/z; 478.5 [M+H]+ 2minLowpH_v2

### Example 1.43: N-(2,6-Difluorobenzyl)-2-(3-(3-(Pentan-3-Ylcarbamoyl)-1H-Pyrazol-5-yl)Phenyl)Oxazole-5-Carboxamide

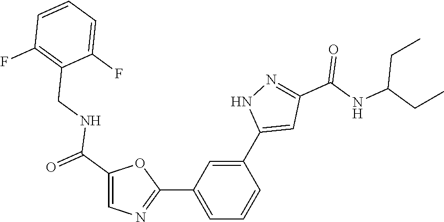 [SMILES: CCC(CC)NC(=O)c1cc(-c2cccc(-c3ncc(C(=O)NCc4c(F)cccc4F)o3)c2)[nH]n1]

LCMS Rt: 1.20 mins MS m/z; 494.4 [M+H]+ 2minLowpH_v2

### Example 1.44: (R)-Ethyl 3-Methyl-2-(2-(3-(5-(Pentan-3-Ylcarbamoyl)-1H-Pyrazol-3-yl)Phenyl)Oxazole-5-Carboxamido)Butanoate

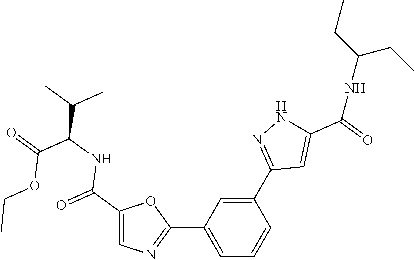 [SMILES: CCOC(=O)[C@H](NC(=O)c1cnc(-c2cccc(-c3cc(C(=O)NC(CC)CC)[nH]n3)c2)o1)C(C)C]

LCMS Rt: 1.38 mins MS m/z; 496.4 [M+H]+ 2minLowpH_v03

¹H NMR (400 MHz, DMSO-d₆) δ ppm 13.61-13.93 (m, 1H) 8.83-9.01 (m, 1H) 8.52 (t, J=1.47 Hz, 1H) 8.12 (br d, J=7.82 Hz, 1H) 8.09 (s, 1H) 7.99-8.03 (m, 1H) 7.64-7.73 (m, 1H) 4.33 (t, J=7.70 Hz, 1H) 4.11-4.23 (m, 3H) 3.73-3.84 (m, 1H) 2.14-2.28 (m, 1H) 1.42-1.63 (m, 5H) 1.23 (t, J=7.09 Hz, 3H) 0.99 (dd, J=14.67, 6.85 Hz, 6H) 0.88 (t, J=7.34 Hz, 6H)

### Example 1.45: (S)-N-(Sec-Butyl)-2-(3-(3-(Pentan-3-Ylcarbamoyl)-1H-Pyrazol-5-Yl)Phenyl)Oxazole-5-Carboxamide

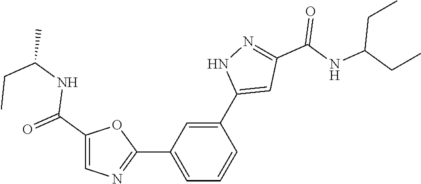 [SMILES: CCC(CC)NC(=O)c1cc(-c2cccc(-c3ncc(C(=O)N[C@@H](C)CC)o3)c2)[nH]n1]

LCMS Rt: 1.17 mins MS m/z; 424.4 [M+H]+ 2minLowpH_v2

### Example 1.46: (R)-N-(3-Methylbutan-2-Yl)-2-(3-(3-(Pentan-3-Ylcarbamoyl)-1 H-Pyrazol-5-yl)Phenyl)Oxazole-5-Carboxamide

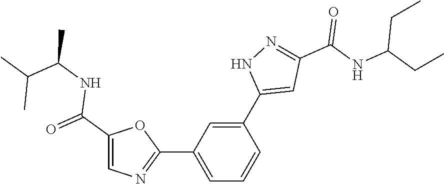 [SMILES: CCC(CC)NC(=O)c1cc(-c2cccc(-c3ncc(C(=O)N[C@H](C)C(C)C)o3)c2)[nH]n1]

LCMS Rt: 1.21 mins MS m/z; 438.4 [M+H]+ 2minLowpH_v2

### Example 1.47: (S)-Methyl 3-Methyl-2-(2-(3-(3-(Pentan-3-Ylcarbamoyl)-1H-Pyrazol-5-yl)Phenyl)Oxazole-5-Carboxamido)Butanoate

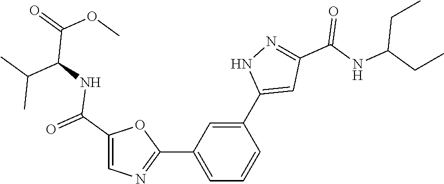 [SMILES: CCC(CC)NC(=O)c1cc(-c2cccc(-c3ncc(C(=O)N[C@H](C(=O)OC)C(C)C)o3)c2)[nH]n1]

LCMS Rt: 1.19 mins MS m/z; 482.4 [M+H]+ 2minLowpH_v2

### Example 1.48: 2-(3-(5-(((S)-1-Cyclopropylethyl)Carbamoyl)-1H-Pyrazol-3-Yl)Phenyl)-N-(1-Cyclopropylpropyl)Oxazole-5-Carboxamide

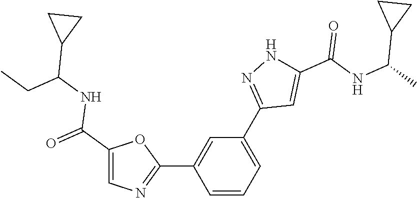 [SMILES: CCC(NC(=O)c1cnc(-c2cccc(-c3cc(C(=O)N[C@@H](C)C4CC4)[nH]n3)c2)o1)C1CC1]

LCMS Rt: 1.10 mins MS m/z; 448.2 [M+H]+ RXNMON-Acidic

¹H NMR (400 MHz, DMSO-d₆) δ 13.79 (d, J=42.7 Hz, 1H), 8.61-8.48 (m, 2H), 8.45-7.96 (m, 3H), 7.93 (d, J=8.4 Hz, 1H), 7.75-7.61 (m, 1H), 7.32 (d, J=106.4 Hz, 1H), 3.58-3.37 (m, 1H), 3.31-3.20 (m, 1H), 1.80-1.56 (m, 2H), 1.24 (d, J=6.6 Hz, 3H), 1.04-0.95 (m, 2H), 0.92 (t, J=7.4 Hz, 3H), 0.59-0.13 (m, 8H).

### Example 1.49: (S)-Methyl 1-(2-(3-(5-((1-Cyclopropylethyl)Carbamoyl)-1H-Pyrazol-3-Yl)Phenyl)Oxazole-5-Carboxamido)Cyclobutanecarboxylate

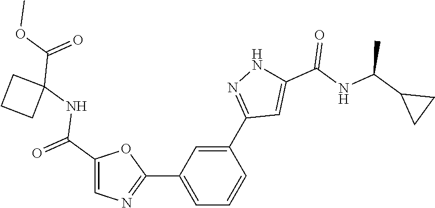 [SMILES: COC(=O)C1(NC(=O)c2cnc(-c3cccc(-c4cc(C(=O)N[C@@H](C)C5CC5)[nH]n4)c3)o2)CCC1]

LCMS Rt: 1.22 mins MS m/z; 477.8 [M+H]+ RXNMON-Basic

¹H NMR (400 MHz, Methanol-d4) δ 8.36 (t, J=1.6 Hz, 1H), 7.98 (dt, J=7.8, 1.2 Hz, 1H), 7.75 (dt, J=7.8, 1.1 Hz, 1H), 7.70 (s, 1H), 7.45 (t, J=7.8 Hz, 1H), 7.06 (s, 1H), 3.69 (s, 3H), 3.46 (dd, J=8.4, 6.7 Hz, 1H), 2.67 (dtt, J=13.2, 5.7, 2.4 Hz, 2H), 2.40 (ddd, J=13.0, 9.8, 7.7 Hz, 2H), 2.03 (dtd, J=13.5, 9.8, 8.6, 3.4 Hz, 2H), 1.22 (d, J=6.6 Hz, 3H), 0.86 (dt, J=8.3, 4.9 Hz, 1H), 0.45 (ddd, J=8.5, 5.5, 4.2 Hz, 1H), 0.42-0.35 (m, 1H), 0.31 (dd, J=9.6, 4.6 Hz, 1H), 0.19 (dt, J=9.3, 4.5 Hz, 1H).

### Example 1.50: (S)-Tert-Butyl 3-(Tert-Butoxy)-2-(2-(3-(3-(Pentan-3-Ylcarbamoyl)-1H-Pyrazol-5-Yl)Phenyl)Oxazole-5-Carboxamido)Propanoate Trifluoroacetate

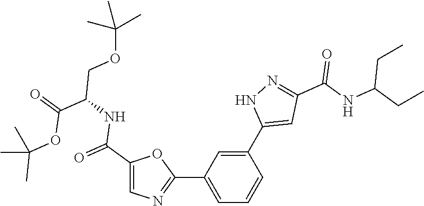 [SMILES: CCC(CC)NC(=O)c1cc(-c2cccc(-c3ncc(C(=O)N[C@@H](COC(C)(C)C)C(=O)OC(C)(C)C)o3)c2)[nH]n1]

LCMS Rt: 4.85 mins MS m/z; 456.5 [M+H]+ 8minLowpHv02

### Example 1.51: Ethyl 2-Methyl-2-(2-(3-(3-(Pentan-3-Ylcarbamoyl)-1H-Pyrazol-5-Yl)Phenyl)Oxazole-5-Carboxamido)Propanoate Trifluoroacetate

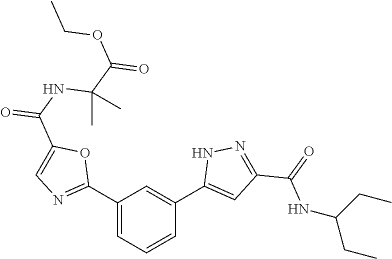 [SMILES: CCOC(=O)C(C)(C)NC(=O)c1cnc(-c2cccc(-c3cc(C(=O)NC(CC)CC)n[nH]3)c2)o1]

LCMS Rt: 1.43 mins MS m/z; 482.6 [M+H]+ 2minLowpH_v2

### Example 2.0 of the Present Invention May be Prepared According to Scheme 8

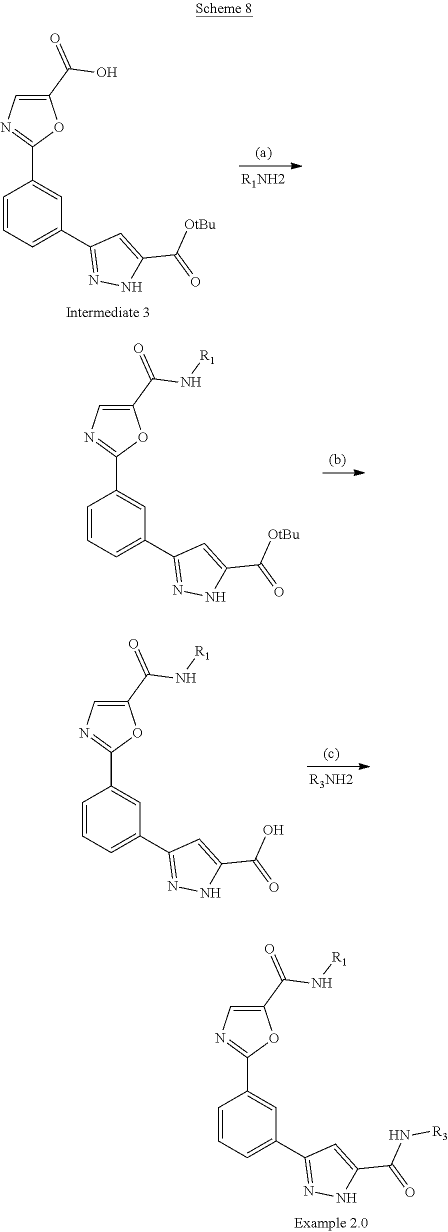

Step (a) involves reaction of an amine(R₁NH2) with Intermediate 3 in a suitable solvent such as DMF or ethyl acetate with a suitable base such as diisopropylethylamine or triethylamine and an amide coupling reagent such as T3P or pyBOP.
Step (b) involves conversion of the tert-butyl ester to a carboxylic acid using a suitable acid such as TFA or HCl in a suitable solvent such as DCM or dioxane.
Step (c) involves reaction of an amine(R₃NH2) with the free acid in a suitable solvent such as DMF or ethyl acetate with a suitable base such as diisopropylethylamine or triethylamine and an amide coupling reagent such as T3P or pyBOP.

### Example 2.0: (S)-Ethyl 2-(2-(3-(5-(((R)-1-Methoxy-3-Methyl-1-Oxobutan-2-Yl)Carbamoyl)-1 H-Pyrazol-3-Yl)Phenyl)Oxazole-5-Carboxamido)-3-Methylbutanoate

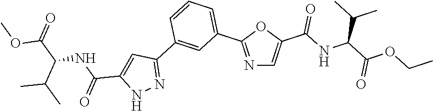 [SMILES: CCOC(=O)[C@@H](NC(=O)c1cnc(-c2cccc(-c3cc(C(=O)N[C@@H](C(=O)OC)C(C)C)[nH]n3)c2)o1)C(C)C]

Step 1: (S)-tert-butyl 3-(3-(5-((1-ethoxy-3-methyl-1-oxobutan-2-yl)carbamoyl)oxazol-2-yl)phenyl)-1 H-pyrazole-5-carboxylate: T3P (50% solution in EtOAc, 4.66 mL, 7.82 mmol) was added dropwise to a stirred suspension of 2-(3-(5-(tert-butoxycarbonyl)-1 H-pyrazol-3-yl)phenyl)oxazole-5-carboxylic acid (2-(3-(5-(tert-butoxycarbonyl)-1 H-pyrazol-3-yl)phenyl)oxazole-5-carboxylic acid Intermediate 3; (1.39 g, 3.91 mmol), S-valine ethyl ester hydrochloride (1.066 g, 5.87 mmol), and TEA (3.27 mL, 23.47 mmol) in EtOAc (19.5 mL) and the RM left to stir at room temperature for 18 h. The reaction was monitored by LCMS adding additional aliquots of reagents until complete. The RM was diluted with EtOAc and water. The organic phase was separated and washed with brine then dried over MgSO₄ and filtered. The crude material was purified by FCC (10-60% EtOAc/heptane) to afford 1.28 g (67%) of (S)-tert-butyl 3-(3-(5-((1-ethoxy-3-methyl-1-oxobutan-2-yl)carbamoyl)oxazol-2-yl)phenyl)-1 H-pyrazole-5-carboxylate. LCMS Rt: 1.25 mins MS m/z; 483.2 [M+H]+ RXNMON-Acidic 1H NMR (400 MHz, DMSO-d₆) δ 14.05 (d, J=13.5 Hz, 1H), 8.88 (dd, J=17.0, 8.1 Hz, 1H), 8.56 (d, J=28.8 Hz, 1H), 8.19-7.99 (m, 3H), 7.67 (dt, J=23.3, 7.8 Hz, 1H), 7.27 (dd, J=47.2, 1.8 Hz, 1H), 4.39-4.28 (m, 1H), 4.24-4.09 (m, 2H), 2.21 (dq, J=13.7, 6.8 Hz, 1H), 1.56 (d, J=7.7 Hz, 9H), 1.22 (t, J=7.1 Hz, 3H), 0.99 (dd, J=15.3, 6.8 Hz, 6H).

Step 2: (S)-3-(3-(5-((1-ethoxy-3-methyl-1-oxobutan-2-yl)carbamoyl)oxazol-2-yl)phenyl)-1 H-pyrazole-5-carboxylic acid: TFA (4.09 mL, 53.1 mmol) was added to a stirred solution of (S)-tert-butyl 3-(3-(5-((1-ethoxy-3-methyl-1-oxobutan-2-yl)carbamoyl)oxazol-2-yl)phenyl)-1 H-pyrazole-5-carboxylate, 1.28 g, 2.65 mmol) in DCM (13.3 mL) and the RM left to stir at room temperature for 24 h. The RM was concentrated to afford 1.374 g (96%) of (S)-3-(3-(5-((1-ethoxy-3-methyl-1-oxobutan-2-yl)carbamoyl)oxazol-2-yl)phenyl)-1 H-pyrazole-5-carboxylic acid as an off-white solid.
LCMS Rt: 0.93 mins MS m/z; 427.2 [M+H]+ RXNMON-Acidic
1H NMR (400 MHz, DMSO-d₆) δ 8.89 (d, J=8.1 Hz, 1H), 8.57 (t, J=1.5 Hz, 1H), 8.12 (dt, J =7.8, 1.3 Hz, 1H), 8.09-8.03 (m, 2H), 7.66 (t, J=7.8 Hz, 1H), 7.32 (s, 1H), 4.32 (t, J=7.8 Hz, 1H), 4.23-4.09 (m, 2H), 2.21 (dq, J=13.7, 6.8 Hz, 1H), 1.22 (t, J=7.1 Hz, 3H), 0.99 (dd, J=15.0, 6.8 Hz, 6H).
Step 3: (S)-ethyl 2-(2-(3-(5-(((R)-1-methoxy-3-methyl-1-oxobutan-2-yl)carbamoyl)-1H-pyrazol-3-yl)phenyl)oxazole-5-carboxamido)-3-methylbutanoate: T3P (50% solution in EtOAc, 84 μL, 0.141 mmol) was added dropwise to a stirred solution of (S)-3-(3-(5-((1-ethoxy-3-methyl-1-oxobutan-2-yl)carbamoyl)oxazol-2-yl)phenyl)-1 H-pyrazole-5-carboxylic acid ((S)-3-(3-(5-((1-ethoxy-3-methyl-1-oxobutan-2-yl)carbamoyl)oxazol-2-yl)phenyl)-1H-pyrazole-5-carboxylic acid, 38 mg, 0.07 mmol), TEA (59 μL, 0.422 mmol) and (R)-methyl 2-amino-3-methylbutanoate hydrochloride (18 mg, 0.105 mmol) in EtOAc (0.7 mL) and the RM left to stir at room temperature for 2.5 h. The RM was diluted with 50% saturated NaHCO₃ (20 mL) and extracted with EtOAc (30 mL). The organic phase was separated, dried over MgSO₄, and filtered. The filtrate was concentrated and purified by FCC (20-70% EtOAc/heptane) to afford 32 mg (83%) of (S)-ethyl 2-(2-(3-(5-(((R)-1-methoxy-3-methyl-1-oxobutan-2-yl)carbamoyl)-1 H-pyrazol-3-yl)phenyl)oxazole-5-carboxamido)-3-methylbutanoate, Example 2.0, as a clear glass-like solid.
1H NMR (400 MHz, DMSO-d₆) δ 14.10-13.69 (m, 1H), 8.90 (s, 1H), 8.73-7.93 (m, 5H), 7.80-7.21 (m, 2H), 4.35 (dt, J=18.4, 7.7 Hz, 2H), 4.25-4.08 (m, 2H), 3.69 (s, 3H), 2.29-2.13 (m, 2H), 1.23 (t, J=7.1 Hz, 3H), 1.07-0.89 (m, 12H). LCMS Rt: 1.14 mins MS m/z; 540.1 [M+H]+ RXNMON-Acidic
Examples 2.1 to 2.5 were prepared by a similar method to that of Example 2.0 by replacing the amines in Step 1 and Step 3 with the appropriate amines.

### Example 2.1: (S)-Methyl 2-(5-(3-(5-(((S)-1-Ethoxy-3-Methyl-1-Oxobutan-2-yl)Carbamoyl)Oxazol-2-Yl)Phenyl)-1H-Pyrazole-3-Carboxamido)-4-Methylpentanoate

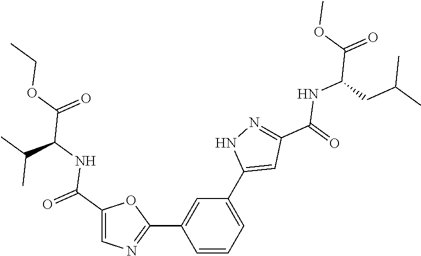 [SMILES: CCOC(=O)[C@@H](NC(=O)c1cnc(-c2cccc(-c3cc(C(=O)N[C@@H](CC(C)C)C(=O)OC)n[nH]3)c2)o1)C(C)C]

LCMS Rt: 1.42 mins MS m/z; 554.5 [M+H]+ 2minLowpHv03

### Example 2.2: (S)-Ethyl 2-(2-(3-(3-(((S)-1-Ethoxy-3-Methyl-1-Oxobutan-2-Yl)Carbamoyl)-1 H-Pyrazol-5-Yl)Phenyl)Oxazole-5-Carboxamido)-3-Methylbutanoate

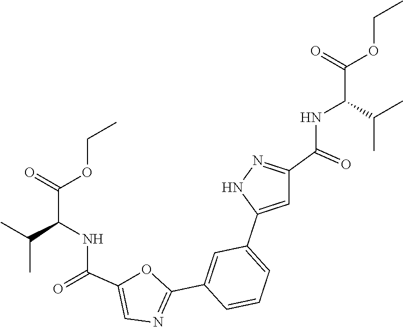 [SMILES: CCOC(=O)[C@@H](NC(=O)c1cc(-c2cccc(-c3ncc(C(=O)N[C@H](C(=O)OCC)C(C)C)o3)c2)[nH]n1)C(C)C]

LCMS Rt: 1.42 mins MS m/z; 554.5 [M+H]+ 2minLowpHv03

¹H NMR (400 MHz, DMSO-d₆) δ ppm 13.77-14.03 (m, 1H) 8.91 (br s, 1H) 8.51-8.55 (m, 1H) 8.12-8.19 (m, 1H) 8.09 (s, 1H) 8.03 (d, J=8.07 Hz, 1H) 7.66-7.74 (m, 1H) 4.30-4.39 (m, 2H) 4.10-4.22 (m, 4H) 2.17-2.26 (m, 2H) 1.23 (td, J=7.09, 1.22 Hz, 6H) 0.93-1.05 (m, 12H)

### Example 2.3 (i) and Example 2.3 (ii): (2S)-Ethyl 2-(2-(3-(5-((1-Cyclopropyl-2,2,2-Trifluoroethyl)Carbamoyl)-1 H-Pyrazol-3-Yl)Phenyl)Oxazole-5-Carboxamido)-3-Methylbutanoate and (2S)-Ethyl 2-(2-(3-(5-((1-Cyclopropyl-2,2,2-Trifluoroethyl)Carbamoyl)-1 H-Pyrazol-3-Yl)Phenyl)Oxazole-5-Carboxamido)-3-Methylbutanoate

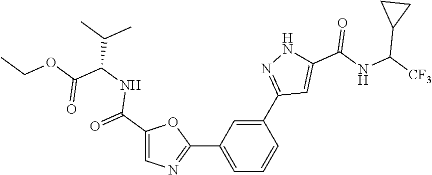 [SMILES: CCOC(=O)[C@@H](NC(=O)c1cnc(-c2cccc(-c3cc(C(=O)NC(C4CC4)C(F)(F)F)[nH]n3)c2)o1)C(C)C]

The isomers were separated using SFC Method 3.

### Example 2.3 (i): (2S)-Ethyl 2-(2-(3-(5-((1-Cyclopropyl-2,2,2-Trifluoroethyl)Carbamoyl)-1H-Pyrazol-3-Yl)Phenyl)Oxazole-5-Carboxamido)-3-Methylbutanoate

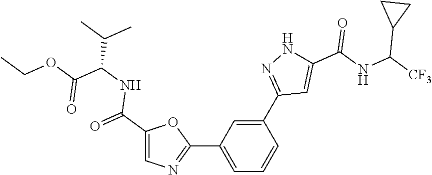 [SMILES: CCOC(=O)[C@@H](NC(=O)c1cnc(-c2cccc(-c3cc(C(=O)NC(C4CC4)C(F)(F)F)[nH]n3)c2)o1)C(C)C]

The faster eluting peak by SFC separation.
LCMS Rt: 1.40 mins MS m/z; 548.4 [M+H]+ ProductAnalysis-Basic

¹H NMR (400 MHz, Methanol-d4) δ 8.52 (s, 1H), 8.17 (d, J=7.9 Hz, 1H), 7.94 (s, 1H), 7.91 (d, J=7.8 Hz, 1H), 7.64 (t, J=7.9 Hz, 1H), 7.22 (s, 1H), 4.52 (d, J=7.0 Hz, 1H), 4.25 (qd, J =7.1, 4.4 Hz, 2H), 4.17-4.06 (m, 1H), 2.36-2.26 (m, 1H), 1.34-1.27 (m, 4H), 1.07 (dd, J=8.7, 6.8 Hz, 6H), 0.79 (dt, J=8.4, 5.1 Hz, 1H), 0.64 (tt, J=10.7, 5.2 Hz, 2H), 0.47-0.38 (m, 1H).

### Example 2.3 (ii): (2S)-Ethyl 2-(2-(3-(5-((1-Cyclopropyl-2,2,2-Trifluoroethyl)Carbamoyl)-1H-Pyrazol-3-Yl)Phenyl)Oxazole-5-Carboxamido)-3-Methylbutanoate

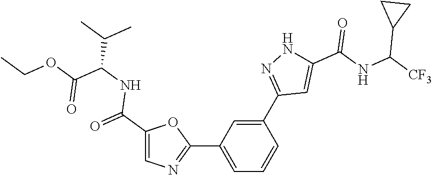 [SMILES: CCOC(=O)[C@@H](NC(=O)c1cnc(-c2cccc(-c3cc(C(=O)NC(C4CC4)C(F)(F)F)[nH]n3)c2)o1)C(C)C]

The slower eluting peak by SFC separation.
LCMS Rt: 1.41 mins MS m/z; 548.3 [M+H]+ ProductAnalysis-Basic

¹H NMR (400 MHz, Methanol-d4) δ 8.52 (s, 1H), 8.17 (d, J=7.7 Hz, 1H), 7.94 (s, 1H), 7.90 (d, J=7.6 Hz, 1H), 7.64 (t, J=7.8 Hz, 1H), 7.22 (s, 1H), 4.52 (d, J=7.0 Hz, 1H), 4.25 (qd, J =7.1, 4.5 Hz, 2H), 4.18-4.06 (m, 1H), 2.31 (h, J=6.8 Hz, 1H), 1.31 (t, J=7.1 Hz, 4H), 1.07 (dd, J=8.7, 6.8 Hz, 6H), 0.80 (s, 1H), 0.64 (dt, J=12.4, 5.3 Hz, 2H), 0.46-0.40 (m, 1H).

### Example 2.4: (2S)-Ethyl 2-(2-(3-(5-((1-Cyclopropyl-2,2-Difluoroethyl)Carbamoyl)-1H-Pyrazol-3-Yl)Phenyl)Oxazole-5-Carboxamido)-3-Methylbutanoate

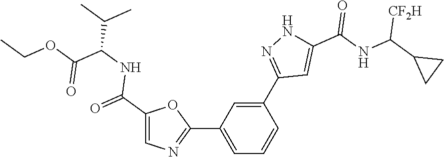 [SMILES: CCOC(=O)[C@@H](NC(=O)c1cnc(-c2cccc(-c3cc(C(=O)NC(C(F)F)C4CC4)[nH]n3)c2)o1)C(C)C]

LCMS Rt: 2.37 mins MS m/z; 530.1[M+H]+ ProductAnalysis-Basic

¹H NMR (400 MHz, Chloroform-d) δ 12.58 (s, 1H), 8.32 (d, J=10.9 Hz, 1H), 8.03 (d, J=7.8 Hz, 1H), 7.78 (d, J=9.3 Hz, 2H), 7.54 (t, J=7.8 Hz, 1H), 7.36-7.28 (m, 1H), 7.18-7.13 (m, 1H), 6.04 (t, J=56.0 Hz, 1H), 4.75 (ddd, J=8.7, 6.0, 2.9 Hz, 1H), 4.35-4.07 (m, 2H), 3.92 (d, J=9.3 Hz, 1H), 2.30 (ddt, J=13.4, 10.8, 6.7 Hz, 1H), 1.30 (td, J=7.1, 3.1 Hz, 3H), 1.25-1.09 (m, 1H), 1.09-1.02 (m, 6H), 0.74 (tt, J=8.3, 4.3 Hz, 1H), 0.55 (dddt, J=30.1, 17.8, 8.7, 4.4 Hz, 3H).

### Example 2.5: N-((S)-1-Cyclopropylethyl)-2-(3-(3-(((S)-1-Cyclopropylethyl)Carbamoyl)-1H-Pyrazol-5-Yl)Phenyl)Oxazole-5-Carboxamide

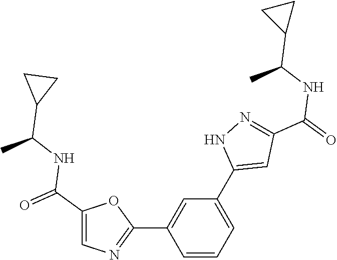 [SMILES: C[C@H](NC(=O)c1cc(-c2cccc(-c3ncc(C(=O)N[C@@H](C)C4CC4)o3)c2)[nH]n1)C1CC1]

LCMS Rt: 1.27 mins MS m/z; 434.4 [M+H]+ 2minLowpHv03

¹H NMR (400 MHz, DMSO-d₆) δ ppm 13.66-13.89 (m, 1H) 8.61-8.70 (m, 1H) 8.51 (t, J=1.59 Hz, 1H) 8.12 (br d, J=7.58 Hz, 1H) 7.99 (d, J=7.83 Hz, 1H) 7.91 (s, 1H) 7.67 (br t, J=7.70 Hz, 1H) 3.38-3.52 (m, 2H) 1.25 (dd, J=11.74, 6.60 Hz, 6H) 0.96-1.07 (m, 2H) 0.46-0.57 (m, 2H) 0.37-0.44 (m, 2H) 0.31 (dt, J=9.11, 4.62 Hz, 2H) 0.24 (dt, J=9.41, 4.58 Hz, 2H)

Example 3 of the present invention may be prepared according to Scheme 9.

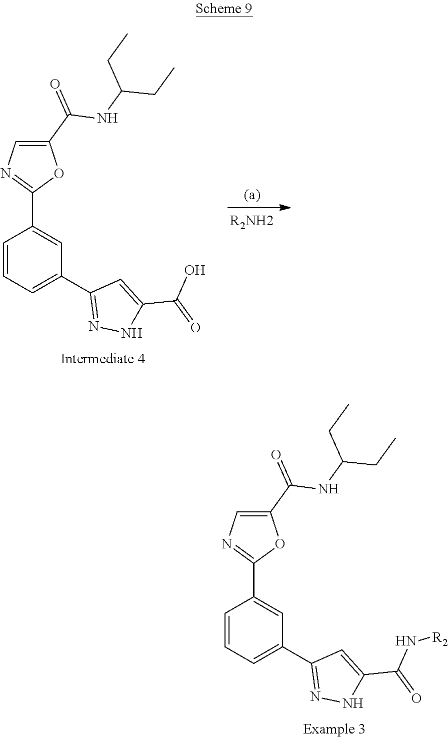

Step (a) involves reaction of an amine (R₃NH2) with Intermediate 4 in a suitable solvent such as DMF or ethyl acetate with a suitable base such as diisopropylethylamine or triethylamine and an amide coupling reagent such as T3P or pyBOP.

### Example 3.0(i) and 3.0(ii): N-(Pentan-3-Yl)-2-(3-(3-(((S)-1-((R)-Tetrahydrofuran-2-Yl)Ethyl)Carbamoyl)-1 H-Pyrazol-5-Yl)Phenyl)Oxazole-5-Carboxamide and, N-(Pentan-3-Yl)-2-(3-(3-(((S)-1-((S)-Tetrahydrofuran-2-Yl)Ethyl)Carbamoyl)-1H-Pyrazol-5-Yl)Phenyl)Oxazole-5-Carboxamide

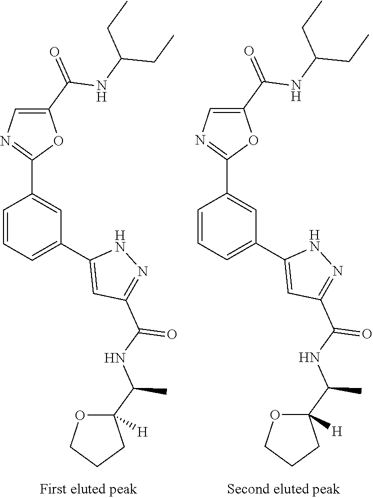 [SMILES: CCC(CC)NC(=O)c1cnc(-c2cccc(-c3cc(C(=O)N[C@@H](C)[C@H]4CCCO4)n[nH]3)c2)o1 ; CCC(CC)NC(=O)c1cnc(-c2cccc(-c3cc(C(=O)N[C@@H](C)[C@@H]4CCCO4)n[nH]3)c2)o1]

To a suspension of 5-(3-(5-(pentan-3-ylcarbamoyl)oxazol-2-yl)phenyl)-1 H-pyrazole-3-carboxylic acid (Intermediate 4) (2.6 g, 5.39 mmol) in EtOAc (200 mL) was added (1 S)-1-(tetrahydrofuran-2-yl)ethanamine (1.512 g, 9.97 mmol), TEA (3.76 mL, 26.9 mmol) and T3P (50% in EtOAc) (6.35 mL, 10.78 mmol). After 3.5 h 10% citric acid was added and the RM was extracted 2×with EtOAc. The combined organic layers were sequentially washed with water and brine, dried over Na2SO4 and concentrated. The crude material was purified by FCC (2-7% MeOH in DCM) to afford 1.03 g (40%) of a mixture of stereoisomers. The stereoisomers were separated by SFC Method 1.

### Example 3.0(i): N-(Pentan-3-Yl)-2-(3-(3-(((S)-1-((R)-Tetrahydrofuran-2-Yl)Ethyl)Carbamoyl)-1 H-Pyrazol-5-Yl)Phenyl)Oxazole-5-Carboxamide

First eluted peak Rt=6.8 minutes. (701 mg, 1.506 mmol, 27.9% yield). Stereochemistry confirmed by x-ray crystal structure referencing to known stereocenter.
LCMS Rt: 2.08 mins MS m/z; 466.5 [M+H]+ ProductAnalysis-Acidic

¹H NMR (400 MHz, DMSO-d₆) TFA δ ppm 8.51 (t, J=1.59 Hz, 1H) 8.30 (d, J=8.80 Hz, 1H) 8.09-8.19 (m, 2H) 8.00 (dt, J=8.07, 1.22 Hz, 1H) 7.93 (s, 1H) 7.67 (t, J=7.95 Hz, 1H) 7.34 (s, 1H) 4.00 (dt, J=8.93, 7.03 Hz, 1H) 3.74-3.87 (m, 3H) 3.59-3.71 (m, 1H) 1.78-1.96 (m, 2H) 1.44-1.73 (m, 4H) 1.18 (d, J=6.85 Hz, 3H) 0.89 (t, J=7.34 Hz, 6H)

### Example 3.0(ii): N-(Pentan-3-Yl)-2-(3-(3-(((S)-1-((S)-Tetrahydrofuran-2-yl)Ethyl)Carbamoyl)-1 H-Pyrazol-5-Yl)Phenyl)Oxazole-5-Carboxamide

Second eluted peak Rt=11.3 minutes. (580 mg, 1.246 mmol, 23.12% yield) Stereochemistry confirmed by x-ray crystal structure referencing to known stereocenter.
LCMS Rt: 2.08 mins MS m/z; 466.5 [M+H]+ ProductAnalysis-Acidic 1H NMR (400 MHz, DMSO-d₆) TFA δ ppm 8.51 (s, 1H) 8.30 (d, J=8.80 Hz, 1H) 8.12 (dt, J=7.98, 1.27 Hz, 1H) 8.00 (dt, J=8.07, 1.28 Hz, 1H) 7.95 (br d, J=8.93 Hz, 1H) 7.92 (s, 1H) 7.67 (t, J=7.83 Hz, 1H) 7.38 (s, 1H) 4.08 (s, 1H) 3.76-3.89 (m, 3H) 3.61-3.68 (m, 1H) 1.86-1.97 (m, 1H) 1.76-1.86 (m, 2H) 1.55-1.65 (m, 3H) 1.44-1.55 (m, 2H) 1.17 (d, J=6.85 Hz, 3H) 0.89 (t, J=7.40 Hz, 6H).
Examples 3.1 to 3.69 were prepared by a similar method to that of Example 3.0 by replacement of (1S)-1-(tetrahydrofuran-2-yl)ethanamine with the appropriate amine.

### Example 3.1: 2-(3-(3-((1-Cyclopropyl-2-Methoxyethyl)Carbamoyl)-1H-Pyrazol-5-Yl)Phenyl)-N-(Pentan-3-Yl)Oxazole-5-Carboxamide

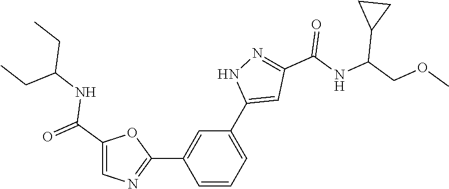 [SMILES: CCC(CC)NC(=O)c1cnc(-c2cccc(-c3cc(C(=O)NC(COC)C4CC4)n[nH]3)c2)o1]

LCMS Rt: 1.27 mins MS m/z; 466.4 [M+H]+ 2minLowpHv03

¹H NMR (400 MHz, CHLOROFORM-d) δ ppm 8.43 (s, 1H) 8.06-8.12 (m, 1H) 7.87 (s, 1H) 7.83 (br d, J=7.83 Hz, 1H) 7.56 (t, J=7.83 Hz, 1H) 7.39 (br d, J=7.58 Hz, 1H) 7.07 (s, 1H) 6.38 (br d, J=9.05 Hz, 1H) 4.00-4.11 (m, 1H) 3.65-3.75 (m, 3H) 3.44 (s, 3H) 1.66-1.79 (m, 2H) 1.57 (dt, J=14.31, 7.03 Hz, 2H) 1.13-1.23 (m, 1H) 1.00 (t, J=7.46 Hz, 6H) 0.50-0.65 (m, 3H) 0.34-0.43 (m, 1H)

### Example 3.2: 2-(3-(3-(Heptan-4-Ylcarbamoyl)-1 H-Pyrazol-5-Yl)Phenyl)-N-(Pentan-3-yl)Oxazole-5-Carboxamide

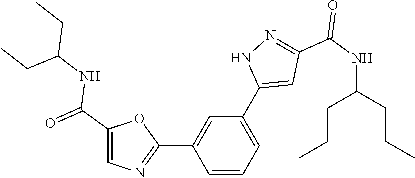 [SMILES: CCCC(CCC)NC(=O)c1cc(-c2cccc(-c3ncc(C(=O)NC(CC)CC)o3)c2)[nH]n1]

LCMS Rt: 1.48 mins MS m/z; 466.6 [M+H]+ 2minLowpHv03

¹H NMR (400 MHz, DMSO-d₆) δ ppm 13.48-13.98 (m, 1H) 8.51 (s, 1H) 8.27-8.37 (m, 1H) 8.12 (br d, J=7.58 Hz, 1H) 7.97-8.05 (m, 1H) 7.93 (s, 1H) 7.56-7.79 (m, 1H) 7.15-7.55 (m, 1H) 3.94-4.04 (m, 1H) 3.74-3.85 (m, 1H) 1.55-1.66 (m, 2H) 1.41-1.54 (m, 7H) 1.24-1.38 (m, 4H) 0.89 (br t, J=7.21 Hz, 12H)

### Example 3.3: (S)-Benzyl 3-Methyl-2-(5-(3-(5-(Pentan-3-Ylcarbamoyl)Oxazol-2-Yl)Phenyl)-1 H-Pyrazole-3-Carboxamido)Butanoate

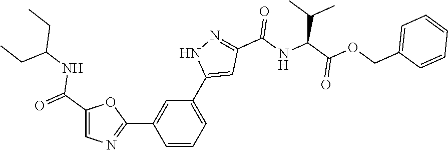 [SMILES: CCC(CC)NC(=O)c1cnc(-c2cccc(-c3cc(C(=O)N[C@H](C(=O)OCc4ccccc4)C(C)C)n[nH]3)c2)o1]

LCMS Rt: 1.50 mins MS m/z; 558.5 [M+H]+ 2minLowpHv03

¹H NMR (400 MHz, DMSO-d₆) δ ppm 13.78-13.95 (m, 1H) 8.52 (s, 1H) 8.28-8.34 (m, 1H) 8.13 (br d, J=7.09 Hz, 1H) 8.01 (br d, J=8.07 Hz, 1H) 7.93 (s, 1H) 7.65-7.74 (m, 1H) 7.31-7.44 (m, 5H) 5.19 (d, J=1.47 Hz, 2H) 4.42 (t, J=7.34 Hz, 1H) 3.75-3.85 (m, 1H) 2.18-2.28 (m, 1H) 1.56-1.65 (m, 2H) 1.44-1.56 (m, 2H) 0.96-1.00 (m, 2H) 0.94 (br d, J=6.60 Hz, 2H) 0.89 (t, J=7.34 Hz, 6H)

### Example 3.4 (i), 3.4 (ii), and 3.4 (iii) and 3.4 (iv): 2-(3-(3-((Cyclopropyl-(Tetrahydrofuran-2-Yl)Methyl)Carbamoyl)-1 H-Pyrazol-5-Yl)Phenyl)-N-(Pentan-3-Yl)Oxazole-5-Carboxamide

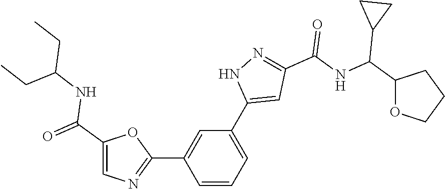 [SMILES: CCC(CC)NC(=O)c1cnc(-c2cccc(-c3cc(C(=O)NC(C4CC4)C4CCCO4)n[nH]3)c2)o1]

The stereoisomers were separated by SFC Method 4.

### Example 3.4 (i): 2-(3-(3-((Cyclopropyl(Tetrahydrofuran-2-Yl)Methyl)Carbamoyl)-1H-Pyrazol-5-Yl)Phenyl)-N-(Pentan-3-Yl)Oxazole-5-Carboxamide

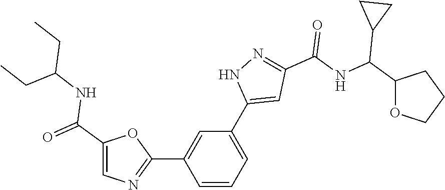 [SMILES: CCC(CC)NC(=O)c1cnc(-c2cccc(-c3cc(C(=O)NC(C4CC4)C4CCCO4)n[nH]3)c2)o1]

Peak eluted at 3.20 min.
LCMS Rt: 2.25 min MS m/z; 492.6 [M+H]+ ProductAnalysis-Acidic

¹H NMR (400 MHz, DMSO-d6) (TFA added) δ ppm 8.50 (t, J=1.53 Hz, 1H) 8.29 (br d, J=8.31 Hz, 1H) 8.12 (br d, J=8.07 Hz, 1H) 7.97-8.03 (m, 1H) 7.92 (s, 1H) 7.67 (t, J=7.83 Hz, 1H) 4.01 (br d, J=4.89 Hz, 1H) 3.74-3.86 (m, 2H) 3.62-3.70 (m, 1H) 1.88-2.00 (m, 1H) 1.81 (t, J=7.03 Hz, 2H) 1.43-1.68 (m, 5H) 1.04-1.16 (m, 1H) 0.88 (t, J=7.34 Hz, 6H) 0.46-0.56 (m, 1H) 0.31-0.44 (m, 1H) 0.25-0.31 (m, 1H).

### Example 3.4 (ii): 2-(3-(3-((Cyclopropyl(Tetrahydrofuran-2-Yl)Methyl)Carbamoyl)-1H-Pyrazol-5-Yl)Phenyl)-N-(Pentan-3-Yl)Oxazole-5-Carboxamide

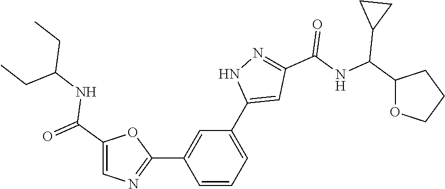 [SMILES: CCC(CC)NC(=O)c1cnc(-c2cccc(-c3cc(C(=O)NC(C4CC4)C4CCCO4)n[nH]3)c2)o1]

Peak eluted at 3.67 min.
LCMS Rt: 2.23 min MS m/z; 492.6 [M+H]+ ProductAnalysis-Acidic

¹H NMR (400 MHz, DMSO-d6) (TFA added) δ ppm 8.50 (s, 1H) 8.24-8.34 (m, 1H) 8.07-8.16 (m, 1H) 7.96-8.03 (m, 1H) 7.92 (s, 1H) 7.63-7.71 (m, 1H) 3.92-4.03 (m, 1H) 3.73-3.85 (m, 2H) 3.62-3.72 (m, 1H) 3.50-3.59 (m, 1H) 1.72-1.95 (m, 4H) 1.54-1.65 (m, 2H) 1.42-1.54 (m, 2H) 1.00-1.14 (m, 1H) 0.88 (t, J=7.40 Hz, 6H) 0.50-0.59 (m, 1H) 0.29-0.39 (m, 2H) 0.20-0.27 (m, 1H).

### Example 3.4 (iii): 2-(3-(3-((Cyclopropyl(Tetrahydrofuran-2-Yl)Methyl)Carbamoyl)-1H-Pyrazol-5-Yl)Phenyl)-N-(Pentan-3-Yl)Oxazole-5-Carboxamide

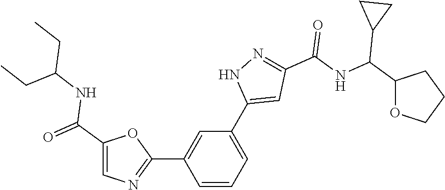 [SMILES: CCC(CC)NC(=O)c1cnc(-c2cccc(-c3cc(C(=O)NC(C4CC4)C4CCCO4)n[nH]3)c2)o1]

Peak eluted at 3.75 min.
LCMS Rt: 2.25 min MS m/z; 492.6 [M+H]+ ProductAnalysis-Acidic

¹H NMR (400 MHz, DMSO-d₆) (tautomers) δ ppm 13.88 (br s, 0.5 H) 13.72 (br s, 0.5 H) 8.51 (t, J=1.47 Hz, 1H) 8.31 (br t, J=7.64 Hz, 1H) 8.12-8.28 (m, 1H) 7.90-8.11 (m, 3H) 7.59-7.74 (m, 1H) 7.55 (s, 0.5 H) 7.21 (s, 0.5 H) 3.95-4.06 (m, 1H) 3.76-3.84 (m, 2H) 3.61-3.70 (m, 1H) 3.39-3.48 (m, 1H) 1.88-2.02 (m, 1H) 1.81 (quin, J=7.12 Hz, 2H) 1.55-1.69 (m, 3H) 1.43-1.55 (m, 3H) 1.05-1.12 (m, 1H) 0.88 (t, J=7.40 Hz, 7H) 0.47-0.56 (m, 1H) 0.37-0.45 (m, 1H) 0.24-0.36 (m, 2H)

### Example 3.4 (iv): 2-(3-(3-((Cyclopropyl(Tetrahydrofuran-2-Yl)Methyl)Carbamoyl)-1H-Pyrazol-5-Yl)Phenyl)-N-(Pentan-3-Yl)Oxazole-5-Carboxamide

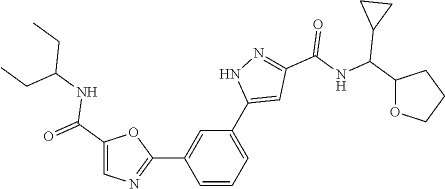 [SMILES: CCC(CC)NC(=O)c1cnc(-c2cccc(-c3cc(C(=O)NC(C4CC4)C4CCCO4)n[nH]3)c2)o1]

Peak eluted at 3.99 min.
LCMS Rt: 2.23 min MS m/z; 492.6 [M+H]+ ProductAnalysis-Acidic ¹H NMR (400 MHz, DMSO-d₆) (TFA added) δ ppm 8.31 (br d, J=8.80 Hz, 2H) 8.09-8.15 (m, 2H) 8.01 (dt, J=8.04, 1.30 Hz, 1H) 7.93 (s, 1H) 7.68 (t, J=7.76 Hz, 1H) 7.37 (s, 1H) 3.99 (q, J=6.48 Hz, 1H) 3.76-3.85 (m, 2H) 3.65-3.72 (m, 1H) 3.50-3.59 (m, 1H) 1.87-1.97 (m, 1H) 1.74-1.87 (m, 3H) 1.55-1.67 (m, 2H) 1.44-1.54 (m, 2H) 1.02-1.13 (m, 1H) 0.89 (t, J=7.40 Hz, 6H) 0.50-0.58 (m, 1H) 0.30-0.38 (m, 2H) 0.22-0.30 (m, 1H). (some aromatic protons obscured by TFA peak)

### Example 3.5: 2-(3-(3-((Cyclobutylmethyl)Carbamoyl)-1H-Pyrazol-5-Yl)Phenyl)-N-(Pentan-3-Yl)Oxazole-5-Carboxamide

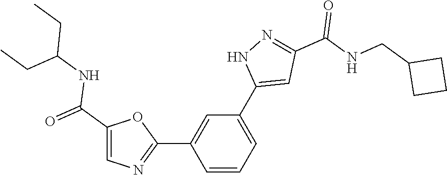 [SMILES: CCC(CC)NC(=O)c1cnc(-c2cccc(-c3cc(C(=O)NCC4CCC4)n[nH]3)c2)o1]

LCMS Rt: 1.36 min MS m/z; 436.3 [M+H]+ 2minLowpHv03

¹H NMR (400 MHz, CHLOROFORM-d) δ ppm 8.41 (s, 1H) 8.06 (d, J=7.82 Hz, 1H) 7.81-7.88 (m, 2H) 7.56 (t, J=7.83 Hz, 1H) 7.11 (s, 1H) 6.94 (br t, J=5.38 Hz, 1H) 6.24 (br d, J=9.29 Hz, 1H) 3.99-4.13 (m, 1H) 3.52-3.57 (m, 2H) 2.55-2.69 (m, 1H) 2.08-2.20 (m, 2H) 1.88-2.00 (m, 2H) 1.76-1.86 (m, 2H) 1.67-1.75 (m, 2H) 1.51-1.63 (m, 2H) 1.00 (t, J=7.46 Hz, 6H)

### Example 3.6: 2-(3-(3-([1,1′-Bi(Cyclopropan)]-1-Ylcarbamoyl)-1H-Pyrazol-5-Yl)Phenyl)-N-(Pentan-3-Yl)Oxazole-5-Carboxamide

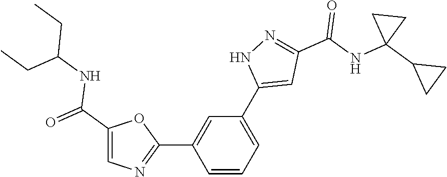 [SMILES: CCC(CC)NC(=O)c1cnc(-c2cccc(-c3cc(C(=O)NC4(C5CC5)CC4)n[nH]3)c2)o1]

LCMS Rt: 1.30 min MS m/z; 448.4 [M+H]+ 2minLowpHv03

¹H NMR (400 MHz, CHLOROFORM-d) δ ppm 8.35-8.40 (m, 1H) 8.04 (d, J=8.07 Hz, 1H) 7.81 (s, 2H) 7.54 (t, J=7.83 Hz, 1H) 7.32 (s, 1H) 7.09 (s, 1H) 6.22 (br d, J=9.05 Hz, 1H) 3.97-4.10 (m, 1H) 1.64-1.78 (m, 2H) 1.48-1.62 (m, 3H) 0.98 (t, J=7.46 Hz, 6H) 0.83-0.88 (m, 2H) 0.69-0.74 (m, 2H) 0.41-0.48 (m, 2H) 0.21-0.27 (m, 2H)

### Example 3.7: 2-(3-(3-((2-Cyclopropylpropan-2-Yl)Carbamoyl)-1H-Pyrazol-5-Yl)Phenyl)-N-(Pentan-3-Yl)Oxazole-5-Carboxamide

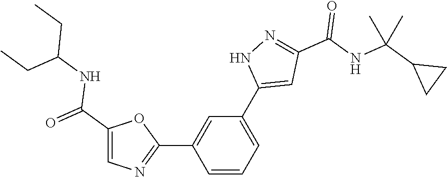 [SMILES: CCC(CC)NC(=O)c1cnc(-c2cccc(-c3cc(C(=O)NC(C)(C)C4CC4)n[nH]3)c2)o1]

LCMS Rt: 1.40 min MS m/z; 450.4 [M+H]+ 2minLowpHv03

¹H NMR (400 MHz, CHLOROFORM-d) δ ppm 8.38 (s, 1H) 8.04 (d, J=8.07 Hz, 1H) 7.80-7.86 (m, 2H) 7.53 (t, J=7.82 Hz, 1H) 7.06 (s, 1H) 6.80 (s, 1H) 6.30 (d, J=9.29 Hz, 1H) 4.04 (dt, J=8.99, 5.29 Hz, 1H) 1.65-1.77 (m, 2H) 1.56 (dquin, J=14.52, 7.37, 7.37, 7.37, 7.37 Hz, 2H) 1.32-1.40 (m, 1H) 0.98 (t, J=7.46 Hz, 6H) 0.42-0.53 (m, 4H)

### Example 3.8: 2-(3-(3-((2-Cyclopropyl-1,1,1-Trifluoropropan-2-Yl)Carbamoyl)-1H-Pyrazol-5-Yl)Phenyl)-N-(Pentan-3-Yl)Oxazole-5-Carboxamide

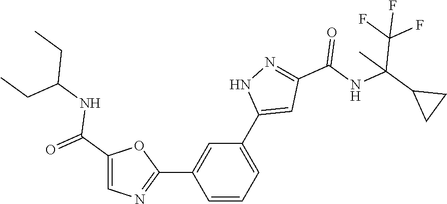 [SMILES: CCC(CC)NC(=O)c1cnc(-c2cccc(-c3cc(C(=O)NC(C)(C4CC4)C(F)(F)F)n[nH]3)c2)o1]

LCMS Rt: 1.45 min MS m/z; 504.4 [M+H]+ 2minLowpHv03

¹H NMR (400 MHz, CHLOROFORM-d) δ ppm 8.35 (t, J=1.47 Hz, 1H) 8.10 (dt, J=7.95, 1.16 Hz, 1H) 7.83 (s, 1H) 7.76-7.80 (m, 1H) 7.60 (t, J=7.83 Hz, 1H) 7.11 (s, 1H) 6.82 (s, 1H) 6.08 (br d, J=9.29 Hz, 1H) 4.00-4.12 (m, 1H) 1.68-1.81 (m, 2H) 1.64 (s, 3H) 1.58 (br dd, J=14.55, 6.97 Hz, 3H) 1.00 (t, J=7.46 Hz, 6H) 0.57-0.71 (m, 4H)

### Example 3.9: 2-(3-(3-((2-Isopropoxyethyl)Carbamoyl)-1H-Pyrazol-5-Yl)Phenyl)-N-(Pentan-3-Yl)Oxazole-5-Carboxamide

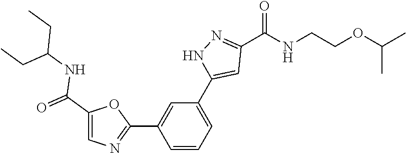 [SMILES: CCC(CC)NC(=O)c1cnc(-c2cccc(-c3cc(C(=O)NCCOC(C)C)n[nH]3)c2)o1]

LCMS Rt: 1.28 min MS m/z; 454.3 [M+H]+ 2minLowpHv03

¹H NMR (400 MHz, CHLOROFORM-d) δ ppm 8.44 (s, 1H) 8.10 (d, J=7.82 Hz, 1H) 7.82-7.87 (m, 2H) 7.69 (br s, 1H) 7.58 (t, J=7.83 Hz, 1H) 7.12 (s, 1H) 6.25 (br d, J=9.05 Hz, 1 H) 3.99-4.12 (m, 1H) 3.69-3.73 (m, 4H) 1.65-1.79 (m, 2H) 1.57 (dquin, J=14.52, 7.37, 7.37, 7.37, 7.37 Hz, 2H) 1.20 (d, J=6.11 Hz, 6H) 1.00 (t, J=7.46 Hz, 6H)

### Example 3.10: 2-(3-(3-((1-Cyclobutylethyl)Carbamoyl)-1H-Pyrazol-5-Yl)Phenyl)-N-(Pentan-3-Yl)Oxazole-5-Carboxamide

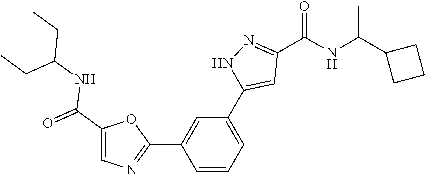 [SMILES: CCC(CC)NC(=O)c1cnc(-c2cccc(-c3cc(C(=O)NC(C)C4CCC4)n[nH]3)c2)o1]

LCMS Rt: 1.37 min MS m/z; 450.4 [M+H]+ 2minLowpHv03

¹H NMR (400 MHz, CHLOROFORM-d) δ ppm 8.41 (s, 1H) 8.00 (d, J=8.07 Hz, 1H) 7.86 (s, 1H) 7.82 (br d, J=7.82 Hz, 1H) 7.48 (t, J=7.83 Hz, 1H) 7.10 (s, 1H) 6.91 (br d, J=9.05 Hz, 1H) 6.52 (br d, J=9.05 Hz, 1H) 4.19-4.31 (m, 1H) 3.99-4.10 (m, 1H) 2.35-2.46 (m, 1H) 1.98-2.12 (m, 2H) 1.77-1.93 (m, 4H) 1.64-1.75 (m, 2H) 1.50-1.62 (m, 2H) 1.17 (d, J=6.60 Hz, 3H) 0.98 (t, J=7.34 Hz, 6H)

### Example 3.11: (S)-2-(3-(3-((1-Methoxypropan-2-Yl)Carbamoyl)-1H-Pyrazol-5-Yl)Phenyl)-N-(Pentan-3-Yl)Oxazole-5-Carboxamide

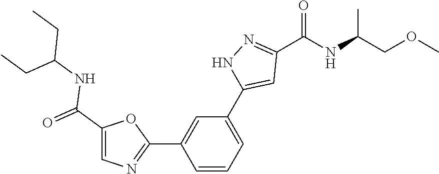 [SMILES: CCC(CC)NC(=O)c1cnc(-c2cccc(-c3cc(C(=O)N[C@@H](C)COC)n[nH]3)c2)o1]

LCMS Rt: 1.19 min MS m/z; 440.4 [M+H]+ 2minLowpHv03

¹H NMR (400 MHz, CHLOROFORM-d) δ ppm 8.39 (s, 1H) 8.07 (d, J=8.07 Hz, 1H) 7.86 (s, 1H) 7.80 (d, J=7.82 Hz, 1H) 7.54 (t, J=7.83 Hz, 1H) 7.23-7.27 (m, 1H) 7.03 (s, 1H) 6.51 (br d, J=9.29 Hz, 1H) 4.49 (ddd, J=8.56, 4.52, 2.08 Hz, 1H) 3.99-4.11 (m, 1H) 3.56 (t, J=5.01 Hz, 2H) 1.66-1.76 (m, 2H) 1.51-1.63 (m, 2H) 1.34 (d, J=6.85 Hz, 3H) 1.00 (td, J=7.46, 3.42 Hz, 6H)

### Example 3.12: 2-(3-(3-((2-Methoxy-2-Methylpropyl)Carbamoyl)-1H-Pyrazol-5-Yl)Phenyl)-N-(Pentan-3-Yl)Oxazole-5-Carboxamide

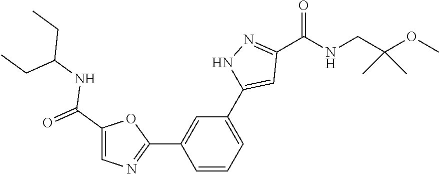 [SMILES: CCC(CC)NC(=O)c1cnc(-c2cccc(-c3cc(C(=O)NCC(C)(C)OC)n[nH]3)c2)o1]

LCMS Rt: 1.23 min MS m/z; 454.4 [M+H]+ 2minLowpHv03

¹H NMR (400 MHz, CHLOROFORM-d) δ ppm 8.51 (s, 1H) 8.14 (d, J=7.58 Hz, 1H) 7.88 (d, J=7.82 Hz, 1H) 7.86 (s, 1H) 7.63 (d, J=7.58 Hz, 1H) 7.14 (s, 1H) 6.98-7.06 (m, 1H) 6.09-6.15 (m, 1H) 3.99-4.12 (m, 1H) 3.54 (d, J=5.87 Hz, 2H) 3.30 (s, 3H) 1.68-1.79 (m, 3H) 1.60 (br dd, J=14.67, 7.09 Hz, 3H) 1.27 (s, 6H) 1.01 (t, J=7.34 Hz, 6H)

### Example 3.13: 2-(3-(3-(((1-Methylcyclopropyl)Methyl)Carbamoyl)-1H-Pyrazol-5-Yl)Phenyl)-N-(Pentan-3-Yl)Oxazole-5-Carboxamide

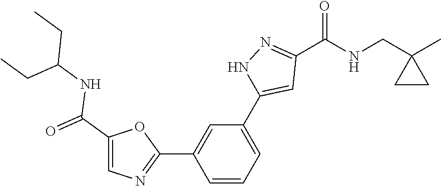 [SMILES: CCC(CC)NC(=O)c1cnc(-c2cccc(-c3cc(C(=O)NCC4(C)CC4)n[nH]3)c2)o1]

LCMS Rt: 1.02 min MS m/z; 436.4 [M+H]+ 2minLowpHv03

¹H NMR (400 MHz, CHLOROFORM-d) δ ppm 8.44 (s, 1H) 8.05 (d, J=7.83 Hz, 1H) 7.82-7.89 (m, 2H) 7.55 (t, J=7.82 Hz, 1H) 7.13 (s, 1H) 7.02 (br t, J=5.26 Hz, 1H) 6.28 (br d, J=9.05 Hz, 1H) 4.05 (dt, J=9.05, 5.26 Hz, 1H) 3.39 (d, J=5.62 Hz, 2H) 1.65-1.79 (m, 2H) 1.52-1.64 (m, 2H) 1.18 (s, 3H) 0.99 (t, J=7.46 Hz, 6H) 0.54 (s, 2H) 0.37-0.43 (m, 2H)

### Example 3.14: N-(Pentan-3-Yl)-2-(3-(3-((3-(Trifluoromethoxy)Phenyl)Carbamoyl)-1 H-Pyrazol-5-Yl)Phenyl)Oxazole-5-Carboxamide

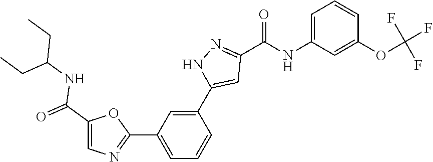 [SMILES: CCC(CC)NC(=O)c1cnc(-c2cccc(-c3cc(C(=O)Nc4cccc(OC(F)(F)F)c4)n[nH]3)c2)o1]

LCMS Rt: 1.35 min MS m/z; 528.4 [M+H]+ 2minLowpHv03

### Example 3.15: 2-(3-(3-(3,3-Dimethylpiperidine-1-Carbonyl)-1H-Pyrazol-5-Yl)Phenyl)-N-(Pentan-3-Yl)Oxazole-5-Carboxamide

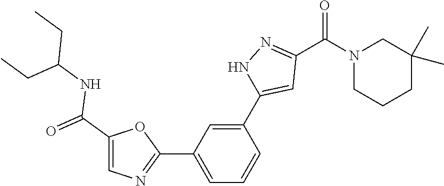 [SMILES: CCC(CC)NC(=O)c1cnc(-c2cccc(-c3cc(C(=O)N4CCCC(C)(C)C4)n[nH]3)c2)o1]

LCMS Rt: 1.23 min MS m/z; 464.5 [M+H]+ 2minLowpHv03

### Example 3.16: 2-(3-(3-(Benzylcarbamoyl)-1H-Pyrazol-5-Yl)Phenyl)-N-(Pentan-3-Yl)Oxazole-5-Carboxamide

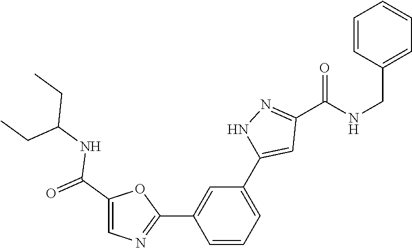 [SMILES: CCC(CC)NC(=O)c1cnc(-c2cccc(-c3cc(C(=O)NCc4ccccc4)n[nH]3)c2)o1]

LCMS Rt: 1.30 min MS m/z; 458.3 [M+H]+ 2minLowpHv03

### Example 3.17: (S)-Ethyl 3-Cyclohexyl-2-(5-(3-(5-(Pentan-3-Ylcarbamoyl)Oxazol-2-Yl)Phenyl)-1 H-Pyrazole-3-Carboxamido)Propanoate

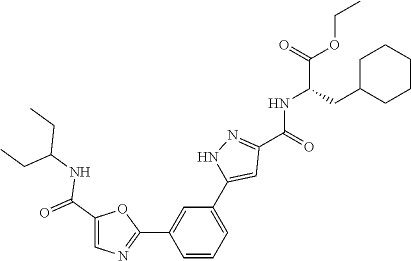 [SMILES: CCOC(=O)[C@H](CC1CCCCC1)NC(=O)c1cc(-c2cccc(-c3ncc(C(=O)NC(CC)CC)o3)c2)[nH]n1]

LCMS Rt: 1.55 min MS m/z; 550.5 [M+H]+ 2minLowpHv03¹H NMR (400 MHz, DMSO-d₆) δ ppm 13.66-14.07 (m, 1H) 8.52 (s, 1H) 8.31 (br d, J=8.56 Hz, 1H) 8.13 (br d, J=7.09 Hz, 1H) 8.01 (d, J=7.82 Hz, 1H) 7.93 (s, 1H) 7.69 (t, J=7.70 Hz, 1H) 4.41-4.61 (m, 1H) 4.05-4.22 (m, 2H) 3.74-3.87 (m, 1H) 1.73-1.80 (m, 2H) 1.63-1.72 (m, 4H) 1.56-1.63 (m, 3H) 1.46-1.55 (m, 2H) 1.34-1.43 (m, 1H) 1.20 (t, J=7.09 Hz, 3H) 1.10-1.18 (m, 2H) 0.93-1.03 (m, 1H) 0.89 (t, J=7.34 Hz, 6H)

### Example 3.18: 2-(3-(3-((Cyclohexylmethyl)Carbamoyl)-1H-Pyrazol-5-Yl)Phenyl)-N-(Pentan-3-Yl)Oxazole-5-Carboxamide

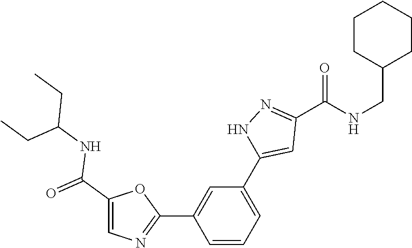 [SMILES: CCC(CC)NC(=O)c1cnc(-c2cccc(-c3cc(C(=O)NCC4CCCCC4)n[nH]3)c2)o1]

LCMS Rt: 1.42 min MS m/z; 464.3 [M+H]+ 2minLowpHv03

¹H NMR (400 MHz, CHLOROFORM-d) δ ppm 8.48 (br s, 1H) 8.13 (br d, J=7.34 Hz, 1H) 7.84 (s, 2H) 7.61 (br d, J=7.34 Hz, 1H) 7.18 (br s, 1H) 7.06-7.14 (m, 1H) 6.19-6.32 (m, 1H) 4.00-4.11 (m, 1H) 3.35 (br t, J=5.26 Hz, 2H) 1.67-1.87 (m, 7H) 1.51-1.65 (m, 3H) 1.16-1.35 (m, 3H) 1.03-1.10 (m, 1H) 0.99 (br t, J=7.46 Hz, 6H)

### Example 3.19: N-(Pentan-3-Yl)-2-(3-(3-((4-(3-(Trifluoromethyl)-3H-Diazirin-3-Yl)Benzyl)Carbamoyl)-1 H-Pyrazol-5-Yl)Phenyl)Oxazole-5-Carboxamide

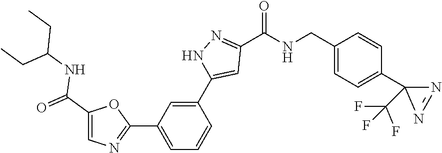 [SMILES: CCC(CC)NC(=O)c1cnc(-c2cccc(-c3cc(C(=O)NCc4ccc(C5(C(F)(F)F)N=N5)cc4)n[nH]3)c2)o1]

LCMS Rt: 1.50 min MS m/z; 566.7 [M+H]+ 2minLowpHv03

### Example 3.20: Ethyl 3-Methyl-1-(5-(3-(5-(Pentan-3-Ylcarbamoyl)Oxazol-2-Yl)Phenyl)-1H-Pyrazole-3-Carbonyl)Pyrrolidine-3-Carboxylate

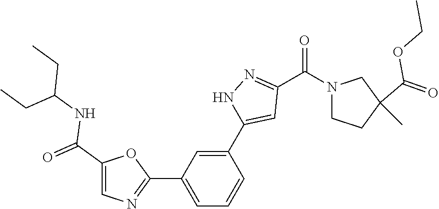 [SMILES: CCOC(=O)C1(C)CCN(C(=O)c2cc(-c3cccc(-c4ncc(C(=O)NC(CC)CC)o4)c3)[nH]n2)C1]

LCMS Rt: 1.19 min MS m/z; 508.5 [M+H]+ 2minLowpHvO3

### Example 3.21: 2-(3-(3-((3-Isopropoxyphenyl)Carbamoyl)-1H-Pyrazol-5-Yl)Phenyl)-N-(Pentan-3-Yl)Oxazole-5-Carboxamide

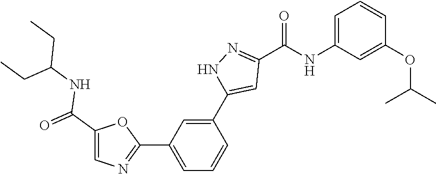 [SMILES: CCC(CC)NC(=O)c1cnc(-c2cccc(-c3cc(C(=O)Nc4cccc(OC(C)C)c4)n[nH]3)c2)o1]

LCMS Rt: 1.33 min MS m/z; 502.5 [M+H]+ 2minLowpHvO3

### Example 3.22: 2-(3-(3-((Benzo[d][1,3]Dioxol-5-Ylmethyl)Carbamoyl)-1H-Pyrazol-5-Yl)Phenyl)-N-(Pentan-3-Yl)Oxazole-5-Carboxamide

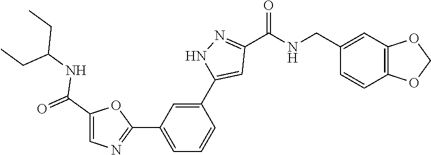 [SMILES: CCC(CC)NC(=O)c1cnc(-c2cccc(-c3cc(C(=O)NCc4ccc5c(c4)OCO5)n[nH]3)c2)o1]

LCMS Rt: 1.17 min MS m/z; 502.4 [M+H]+ 2minLowpHv03

### Example 3.23: 2-(3-(3-((2-(Tert-Butylthio)Ethyl)Carbamoyl)-1H-Pyrazol-5-Yl)Phenyl)-N-(Pentan-3-Yl)Oxazole-5-Carboxamide

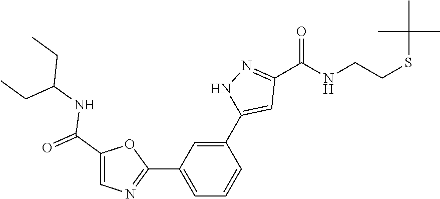 [SMILES: CCC(CC)NC(=O)c1cnc(-c2cccc(-c3cc(C(=O)NCCSC(C)(C)C)n[nH]3)c2)o1]

LCMS Rt: 1.24 min MS m/z; 484.4 [M+H]+ 2minLowpHvO3

### Example 3.24: (S)-2-(3-(3-(Sec-Butylcarbamoyl)-1H-Pyrazol-5-Yl)Phenyl)-N-(Pentan-3-Yl)Oxazole-5-Carboxamide

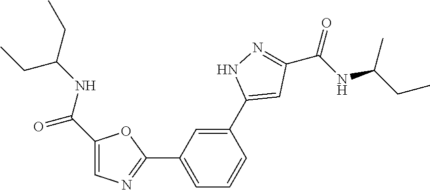 [SMILES: CCC(CC)NC(=O)c1cnc(-c2cccc(-c3cc(C(=O)N[C@@H](C)CC)n[nH]3)c2)o1]

LCMS Rt: 1.16 min MS m/z; 424.4 [M+H]+ 2minLowpHv03

### Pyrazole Example 3.25: (S)-2-(3-(3-((1-Cyclopropylethyl)Carbamoyl)-1H-Pyrazol-5-Yl)Phenyl)-N-(Pentan-3-Yl)Oxazole-5-Carboxamide

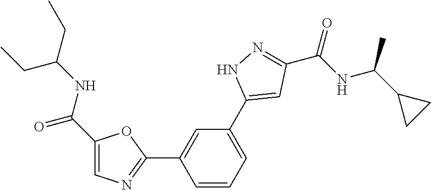 [SMILES: CCC(CC)NC(=O)c1cnc(-c2cccc(-c3cc(C(=O)N[C@@H](C)C4CC4)n[nH]3)c2)o1]

To a suspension of 5-(3-(5-(pentan-3-ylcarbamoyl)oxazol-2-yl)phenyl)-1 H-pyrazole-3-carboxylic acid (7.83 g, 21.25 mmol) in EtOAc (100 mL) was added (S)-1-cyclopropylethanamine (3.40 mL, 31.9 mmol), TEA (8.89 mL, 63.8 mmol) and T3P (50% in EtOAc) (25.05 mL, 42.5 mmol). The RM was monitored by LCMS and worked up after 1.25 h. The RM was quenched with 100 mL of 10% citric acid. The aqueous phase was washed with EtOAc. The combined organics were washed sequentially with water and brine, and then dried over Na2SO4. The crude material was purified by FCC (2-8% MeOH in DCM) to afford 8.03 g (87% yield) of (S)-2-(3-(3-((1-cyclopropylethyl)carbamoyl)-1 H-pyrazol-5-yl)phenyl)-N-(pentan-3-yl)oxazole-5-carboxamide.
LCMS Rt: 1.01 mins MS m/z; 436.6 [M+H]+ RXNMON-Acidic
1H NMR (400 MHz, DMSO-d₆) (TFA added) δ ppm 8.30 (d, J=8.93 Hz, 1H) 8.25 (d, J=8.44 Hz, 1H) 8.12 (dt, J=8.07, 1.22 Hz, 1H) 8.00 (dt, J=8.07, 1.28 Hz, 1H) 7.92 (s, 1H) 7.64-7.70 (m, 1H) 7.34 (s, 1H) 3.79 (br d, J=8.68 Hz, 1H) 3.46 (br d, J=6.72 Hz, 1H) 1.58 (br dd, J=7.46, 5.14 Hz, 2H) 1.44-1.54 (m, 2H) 1.24 (d, J=6.72 Hz, 3H) 0.96-1.08 (m, 1H) 0.89 (t, J=7.40 Hz, 6H) 0.44-0.53 (m, 1H) 0.36-0.43 (m, 1H) 0.28-0.35 (m, 1H) 0.18-0.26 (m, 1H)

### Pyrazole Example 3.25-Methanesulfonate: (S)-2-(3-(3-((1-Cyclopropylethyl)Carbamoyl)-1 H-Pyrazol-5-Yl)Phenyl)-N-(Pentan-3-Yl)Oxazole-5-Carboxamide Methanesulfonate

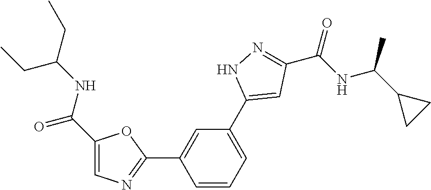 [SMILES: CCC(CC)NC(=O)c1cnc(-c2cccc(-c3cc(C(=O)N[C@@H](C)C4CC4)n[nH]3)c2)o1]

(S)-2-(3-(3-((1-cyclopropylethyl)carbamoyl)-1 H-pyrazol-5-yl)phenyl)-N-(pentan-3-yl)oxazole-5-carboxamide (1.755 g, 4.03 mmol) was dissolved in a mixture of Acetonitrile (60 mL) and 2-Propanol (5 mL) with heating. Methanesulfonic acid (0.275 mL, 4.23 mmol) was added and the resulting mixture was concentrated on the rotovap.
LCMS Rt: 2.25 mins MS m/z; 436.5 [M+H]+ ProdAnalysis-Acidic

¹H NMR (400 MHz, DMSO-d₆) δ ppm 8.50 (t, J=1.53 Hz, 1H) 8.30 (d, J=8.93 Hz, 1H) 8.25 (br d, J=8.19 Hz, 1H) 8.11 (dt, J=7.76, 1.38 Hz, 1H) 7.99 (dt, J=8.07, 1.28 Hz, 1H) 7.92 (s, 1H) 7.67 (t, J=7.83 Hz, 1H) 7.34 (s, 1H) 3.73-3.84 (m, 1H) 3.39-3.50 (m, 1H) 2.33 (s, 3H) 1.55-1.65 (m, 2H) 1.44-1.54 (m, 2H) 1.24 (d, J=6.72 Hz, 3H) 0.97-1.07 (m, 1H) 0.88 (t, J=7.40 Hz, 6H) 0.44-0.52 (m, 1H) 0.36-0.43 (m, 1H) 0.27-0.35 (m, 1H) 0.19-0.26 (m, 1H).

### Pyrazole Example 3.25-Sulfate Salt: (S)-2-(3-(3-((1-Cyclopropylethyl)Carbamoyl)-1H-Pyrazol-5-Yl)Phenyl)-N-(Pentan-3-Yl)Oxazole-5-Carboxamide Sulfate

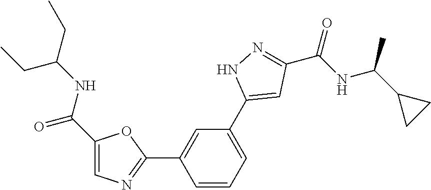 [SMILES: CCC(CC)NC(=O)c1cnc(-c2cccc(-c3cc(C(=O)N[C@@H](C)C4CC4)n[nH]3)c2)o1]

(S)-2-(3-(3-((1-cyclopropylethyl)carbamoyl)-1 H-pyrazol-5-yl)phenyl)-N-(pentan-3-yl)oxazole-5-carboxamide (1.199 g, 2.75 mmol) was dissolved in a mixture of Acetonitrile (40 mL) and 2-Propanol (5 mL) with heating. Added H2SO4 (0.161 mL, 2.89 mmol) dropwise and the resulting mixture was concentrated on the rotovap.
LCMS Rt: 2.25 mins MS m/z; 436.5 [M+H]+ ProdAnalysis-Acidic

¹H NMR (400 MHz, DMSO-d₆) δ ppm 8.51 (t, J=1.47 Hz, 1H) 8.21-8.35 (m, 2H) 8.12 (dt, J=7.82, 1.34 Hz, 1H) 7.97-8.02 (m, 1H) 7.93 (s, 1H) 7.67 (t, J=7.82 Hz, 1H) 7.34 (s, 1H) 4.28 (dt, J=12.47, 6.24 Hz, 1H) 3.73-3.87 (m, 1H) 3.38-3.51 (m, 1H) 1.44-1.67 (m, 4H) 1.24 (d, J=6.85 Hz, 3H) 1.12 (d, J=6.36 Hz, 6H) 0.98-1.07 (m, 1H) 0.89 (t, J=7.34 Hz, 6H) 0.44-0.52 (m, 1H) 0.37-0.44 (m, 1H) 0.28-0.37 (m, 1H) 0.18-0.28 (m, 1H).

### Example 3.26: (R)-2-(3-(3-((1-Cyanopropyl)Carbamoyl)-1H-Pyrazol-5-Yl)Phenyl)-N-(Pentan-3-Yl)Oxazole-5-Carboxamide or (S)-2-(3-(3-((1-Cyanopropyl)Carbamoyl)-1H-Pyrazol-5-Yl)Phenyl)-N-(Pentan-3-Yl)Oxazole-5-Carboxamide

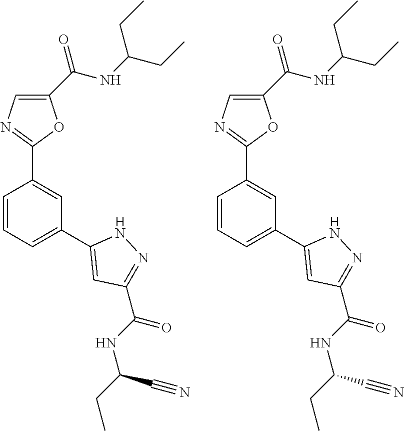 [SMILES: CCC(CC)NC(=O)c1cnc(-c2cccc(-c3cc(C(=O)N[C@@H](C#N)CC)n[nH]3)c2)o1 ; CCC(CC)NC(=O)c1cnc(-c2cccc(-c3cc(C(=O)N[C@H](C#N)CC)n[nH]3)c2)o1]

Separation of the desired stereoisomer by SFC Method 3 using the following conditions afforded 159 mg (43%) of the desired isomer eluting at Rt=1.95 min with the enatiomer eluting at Rt=3.62 min.
LCMS Rt: 2.08 mins MS m/z; 435.3 [M+H]+ ProductAnalysis-Acidic
1H NMR (400 MHz, DMSO-d₆) with TFA δ ppm 9.12 (d, J=8.1 Hz, 1H) 8.53 (t, J=1.6 Hz, 1H) 8.29 (d, J=8.8 Hz, 1H) 8.14 (dt, J=8.1, 1.2 Hz, 1H) 8.02 (dt, J=8.2, 1.2 Hz, 1H) 7.93 (s, 1H) 7.69 (t, J=7.8 Hz, 1H) 7.36 (s, 1H) 4.90 (q, J=7.7 Hz, 1H) 3.70-3.86 (m, 1H) 1.85-2.03 (m, 2H) 1.55-1.66 (m, 2H) 1.45-1.55 (m, 2H) 1.13 (d, J=15.4 Hz, 1H) 1.01 (t, J=7.5 Hz, 3H) 0.89 (t, J=7.5 Hz, 6H).

### Example 3.27: (S)-Methyl 4-Methyl-2-(5-(3-(5-(Pentan-3-Ylcarbamoyl)Oxazol-2-Yl)Phenyl)-1H-Pyrazole-3-Carboxamido)Pentanoate

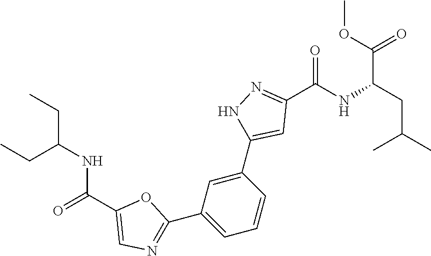 [SMILES: CCC(CC)NC(=O)c1cnc(-c2cccc(-c3cc(C(=O)N[C@@H](CC(C)C)C(=O)OC)n[nH]3)c2)o1]

LCMS Rt: 1.36 min MS m/z; 496.6 [M+H]+ 2minLowpHv03

¹H NMR (400 MHz, CHLOROFORM-d) δ ppm 8.31 (s, 1H) 8.17 (br d, J=8.07 Hz, 1H) 8.08 (br d, J=7.82 Hz, 1H) 7.83 (s, 1H) 7.72 (br d, J=7.82 Hz, 1H) 7.48-7.57 (m, 1H) 7.02 (s, 1H) 6.61 (br d, J=9.29 Hz, 1H) 4.90 (br d, J=7.09 Hz, 1H) 3.99-4.10 (m, 1H) 3.80 (s, 3H) 1.82 (br d, J=6.36 Hz, 1H) 1.74-1.79 (m, 2H) 1.65-1.73 (m, 2H) 1.58 (td, J=13.75, 6.97 Hz, 2H) 0.96-1.04 (m, 12H)

### Example 3.28: Methyl 2-(5-(3-(5-(Pentan-3-Ylcarbamoyl)Oxazol-2-Yl)Phenyl)-1H-Pyrazole-3-Carboxamido)-3-Phenylpropanoate

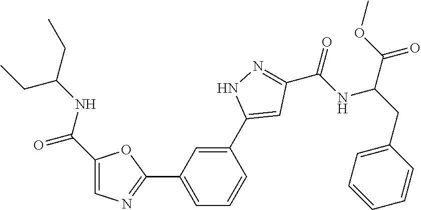 [SMILES: CCC(CC)NC(=O)c1cnc(-c2cccc(-c3cc(C(=O)NC(Cc4ccccc4)C(=O)OC)n[nH]3)c2)o1]

LCMS Rt: 1.24 min MS m/z; 530.5 [M+H]+ 2minLowpHv03

### Example 3.29: 2-(3-(3-((2-Methylpentan-3-Yl)Carbamoyl)-1H-Pyrazol-5-Yl)Phenyl)-N-(Pentan-3-Yl)Oxazole-5-Carboxamide

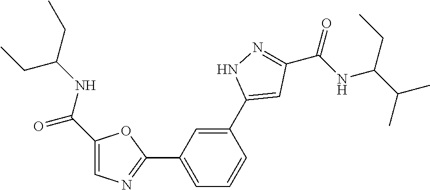 [SMILES: CCC(CC)NC(=O)c1cnc(-c2cccc(-c3cc(C(=O)NC(CC)C(C)C)n[nH]3)c2)o1]

LCMS Rt: 1.25 min MS m/z; 452.5 [M+H]+ 2minLowpHv03

### Example 3.30: Methyl 1-(5-(3-(5-(Pentan-3-Ylcarbamoyl)Oxazol-2-Yl)Phenyl)-1H-Pyrazole-3-Carbonyl)Pyrrolidine-3-Carboxylate

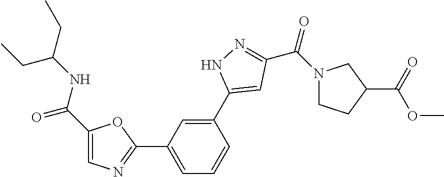 [SMILES: CCC(CC)NC(=O)c1cnc(-c2cccc(-c3cc(C(=O)N4CCC(C(=O)OC)C4)n[nH]3)c2)o1]

LCMS Rt: 1.09 min MS m/z; 480.4 [M+H]+ 2minLowpHv03

### Example 3.31: 2-(3-(3-(2-Isopropylpyrrolidine-1-Carbonyl)-1H-Pyrazol-5-Yl)Phenyl)-N-(Pentan-3-Yl)Oxazole-5-Carboxamide

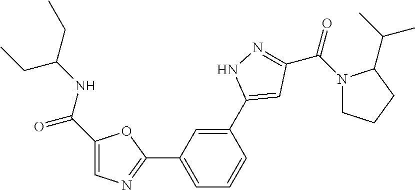 [SMILES: CCC(CC)NC(=O)c1cnc(-c2cccc(-c3cc(C(=O)N4CCCC4C(C)C)n[nH]3)c2)o1]

LCMS Rt: 1.26 min MS m/z; 464.5 [M+H]+ 2minLowpHv03

### Example 3.32: 2-(3-(3-((Cyclopropylmethyl)Carbamoyl)-1 H-Pyrazol-5-Yl)Phenyl)-N-(Pentan-3-Yl)Oxazole-5-Carboxamide

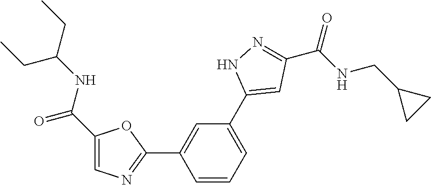 [SMILES: CCC(CC)NC(=O)c1cnc(-c2cccc(-c3cc(C(=O)NCC4CC4)n[nH]3)c2)o1]

LCMS Rt: 1.24 mins MS m/z; 422.4 [M+H]+ 2minLowpHv03

¹H NMR (400 MHz, CHLOROFORM-d) δ ppm 8.43 (s, 1H) 8.06 (d, J=7.82 Hz, 1H) 7.85 (s, 2H) 7.55 (s, 1H) 7.12 (s, 1H) 7.05-7.10 (m, 1H) 6.24-6.33 (m, 1H) 4.00-4.10 (m, 1H) 3.35-3.42 (m, 2H) 1.65-1.78 (m, 2H) 1.50-1.64 (m, 2H) 1.06-1.19 (m, 1H) 0.99 (t, J=7.46 Hz, 6H) 0.59 (br dd, J=7.95, 1.10 Hz, 2H) 0.32 (d, J=5.38 Hz, 2H)

### Example 3.33: N-(Pentan-3-Yl)-2-(3-(3-(Piperidine-1-Carbonyl)-1H-Pyrazol-5-Yl)Phenyl)Oxazole-5-Carboxamide

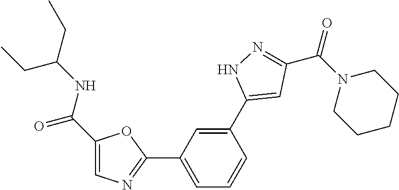 [SMILES: CCC(CC)NC(=O)c1cnc(-c2cccc(-c3cc(C(=O)N4CCCCC4)n[nH]3)c2)o1]

LCMS Rt: 1.13 mins MS m/z; 396.4 [M+H]+ 2minLowpHv03

### Example 3.34: 2-(3-(3-((2,6-Difluorobenzyl)Carbamoyl)-1 H-Pyrazol-5-Yl)Phenyl)-N-(Pentan-3-Yl)Oxazole-5-Carboxamide

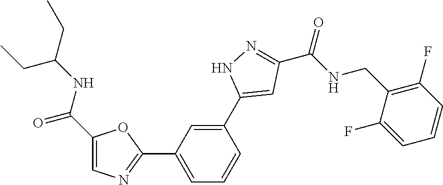 [SMILES: CCC(CC)NC(=O)c1cnc(-c2cccc(-c3cc(C(=O)NCc4c(F)cccc4F)n[nH]3)c2)o1]

LCMS Rt: 1.19 min MS m/z; 422.3 [M+H]+ 2minLowpHv03

### Example 3.35: (S)-2-(3-(3-((3,3-Dimethylbutan-2-Yl)Carbamoyl)-1H-Pyrazol-5-Yl)Phenyl)-N-(Pentan-3-Yl)Oxazole-5-Carboxamide Trifluoroacetate

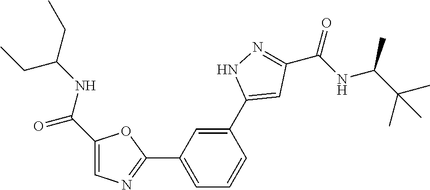 [SMILES: CCC(CC)NC(=O)c1cnc(-c2cccc(-c3cc(C(=O)N[C@@H](C)C(C)(C)C)n[nH]3)c2)o1]

LCMS Rt: 1.37 min MS m/z; 452.4 [M+H]+ 2minLowpHv03

¹H NMR (400 MHz, DMSO-d₆) δ ppm 8.51 (s, 1H) 8.32 (br d, J=8.80 Hz, 1H) 8.12 (br d, J=7.83 Hz, 1H) 8.01 (br d, J=7.83 Hz, 1H) 7.93 (s, 1H) 7.81 (br d, J=8.31 Hz, 1H) 7.43 (br s, 1H) 3.92-4.01 (m, 2H) 3.80 (br d, J=4.89 Hz, 2H) 1.56-1.65 (m, 2H) 1.51 (br dd, J=14.67, 7.09 Hz, 2H) 1.12 (d, J=7.09 Hz, 3H) 0.92 (s, 9H) 0.89 (br t, J=7.34 Hz, 6H)

### Example 3.36: 2-(3-(3-((1-Methoxy-3-Methylbutan-2-Yl)Carbamoyl)-1H-Pyrazol-5-Yl)Phenyl)-N-(Pentan-3-Yl)Oxazole-5-Carboxamide

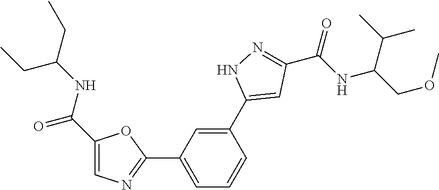 [SMILES: CCC(CC)NC(=O)c1cnc(-c2cccc(-c3cc(C(=O)NC(COC)C(C)C)n[nH]3)c2)o1]

LCMS Rt: 1.19 min MS m/z; 468.5 [M+H]+ 2minLowpHv03

¹H NMR (400 MHz, CHLOROFORM-d) δ ppm 8.44 (s, 1H) 8.10 (d, J=7.82 Hz, 1H) 7.87 (s, 1H) 7.84 (br d, J=8.07 Hz, 1H) 7.58 (t, J=7.83 Hz, 1H) 7.15 (br d, J=9.29 Hz, 1H) 7.10 (s, 1H) 6.27 (br d, J=9.54 Hz, 1H) 4.13 (td, J=8.56, 4.65 Hz, 1H) 4.01-4.09 (m, 1H) 3.71 (dd, J=9.90, 4.52 Hz, 1H) 3.55 (dd, J=9.78, 3.91 Hz, 1H) 3.41 (s, 3H) 1.99-2.13 (m, 1H) 1.66-1.80 (m, 2H) 1.51-1.65 (m, 2H) 1.05 (t, J=6.36 Hz, 6H) 1.01 (t, J=7.46 Hz, 6H)

### Example 3.37: (R)-2-(3-(3-((3-Methylbutan-2-Yl)Carbamoyl)-1H-Pyrazol-5-Yl)Phenyl)-N-(Pentan-3-Yl)Oxazole-5-Carboxamide

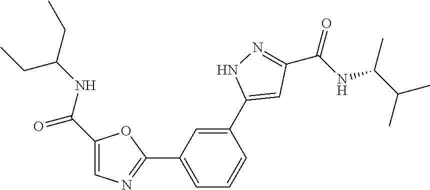 [SMILES: CCC(CC)NC(=O)c1cnc(-c2cccc(-c3cc(C(=O)N[C@H](C)C(C)C)n[nH]3)c2)o1]

LCMS Rt: 1.37 min MS m/z; 437.5 [M+H]+ 2minLowpHv03

### Example 3.38: (S)-N-(Pentan-3-Yl)-2-(3-(3-((1-Phenylethyl)Carbamoyl)-1H-Pyrazol-5-yl)Phenyl)Oxazole-5-Carboxamide

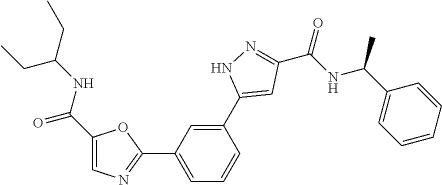 [SMILES: CCC(CC)NC(=O)c1cnc(-c2cccc(-c3cc(C(=O)N[C@@H](C)c4ccccc4)n[nH]3)c2)o1]

LCMS Rt: 1.23 min MS m/z; 472.5 [M+H]+ 2minLowpHv03

### Example 3.39: 2-(3-(3-((2-Methyl-4-Phenylbutan-2-Yl)Carbamoyl)-1H-Pyrazol-5-yl)Phenyl)-N-(Pentan-3-Yl)Oxazole-5-Carboxamide

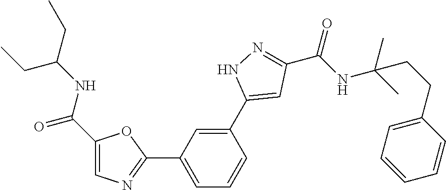 [SMILES: CCC(CC)NC(=O)c1cnc(-c2cccc(-c3cc(C(=O)NC(C)(C)CCc4ccccc4)n[nH]3)c2)o1]

LCMS Rt: 1.37 min MS m/z; 514.0 [M+H]+ 2minLowpHv03

### Example 3.40: 2-(3-(3-(Cyclohexylcarbamoyl)-1 H-Pyrazol-5-Yl)Phenyl)-N-(Pentan-3-yl)Oxazole-5-Carboxamide

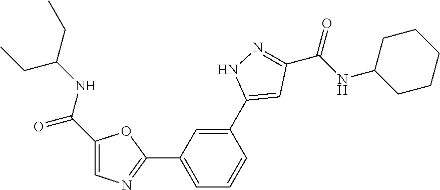 [SMILES: CCC(CC)NC(=O)c1cnc(-c2cccc(-c3cc(C(=O)NC4CCCCC4)n[nH]3)c2)o1]

LCMS Rt: 1.24 min MS m/z; 450.5 [M+H]+ 2minLowpHv03

### Example 3.41: (S)-Methyl 3-Methyl-2-(5-(3-(5-(Pentan-3-Ylcarbamoyl)Oxazol-2-Yl)Phenyl)-1H-Pyrazole-3-Carboxamido)Butanoate

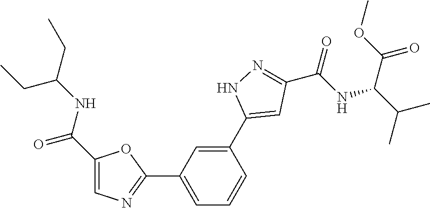 [SMILES: CCC(CC)NC(=O)c1cnc(-c2cccc(-c3cc(C(=O)N[C@H](C(=O)OC)C(C)C)n[nH]3)c2)o1]

LCMS Rt: 1.19 min MS m/z; 482.5 [M+H]+ 2minLowpHv03

### Example 3.42: (S)-Methyl 2-(5-(3-(5-(Pentan-3-Ylcarbamoyl)Oxazol-2-Yl)Phenyl)-1H-Pyrazole-3-Carboxamido)Propanoate

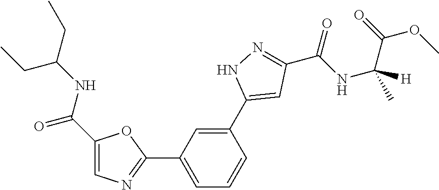 [SMILES: CCC(CC)NC(=O)c1cnc(-c2cccc(-c3cc(C(=O)N[C@@H](C)C(=O)OC)n[nH]3)c2)o1]

LCMS Rt: 1.09 min MS m/z; 454.4 [M+H]+ 2minLowpHv03

### Example 3.43: 2-(3-(3-(Tert-Butylcarbamoyl)-1 H-Pyrazol-5-Yl)Phenyl)-N-(Pentan-3-yl)Oxazole-5-Carboxamide

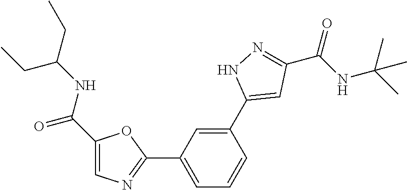 [SMILES: CCC(CC)NC(=O)c1cnc(-c2cccc(-c3cc(C(=O)NC(C)(C)C)n[nH]3)c2)o1]

LCMS Rt: 1.30 min MS m/z; 424.4 [M+H]+ 2minLowpHv03

### Example 3.44: (R)-2-(3-(3-((1-Cyclohexylethyl)Carbamoyl)-1H-Pyrazol-5-Yl)Phenyl)-N-(Pentan-3-Yl)Oxazole-5-Carboxamide

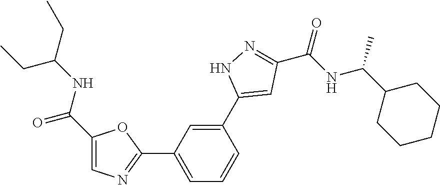 [SMILES: CCC(CC)NC(=O)c1cnc(-c2cccc(-c3cc(C(=O)N[C@H](C)C4CCCCC4)n[nH]3)c2)o1]

LCMS Rt: 1.48 min MS m/z; 477.6 [M+H]+ 2minLowpHv03

### Example 3.45: 2-(3-(3-((4-Fluorobenzyl)Carbamoyl)-1 H-Pyrazol-5-Yl)Phenyl)-N-(Pentan-3-yl)Oxazole-5-Carboxamide

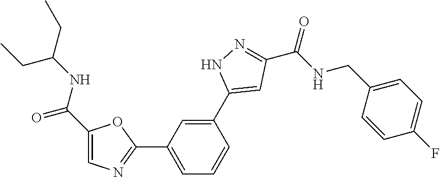 [SMILES: CCC(CC)NC(=O)c1cnc(-c2cccc(-c3cc(C(=O)NCc4ccc(F)cc4)n[nH]3)c2)o1]

LCMS Rt: 4.16 Min MS m/z; 476.5 [M+H]+ 8minLowpHv01

### Example 3.46: (R)-Methyl 4-(Methylthio)-2-(5-(3-(5-(Pentan-3-Ylcarbamoyl)Oxazol-2-yl)Phenyl)-1H-Pyrazole-3-Carboxamido)Butanoate

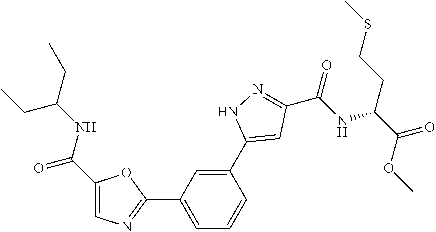 [SMILES: CCC(CC)NC(=O)c1cnc(-c2cccc(-c3cc(C(=O)N[C@H](CCSC)C(=O)OC)n[nH]3)c2)o1]

LCMS Rt: 4.15 min MS m/z; 514.5 [M+H]+ 8minLowpHv01

### Example 3.47: 2-(3-(3-(4-Methoxy-4-Methylpiperidine-1-Carbonyl)-1H-Pyrazol-5-yl)Phenyl)-N-(Pentan-3-Yl)Oxazole-5-Carboxamide

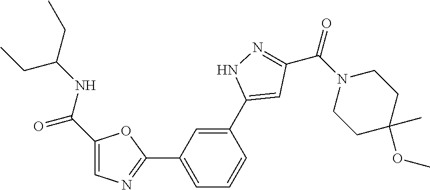 [SMILES: CCC(CC)NC(=O)c1cnc(-c2cccc(-c3cc(C(=O)N4CCC(C)(OC)CC4)n[nH]3)c2)o1]

LCMS Rt: 1.07 min MS m/z; 433.4 [M+H]+ 2minLowpHv03

### Example 3.48: 2-(3-(3-(Cyclopentylcarbamoyl)-1 H-Pyrazol-5-Yl)Phenyl)-N-(Pentan-3-yl)Oxazole-5-Carboxamide

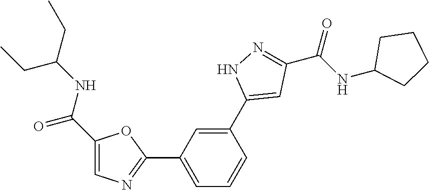 [SMILES: CCC(CC)NC(=O)c1cnc(-c2cccc(-c3cc(C(=O)NC4CCCC4)n[nH]3)c2)o1]

LCMS Rt: 1.29 min MS m/z; 436.4 [M+H]+ 2minLowpHv03

### Example 3.49: (S)-2-(3-(3-((1-Cyclohexylethyl)Carbamoyl)-1H-Pyrazol-5-Yl)Phenyl)-N-(Pentan-3-Yl)Oxazole-5-Carboxamide

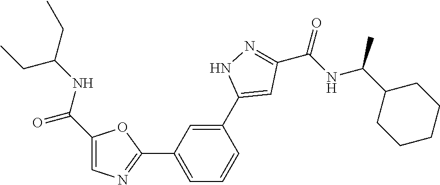 [SMILES: CCC(CC)NC(=O)c1cnc(-c2cccc(-c3cc(C(=O)N[C@@H](C)C4CCCCC4)n[nH]3)c2)o1]

LCMS Rt: 1.33 min MS m/z; 478.5 [M+H]+ 2minLowpHv03

### Example 3.50: (S)-Methyl 2-(5-(3-(5-(Pentan-3-Ylcarbamoyl)Oxazol-2-Yl)Phenyl)-1H-Pyrazole-3-Carboxamido)-3-Phenylpropanoate

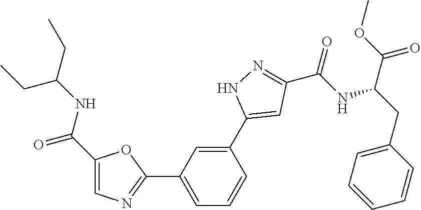 [SMILES: CCC(CC)NC(=O)c1cnc(-c2cccc(-c3cc(C(=O)N[C@@H](Cc4ccccc4)C(=O)OC)n[nH]3)c2)o1]

LCMS Rt: 1.24 min MS m/z; 530.5 [M+H]+ 2minLowpHv03

### Example 3.51: (R)-N-(Pentan-3-Yl)-2-(3-(3-((1-Phenylethyl)Carbamoyl)-1H-Pyrazol-5-Yl)Phenyl)Oxazole-5-Carboxamide

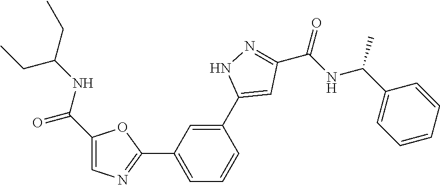 [SMILES: CCC(CC)NC(=O)c1cnc(-c2cccc(-c3cc(C(=O)N[C@H](C)c4ccccc4)n[nH]3)c2)o1]

LCMS Rt: 1.35 min MS m/z; 472.4 [M+H]+ 2minLowpHv03

### Example 3.52: (S)-Ethyl 3-Methyl-2-(5-(3-(5-(Pentan-3-Ylcarbamoyl)Oxazol-2-Yl)Phenyl)-1 H-Pyrazole-3-Carboxamido)Butanoate

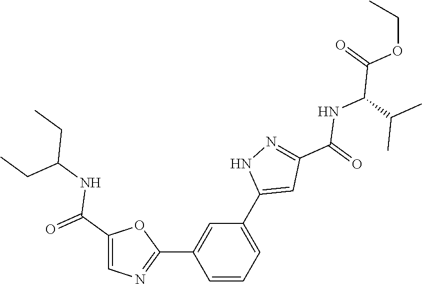 [SMILES: CCOC(=O)[C@@H](NC(=O)c1cc(-c2cccc(-c3ncc(C(=O)NC(CC)CC)o3)c2)[nH]n1)C(C)C]

LCMS Rt: 1.39 mins MS m/z; 496.4 [M+H]+ 2minLowpHv03

¹H NMR (400 MHz, DMSO-d₆) δ ppm 8.52 (s, 1H) 8.27-8.35 (m, 1H) 8.13 (br d, J=7.58 Hz, 1H) 8.02 (br d, J=7.58 Hz, 1H) 7.93 (s, 1H) 7.65-7.74 (m, 1H) 4.36 (br t, J=7.34 Hz, 1H) 4.16 (br dd, J=6.97, 5.50 Hz, 2H) 3.75-3.84 (m, 1H) 2.16-2.27 (m, 1H) 1.59 (br dd, J=14.18, 6.36 Hz, 2H) 1.43-1.54 (m, 2H) 1.23 (t, J=7.09 Hz, 3H) 0.94-1.01 (m, 6H) 0.89 (t, J=7.34 Hz, 6H)

### Example 3.53: 2-(3-(3-(((R)-1-((2R,5R)-5-Methyltetrahydrofuran-2-Yl)Propyl)Carbamoyl)-1 H-Pyrazol-5-Yl)Phenyl)-N-(Pentan-3-Yl)Oxazole-5-Carboxamide

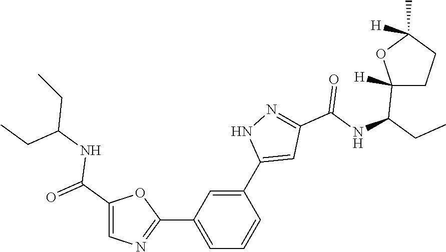 [SMILES: CCC(CC)NC(=O)c1cnc(-c2cccc(-c3cc(C(=O)N[C@H](CC)[C@H]4CC[C@@H](C)O4)n[nH]3)c2)o1]

For preparation of amine used see: Preparation of arylamino heterocyclylmethyl cyclobutenediones as anti-inflammatories.

### Press, Neil John; Watson, Simon, James; Porter, David

Assignee Novartis A.-G., Switz. WO 2008148790
LCMS Rt: 1.23 min MS m/z; 494.5 [M+H]+ 2minLowpHv03

### Example 3.54: N-(Pentan-3-Yl)-2-(3-(3-((2-Phenylpropan-2-Yl)Carbamoyl)-1H-Pyrazol-5-Yl)Phenyl)Oxazole-5-Carboxamide

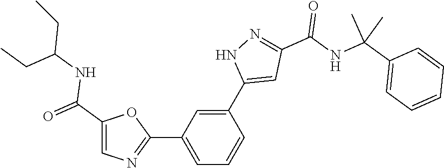 [SMILES: CCC(CC)NC(=O)c1cnc(-c2cccc(-c3cc(C(=O)NC(C)(C)c4ccccc4)n[nH]3)c2)o1]

LCMS Rt: 1.27 min MS m/z; 486.5 [M+H]+ 2minLowpHv03

### Example 3.55: (2S)-Ethyl 3-Methyl-2-(2-(3-(5-((1,1,1-Trifluoropropan-2-Yl)Carbamoyl)-1H-Pyrazol-3-Yl)Phenyl)Oxazole-5-Carboxamido)Butanoate

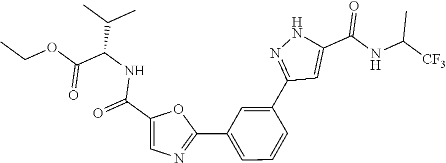 [SMILES: CCOC(=O)[C@@H](NC(=O)c1cnc(-c2cccc(-c3cc(C(=O)NC(C)C(F)(F)F)[nH]n3)c2)o1)C(C)C]

LCMS Rt: 1.34 min MS m/z; 522.2 [M+H]+ RXNMON_Basic.

¹H NMR (400 MHz, Methanol-d4) δ 8.57 (t, J=1.5 Hz, 1H), 8.18 (dt, J=7.8, 1.1 Hz, 1H), 7.98-7.89 (m, 2H), 7.64 (t, J=7.9 Hz, 1H), 7.25 (s, 1H), 4.50 (d, J=7.0 Hz, 1H), 4.24 (qq, J =7.2, 3.7 Hz, 2H), 2.30 (hept, J=6.8 Hz, 1H), 1.45 (d, J=7.1 Hz, 3H), 1.30 (t, J=7.1 Hz, 3H), 1.06 (dd, J=8.3, 6.8 Hz, 6H).

### Example 3.56: (R)-2-(3-(5-((1-Cyclopropylethyl)Carbamoyl)-1H-Pyrazol-3-Yl)Phenyl)-N-(Pentan-3-Yl)Oxazole-5-Carboxamide

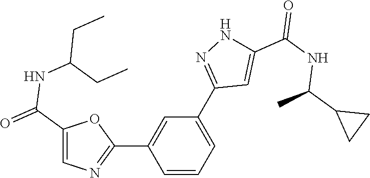 [SMILES: CCC(CC)NC(=O)c1cnc(-c2cccc(-c3cc(C(=O)N[C@H](C)C4CC4)[nH]n3)c2)o1]

LCMS Rt: 1.31 min MS m/z; 436.4 [M+H]+ 2minLowpHv03

¹H NMR (400 MHz, DMSO-d₆) δ ppm 13.70-13.80 (m, 1H) 8.51 (s, 1H) 8.26-8.36 (m, 1H) 8.09-8.16 (m, 1H) 8.00 (br d, J=7.34 Hz, 1H) 7.93 (s, 1H) 7.64-7.73 (m, 1H) 3.74-3.85 (m, 1H) 3.41-3.52 (m, 1H) 1.46-1.65 (m, 4H) 1.25 (d, J=6.85 Hz, 3H) 0.98-1.08 (m, 1H) 0.89 (t, J=7.46 Hz, 6H) 0.45-0.52 (m, 1H) 0.37-0.43 (m, 1H) 0.30-0.36 (m, 1H) 0.20-0.27 (m, 1H)

### Example 3.57: (S)-Tert-Butyl 2-(4-Methyl-2-(5-(3-(5-(Pentan-3-Ylcarbamoyl)Oxazol-2-Yl)Phenyl)-1H-Pyrazole-3-Carboxamido)Pentanamido)Acetate

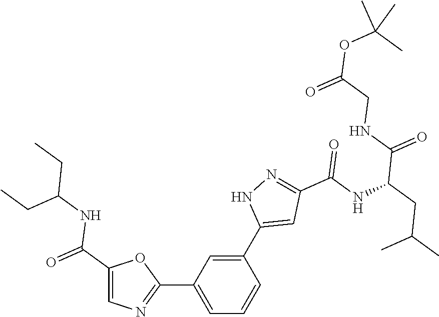 [SMILES: CCC(CC)NC(=O)c1cnc(-c2cccc(-c3cc(C(=O)N[C@@H](CC(C)C)C(=O)NCC(=O)OC(C)(C)C)n[nH]3)c2)o1]

LCMS Rt: 1.25 min MS m/z; 539.5 [M-Boc]+2minLowpHv03

### Example 3.58: (S)-Methyl 2-(2-(3-(3-(((S)-1-Methoxy-4-Methyl-1-Oxopentan-2-Yl)Carbamoyl)-1H-Pyrazol-5-Yl)Phenyl)Oxazole-5-Carboxamido)-4-Methylpentanoate

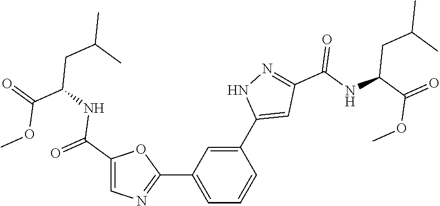 [SMILES: COC(=O)[C@H](CC(C)C)NC(=O)c1cc(-c2cccc(-c3ncc(C(=O)N[C@@H](CC(C)C)C(=O)OC)o3)c2)[nH]n1]

LCMS Rt: 1.27 min MS m/z; 554.5 [M+H]+ 2minLowpHv03

¹H NMR (400 MHz, CHLOROFORM-d) δ ppm 12.20-12.60 (m, 1H) 8.18 (br s, 1H) 8.09 (br d, J=7.82 Hz, 1H) 7.70-7.81 (m, 3H) 7.63 (s, 1H) 7.56 (br t, J=7.95 Hz, 1H) 7.04 (s, 1H) 4.87-5.00 (m, 2H) 3.80 (s, 3H) 3.72 (s, 3H) 1.70-1.93 (m, 6H) 1.27-1.50 (m, 1H) 1.01 (br d, J=6.11 Hz, 12H)

### Example 3.59: (R)-Ethyl 2-(5-(3-(5-(Pentan-3-Ylcarbamoyl)Oxazol-2-Yl)Phenyl)-1H-Pyrazole-3-Carboxamido)-2-Phenylacetate Trifluoroacetate

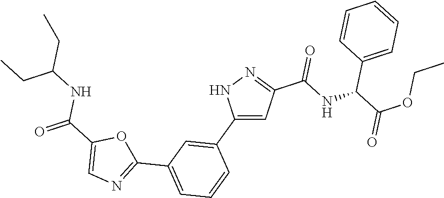 [SMILES: CCOC(=O)[C@H](NC(=O)c1cc(-c2cccc(-c3ncc(C(=O)NC(CC)CC)o3)c2)[nH]n1)c1ccccc1]

LCMS Rt: 1.26 min MS m/z; 530.5 [M+H]+ 2minLowpHv03

### Example 3.60: (S)-Ethyl 4-Methyl-2-(5-(3-(5-(Pentan-3-Ylcarbamoyl)Oxazol-2-Yl)Phenyl)-1H-Pyrazole-3-Carboxamido)Pentanoate Trifluoroacetate

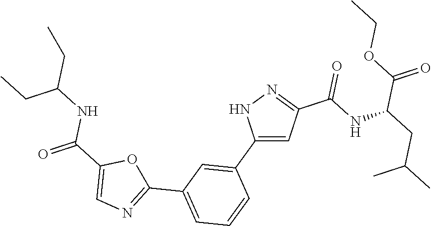 [SMILES: CCOC(=O)[C@H](CC(C)C)NC(=O)c1cc(-c2cccc(-c3ncc(C(=O)NC(CC)CC)o3)c2)[nH]n1]

LCMS Rt: 1.28 min MS m/z; 510.5 [M+H]+ 2minLowpHv03

¹H NMR (400 MHz, DMSO-d₆) b ppm 13.72-14.04 (m, 1H) 8.60-8.79 (m, 1H) 8.52 (s, 1H) 8.31 (br d, J=8.56 Hz, 1H) 8.13 (br d, J=7.58 Hz, 1H) 8.01 (br d, J=7.58 Hz, 1H) 7.93 (s, 1H) 7.69 (br t, J=7.83 Hz, 1H) 7.26-7.53 (m, 1H) 4.46-4.58 (m, 1H) 4.13 (q, J=7.09 Hz, 2H) 3.75-3.84 (m, 1H) 1.76-1.87 (m, 1H) 1.67-1.75 (m, 1H) 1.55-1.64 (m, 3H) 1.45-1.55 (m, 2H) 1.21 (t, J=7.09 Hz, 3H) 0.85-0.99 (m, 12H)

### Example 3.61:(S)-Tert-Butyl 3-(Tert-Butoxy)-2-(5-(3-(5-(Pentan-3-Ylcarbamoyl)Oxazol-2-Yl)Phenyl)-1 H-Pyrazole-3-Carboxamido)Propanoate Trifluoroacetate

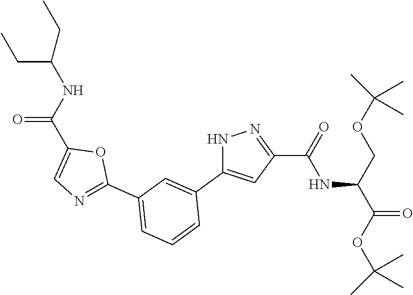 [SMILES: CCC(CC)NC(=O)c1cnc(-c2cccc(-c3cc(C(=O)N[C@@H](COC(C)(C)C)C(=O)OC(C)(C)C)n[nH]3)c2)o1]

LCMS Rt: 1.56 min MS m/z; 456.3 [M+H-2tBu]+2minLowpHv03

¹H NMR (400 MHz, CHLOROFORM-d) δ ppm 8.28 (s, 1H) 8.12 (br d, J=7.82 Hz, 2H) 7.85 (s, 1H) 7.73 (br d, J=7.58 Hz, 1H) 7.54 (t, J=7.70 Hz, 1H) 6.87-6.95 (m, 2H) 4.86 (br d, J=8.31 Hz, 1H) 4.02-4.11 (m, 1H) 3.98 (br dd, J=8.80, 2.20 Hz, 1H) 3.75 (br dd, J=8.80, 2.45 Hz, 1H) 1.70 (br d, J=5.62 Hz, 2H) 1.52-1.65 (m, 2H) 1.44 (s, 9H) 1.19 (s, 9H) 0.96-1.08 (m, 6H)

### Example 3.62:Tert-Butyl 1-(5-(3-(5-(Pentan-3-Ylcarbamoyl)Oxazol-2-Yl)Phenyl)-1H-Pyrazole-3-Carbonyl)Pyrrolidine-3-Carboxylate Trifluoroacetate

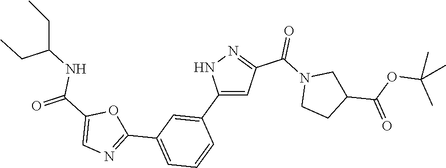 [SMILES: CCC(CC)NC(=O)c1cnc(-c2cccc(-c3cc(C(=O)N4CCC(C(=O)OC(C)(C)C)C4)n[nH]3)c2)o1]

LCMS Rt: 1.36 min MS m/z; 522.7 [M+H]+ 2minLowpHv03

### Example 3.63:(S)-Ethyl 2-(5-(3-(5-(Pentan-3-Ylcarbamoyl)Oxazol-2-Yl)Phenyl)-1H-Pyrazole-3-Carboxamido)Propanoate Trifluoroacetate

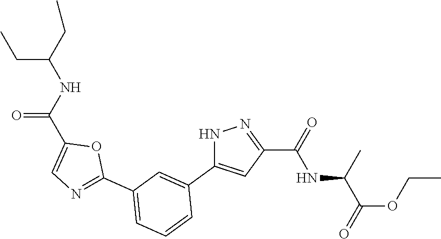 [SMILES: CCOC(=O)[C@H](C)NC(=O)c1cc(-c2cccc(-c3ncc(C(=O)NC(CC)CC)o3)c2)[nH]n1]

LCMS Rt: 1.14 min MS m/z; 466.5 [M+H]+ 2minLowpHv03

### Example 3.64:(S)-Benzyl 2-(5-(3-(5-(Pentan-3-Ylcarbamoyl)Oxazol-2-Yl)Phenyl)-1H-Pyrazole-3-Carboxamido)Propanoate Trifluoroacetate

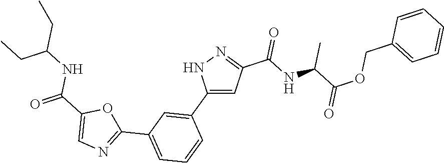 [SMILES: CCC(CC)NC(=O)c1cnc(-c2cccc(-c3cc(C(=O)N[C@@H](C)C(=O)OCc4ccccc4)n[nH]3)c2)o1]

LCMS Rt: 1.25 min MS m/z; 530.5 [M+H]+ 2minLowpHv03

### Example 3.65:(R)-Methyl 2-(5-(3-(5-(Pentan-3-Ylcarbamoyl)Oxazol-2-Yl)Phenyl)-1H-Pyrazole-3-Carboxamido)-2-Phenylacetate Trifluoroacetate

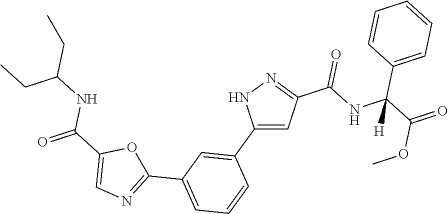 [SMILES: CCC(CC)NC(=O)c1cnc(-c2cccc(-c3cc(C(=O)N[C@@H](C(=O)OC)c4ccccc4)n[nH]3)c2)o1]

LCMS Rt: 1.21 min MS m/z; 516.5 [M+H]+ 2minLowpHv03

### Example 3.66:(S)-Ethyl 2-(5-(3-(5-(Pentan-3-Ylcarbamoylyl-Carbamoyl)Oxazol-2-Yl)Phenyl)-1H-Pyrazole-3-Carboxamido)-3-Phenylpropanoate Trifluoroacetate

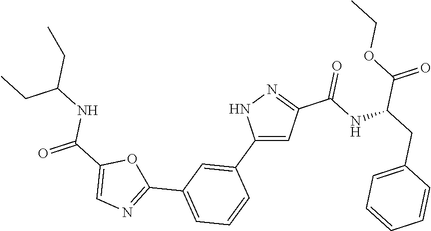 [SMILES: CCOC(=O)[C@H](Cc1ccccc1)NC(=O)c1cc(-c2cccc(-c3ncc(C(=O)NC(CC)CC)o3)c2)[nH]n1]

LCMS Rt: 1.27 min MS m/z; 544.5 [M+H]+ 2minLowpHv03

### Example 3.67: 2-(3-(3-(((1-Morpholinocyclohexyl)Methyl)Carbamoyl)-1H-Pyrazol-5-Yl)Phenyl)-N-(Pentan-3-Yl)Oxazole-5-Carboxamide Trifluoroacetate

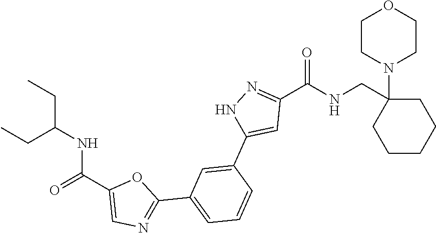 [SMILES: CCC(CC)NC(=O)c1cnc(-c2cccc(-c3cc(C(=O)NCC4(N5CCOCC5)CCCCC4)n[nH]3)c2)o1]

LCMS Rt: 0.94 min MS m/z; 549.6 [M+H]+ 2minLowpHv03

### Example 3.68: N-(2-Methyl-4-Phenylbutan-2-Yl)-2-(3-(3-(Pentan-3-Ylcarbamoylyl-Carbamoyl)-1H-Pyrazol-5-Yl)Phenyl)Oxazole-5-Carboxamide

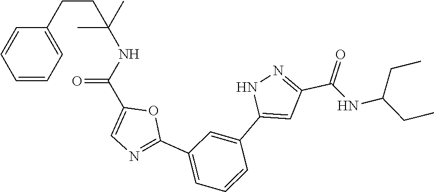 [SMILES: CCC(CC)NC(=O)c1cc(-c2cccc(-c3ncc(C(=O)NC(C)(C)CCc4ccccc4)o3)c2)[nH]n1]

LCMS Rt: 1.36 min MS m/z; 514.5 [M+H]+ 2minLowpHv03

### Example 3.69: Tert-Butyl 2-Methyl-2-(2-(3-(3-(Pentan-3-Ylcarbamoyl)-1H-Pyrazol-5-Yl)Phenyl)Oxazole-5-Carboxamido)Propanoatehexafluorophosphatepropanoate Hexafluorophosphate

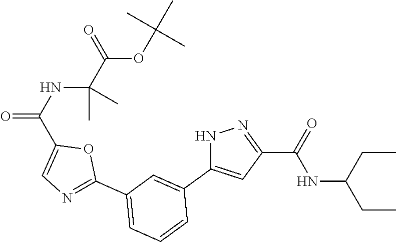 [SMILES: CCC(CC)NC(=O)c1cc(-c2cccc(-c3ncc(C(=O)NC(C)(C)C(=O)OC(C)(C)C)o3)c2)[nH]n1]

LCMS Rt: 1.27 min MS m/z; 510.7 [M+H]+ 2minLowpHv03
Example 4.0 of the present invention may be prepared according to Scheme 10.

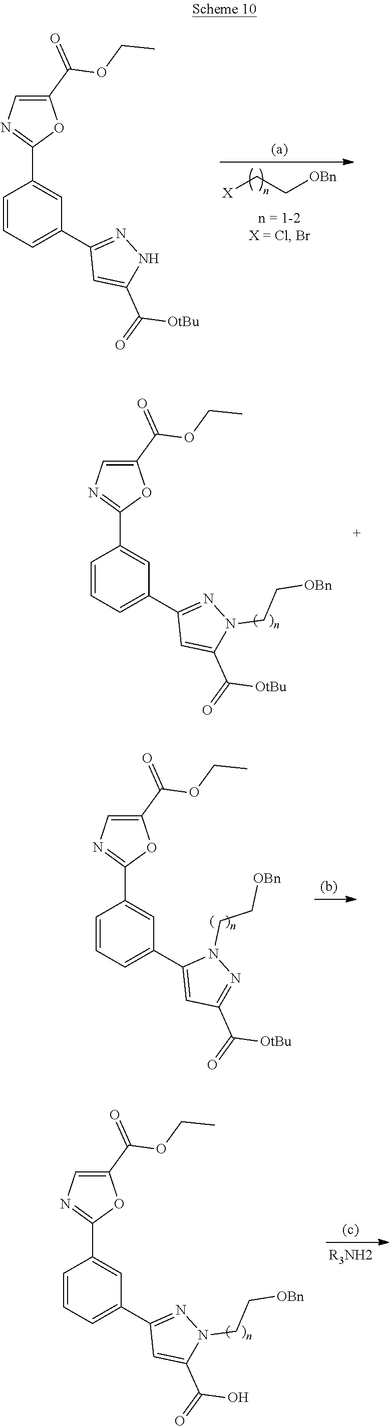

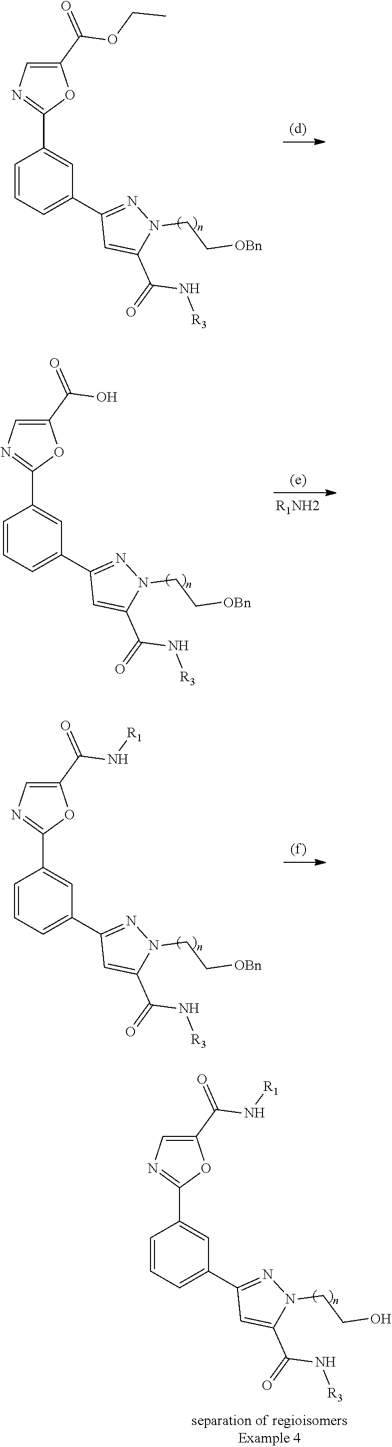

Step (a) involves alkylation of Intermediate 1 with haloalkylbenzyl ether to give varying chain lengths in the presence of a base such as Cs2CO3, NEt3, Na2CO3 or K2CO3 in a solvent such as THE or DMF to give a mixture of inseparable regioisomeric products.
Step (b) involves conversion of the mixture of regioisomeric tert-butyl esters to carboxylic acids by treatment with an acid such as TFA or HCl in a solvent such as DCM or dioxane.
Step (c) involves reaction of an amine(R₃NH2) with the mixture of regioisomeric carboxylic acids in a suitable solvent such as DMF or ethyl acetate with a suitable base such as diisopropylethylamine or triethylamine and an amide coupling reagent such as T3P or pyBOP.
Step (d) of Scheme 9 involves conversion of the ethyl ester to a carboxylic acid using a suitable base such as NaOH, KOH or KOTMS in a solvent such as THF, methanol or water.
Step (e) involves reaction of an amine(R₁NH2) with the mixture of regioisomeric free acids in a suitable solvent such as DMF or ethyl acetate with a suitable base such as diisopropylethylamine or triethylamine and an amide coupling reagent such as T3P or pyBOP.
Step (f) involves hydrogenation to liberate the alcohol of the tether from the benzyl protective group using a suitable palladium catalyst such as Pd (0) on carbon black in a suitable solvent such as methanol, ethanol followed by separation of regioisomers by chromatography to obtain the desired regioisomer.

### Example 4.0: (S)-N-(1-Cyclopropylethyl)-2-(3-(1-(2-Hydroxyethyl)-5-(Pentan-3-Ylcarbamoyl)-1 H-Pyrazol-3-Yl)Phenyl)Oxazole-5-Carboxamide

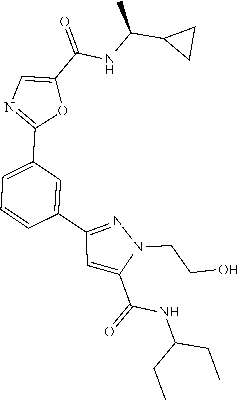 [SMILES: CCC(CC)NC(=O)c1cc(-c2cccc(-c3ncc(C(=O)N[C@@H](C)C4CC4)o3)c2)nn1CCO]

Step 1: Ethyl 2-(3-(1-(2-(benzyloxy)ethyl)-5-(tert-butoxycarbonyl)-1 H-pyrazol-3-yl)phenyl)oxazole-5-carboxylate: A mixture of ethyl 2-(3-(3-(tert-butoxycarbonyl)-1 H-pyrazol-5-yl)phenyl)oxazole-5-carboxylate Intermediate 1 (2.00 g, 5.22 mmol), ((2-bromoethoxy)methyl)benzene (4.13 mL, 26.1 mmol) and Na2CO3 (2.76 g, 26.1 mmol) in DMF (50 mL) was split equally across 3×10-20 mL microwave vials which were then flushed with nitrogen, sealed and heated by microwave at 110° C. for 4 hrs per vial. The RMs were then decanted and combined. The organics were then washed with water (3×), brine, dried over MgSO4 and concentrated. The crude material was purified by FCC (0-10% EtOAc/iso-hexane) to afford 1.90 g (66.9%) of ethyl 2-(3-(1-(2-(benzyloxy)ethyl)-5-(tert-butoxycarbonyl)-1H-pyrazol-3-yl)phenyl)oxazole-5-carboxylate. LCMS Rt: 1.80 min; MS m/z 518.6 [M+H]+ 2minLowpHv03 ¹H NMR (400 MHz, DMSO-d6) δ 8.54 (t, J=1.5 Hz, 1H), 8.17 (s, 1H), 8.13-8.08 (m, 1H), 8.05-8.00 (m, 1H), 7.66 (t, J=7.8 Hz, 1H), 7.42 (s, 1H), 7.29-7.17 (m, 5H), 4.81 (t, J=5.3 Hz, 2H), 4.46 (s, 2H), 4.38 (q, J=7.1 Hz, 2H), 3.83 (t, J=5.4 Hz, 2H), 1.53 (s, 9H), 1.34 (t, J=7.1 Hz, 3H).
Step 2: 1-(2-(benzyloxy)ethyl)-3-(3-(5-(ethoxvcarbonyl)oxazol-2-yl)phenyl)-1 H-pyrazole-5-carboxylic acid: A mixture of ethyl 2-(3-(1-(2-(benzyloxy)ethyl)-5-(tert-butoxycarbonyl)-1 H-pyrazol-3-yl)phenyl)oxazole-5-carboxylate (950 mg, 1.835 mmol) and TFA (5.66 mL, 73.4 mmol) in DCM (18.4 mL) was stirred at RT for 72h. The RM was concentrated to afford 850 mg (quantitative yield) of 1-(2-(benzyloxy)ethyl)-3-(3-(5-(ethoxycarbonyl)oxazol-2-yl)phenyl)-1 H-pyrazole-5-carboxylic acid as a pale yellow solid. LCMS Rt: 1.51 min; MS m/z 462.5 [M+H]+ 2minLowpHv03. 1H NMR (400 MHz, Methanol-d4) δ 8.59 (s, 1H), 8.05 (dd, J=22.0, 7.9 Hz, 2H), 7.95 (s, 1H), 7.60 (t, J=7.8 Hz, 1H), 7.32 (s, 1H), 7.21 (q, J=8.5, 7.1 Hz, 5H), 4.89 (d, J=5.5 Hz, 2H), 4.50 (s, 2H), 4.43 (q, J=7.1 Hz, 2H), 3.91 (t, J=5.5 Hz, 2H), 1.41 (t, J=7.1 Hz, 3H).
Step 3: Ethyl 2-(3-(1-(2-(benzyloxy)ethyl)-5-(pentan-3-ylcarbamoyl)-1H-pyrazol-3-yl)phenyl)oxazole-5-carboxylate: A mixture of 1-(2-(benzyloxy)ethyl)-3-(3-(5-(ethoxycarbonyl)oxazol-2-yl)phenyl)-1 H-pyrazole-5-carboxylic acid (850 mg, 1.842 mmol), pentan-3-amine (0.322 mL, 1.842 mmol), T3P 50% in EtOAc (0.822 mL, 2.76 mmol) and triethylamine (7.7 mL, 55.2 mmol) in EtOAc (20 mL) was stirred at RT for 18 h. The reaction was monitored by LCMS adding additional aliquots of T3P as needed. The RM was diluted with EtOAc (20 mL) and washed with water, sat. NaHCO₃, and brine, dried over MgSO4 and concentrated to afford 1.08 g (quantitative yield) of ethyl 2-(3-(1-(2-(benzyloxy)ethyl)-5-(pentan-3-ylcarbamoyl)-1 H-pyrazol-3-yl)phenyl)oxazole-5-carboxylate. LC-MS Rt: 1.66 min; MS m/z 531.6 [M+H]+ 2minLowpHv03. ¹H NMR (400 MHz, METHANOL-d₄) δ ppm 8.60 (d, J=1.26 Hz, 1H) 8.10-8.14 (m, 1H) 8.03-8.07 (m, 1H) 7.99 (s, 1H) 7.64 (t, J=7.83 Hz, 1 H) 7.19-7.28 (m, 5H) 4.84 (s, 2H) 4.50 (s, 2H) 4.45 (d, J=7.07 Hz, 2H) 3.90 (t, J=5.43 Hz, 2H) 3.84 (br t, J=4.67 Hz, 1H) 1.58-1.69 (m, 2H) 1.45-1.56 (m, 2H) 1.43 (t, J=7.20 Hz, 3H) 0.95 (t, J=7.45 Hz, 6H)
Step 4: 2-(3-(1-(2-(benzyloxy)ethyl)-5-(pentan-3-ylcarbamoyl)-1 H-pyrazol-3-yl)phenyl)oxazole-5-carboxylic acid: A mixture of ethyl 2-(3-(1-(2-(benzyloxy)ethyl)-5-(pentan-3-ylcarbamoyl)-1 H-pyrazol-3-yl)phenyl)oxazole-5-carboxylate (1 g, 1.885 mmol) and TMSOK (280 mg, 2.83 mmol) in dry THE was stirred under nitrogen at RT for 18 h. The RM was concentrated to give 1.06 g (quantitative yield) of 2-(3-(1-(2-(benzyloxy)ethyl)-5-(pentan-3-ylcarbamoyl)-1 H-pyrazol-3-yl)phenyl)oxazole-5-carboxylic acid as a pale yellow solid. LCMS Rt: 1.54 min; MS m/z 503.6 [M+H]+ 2minLowpHv03. ¹H NMR (400 MHz, METHANOL-d₄) δ ppm 8.53 (t, J=1.39 Hz, 1H) 8.00-8.06 (m, 1H) 7.96 (dd, J=7.96, 1.14 Hz, 1H) 7.56 (s, 1H) 7.52 (t, J=7.71 Hz, 1H) 7.18 (s, 1H) 7.09-7.17 (m, 5H) 4.75 (t, J=5.31 Hz, 2H) 4.41 (s, 2H) 3.81 (t, J=5.31 Hz, 2H) 3.72-3.79 (m, 1H) 1.50-1.63 (m, 2H) 1.34-1.48 (m, 2H) 0.87 (t, J=7.33 Hz, 6H)
Step 5: (S)-2-(3-(1-(2-(benzyloxy)ethyl)-5-(pentan-3-ylcarbamoyl)-1H-pyrazol-3-yl)phenyl)-N-(1-cycloPropylethyl)oxazole-5-carboxamide: A mixture of 2-(3-(1-(2-(benzyloxy)ethyl)-5-(pentan-3-ylcarbamoyl)-1 H-pyrazol-3-yl)phenyl)oxazole-5-carboxylic acid (50 mg, 0.099 mmol), (S)-1-cyclopropylethan-1-amine (0.298 mmol), T3P 50% in EtOAc (0.089 mL, 0.149 mmol) and triethylamine (0.083 mL, 0.597 mmol) in EtOAc (1 mL) was stirred at RT adding additional aliquots of T3P as needed to drive the reaction to completion. The RM was diluted with EtOAc (20 mL) and washed with water, sat NaHCO₃, brine, dried over MgSO4 and concentrated to afford (S)-2-(3-(1-(2-(benzyloxy)ethyl)-5-(pentan-3-ylcarbamoyl)-1H-pyrazol-3-yl)phenyl)-N-(1-cyclopropylethyl)oxazole-5-carboxamide which was used crude for the next step. LCMS Rt: 1.59 min; MS m/z 570.6 [M+H]+ 2minLowpHv03.
Step 6: A solution of (S)-2-(3-(1-(2-(benzyloxy)ethyl)-5-(pentan-3-ylcarbamoyl)-1 H-pyrazol-3-yl)phenyl)-N-(1-cyclopropylethyl)oxazole-5-carboxamide (81 mg, 0.142 mmol) in ethanol (10 mL) was passed through a 10% Pd/C CatCart using the H-CUBE system. Conditions: Full H2, 60° C. The RM was recirculated through the system for 2 hours. The RM was concentrated to give 43 mg (56.8%) of (S)-N-(1-cyclopropylethyl)-2-(3-(1-(2-hydroxyethyl)-5-(pentan-3-ylcarbamoyl)-1 H-pyrazol-3-yl)phenyl)oxazole-5-carboxamide Example 4.0; as a white solid. LCMS Rt: 1.30, MS m/z 480.5 [M+H]+ 2minLowpHv03. ¹H NMR (400 MHz, METHANOL-d₄) δ ppm 8.66 (t, J=1.39 Hz, 1H) 8.13-8.20 (m, 1H) 8.04 (dd, J=7.70, 1.14 Hz, 1H) 7.85 (s, 1H) 7.62 (t, J=7.83 Hz, 1H) 7.23 (s, 1H) 4.70 (t, J=5.56 Hz, 2H) 3.98 (t, J=5.68 Hz, 2H) 3.88 (s, 1H) 3.50 (dd, J=8.97, 6.69 Hz, 1H) 1.63-1.76 (m, 2H) 1.49-1.61 (m, 2H) 1.37 (d, J=6.82 Hz, 3H) 1.05-1.13 (m, 1H) 1.00 (t, J=7.33 Hz, 6H) 0.56-0.66 (m, 1H) 0.48-0.55 (m, 1H) 0.41 (s, 1H) 0.28-0.36 (m, 1H).
Examples 4.1 to 4.5 were prepared by a similar method to that of Example 4.0 by replacing with the appropriate commercially available amines in Step 3 and Step 5.

### Example 4.1: (S)-Ethyl 2-(2-(3-(1-(2-Hydroxyethyl)-5-(Pentan-3-Ylcarbamoyl)-1H-Pyrazol-3-Yl)Phenyl)Oxazole-5-Carboxamido)-3-Methylbutanoate

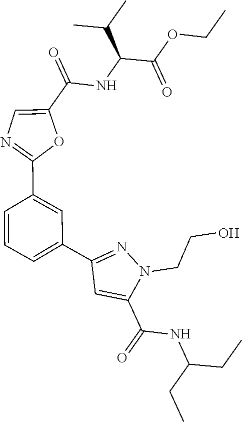 [SMILES: CCOC(=O)[C@@H](NC(=O)c1cnc(-c2cccc(-c3cc(C(=O)NC(CC)CC)n(CCO)n3)c2)o1)C(C)C]

LCMS Rt: 4.36 min; MS m/z 540.6 [M+H]+ 8minLowpHv01

¹H NMR (400 MHz, METHANOL-d₄) δ ppm 8.66 (s, 1H) 8.16 (d, J=7.82 Hz, 1H) 8.05 (br d, J=8.07 Hz, 1H) 7.96 (s, 1H) 7.63 (t, J=7.82 Hz, 1H) 7.24 (s, 1H) 4.71 (t, J=5.62 Hz, 2H) 4.52 (d, J=7.09 Hz, 1H) 4.26 (qd, J=7.09, 3.18 Hz, 2H) 3.98 (t, J=5.62 Hz, 2H) 3.83-3.94 (m, 1H) 2.27-2.39 (m, 1H) 1.63-1.77 (m, 2H) 1.52-1.62 (m, 2H) 1.33 (t, J=7.09 Hz, 3H) 1.08 (t, J=7.34 Hz, 6H) 1.00 (t, J=7.46 Hz, 6H)

### Example 4.2: (S)-Ethyl 2-(2-(3-(5-(((S)-1-Cyclopropylethyl)Carbamoyl)-1-(2-Hydroxyethyl)-1H-Pyrazol-3-Yl)Phenyl)Oxazole-5-Carboxamido)-3-Methylbutanoate

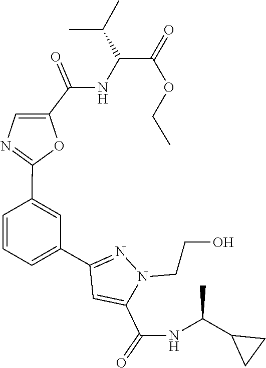

LCMS Rt: 1.43 min; MS m/z 538.1 [M+H]+ RXNMON_Acidic

¹H NMR (400 MHz, METHANOL-d₄) Q ppm 8.64 (t, J=1.53 Hz, 1H) 8.14 (dt, J=7.83, 1.41 Hz, 1H) 8.03 (dt, J=8.07, 1.28 Hz, 1H) 7.94 (s, 1H) 7.61 (t, J=7.64 Hz, 1H) 7.22 (s, 1H) 4.68 (t, J=5.62 Hz, 2H) 4.50 (d, J=6.97 Hz, 1H) 4.19-4.29 (m, 2H) 3.96 (t, J=5.62 Hz, 2H) 2.30 (dq, J=13.69, 6.85 Hz, 1H) 1.32 (app. t, J=6.97 Hz, 6H) 1.29 (s, 2H) 1.03-1.09 (m, 6H) 0.98-1.02 (m, 1H) 0.83-0.93 (m, 2H) 0.53-0.62 (m, 1H) 0.44-0.53 (m, 1H) 0.36-0.43 (m, 1H) 0.25-0.32 (m, 1H)

### Example 4.3: (S)-Methyl 2-(2-(3-(5-((Dicyclopropylmethyl)Carbamoyl)-1-(2-Hydroxyethyl)-1H-Pyrazol-3-Yl)Phenyl)Oxazole-5-Carboxamido)-3-Methylbutanoate

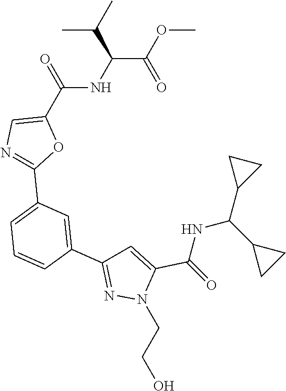 [SMILES: COC(=O)[C@@H](NC(=O)c1cnc(-c2cccc(-c3cc(C(=O)NC(C4CC4)C4CC4)n(CCO)n3)c2)o1)C(C)C]

LCMS Rt: 1.35 min; MS m/z 550.4 [M+H]+ RXNMON_Basic

¹H NMR (400 MHz, Chloroform-d) δ 8.31 (s, 1H), 7.94-7.88 (m, 2H), 7.76 (s, 1H), 7.44 (t, J=7.8 Hz, 1H), 6.93 (d, J=8.7 Hz, 1H), 6.90 (s, 1H), 6.80 (d, J=8.4 Hz, 1H), 4.71-4.66 (m, 1H), 4.66-4.62 (m, 2H), 4.01-3.96 (m, 2H), 3.72 (s, 3H), 3.15 (q, J=8.2 Hz, 1H), 2.29-2.18 (m, 1H), 0.95 (dd, J=6.8, 2.7 Hz, 8H), 0.52 (dt, J=8.4, 4.4 Hz, 2H), 0.45-0.28 (m, 6H).

### Example 4.4 (S)-Ethyl 2-(2-(3-(5-((Dicyclopropylmethyl)Carbamoyl)-1-(2-Hydroxyethyl)-1 H-Pyrazol-3-Yl)Phenyl)Oxazole-5-Carboxamido)-3-Methylbutanoate

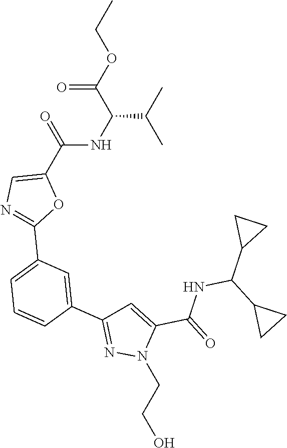 [SMILES: CCOC(=O)[C@@H](NC(=O)c1cnc(-c2cccc(-c3cc(C(=O)NC(C4CC4)C4CC4)n(CCO)n3)c2)o1)C(C)C]

LCMS Rt: 1.41 min; MS m/z 564.4 [M+1]+ RXNMON_Basic
1H NMR (400 MHz, Methanol-d4) δ 8.64 (t, J=1.6 Hz, 1H), 8.17-8.10 (m, 1H), 8.03 (dt, J=7.8, 1.2 Hz, 1H), 7.94 (s, 1H), 7.60 (t, J=7.8 Hz, 1H), 7.23 (s, 1H), 4.67 (t, J=5.6 Hz, 2H), 4.49 (d, J=7.1 Hz, 1H), 4.24 (tq, J=7.1, 3.4 Hz, 2H), 3.96 (t, J=5.6 Hz, 2H), 3.06 (t, J=8.3 Hz, 1H), 2.31 (dp, J=13.7, 7.2, 6.8 Hz, 1H), 1.39-1.16 (m, 5H), 1.13 (tdd, J=8.2, 4.9, 3.2 Hz, 2H), 1.06 (dd, J=8.4, 6.8 Hz, 6H), 0.67-0.52 (m, 2H), 0.51-0.32 (m, 6H).

### Example 4.5 (S)-Ethyl 2-(2-(3-(5-((Dicyclopropylmethyl)Carbamoyl)-1-(3-Hydroxypropyl)-1 H-Pyrazol-3-Yl)Phenyl)Oxazole-5-Carboxamido)-3-Methylbutanoate

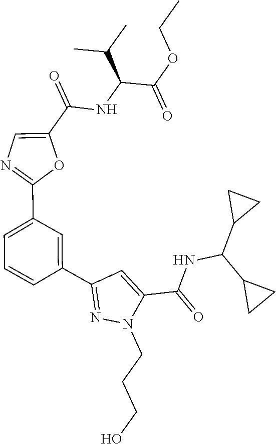 [SMILES: CCOC(=O)[C@@H](NC(=O)c1cnc(-c2cccc(-c3cc(C(=O)NC(C4CC4)C4CC4)n(CCCO)n3)c2)o1)C(C)C]

LCMS Rt: 1.43 min; MS m/z 578.3 [M+1]+ RXNMON_Basic
1H NMR (400 MHz, Methanol-d4) δ 8.59 (t, J=1.7 Hz, 1H), 8.10 (dt, J=7.8, 1.3 Hz, 1H), 8.00 (dt, J=7.8, 1.5 Hz, 1H), 7.93 (s, 1H), 7.58 (t, J=7.8 Hz, 1H), 7.21 (s, 1H), 4.64 (t, J=6.9 Hz, 2H), 4.50 (d, J=6.9 Hz, 1H), 4.24 (qq, J=7.4, 3.7 Hz, 2H), 3.57 (t, J=6.3 Hz, 2H), 3.04 (t, J=8.3 Hz, 1H), 2.29 (hept, J=6.8 Hz, 1H), 2.10 (p, J=6.6 Hz, 2H), 2.01 (s, 1H), 1.35-1.21 (m, 3H), 1.24-1.08 (m, 2H), 1.06 (dd, J=8.5, 6.8 Hz, 6H), 0.68-0.52 (m, 2H), 0.46 (tt, J=8.0, 1.7 Hz, 2H), 0.46-0.32 (m, 4H).

Example 5 of the present invention may be prepared according to Scheme 11.

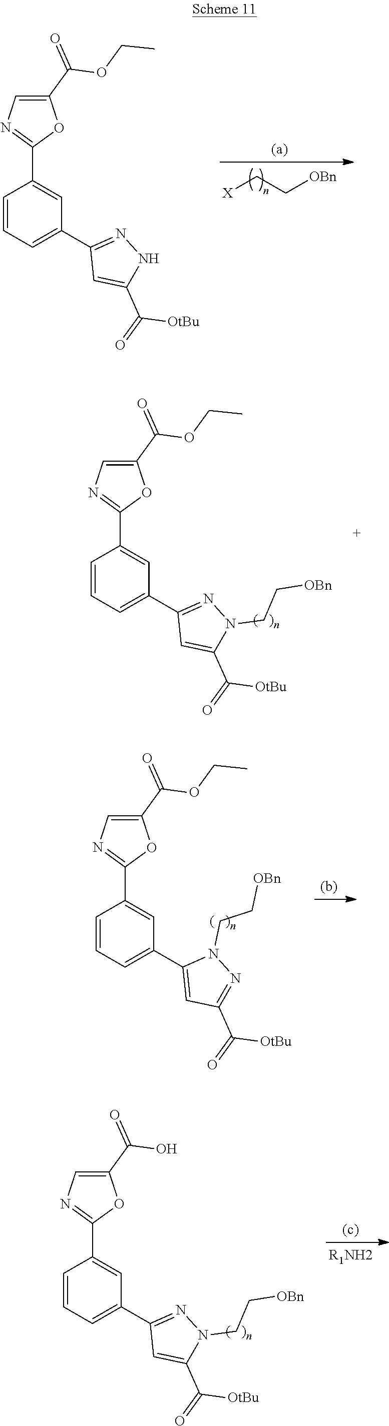

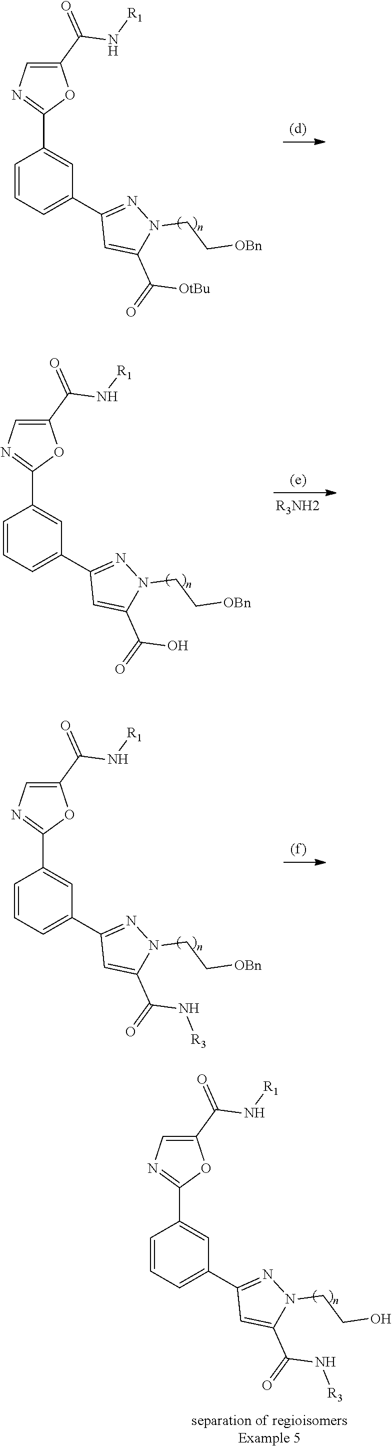

Step (a) involves alkylation of Intermediate 1 with haloalkybenzyl ether to give varying chain lengths in the presence of a base such as Cs2CO3, NEt3, Na2CO3 or K2CO3 in a solvent such as THE or DMF to give a mixture of inseparable regioisomeric products.
Step (b) of Scheme 10 involves conversion of the mixture of regioisomeric ethyl esters to carboxylic acids using a suitable base such as NaOH, KOH or KOTMS in a solvent such as THF, methanol or water.
Step (c) involves reaction of an amine(R₁NH2) with the mixture of regioisomeric carboxylic acids in a suitable solvent such as DMF or ethyl acetate with a suitable base such as diisopropylethylamine or triethylamine and an amide coupling reagent such as T3P or pyBOP.
Step (d) involves conversion of the mixture of regioisomeric tert-butyl esters to carboxylic acids by treatment with an acid such as TFA or HCl in a solvent such as DCM or dioxane.
Step (e) involves reaction of an amine(R₃NH2) with the mixture of regioisomeric free acids in a suitable solvent such as DMF or ethyl acetate with a suitable base such as diisopropylethylamine or triethylamine and an amide coupling reagent such as T3P or pyBOP.
Step (f) involves hydrogenation to liberate the alcohol of the tether using a suitable palladium catalyst such as Pd (0) on carbon black in a suitable solvent such as methanol, ethanol followed by separation by chromatography to obtain the desired regioisomer.
Alternatively, in step (b), prolonged treatment with base may provide doubly deprotected di-acid which may then be subjected to simultaneous double amide formation using the conditions previously described.

### Example 5.0: (S)-Ethyl 2-(1-(2-Hydroxyethyl)-3-(3-(5-(Pentan-3-Ylcarbamoyl)Oxazol-2-Yl)Phenyl)-1H-Pyrazole-5-Carboxamido)-3-Methylbutanoate

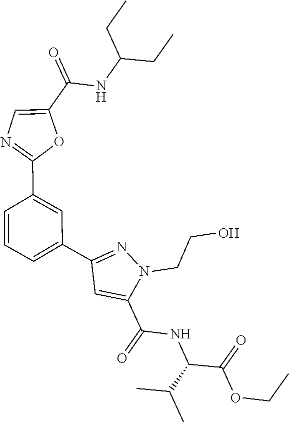 [SMILES: CCOC(=O)[C@@H](NC(=O)c1cc(-c2cccc(-c3ncc(C(=O)NC(CC)CC)o3)c2)nn1CCO)C(C)C]

Step 1: 2-(3-(1-(2-(benzyloxy)ethyl)-5-(tert-butoxycarbonyl)-1H-pyrazol-3-yl)phenyl)oxazole-5-carboxylic acid: A mixture of ethyl 2-(3-(1-(2-(benzyloxy)ethyl)-5-(tert-butoxycarbonyl)-1 H-pyrazol-3-yl)phenyl)oxazole-5-carboxylate (Intermediate from Step 1 of synthesis of Example 4.0) (275 mg, 0.31 mmol) and TMSOK (114 mg, 0.797 mmol) in dry THE (5 mL) was stirred under nitrogen overnight. The RM was concentrated under reduced pressure to give 300 mg of 2-(3-(1-(2-(benzyloxy)ethyl)-5-(tert-butoxycarbonyl)-1 H-pyrazol-3-yl)phenyl)oxazole-5-carboxylic acid as a pale yellow solid. LCMS Rt: 1.68 mins MS m/z; 490.4 [M+H]+ 2minLowpHv03.
Step 2: Tert-butyl 1-(2-(benzyloxy)ethyl)-3-(3-(5-(pentan-3-ylcarbamoyl)oxazol-2-yl)phenyl)-1 H-pyrazole-5-carboxylate; A mixture of 2-(3-(1-(2-(benzyloxy)ethyl)-5-(tert-butoxycarbonyl)-1 H-pyrazol-3-yl)phenyl)oxazole-5-carboxylic acid (300 mg, 0.568 mmol), pentan-3-amine (99 μL, 0.851 mmol), T3P 50% in EtOAc (507 μL, 0.851 mmol) and TEA (237 μL, 1.703 mmol) in EtOAc (5 ml) was stirred at RT overnight. The RM was diluted with EtOAc (20 mL) and washed with water, sat NaHCO₃, brine, dried over MgSO4 and concentrated. The crude material was purified by FCC:(0-50% EtOAc/iso-hexane) to afford 169 mg (53.3%) of tert-butyl 1-(2-(benzyloxy)ethyl)-3-(3-(5-(pentan-3-ylcarbamoyl)oxazol-2-yl)phenyl)-1 H-pyrazole-5-carboxylate. LCMS Rt: 1.75 mins MS m/z; 559.6 [M+H]+ 2minLowpHv03.¹H NMR (400 MHz, METHANOL-d₄) δ ppm 8.65 (t, J=1.52 Hz, 1H) 8.11-8.18 (m, 1H) 8.05 (dd, J=7.83, 1.26 Hz, 1H) 7.86 (s, 1H) 7.61 (t, J=7.83 Hz, 1H) 7.27 (s, 1H) 7.18-7.26 (m, 5H) 4.86 (t, J=5.43 Hz, 2H) 4.49 (s, 2H) 3.91-3.98 (m, 1H) 3.89 (t, J=5.56 Hz, 2H) 1.66-1.76 (m, 2H) 1.60-1.65 (m, 1H) 1.58 (s, 9H) 0.97-1.01 (m, 6H)
Step 3: 1-(2-(benzyloxy)ethyl)-3-(3-(5-(pentan-3-ylcarbamoyl)oxazol-2-yl)phenyl)-1H-pyrazole-5-carboxylic acid A mixture of tert-butyl 1-(2-(benzyloxy)ethyl)-3-(3-(5-(pentan-3-ylcarbamoyl)oxazol-2-yl)phenyl)-1 H-pyrazole-5-carboxylate (169 mg, 0.303 mmol) and TFA (699 μL, 9.08 mmol) in DCM (3 mL) was stirred at RT overnight. The RM was concentrated to give 207 mg of 1-(2-(benzyloxy)ethyl)-3-(3-(5-(pentan-3-ylcarbamoyl)oxazol-2-yl)phenyl)-1 H-pyrazole-5-carboxylic acid as a white solid. LCMS Rt: 1.50 mins MS m/z; 503.5 [M+H]+ 2minLowpHv03
Step 4: (S)-ethyl 2-(1-(2-(benzyloxy)ethyl)-3-(3-(5-(pentan-3-ylcarbamoyl)oxazol-2-yl)phenyl)-1H-pyrazole-5-carboxamido)-3-methylbutanoate A mixture of 1-(2-(benzyloxy)ethyl)-3-(3-(5-(pentan-3-ylcarbamoyl)oxazol-2-yl)phenyl)-1 H-pyrazole-5-carboxylic acid (100 mg, 0.199 mmol), (S)-ethyl 2-amino-3-methylbutanoate.HCl (34.7 mg, 0.239 mmol), Triethylamine (0.111 mL, 0.796 mmol), and T3P (50% in EtOAc) (0.178 mL, 0.298 mmol) was stirred in EtOAc for 18 h. The RM was diluted with EtOAc and washed with water. The aqueous layer was separated and then extracted with EtOAc (2×). The combined organics were then washed with sat NaHCO₃, brine, dried over MgSO4 and concentrated to afford 68 mg (51.6%) of (S)-ethyl 2-(1-(2-(benzyloxy)ethyl)-3-(3-(5-(pentan-3-ylcarbamoyl)oxazol-2-yl)phenyl)-1 H-pyrazole-5-carboxamido)-3-methylbutanoate. LCMS Rt: 1.65 mins MS m/z; 630.7 [M+H]+ 2minLowpHv03.¹H NMR (400 MHz, METHANOL-d₄) δ ppm 8.67 (t, J=1.47 Hz, 1H) 8.18 (d, J=8.07 Hz, 1H) 8.03-8.07 (m, 1H) 7.87 (s, 1H) 7.64 (t, J=7.95 Hz, 1H) 7.34 (s, 1H) 7.18-7.27 (m, 5H) 4.79-4.83 (m, 2H) 4.52 (s, 2H) 4.48 (d, J=6.36 Hz, 1H) 4.18-4.29 (m, 3H) 3.92 (t, J=5.14 Hz, 3H) 2.18-2.31 (m, 1H) 1.66-1.77 (m, 2H) 1.53-1.64 (m, 2H) 1.24-1.34 (m, 3H) 0.97-1.05 (m, 12H)
Step 5: A solution of S)-ethyl 2-(1-(2-(benzyloxy)ethyl)-3-(3-(5-(pentan-3-ylcarbamoyl)oxazol-2-yl)phenyl)-1 H-pyrazole-5-carboxamido)-3-methylbutanoate in ethanol (10 mL) was passed through a 10% Pd/C CatCart using the H-CUBE system. Conditions: Full H2, 60° C. The crude material was purified by FCC (0-50% EtOAc/iso-hexane) to give 24 mg (40.0%) of (S)-ethyl 2-(1-(2-hydroxyethyl)-3-(3-(5-(pentan-3-ylcarbamoyl)oxazol-2-yl)phenyl)-1H-pyrazole-5-carboxamido)-3-methylbutanoate Example 5.0 as a white solid. LCMS Rt: 0.6 min; MS m/z 540.7 [M+H]+ 2minLowpHv03.¹H NMR (400 MHz, METHANOL-d₄) δ ppm 8.67 (t, J=1.47 Hz, 1H) 8.16 (dt, J=8.01, 1.25 Hz, 1H) 8.06 (dt, J=8.07, 1.22 Hz, 1H) 7.86 (s, 1H) 7.62 (t, J=7.82 Hz, 1H) 7.31 (s, 1H) 4.70 (dt, J=8.93, 5.44 Hz, 2H) 4.52 (d, J=6.36 Hz, 1H) 4.21-4.32 (m, 2H) 4.00 (t, J=5.75 Hz, 2H) 3.93 (s, 1H) 2.26-2.37 (m, 1H) 1.66-1.78 (m, 2H) 1.60 (ddd, J=13.94, 8.68, 7.46 Hz, 2H) 1.33 (t, J=7.09 Hz, 3H) 1.07 (dd, J=6.72, 1.34 Hz, 6H) 1.00 (t, J=7.34 Hz, 6H)
Example 5.1 and 5.2 were prepared by a similar method to that of Example 5.0 by replacing with the appropriate commercially available amines in Step 5.

### Example 5.1: (R)-2-(3-(5-((1-Cyclopropylethyl)Carbamoyl)-1-(2-Hydroxyethyl)-1H-Pyrazol-3-Yl)Phenyl)-N-(Pentan-3-Yl)Oxazole-5-Carboxamide

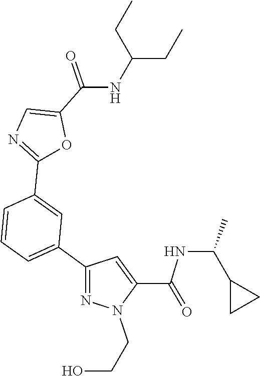 [SMILES: CCC(CC)NC(=O)c1cnc(-c2cccc(-c3cc(C(=O)N[C@H](C)C4CC4)n(CCO)n3)c2)o1]

1H NMR (400 MHz, Methanol-d4) δ 8.65 (s, 1H), 8.15 (d, J=7.7 Hz, 1H), 8.03 (d, J=7.6 Hz, 1H), 7.86 (s, 1H), 7.61 (t, J=7.8 Hz, 1H), 7.23 (s, 1H), 4.70 (t, J=5.4 Hz, 2H), 3.98 (t, J=5.4 Hz, 2H), 3.96-3.89 (m, 1H), 3.54-3.45 (m, 1H), 1.70 (dq, J=14.1, 7.4, 7.0 Hz, 2H), 1.58 (dq, J=15.1, 7.6 Hz, 2H), 1.35 (d, J=6.6 Hz, 3H), 1.09-1.02 (m, 1H), 0.99 (t, J=7.3 Hz, 6H), 0.63-0.55 (m, 1H), 0.52 (dt, J=8.1, 4.9 Hz, 1H), 0.42 (dd, J=9.2, 4.5 Hz, 1H), 0.30 (dd, J=9.1, 4.4 Hz, 1H). LCMS: Rt 1.38 min; MS m/z 480.4 [M+H]+ RXNMON Acidic NonPolar

### Example 5.2: N-Cyclopentyl-2-(3-(5-(Cyclopentylcarbamoyl)-1-(3-Hydroxypropyl)-1H-Pyrazol-3-Yl)Phenyl)Oxazole-5-Carboxamide

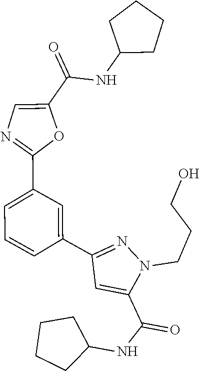 [SMILES: O=C(NC1CCCC1)c1cnc(-c2cccc(-c3cc(C(=O)NC4CCCC4)n(CCCO)n3)c2)o1]

LCMS: Rt=1.32 minutes; MS m/z 492 [M+1]*; RXNMON_Basic.

¹H NMR (400 MHz, Chloroform-d) δ 8.38 (s, 1H), 7.99 (dd, J=12.3, 7.9 Hz, 2H), 7.81 (s, 1H), 7.53 (t, J=7.8 Hz, 1H), 7.28 (s, 1H), 6.89 (s, 1H), 6.62 (d, J=7.3 Hz, 1H), 6.52 (d, J=7.5 Hz, 1H), 4.76-4.68 (m, 2H), 4.51-4.34 (m, 2H), 3.53 (q, J=5.1 Hz, 2H), 2.23-2.07 (m, 6H), 1.75 (dddd, J=30.8, 15.0, 8.1, 3.0 Hz, 8H), 1.59 (dp, J =14.4, 7.5, 6.9 Hz, 4H).

Example 6 of the present invention may be prepared according to Scheme 12.

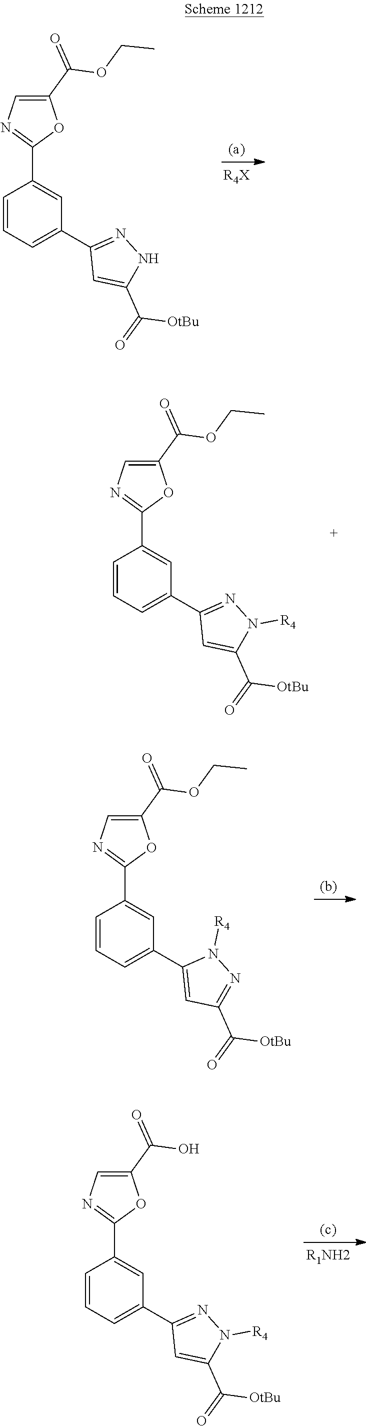

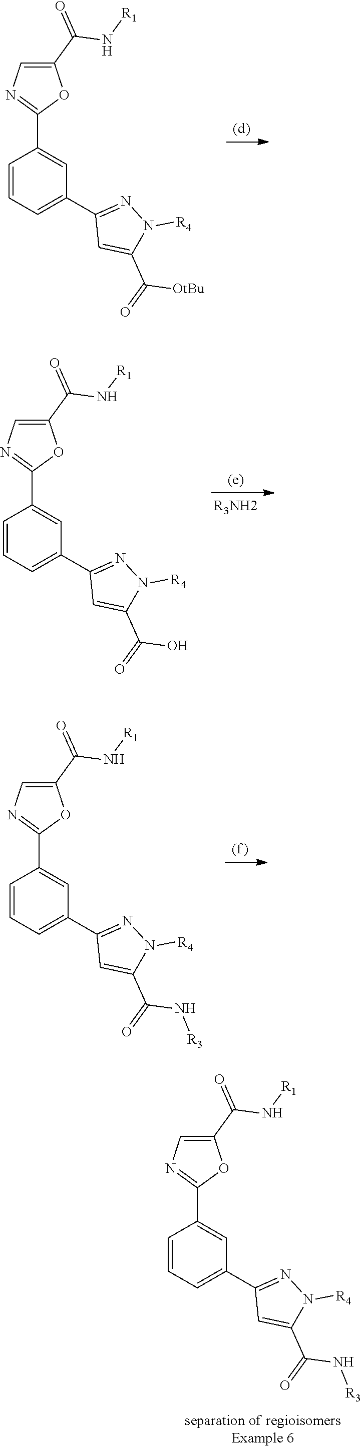

Step (a) involves alkylation of Intermediate 1 with a haloalkane (R-X) in the presence of a base such as Cs2CO3, Net3, Na2CO3 or K2CO3 in a solvent such as THE or DMF to give a mixture of inseparable regioisomeric products.
Step (b) of Scheme 11 involves conversion of the mixture of regioisomeric ethyl esters to carboxylic acids using a suitable base such as NaOH, KOH or KOTMS in a solvent such as THF, methanol or water.
Step (c) involves reaction of an amine(R₁NH2) with the mixture of regioisomeric carboxylic acids in a suitable solvent such as DMF or ethyl acetate with a suitable base such as diisopropylethylamine or triethylamine and an amide coupling reagent such as T3P, HATU, or pyBOP.
Step (d) involves conversion of the mixture of regioisomeric tert-butyl esters to carboxylic acids by treatment with an acid such as TFA or HCl in a solvent such as DCM or dioxane.
Step (e) involves reaction of an amine(R₃NH2) with the mixture of regioisomeric free acids in a suitable solvent such as DMF or ethyl acetate with a suitable base such as diisopropylethylamine or triethylamine and an amide coupling reagent such as T3P or pyBOP.
Step (f), if necessary, involves hydrogenation of benzyl ether protective group to liberate the alcohol of the tether using a suitable palladium catalyst such as Pd (0) on carbon black in a suitable solvent asuch as methanol, ethanol followed by separation by chromatography to obtain the desired regioisomer.
Alternatively, step (b) ester saponification may be conducted at higher temperature or for longer time in order to convert the material to di-acid. The di-acid may be subjected to symmetric bis amide formation conditions.

### Example 6.0

(S)-ethyl 3-methyl-2-(2-(3-(1-(2-morpholinoethyl)-5-(pentan-3-ylcarbamoyl)-1H-pyrazol-3-yl)phenyl)oxazole-5-carboxamido)butanoate

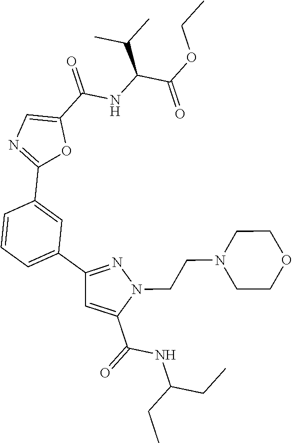 [SMILES: CCOC(=O)[C@@H](NC(=O)c1cnc(-c2cccc(-c3cc(C(=O)NC(CC)CC)n(CCN4CCOCC4)n3)c2)o1)C(C)C]

Step 1: Ethyl 2-(3-(5-(tert-butoxycarbonyl)-1-(2-morpholinoethyl)-1 H-pyrazol-3-yl)phenyl)oxazole-5-carboxylate
To a stirred solution of 4-(2-bromoethyl)morpholine.HBr (86 mg, 0.313 mmol) and triethylamine (44 μL, 0.313 mmol) in dry DMF (2.5 mL) was added ethyl 2-(3-(3-(tert-butoxycarbonyl)-1H-pyrazol-5-yl)phenyl)oxazole-5-carboxylate (Intermediate 1)(100 mg, 0.261 mmol), and sodium carbonate (30 mg, 0.287 mmol) and the resulting reaction mixture was stirred at 110° C. under nitrogen for 18 h. The RM was partitioned between EtOAc and water. The aqueous layer was separated and extracted with EtOAc (2×). The combined organics were then washed with water (2×), brine, dried over MgSO4 and concentrated. The crude product was purified by prep HPLCMethod 2: Low pH 20-50% B to afford 24 mg (17.6%) of ethyl 2-(3-(5-(tert-butoxycarbonyl)-1-(2-morpholinoethyl)-1 H-pyrazol-3-yl)phenyl)oxazole-5-carboxylate.

LCMS Rt: 1.09 min; MS m/z 497.6 [M+H]+ 2minLowpHv03

Step 2: 3-(3-(5-(ethoxycarbonyl)oxazol-2-yl)phenyl)-1-(2-morpholinoethyl)-1H-pyrazole-5-carboxylic acid
A solution of ethyl 2-(3-(5-(tert-butoxycarbonyl)-1-(2-morpholinoethyl)-1 H-pyrazol-3-yl)phenyl)oxazole-5-carboxylate (24 mg, 0.048 mmol) and TFA (149 μL, 1.933 mmol) in DCM (500 μL) was stirred at RT for 18h. The RM was concentrated and the crude material used directly for the next step.
LCMS Rt: 0.92 min; MS m/z 441.5 [M+H]+ 2minLowpHv03.
Step 3: Ethyl 2-(3-(1-(2-morpholinoethyl)-5-(pentan-3-ylcarbamoyl)-1 H-pyrazol-3-yl)phenyl)oxazole-5-carboxylate
A mixture of 3-(3-(5-(ethoxycarbonyl)oxazol-2-yl)phenyl)-1-(2-morpholinoethyl)-1 H-pyrazole-5-carboxylic acid (47 mg, 0.107 mmol), pentan-3-amine (14 μL, 0.117 mmol), T3P 50% in EtOAc (95 μL, 0.160 mmol) and triethylamine (45 μL, 0.320 mmol) in EtOAc (1 mL) was stirred at RT for 3 h. The RM was partitioned between water and EtOAc. The aqueous layer was separated and extracted with EtOAc (2×). The combined organics were then concentrated to give 77 mg of ethyl 2-(3-(1-(2-morpholinoethyl)-5-(pentan-3-ylcarbamoyl)-1 H-pyrazol-3-yl)phenyl)oxazole-5-carboxylate which was used crude for the next step.
LCMS Rt: 1.04 min; MS m/z 510.5 [M+H]+ 2minLowpHv03
Step 4: 2-(3-(1-(2-morpholinoethyl)-5-(pentan-3-ylcarbamoyl)-1 H-pyrazol-3-yl)phenyl)oxazole-5-carboxylic acid
A mixture of ethyl 2-(3-(1-(2-morpholinoethyl)-5-(pentan-3-ylcarbamoyl)-1 H-pyrazol-3-yl)phenyl)oxazole-5-carboxylate (77 mg, 0.151 mmol) and TMSOK (28 mg, 0.196 mmol) was stirred in dry THE (1 mL) overnight. The RM was concentrated to give 73 mg of 2-(3-(1-(2-morpholinoethyl)-5-(pentan-3-ylcarbamoyl)-1 H-pyrazol-3-yl)phenyl)oxazole-5-carboxylic acid which was used crude for the next step.

LCMS Rt: 0.88 min; MS m/z 482.5 [M+H]+ 2minLowpHv03

Step 5: (S)-ethyl 3-methyl-2-(2-(3-(1-(2-morpholinoethyl)-5-(pentan-3-ylcarbamoyl)-1H-pyrazol-3-yl)phenyl)oxazole-5-carboxamido)butanoate
A mixture of 2-(3-(1-(2-morpholinoethyl)-5-(pentan-3-ylcarbamoyl)-1 H-pyrazol-3-yl)phenyl)oxazole-5-carboxylic acid (77 mg, 0.160 mmol), (S)-ethyl 2-amino-3-methylbutanoate.HCl (29 mg, 0.160 mmol), T3P 50% in EtOAc (143 μL, 0.240 mmol) and triethylamine (67 μL, 0.480 mmol) in EtOAc (1.5 mL) was stirred at RT overnight.
The RM was diluted with water and EtOAc. The aqueous layer was extracted with EtOAc (2×) and the combined organics were concentrated. The crude material was purified by prep-HPLC (Method: Low pH 20-50% B) to afford 17 mg (16.6%) of (S)-ethyl 3-methyl-2-(2-(3-(1-(2-morpholinoethyl)-5-(pentan-3-ylcarbamoyl)-1 H-pyrazol-3-yl)phenyl)oxazole-5-carboxamido)butanoate.
LCMS Rt: 1.09 min; MS m/z 609.6 [M+H]+ 2minLowpHv03

¹H NMR (400 MHz, METHANOL-d₄) δ ppm 8.64 (d, J=1.47 Hz, 1H) 8.17 (br d, J=7.82 Hz, 1H) 8.04 (d, J=7.83 Hz, 1H) 7.93-7.96 (m, 1H) 7.63 (t, J=7.83 Hz, 1H) 7.31 (s, 1H) 4.83-4.87 (m, 2H) 4.49-4.57 (m, 1H) 3.89 (s, 1H) 3.73-3.80 (m, 3H) 3.18 (br s, 1H) 2.84 (br s, 3H) 2.33 (br d, J=6.60 Hz, 1H) 1.64-1.77 (m, 2H) 1.51-1.62 (m, 2H) 1.08 (dd, J=6.85, 1.22 Hz, 6H) 1.01 (t, J=7.34 Hz, 6H)

Examples 6.1 to 6.5 were prepared by a similar method to that of Example 6.0 by replacing with the appropriate amines and bromo-alkyl species.

### Example 6.1: (2S)-Methyl 2-(2-(3-(5-((Dicyclopropylmethyl)Carbamoyl)-1-(3,3,3-Trifluoro-2-Hydroxypropyl)-1H-Pyrazol-3-Yl)Phenyl)Oxazole-5-Carboxamido)-3-Methylbutanoate

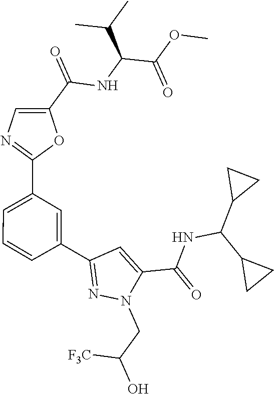 [SMILES: COC(=O)[C@@H](NC(=O)c1cnc(-c2cccc(-c3cc(C(=O)NC(C4CC4)C4CC4)n(CC(O)C(F)(F)F)n3)c2)o1)C(C)C]

LCMS Rt: 1.54 min; MS m/z 618.4 [M+H]+ RXNMON_Basic

¹H NMR (400 MHz, Methanol-d4) δ 8.62 (t, J=1.6 Hz, 1H), 8.13 (dt, J=7.8, 1.3 Hz, 1H), 8.03 (dt, J=7.8, 1.2 Hz, 1H), 7.93 (s, 1H), 7.60 (t, J=7.8 Hz, 1H), 7.26 (s, 1H), 4.77 (ddd, J=14.0, 3.6, 1.0 Hz, 1H), 4.58-4.49 (m, 2H), 3.77 (s, 3H), 3.07 (t, J=8.3 Hz, 1H), 2.38-2.21 (m, J=6.7 Hz, 1H), 2.03 (s, 1H), 1.21-1.09 (m, 2H), 1.05 (dd, J=9.5, 6.8 Hz, 6H), 0.67-0.53 (m, 2H), 0.53-0.43 (m, 2H), 0.39 (qt, J=5.5, 3.4 Hz, 4H).

### Example 6.2: (S)-2-(3-(5-((1-Cyclopropylethyl)Carbamoyl)-1-Methyl-1H-Pyrazol-3-Yl)Phenyl)-N-(Pentan-3-Yl)Oxazole-5-Carboxamide

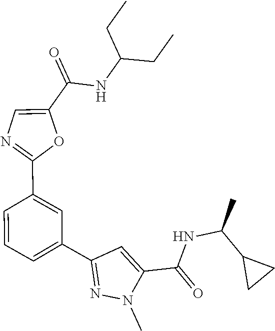 [SMILES: CCC(CC)NC(=O)c1cnc(-c2cccc(-c3cc(C(=O)N[C@@H](C)C4CC4)n(C)n3)c2)o1]

LCMS Rt: 1.19 min; MS m/z 450.4 [M+H]+ RXNMON_Acidic
1H NMR (400 MHz, Chloroform-d) δ 8.49 (t, J=1.5 Hz, 1H), 8.06 (dt, J=7.8, 1.3 Hz, 1H), 7.99 (dt, J=7.8, 1.3 Hz, 1H), 7.83 (s, 1H), 7.57 (t, J=7.8 Hz, 1H), 6.93 (s, 1H), 6.13 (d, J=7.6 Hz, 1H), 6.05 (d, J=9.2 Hz, 1H), 4.27 (s, 3H), 4.10-4.00 (m, 1H), 3.64-3.52 (m, 1H), 1.80-1.68 (m, 2H), 1.63-1.52 (m, 2H), 1.36 (d, J=6.6 Hz, 3H), 1.02 (t, J=7.4 Hz, 6H), 0.67-0.59 (m, 1H), 0.59-0.51 (m, 1H), 0.49-0.42 (m, 1H), 0.38-0.31 (m, 1H).

### Example 6.3: N-((S)-1-Cyclopropylethyl)-2-(3-(5-(((S)-1-Cyclopropylethyl)Carbamoyl)-1-(2-Hydroxy-2-Methylpropyl)-1H-Pyrazol-3-Yl)Phenyl)Oxazole-5-Carboxamide

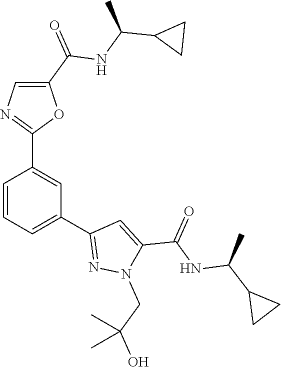 [SMILES: C[C@H](NC(=O)c1cnc(-c2cccc(-c3cc(C(=O)N[C@@H](C)C4CC4)n(CC(C)(C)O)n3)c2)o1)C1CC1]

LCMS Rt: 1.37 min; MS m/z 506.4 [M+H]+ RXNMON_Basic

¹H NMR (400 MHz, Methanol-d4) δ 8.54 (t, J=1.6 Hz, 1H), 8.05 (dt, J=7.8, 1.4 Hz, 1H), 7.94 (dt, J=7.8, 1.3 Hz, 1H), 7.74 (s, 1H), 7.51 (t, J=7.8 Hz, 1H), 7.13 (s, 1H), 4.48 (s, 2H), 3.43-3.33 (m, 2H), 1.26 (d, J=6.7 Hz, 3H), 1.23 (d, J=6.7 Hz, 3H), 1.16 (s, 3H), 1.15 (s, 3H), 1.03-0.85 (m, 2H), 0.55-0.44 (m, 2H), 0.44-0.36 (m, 2H), 0.36-0.24 (m, 2H), 0.25-0.15 (m, 2H).

### Example 6.5: (2S)-Methyl 2-(2-(3-(5-(((S)-1-Methoxy-3-Methyl-1-Oxobutan-2-Yl)Carbamoyl)-1-(3,3,3-Trifluoro-2-Hydroxypropyl)-1 H-Pyrazol-3-Yl)Phenyl)Oxazole-5-Carboxamido)-3-Methylbutanoate

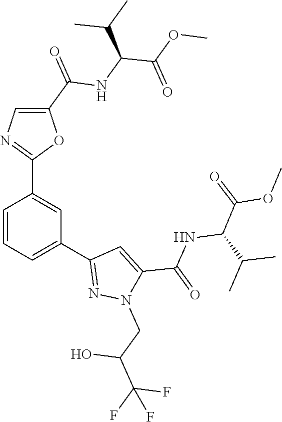 [SMILES: COC(=O)[C@@H](NC(=O)c1cnc(-c2cccc(-c3cc(C(=O)N[C@H](C(=O)OC)C(C)C)n(CC(O)C(F)(F)F)n3)c2)o1)C(C)C]

LCMS Rt: 1.26 min; MS m/z 638.4 [M+H]+ RXNMON_Acidic
1H NMR (400 MHz, DMSO-d6) δ 8.93 (d, J=8.1 Hz, 1H), 8.78 (dd, J=8.0, 2.8 Hz, 1H), 8.52 (s, 1H), 8.13 (dt, J=7.8, 1.2 Hz, 1H), 8.09 (s, 1H), 8.02 (d, J=7.8 Hz, 1H), 7.73-7.65 (m, 2H), 6.65 (s, 1H), 4.85-4.74 (m, 2H), 4.52 (s, 1H), 4.40-4.31 (m, 2H), 3.69 (d, J=2.0 Hz, 6H), 2.26-2.13 (m, 2H), 1.00 (d, J=6.7 Hz, 6H), 0.96 (d, J=6.8 Hz, 6H).

Example 7 of the present invention may be prepared according to Scheme 13.

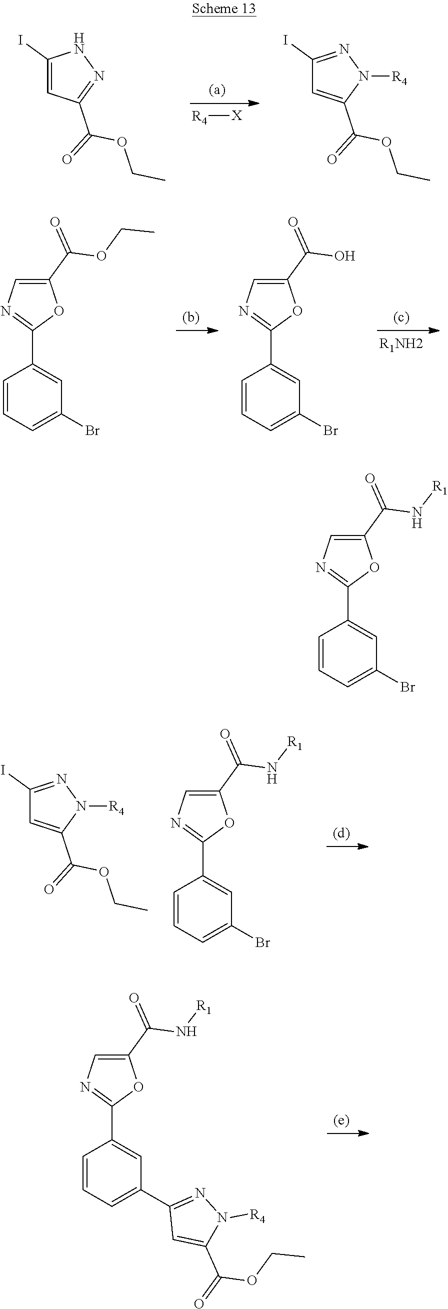

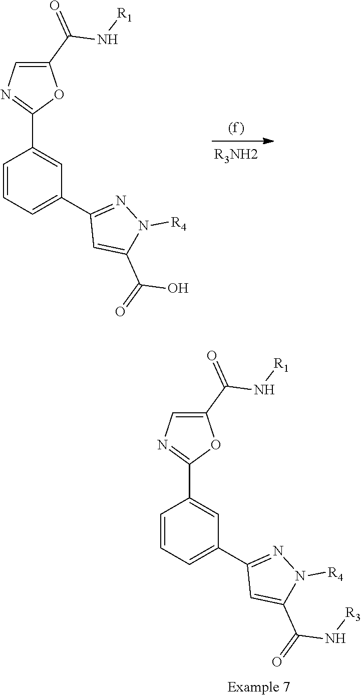

Step (a) involves alkylation of a suitable pyrazole with a haloalkane (R₄—×) in the presence of a base such as NEt3, Na2CO3, Cs2CO3, or K2CO3 in a solvent such as THF or DMF.
Step (b) involves conversion of ethyl ester to carboxylic acid using a suitable base such as NaOH, KOH or KOTMS in a solvent such as THF, methanol or water.
Step (c) involves reaction of an amine(R₁NH2) with carboxylic acid in a suitable solvent such as DMF or ethyl acetate with a suitable base such as diisopropylethylamine or triethylamine and an amide coupling reagent such as T3P or pyBOP.
Step (d) involves C-H insertion reaction of oxazole to haloaromatic in a suitable solvent such as DME, DMA, DMF, THF or toluene in the presence of a suitable palladium catalyst such as Pd(OAc)2 or Pd2(dba)3 and ligand such as Xphos, Sphos, cy-JohnPhos or RuPhos or by using commercially available pre-formed palladium ligand adduct catalysts such as Xphos-Pd-G1, G2 or G3, RuPhos-Pd -G1,G2, G3 in the presence of pivalic acid and suitable base such as Cs2CO3 with heating under inert atmosphere.
Step (e) involves conversion of ethyl ester to carboxylic acid using a suitable base such as NaOH, KOH or KOTMS in a solvent such as THF, methanol or water.
Step (f) involves reaction of an amine(R₃NH2) with free acid in a suitable solvent such as DMF or ethyl acetate with a suitable base such as diisopropylethylamine or triethylamine and an amide coupling reagent such as T3P or pyBOP.

### Example 7.0: (S)-Ethyl 3-Methyl-2-(1-(2-Morpholinoethyl)-3-(3-(5-(Pentan-3-Ylcarbamoyl)Oxazol-2-Yl)Phenyl)-1H-Pyrazole-5-Carboxamido)Butanoate

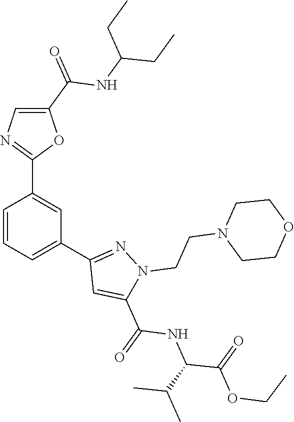 [SMILES: CCOC(=O)[C@@H](NC(=O)c1cc(-c2cccc(-c3ncc(C(=O)NC(CC)CC)o3)c2)nn1CCN1CCOCC1)C(C)C]

Step 1: Ethyl 5-iodo-1-(2-morpholinoethyl)-1H-pyrazole-3-carboxylate, Ethyl 3-iodo-1-(2-morpholinoethyl)-1 H-pyrazole-5-carboxylate
To a mixture of 4-(2-chloroethyl)morpholine.HCl (1.04 g, 5.64 mmol) and triethylamine (786 μL, 5.64 mmol) in DMF (18 mL) was added ethyl 5-iodo-1H-pyrazole-3-carboxylate (500 mg, 1.879 mmol) and Cs₂CO₃ (1.84 g, 5.64 mmol). The resulting mixture was stirred in a microwave at 110° C. for 2 h. A second portion of Cs₂CO₃ (612 mg, 1.879 mmol) was added and the RM was microwaved at 110° C. for 2 h. The RM was filtered to remove solid Cs₂CO₃, washing thoroughly with EtOAc. The organic filtrate was sequentially washed with water, brine, dried over MgSO4 and concentrated. The crude material was purified by FCC: (0-50% EtOAc/iso-hexane) to give 459 mg of ethyl 3-iodo-1-(2-morpholinoethyl)-1H-pyrazole-5-carboxylate.
LCMS Rt: 0.68 min; MS m/z 380.3 [M+H]+ 2minLowpHv03
Step 2: 2-(3-bromophenyl)oxazole-5-carboxylic acid
A mixture of ethyl 2-(3-bromophenyl)oxazole-5-carboxylate (200 mg, 0.675 mmol) and TMSOK (144 mg, 1.013 mmol) in THF (7 mL) was stirred at RT under nitrogen overnight. The RM was concentrated to give 251 mg of 2-(3-bromophenyl)oxazole-5-carboxylic acid as a white solid which was used crude for the next reaction.
LCMS Rt: 1.21 min; MS m/z 268.2 [M+H]+ 2minLowpHv03
Step 3: 2-(3-bromophenyl)-N-(pentan-3-yl)oxazole-5-carboxamide
A mixture of 2-(3-bromophenyl)oxazole-5-carboxylic acid (250 mg, 0.933 mmol), pentan-3-amine (120 μL, 1.026 mmol), T3P 50% EtOAc (833 μL, 1.399 mmol) and TEA (390 μL, 2.80 mmol) was stirred at RT for 72h. The RM was diluted with EtOAc and washed with water. The aqueous layer was separated and extracted with EtOAc (2×). The combined organics were washed with sat. NaHCO₃ solution, brine, dried over MgSO4 and concentrated to give 273 mg of 2-(3-bromophenyl)-N-(pentan-3-yl)oxazole-5-carboxamide as a white solid which was used crude for the next reaction.
LCMS Rt: 1.41 min; MS m/z 339.3 [M+H]+ 2minLowpHv03
Step 4: Ethyl 1-(2-morpholinoethyl)-3-(3-(5-(pentan-3-ylcarbamoyl)oxazol-2-yl)phenyl)-1H-pyrazole-5-carboxylate
A mixture of 2-(3-bromophenyl)-N-(pentan-3-yl)oxazole-5-carboxamide (89 mg, 0.264 mmol), XPhos-Pd-G2 (21 mg, 0.026 mmol), XPhos (25 mg, 0.053 mmol), hypodiboric acid (71 mg, 0.791 mmol) and KOAc (78 mg, 0.791 mmol) in ethanol (3 mL) was stirred at 80° C. under nitrogen for 2 hrs. A solution of ethyl 3-iodo-1-(2-morpholinoethyl)-1H-pyrazole-5-carboxylate (100 mg, 0.264 mmol) in ethanol (500 μL) was then added followed by 2M K₂CO₃ (396 μL, 0.791 mmol). The RM which was then stirred again at 80° C. under nitrogen for 6 h. The RM was partitioned between EtOAc and water. The aqueous layer was separated and extracted with EtOAc (2×). The combined organics were washed with brine, dried over MgSO4, filtered through celite and concentrated to give 158 mg of ethyl 1-(2-morpholinoethyl)-3-(3-(5-(pentan-3-ylcarbamoyl)oxazol-2-yl)phenyl)-1 H-pyrazole-5-carboxylate as a yellow oil which was used crude for the next reaction.
LCMS Rt: 1.01 min; MS m/z 510.6 [M+H]+ 2minLowpHv03
Step 5: 1-(2-morpholinoethyl)-3-(3-(5-(pentan-3-ylcarbamoyl)oxazol-2-yl)phenyl)-1H-pyrazole-5-carboxylic acid
A mixture of ethyl 1-(2-morpholinoethyl)-3-(3-(5-(pentan-3-ylcarbamoyl)oxazol-2-yl)phenyl)-1H-pyrazole-5-carboxylate (158 mg, 0.310 mmol) and TMSOK (119 mg, 0.465 mmol) in dry THF (3 mL) was stirred at RT under nitrogen 18 h. The RM was concentrated under reduced pressure to give a 240 mg of 1-(2-morpholinoethyl)-3-(3-(5-(pentan-3-ylcarbamoyl)oxazol-2-yl)phenyl)-1 H-pyrazole-5-carboxylic acid as a pale yellow solid which was used crude for the next reaction.
LCMS Rt: 0.92 min; MS m/z 482.5 [M+H]+ 2minLowpHv03
Step 6: (S)-ethyl 3-methyl-2-(1-(2-morpholinoethyl)-3-(3-(5-(pentan-3-ylcarbamoyl)oxazol-2-yl)phenyl)-1 H-pyrazole-5-carboxamido)butanoate
A mixture of 1-(2-morpholinoethyl)-3-(3-(5-(pentan-3-ylcarbamoyl)oxazol-2-yl)phenyl)-1H-pyrazole-5-carboxylic acid (240 mg, 0.461 mmol), (S)-ethyl 2-amino-3-methylbutanoate.HCl (74 mg, 0.507 mmol), T3P (50% in EtOAc) (274 μL, 0.461 mmol) and triethylamine (193 μL, 1.383 mmol) in EtOAc (5 mL) was stirred at RT 18 h. The RM was diluted with EtOAc and washed with water. The aqueous layer was then extracted with EtOAc (2×) and the combined organics were washed with sat. NaHCO₃, brine, dried over MgSO4 and concentrated. The crude material was purified by prep HPLC Method: Low pH 20-50% B to give 24 mg (8.13%) of (S)-ethyl 3-methyl-2-(1-(2-morpholinoethyl)-3-(3-(5-(pentan-3-ylcarbamoyl)oxazol-2-yl)phenyl)-1H-pyrazole-5-carboxamido)butanoate as a white solid.
LCMS Rt: 1.07 min; MS m/z 609.7 [M+H]+ 2minLowpHv03
Example 8 of the present invention may be prepared according to Scheme 14.

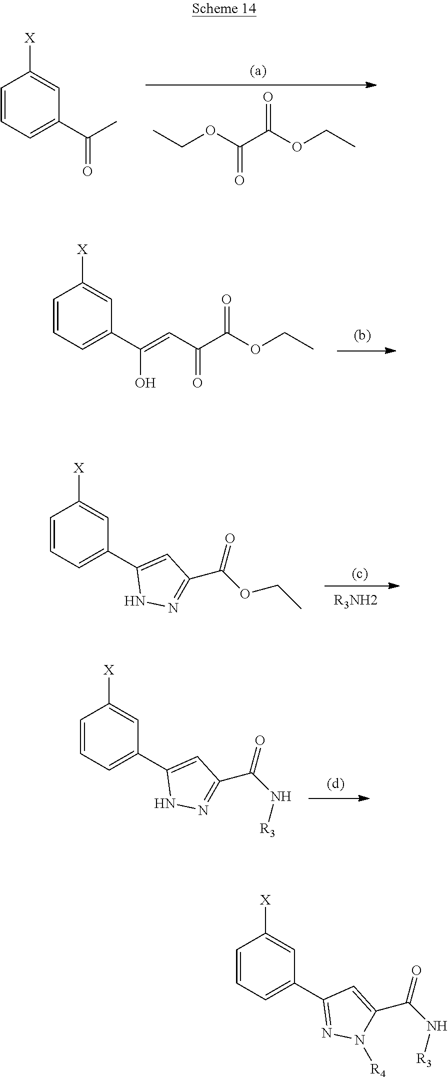

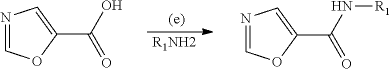

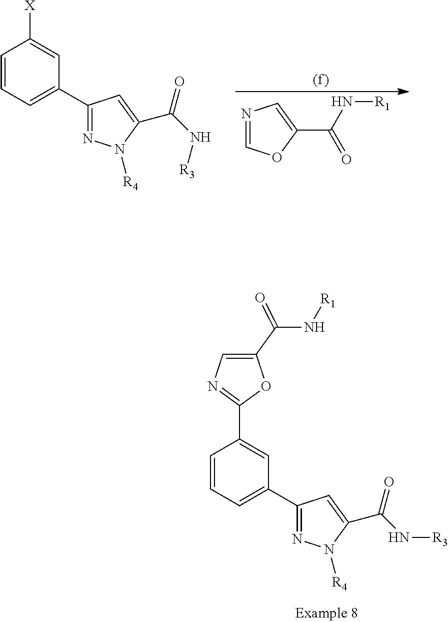

Step (a) involves deprotonation with base such as sodium ethoxide in ethanol at low temperature followed by addition of di-ethyl oxalate.
Step (b) involves formation of the pyrazole ring by treatment of the ethyl enoyl acetate with hydrazine hydrate and an acid such as acetic acid.
Step (c) involves reaction of an amine(R₃NH2) with ethyl ester in a suitable solvent such as THF with a suitable base such as 2,3,4,6,7,8-hexahydro-1H-pyrimido[1,2-a]pyrimidine to give an amide.
Step (d) involves addition to pyrazole of either an alkyl halide (R₄—×) or SN2 opening of a suitable epoxide in the presence of a base such as NEt3, Na2CO3, Cs2CO3, or K2C03 in a solvent such as THF or DMF.
Step (e) involves reaction of an amine(R₁NH2) with free acid in a suitable solvent such as DMF or ethyl acetate with a suitable base such as diisopropylethylamine or triethylamine and an amide coupling reagent such as HATU, T3P or pyBOP.
Step (f) involves C-H insertion reaction of oxazole to halophenylpyrazole in a suitable solvent such as DME, DMA, DMF, THF or toluene in the presence of a suitable palladium catalyst such as Pd(OAc)2 or Pd2(dba)3 and ligand such as Xphos, Sphos, cy-JohnPhos or RuPhos or by using commercially available pre-formed palladium ligand adduct catalysts such as Xphos-Pd-G1, G2 or G3, RuPhos-Pd -G1,G2, G3 in the presence of pivalic acid and suitable base such as Cs2CO3 with heating under inert atmosphere.
Alternatively, step (f) can be performed on a suitable ester substituted oxazole which may then be used to access the desired final amide.

### Example 8.0: (S)-2-(3-(5-((1-Cyclopropylethyl)Carbamoyl)-1-(2-Hydroxy-2-Methylpropyl)-1H-Pyrazol-3-Yl)Phenyl)-N-(Pentan-3-Yl)Oxazole-5-Carboxamide

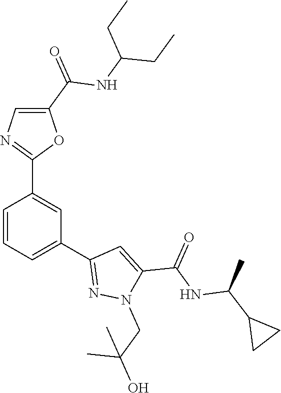 [SMILES: CCC(CC)NC(=O)c1cnc(-c2cccc(-c3cc(C(=O)N[C@@H](C)C4CC4)n(CC(C)(C)O)n3)c2)o1]

(Z)-ethyl 4-(3-bromophenyl)-4-hydroxy-2-oxobut-3-enoate

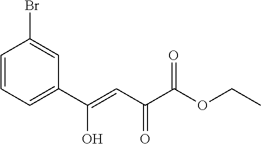 [SMILES: CCOC(=O)C(=O)/C=C(\O)c1cccc(Br)c1]

To a solution of 1-(3-bromophenyl)ethanone (1.00 g, 5.02 mmol) in 20 mL EtOH was added sodium ethoxide solution (2.06 mL, 5.5 mmol) (21% in EtOH) dropwise at 0° C., followed by diethyl oxalate (0.81 g, 5.5 mmol). The RM was stirred overnight at room temperature and then concentrated in vacuo. The residue was dissolved in EtOAc and treated with sat. NH4CI solution. The organic layer was extracted with EtOAc, washed with water, dried over Na2SO4, and concentrated in vacuo. The crude material was purified by FCC (0-100% EtOAc/heptane) to give 1.2 g (80%) of (Z)-ethyl 4-(3-bromophenyl)-4-hydroxy-2-oxobut-3-enoate.
LCMS Rt: 0.70 min; MS m/z 300.6 [M+H]+ RXNMON_Basic 1H NMR (400 MHz, Methanol-d4) δ 8.17 (d, J=1.6 Hz, 1H), 8.01 (d, J=7.8 Hz, 1H), 7.84-7.78 (m, 1H), 7.47 (t, J=7.9 Hz, 1H), 7.08 (s, 1H), 4.37 (q, J=7.1 Hz, 2H), 1.39 (t, J=7.1 Hz, 3H).
Step 2: Ethyl 5-(3-bromophenyl)-1 H-pyrazole-3-carboxylate
To a solution of ethyl 4-(3-bromophenyl)-2,4-dioxobutanoate (1.2 g, 4.01 mmol) in EtOH (15 mL) were added hydrazine monohydrate (0.221 g, 4.41 mmol) and acetic acid (0.253 mL, 4.41 mmol) at 0° C. The mixture was stirred 18 h. The mixture was concentrated and the residue was taken up in DCM. The solution was washed with sat sodium bicarbonate and water, dried over Na₂SO₄, and concentrated. The crude material was purified by FCC (0-100% EtOAc/heptane) to give 1.04 g (88%) of ethyl 5-(3-bromophenyl)-1H-pyrazole-3-carboxylate.
LCMS Rt: 1.43 min; MS m/z 296.5 [M+H]+ RXNMON_Acidic
Step 3: (S)-5-(3-bromophenyl)-N-(1-cyclopropylethyl)-1H-pyrazole-3-carboxamide
To a 5 mL microwave vial were added ethyl 5-(3-bromophenyl)-1H-pyrazole-3-carboxylate (1.0 g, 3.39 mmol), (S)-1-cyclopropylethanamine (1.083 mL, 10.16 mmol), 2,3,4,6,7,8-hexahydro-1H-pyrimido[1,2-a]pyrimidine (0.118 g, 0.847 mmol) and THF (3 mL). The mixture was heated at 140° C. by microwave for 2 h. The mixture was concentrated and purified by FCC (0-100% EtOAc/heptane) to give 0.81 g (71.5%) of (S)-5-(3-bromophenyl)-N-(1-cyclopropylethyl)-1 H-pyrazole-3-carboxamide.
LCMS Rt: 1.39 min; MS m/z 335.7 [M+H]+ RXNMON_Acidic
Step 4: (S)-3-(3-bromophenyl)-N-(1-cyclopropylethyl)-1-(2-hydroxy-2-methylpropyl)-1H-pyrazole-5-carboxamide, (S)-5-(3-bromophenyl)-N-(1-cyclopropylethyl)-1-(2-hydroxy-2-methylpropyl)-1 H-pyrazole-3-carboxamide
To a solution of (S)-5-(3-bromophenyl)-N-(1-cyclopropylethyl)-1H-pyrazole-3-carboxamide in DMF (1 mL) were added 2,2-dimethyloxirane (0.199 mL, 2.244 mmol) and cesium carbonate (487 mg, 1.496 mmol). The mixture was heated at 100° C. for 4 h. After cooling to room temperature, the mixture was diluted with water and extracted with EtOAc (2×). The extracts were dried over Na₂SO₄ and concentrated. The crude material was purified by FCC (0-100% EtOAc/heptane) to give ((S)-3-(3-bromophenyl)-N-(1-cyclopropylethyl)-1-(2-hydroxy-2-methylpropyl)-1H-pyrazole-5-carboxamide.
LCMS Rt: 1.57 min; MS m/z 408.1 [M+H]+ RXNMON_Acidic
Step 5: (S)-2-(3-(5-((1-cyclopropylethyl)carbamoyl)-1-(2-hydroxy-2-methylpropyl)-1 H-pyrazol-3-yl)phenyl)-N-(pentan-3-yl)oxazole-5-carboxamide
Pivalic acid (10.05 mg, 0.098 mmol), K₂CO₃ (102 mg, 0.738 mmol), and RuPhos-Pd-G1 (8.97 mg, 0.012 mmol) were combined in a vial under nitrogen. A solution of (S)-3-(3-bromophenyl)-N-(1-cyclopropylethyl)-1-(2-hydroxy-2-methylpropyl)-1 H-pyrazole-5-carboxamide (100 mg, 0.246 mmol) in toluene (1 mL) was added followed by Intermediate 6 (90 mg, 0.492 mmol). The mixture was stirred for 16 h at 110° C. The mixture was diluted with CH2Cl2, filtered through celite, and concentrated. The crude material was purified by prep HPLC Method 1 to provide 20.5 mg (16.4%) of (S)-2-(3-(5-((1-cyclopropylethyl)carbamoyl)-1-(2-hydroxy-2-methylpropyl)-1H-pyrazol-3-yl)phenyl)-N-(pentan-3-yl)oxazole-5-carboxamide.
LCMS Rt: 1.51 min; MS m/z 508.5 [M+H]+ RXNMON_Acidic_NonPolar
1H NMR (400 MHz, Methanol-d4) δ 8.63 (t, J=1.5 Hz, 1H), 8.18-8.11 (m, 1H), 8.07-8.00 (m, 1H), 7.85 (s, 1H), 7.60 (t, J=7.8 Hz, 1H), 7.22 (s, 1H), 4.57 (s, 2H), 3.91 (tt, J=9.1, 5.0 Hz, 1H), 3.52-3.40 (m, 1H), 1.77-1.64 (m, 2H), 1.64-1.49 (m, 2H), 1.33 (d, J=6.7 Hz, 3H), 1.25 (s, 3H), 1.24 (s, 3H), 1.08-1.00 (m, 1H), 0.97 (t, J=7.4 Hz, 6H), 0.64-0.53 (m, 1H), 0.53-0.44 (m, 1H), 0.39 (dq, J=9.7, 5.0 Hz, 1H), 0.29 (dq, J=9.4, 4.9 Hz, 1H).
Examples 8.1 and 8.2 were prepared by a similar method to that of Example 8.0 by replacing with the appropriate amines and halo-alkyl species.

### Example 8.1(i) and 8.1(ii): (2S)-Methyl 2-(2-(3-(5-(((S)-1-Cyclopropylethyl)Carbamoyl)-1-(3,3,3-Trifluoro-2-Hydroxypropyl)-1H-Pyrazol-3-Yl)Phenyl)Oxazole-5-Carboxamido)-3-Methylbutanoate and (2S)-Methyl 2-(2-(3-(5-(((S)-1-Cyclopropylethyl)Carbamoyl)-1-(3,3,3-Trifluoro-2-Hydroxypropyl)-1H-Pyrazol-3-Yl)Phenyl)Oxazole-5-Carboxamido)-3-Methylbutanoate

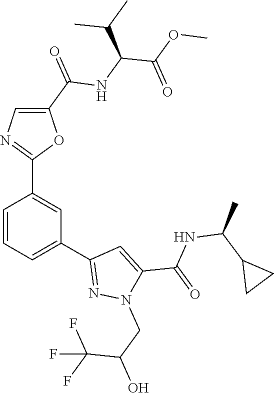 [SMILES: COC(=O)[C@@H](NC(=O)c1cnc(-c2cccc(-c3cc(C(=O)N[C@@H](C)C4CC4)n(CC(O)C(F)(F)F)n3)c2)o1)C(C)C]

The two isomers were separated by SFC Method 5.

### Example 8.1(i): (2S)-Methyl 2-(2-(3-(5-(((S)-1-Cyclopropylethyl)Carbamoyl)-1-(3,3,3-Trifluoro-2-Hydroxypropyl)-1H-Pyrazol-3-Yl)Phenyl)Oxazole-5-Carboxamido)-3-Methylbutanoate: The Faster Eluting Diastereomer by SFC Method

LCMS Rt: 1.26 min; MS m/z 592.1 [M+H]+ RXNMON_Acidic
1H NMR (400 MHz, DMSO-d₆) δ 8.93 (d, J=8.0 Hz, 1H), 8.54 (d, J=8.3 Hz, 1H), 8.51 (t, J=1.6 Hz, 1H), 8.13 (dt, J=7.8, 1.3 Hz, 1H), 8.09 (s, 1H), 8.01 (dt, J=7.8, 1.2 Hz, 1H), 7.68 (t, J=7.8 Hz, 1H), 7.50 (s, 1H), 6.62 (s, 1H), 4.87-4.75 (m, 2H), 4.56 (s, 1H), 4.35 (t, J=7.8 Hz, 1H), 3.69 (s, 3H), 3.55-3.43 (m, 1H), 2.27-2.15 (m, 1H), 1.24 (d, J=6.7 Hz, 3H), 1.06-0.91 (m, 7H), 0.54-0.45 (m, 1H), 0.44-0.37 (m, 1H), 0.37-0.29 (m, 1H), 0.27-0.18 (m, 1H).

### Example 8.1(ii): (2S)-Methyl 2-(2-(3-(5-(((S)-1-Cyclopropylethyl)Carbamoyl)-1-(3,3,3-Trifluoro-2-Hydroxypropyl)-1H-Pyrazol-3-Yl)Phenyl)Oxazole-5-Carboxamido)-3-Methylbutanoate

The later eluting diastereomer by SFC Method 5.
LCMS Rt: 1.26 min; MS m/z 592.1 [M+H]+ RXNMON_Acidic
1H NMR (400 MHz, DMSO-d6) δ 8.93 (d, J=7.9 Hz, 1H), 8.55 (d, J=7.1 Hz, 1H), 8.51 (t, J=1.5 Hz, 1H), 8.13 (dt, J=7.8, 1.1 Hz, 1H), 8.10 (s, 1H), 8.04-7.97 (m, 1H), 7.68 (t, J=7.8 Hz, 1H), 7.51 (s, 1H), 6.64 (s, 1H), 4.89-4.75 (m, 2H), 4.62-4.48 (m, 1H), 4.35 (t, J=7.6 Hz, 1H), 3.69 (s, 3H), 3.55-3.42 (m, 1H), 2.26-2.14 (m, 1H), 1.24 (d, J=6.7 Hz, 3H), 1.05-0.92 (m, 7H), 0.53-0.45 (m, 1H), 0.45-0.36 (m, 1H), 0.36-0.29 (m, 1H), 0.28-0.20 (m, 1H).

### Example 8.2: N-((R)-1-Cyclopropylethyl)-2-(3-(5-(((S)-1-Cyclopropylethyl)Carbamoyl)-1-(3,3,3-Trifluoro-2-Hydroxypropyl)-1H-Pyrazol-3-Yl)Phenyl)Oxazole-5-Carboxamide

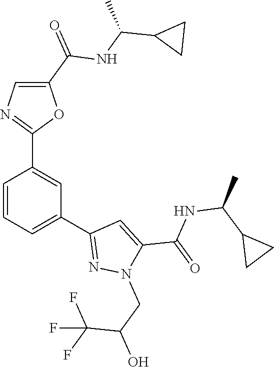 [SMILES: C[C@H](NC(=O)c1cc(-c2cccc(-c3ncc(C(=O)N[C@H](C)C4CC4)o3)c2)nn1CC(O)C(F)(F)F)C1CC1]

LCMS Rt: 1.25 min; MS m/z 544.8 [M+H]+ RXNMON_Acidic
1H NMR (400 MHz, DMSO-d₆) δ 8.64 (d, J=8.3 Hz, 1H), 8.58-8.52 (m, 1H), 8.50 (s, 1H), 8.12 (dt, J=7.8, 1.2 Hz, 1H), 8.00 (d, J=7.9 Hz, 1H), 7.92 (s, 1H), 7.68 (t, J=7.8 Hz, 1H), 7.51 (d, J=2.7 Hz, 1H), 6.63 (d, J=6.8 Hz, 1H), 4.90-4.74 (m, 2H), 4.61-4.50 (m, 1H), 3.55-3.38 (m, 2H), 1.25 (dd, J=9.1, 6.8 Hz, 6H), 1.07-0.94 (m, 2H), 0.56-0.37 (m, 4H), 0.36-0.18 (m, 4H).
Example 9 of the present invention may be prepared according to Scheme 15.

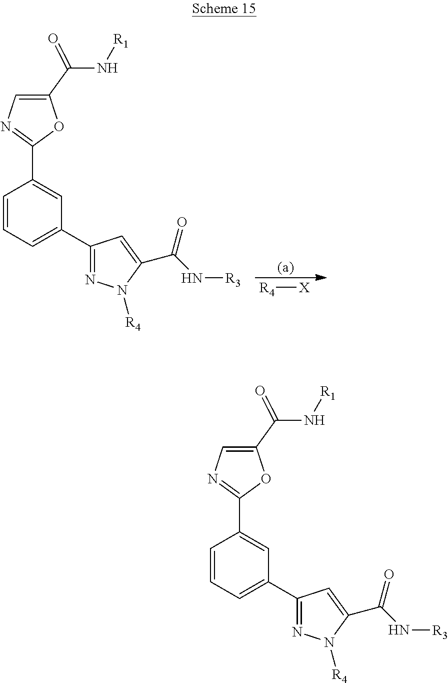

Step (a) involves addition to pyrazole of either an alkyl halide (R₄—×) or SN2 opening of a suitable epoxide in the presence of a base such as NEt3, Na2CO3, Cs2CO3, or K2C03 in a solvent such as THF or DMF. Alternatively, an additional step of hydrogenation involving Pd(0) in a solvent such as methanol or ethanol to remove benzyl protective group may be performed in order to reveal a tether bearing an hydroxyl group.

### Example 9.0: (S)-2-(3-(5-((1-Cyclopropylethyl)Carbamoyl)-1-(2-Isopropoxyethyl)-1 H-Pyrazol-3-Yl)Phenyl)-N-(Pentan-3-Yl)Oxazole-5-Carboxamide

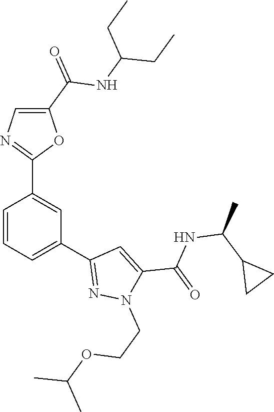 [SMILES: CCC(CC)NC(=O)c1cnc(-c2cccc(-c3cc(C(=O)N[C@@H](C)C4CC4)n(CCOC(C)C)n3)c2)o1]

A stirred suspension of (S)-2-(3-(3-((1-cyclopropylethyl)carbamoyl)-1 H-pyrazol-5-yl)phenyl)-N-(pentan-3-yl)oxazole-5-carboxamide (Example 3.25) (50 mg, 0.115 mmol), 2-(2-chloroethoxy)propane (24 mg, 0.196 mmol), and K₂CO₃ (32 mg, 0.23 mmol) in DMF (0.574 mL) was heated at 90° C. for 42 h. The RM was diluted with 1:1 EtOAc:diethyl ether (70 mL) and washed with water (30 mL). The organic phase was separated, dried over MgSO₄, filtered and concentrated. The crude material was purified by FCC (5-60% EtOAc/heptane) to afford 22 mg (34.9%) of (S)-2-(3-(5-((1-cyclopropylethyl)carbamoyl)-1-(2-isopropoxyethyl)-1 H-pyrazol-3-yl)phenyl)-N-(pentan-3-yl)oxazole-5-carboxamide.
LCMS Rt: 1.33 min; MS m/z 522.4 [M+H]+ RXNMON_Acidic
1H NMR (400 MHz, DMSO-d₆) δ 8.49 (t, J=1.5 Hz, 1H), 8.45 (d, J=8.3 Hz, 1H), 8.30 (d, J=8.8 Hz, 1H), 8.10 (dt, J=7.8, 1.2 Hz, 1H), 7.99 (dt, J=7.8, 1.2 Hz, 1H), 7.93 (s, 1H), 7.66 (t, J=7.8 Hz, 1H), 7.39 (s, 1H), 4.69 (t, J=5.9 Hz, 2H), 3.84-3.76 (m, 1H), 3.74 (t, J=5.9 Hz, 2H), 3.56-3.42 (m, 2H), 1.66-1.43 (m, 4H), 1.24 (d, J=6.7 Hz, 3H), 1.01 (d, J=6.1 Hz, 6H), 0.99-0.94 (m, 1H), 0.88 (t, J=7.4 Hz, 6H), 0.53-0.44 (m, 1H), 0.44-0.36 (m, 1H), 0.33 (dq, J=9.4, 5.1 Hz, 1H), 0.23 (dq, J=9.2, 5.0 Hz, 1H).
Examples 9.1 to 9.8 were prepared by a similar method to that of Example 9.0 by replacing with the appropriate amines and bromo-alkyl species.

### Example 9.1: 2-(3-(5-(((S)-1-Cyclopropylethyl)Carbamoyl)-1-(3,3,3-Trifluoro-2-Hydroxypropyl)-1H-Pyrazol-3-Yl)Phenyl)-N-(Pentan-3-Yl)Oxazole-5-Carboxamide

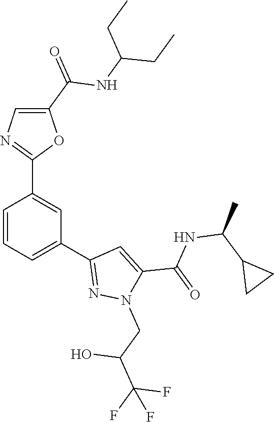 [SMILES: CCC(CC)NC(=O)c1cnc(-c2cccc(-c3cc(C(=O)N[C@@H](C)C4CC4)n(CC(O)C(F)(F)F)n3)c2)o1]

LCMS Rt: 1.28 min; MS m/z 548.3 [M+H]+ RXNMON_Acidic
1H NMR (400 MHz, DMSO-d₆) δ 8.60-8.54 (m, 1H), 8.52-8.48 (m, 1H), 8.33 (d, J=8.8 Hz, 1H), 8.12 (dt, J=7.8, 1.2 Hz, 1H), 8.00 (d, J=7.8 Hz, 1H), 7.94 (s, 1H), 7.68 (t, J=7.8 Hz, 1H), 7.52 (d, J=2.9 Hz, 1H), 6.65 (d, J=6.8 Hz, 1H), 4.89-4.75 (m, 2H), 4.61-4.48 (m, 1H), 3.85-3.72 (m, 1H), 3.54-3.42 (m, 1H), 1.65-1.42 (m, 4H), 1.24 (d, J=6.6 Hz, 3H), 1.04-0.94 (m, 1H), 0.88 (t, J=7.4 Hz, 6H), 0.53-0.45 (m, 1H), 0.44-0.37 (m, 1H), 0.36-0.28 (m, 1H), 0.27-0.19 (m, 1H).

### Example Pyrazole Tether 9.2 (i) and 9.2 (ii): 2-(3-(5-(((S)-1-Cyclopropylethyl)Carbamoyl)-1-(3,3,3-Trifluoro-2-Hydroxypropyl)-1 H-Pyrazol-3-Yl)Phenyl)-N-(Dicyclopropylmethyl)Oxazole-5-Carboxamide and 2-(3-(5-(((S)-1-Cyclopropylethyl)Carbamoyl)-1-(3,3,3-Trifluoro-2-Hydroxypropyl)-1 H-Pyrazol-3-Yl)Phenyl)-N-(Dicyclopropylmethyl)Oxazole-5-Carboxamide

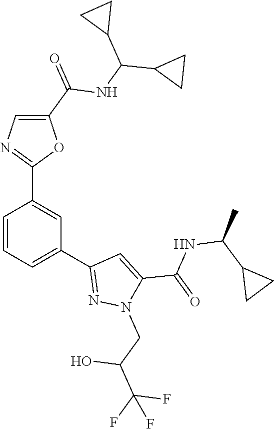 [SMILES: C[C@H](NC(=O)c1cc(-c2cccc(-c3ncc(C(=O)NC(C4CC4)C4CC4)o3)c2)nn1CC(O)C(F)(F)F)C1CC1]

### Example 9.2 (i)

2-(3-(5-(((S)-1-cyclopropylethyl)carbamoyl)-1-(3,3,3-trifluoro-2-hydroxypropyl)-1 H-pyrazol-3-yl)phenyl)-N-(dicyclopropylmethyl)oxazole-5-carboxamide

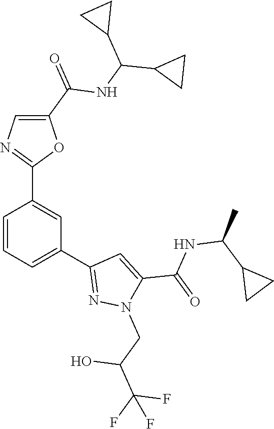 [SMILES: C[C@H](NC(=O)c1cc(-c2cccc(-c3ncc(C(=O)NC(C4CC4)C4CC4)o3)c2)nn1CC(O)C(F)(F)F)C1CC1]

P1-1, the faster eluting diastereomer by SFC Method 6.
LCMS Rt: 1.31 min; MS m/z 572.2 [M+H]+ RXNMON_Acidic

¹H NMR (400 MHz, DMSO-d₆) δ 8.71 (d, J=8.8 Hz, 1H), 8.54 (d, J=8.3 Hz, 1H), 8.52-8.48 (m, 1H), 8.13 (d, J=7.8 Hz, 1H), 8.00 (d, J=7.9 Hz, 1H), 7.94 (s, 1H), 7.68 (t, J=7.8 Hz, 1H), 7.51 (s, 1H), 6.62 (d, J=6.8 Hz, 1H), 4.87-4.77 (m, 2H), 4.60-4.49 (m, 1H), 3.49 (h, J=6.7 Hz, 1H), 2.93 (q, J=8.5 Hz, 1H), 1.24 (d, J=6.7 Hz, 3H), 1.17-1.07 (m, 2H), 1.03-0.94 (m, 1H), 0.59-0.44 (m, 3H), 0.44-0.30 (m, 6H), 0.30-0.18 (m, 3H).

### Example 9.2 (ii)

2-(3-(5-(((S)-1-cyclopropylethyl)carbamoyl)-1-(3,3,3-trifluoro-2-hydroxypropyl)-1 H-pyrazol-3-yl)phenyl)-N-(dicyclopropylmethyl)oxazole-5-carboxamide

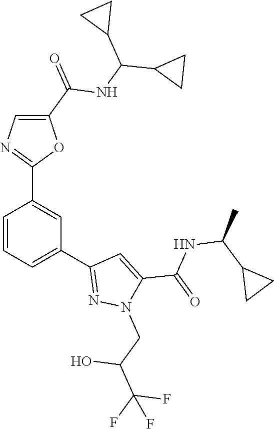 [SMILES: C[C@H](NC(=O)c1cc(-c2cccc(-c3ncc(C(=O)NC(C4CC4)C4CC4)o3)c2)nn1CC(O)C(F)(F)F)C1CC1]

P1-2, the slower eluting diastereomer by SFC Method 6. (2-(3-(5-(((S)-1-cyclopropylethyl)carbamoyl)-1-(3,3,3-trifluoro-2-hydroxypropyl)-1 H-pyrazol-3-yl)phenyl)-N-(dicyclopropylmethyl)oxazole-5-carboxamide
LCMS Rt: 1.31 min; MS m/z 572.2 [M+H]+ RXNMON_Acidic
1H NMR (400 MHz, DMSO-d₆) δ 8.71 (d, J=8.8 Hz, 1H), 8.54 (d, J=8.3 Hz, 1H), 8.50 (t, J=1.6 Hz, 1H), 8.13 (dt, J=7.8, 1.3 Hz, 1H), 8.00 (dt, J=7.8, 1.2 Hz, 1H), 7.94 (s, 1H), 7.68 (t, J=7.8 Hz, 1H), 7.51 (s, 1H), 6.62 (d, J=6.8 Hz, 1H), 4.88-4.75 (m, 2H), 4.60-4.49 (m, 1H), 3.48 (h, J=6.8 Hz, 1H), 2.93 (q, J=8.6 Hz, 1H), 1.24 (d, J=6.7 Hz, 3H), 1.17-1.06 (m, 2H), 1.05-0.94 (m, 1H), 0.59-0.45 (m, 3H), 0.44-0.20 (m, 9H).

### Example 9.3: (S)-2-(3-(5-((1-Cyclopropylethyl)Carbamoyl)-1-(Oxetan-3-Yl)-1H-Pyrazol-3-Yl)Phenyl)-N-(Pentan-3-Yl)Oxazole-5-Carboxamide

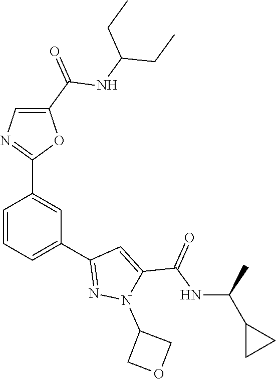 [SMILES: CCC(CC)NC(=O)c1cnc(-c2cccc(-c3cc(C(=O)N[C@@H](C)C4CC4)n(C4COC4)n3)c2)o1]

LCMS Rt: 1.33 min; MS m/z 492.3 [M+H]+ RXNMON_Basic
NMR: 1H NMR (400 MHz, Methanol-d4) δ 8.67 (t, J=1.5 Hz, 1H), 8.12 (ddt, J=28.7, 7.8, 1.1 Hz, 2H), 7.62 (t, J=7.8 Hz, 1H), 6.23-6.11 (m, 1H), 5.22 (t, J=6.5 Hz, 2H), 5.06 (td, J=7.2, 2.1 Hz, 2H), 3.91 (tt, J=9.1, 5.0 Hz, 1H), 3.49-3.36 (m, 1H), 1.77-1.49 (m, 4H), 1.32 (d, J=6.7 Hz, 3H), 1.08-0.93 (m, 7H), 0.63-0.43 (m, 2H), 0.32 (ddq, J=42.6, 9.4, 5.0 Hz, 2H).

### Example 9.4: (S)-Methyl 2-(2-(3-(5-((Dicyclopropylmethyl)Carbamoyl)-1-(2-Hydroxy-2-Methylpropyl)-1 H-Pyrazol-3-Yl)Phenyl)Oxazole-5-Carboxamido)-3-Methylbutanoate

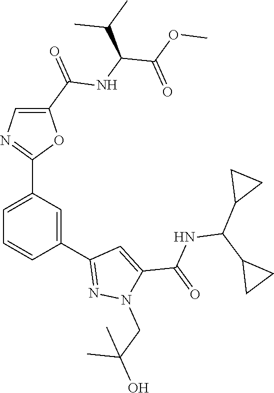 [SMILES: COC(=O)[C@@H](NC(=O)c1cnc(-c2cccc(-c3cc(C(=O)NC(C4CC4)C4CC4)n(CC(C)(C)O)n3)c2)o1)C(C)C]

LCMS Rt: 1.45 min; MS m/z 578.4 [M+H]+ RXNMON_Basic
1H NMR (400 MHz, Methanol-d4) δ 8.64 (t, J=1.5 Hz, 1H), 8.15 (dt, J=7.8, 1.3 Hz, 1H), 8.05 (dt, J=7.8, 1.2 Hz, 1H), 7.95 (s, 1H), 7.61 (t, J=7.8 Hz, 1H), 7.23 (s, 1H), 4.57 (s, 2H), 4.52 (d, J=7.1 Hz, 1H), 3.77 (s, 3H), 3.04 (t, J=8.3 Hz, 1H), 2.29 (hept, J=6.8 Hz, 1H), 1.25 (s, 6H), 1.12 (tdd, J=8.2, 4.9, 3.2 Hz, 2H), 1.05 (dd, J=9.5, 6.8 Hz, 6H), 0.65-0.52 (m, 2H), 0.51-0.42 (m, 2H), 0.38 (hept, J=3.9 Hz, 4H).

### Example 9.5: 2-(3-(5-(((S)-1-Cyclopropylethyl)Carbamoyl)-1-(2-Hydroxypropyl)-1H-Pyrazol-3-Yl)Phenyl)-N-(Pentan-3-Yl)Oxazole-5-Carboxamide

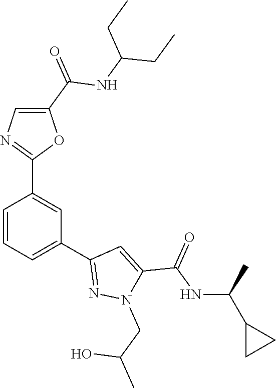 [SMILES: CCC(CC)NC(=O)c1cnc(-c2cccc(-c3cc(C(=O)N[C@@H](C)C4CC4)n(CC(C)O)n3)c2)o1]

LCMS Rt: 1.34 min; MS m/z 494.4 [M+H]+ RXNMON_Basic
NMR: ¹H NMR (400 MHz, Methanol-d4) δ 8.53 (t, J=1.6 Hz, 1H), 8.04 (dt, J=7.8, 1.2 Hz, 1H), 7.92 (dt, J=7.8, 1.2 Hz, 1H), 7.75 (s, 1H), 7.50 (t, J=7.8 Hz, 1H), 7.11 (d, J=1.0 Hz, 1H), 4.51-4.37 (m, 2H), 4.12 (qt, J=9.1, 4.5 Hz, 1H), 3.81 (tt, J=9.0, 5.0 Hz, 1H), 3.44-3.31 (m, 1H), 1.59 (dtd, J=14.9, 7.4, 5.1 Hz, 2H), 1.48 (ddd, J=13.9, 8.7, 7.4 Hz, 2H), 1.23 (dd, J=6.7, 3.7 Hz, 3H), 1.12 (d, J=6.3 Hz, 3H), 0.98-0.90 (m, 1H), 0.88 (t, J=7.4 Hz, 6H), 0.48 (dt, J=8.5, 4.4 Hz, 1H), 0.43-0.36 (m, 1H), 0.34-0.26 (m, 1H), 0.19 (dt, J=9.5, 5.0 Hz, 1H).

### Example 9.6: (S)-Ethyl 2-(2-(3-(5-((Dicyclopropylmethyl)Carbamoyl)-1-(2-Hydroxy-2-Methylpropyl)-1 H-Pyrazol-3-Yl)Phenyl)Oxazole-5-Carboxamido)-3-Methylbutanoate

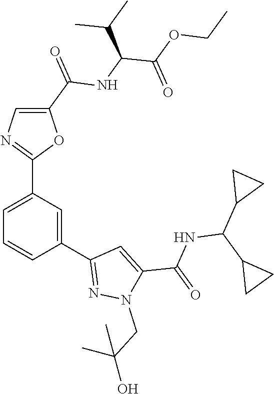 [SMILES: CCOC(=O)[C@@H](NC(=O)c1cnc(-c2cccc(-c3cc(C(=O)NC(C4CC4)C4CC4)n(CC(C)(C)O)n3)c2)o1)C(C)C]

LCMS Rt: 1.51 min; MS m/z 592.4 [M+H]+ RXNMON_Basic
LCMS: MS m/z 592 [M+1]*; HPLC Peak Rt=1.51 minutes; Purity >95%; RXNMON_Basic. 1H NMR (400 MHz, Methanol-d4) δ 8.64 (t, J=1.5 Hz, 1H), 8.14 (dt, J=7.8, 1.2 Hz, 1H), 8.05 (dt, J=7.8, 1.2 Hz, 1H), 7.95 (s, 1H), 7.61 (t, J=7.8 Hz, 1H), 7.23 (s, 1H), 4.57 (s, 2H), 4.24 (qq, J=7.1, 3.7 Hz, 2H), 3.04 (t, J=8.3 Hz, 1H), 2.39-2.22 (m, J=6.8 Hz, 1H), 1.30 (t, J=7.1 Hz, 3H), 1.25 (s, 6H), 1.23-0.94 (m, 9H), 0.65-0.55 (m, 2H), 0.51-0.32 (m, 6H).

### Example 9.7: 2-(3-(1-(2-Hydroxyethyl)-5-(Pentan-3-Ylcarbamoyl)-1H-Pyrazol-3-Yl)Phenyl)-N-(Pentan-3-Yl)Oxazole-5-Carboxamide

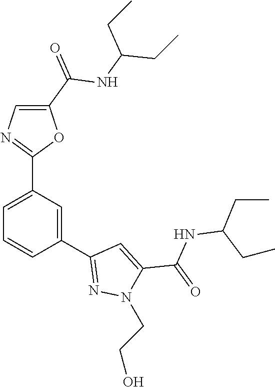 [SMILES: CCC(CC)NC(=O)c1cnc(-c2cccc(-c3cc(C(=O)NC(CC)CC)n(CCO)n3)c2)o1]

LCMS Rt: 1.34 min; MS m/z 482.6 [M+H]+ 2minLowpHv03

¹H NMR (400 MHz, METHANOL-d₄) δ ppm 8.66 (s, 1H) 8.12-8.27 (m, 1H) 8.04 (d, J=7.82 Hz, 1H) 7.86 (s, 1H) 7.58-7.65 (m, 1H) 7.23 (s, 1H) 4.71 (t, J=5.62 Hz, 2H) 3.98 (t, J=5.62 Hz, 2H) 3.84-3.96 (m, 2H) 1.65-1.78 (m, 4H) 1.50-1.64 (m, 4H) 1.00 (td, J=7.46, 2.93 Hz, 12H)

### Example 9.8

(S)-2-(3-(5-((1-cyclopropylethyl)carbamoyl)-1-(2-hydroxyethyl)-1 H-pyrazol-3-yl)phenyl)-N-(pentan-3-yl)oxazole-5-carboxamide

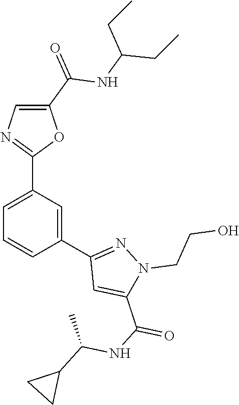 [SMILES: CCC(CC)NC(=O)c1cnc(-c2cccc(-c3cc(C(=O)N[C@@H](C)C4CC4)n(CCO)n3)c2)o1]

LCMS Rt: 1.32 min; MS m/z 480.5 [M+H]+ 2minLowpHv03

¹H NMR (400 MHz, METHANOL-d₄) δ ppm 8.64 (t, J=1.47 Hz, 1H) 8.12-8.17 (m, 1H) 7.99-8.04 (m, 1H) 7.84 (s, 1H) 7.60 (t, J=7.82 Hz, 1H) 7.21 (s, 1H) 4.68 (t, J=5.69 Hz, 2H) 3.97 (t, J=5.62 Hz, 2H) 3.87-3.94 (m, 1H) 3.45-3.51 (m, 1H) 1.64-1.75 (m, 2H) 1.51-1.64 (m, 2H) 1.33 (d, J=6.72 Hz, 3H) 1.01-1.07 (m, 1H) 0.97 (t, J=7.40 Hz, 6H) 0.55-0.61 (m, 1H) 0.45-0.54 (m, 1H) 0.36-0.44 (m, 1H) 0.24-0.32 (m, 1H).

Example 10 of the present invention may be prepared according to Scheme 16.

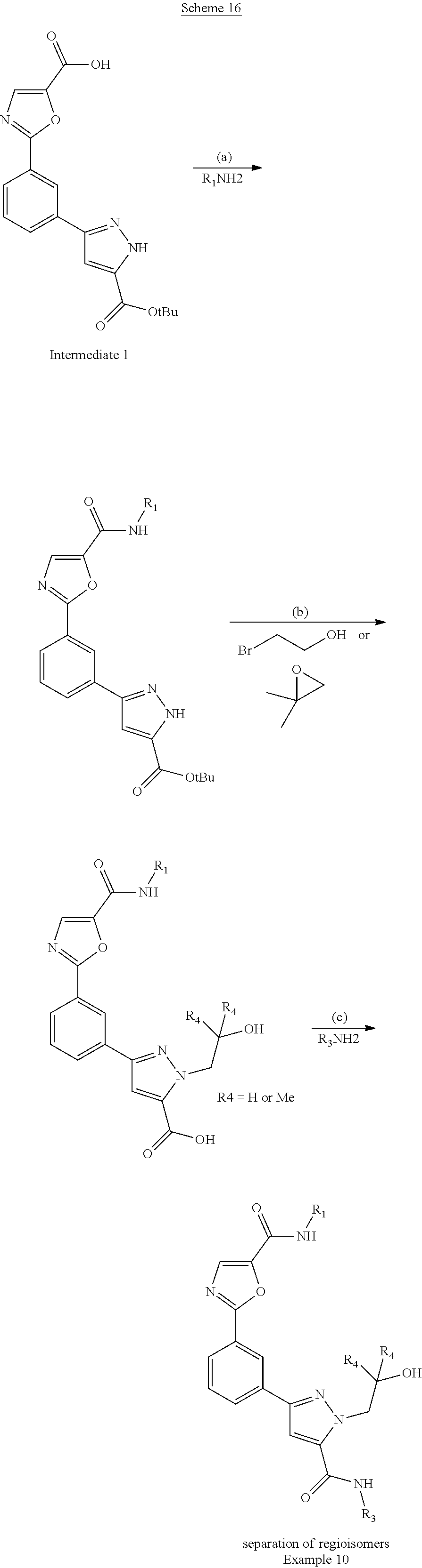

Step (a) involves reaction of an amine(R₁NH2) with Intermediate 1 in a suitable solvent such as DMF or ethyl acetate with a suitable base such as diisopropylethylamine or triethylamine and an amide coupling reagent such as HATU, T3P or pyBOP.
Step (b) involves addition to pyrazole of either an alkyl halide (R₄—×) or SN2 opening of a suitable epoxide in the presence of a base such as Na2CO3, Cs2CO3, or K2C03 in a solvent such as THF or DMF. In the course of facilitating the SN2 reaction, with suitable temperature and time, the base also may hydrolyze the tert-butyl ester to give free acid.
Step (d) involves reaction of an amine(R₃NH2) with free acid in a suitable solvent such as DMF or ethyl acetate with a suitable base such as diisopropylethylamine or triethylamine and an amide coupling reagent such as HATU, T3P or pyBOP.

### Example 10.0: (S)-Ethyl 2-(3-(3-(5-((Dicyclopropylmethyl)Carbamoyl)Oxazol-2-Yl)Phenyl)-1-(2-Hydroxy-2-Methylpropyl)-1 H-Pyrazole-5-Carboxamido)-3-Methylbutanoate

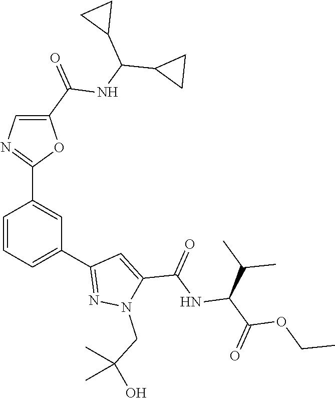 [SMILES: CCOC(=O)[C@@H](NC(=O)c1cc(-c2cccc(-c3ncc(C(=O)NC(C4CC4)C4CC4)o3)c2)nn1CC(C)(C)O)C(C)C]

Step 1 Tert-butyl 5-(3-(5-((dicyclopropylmethyl)carbamoyl)oxazol-2-yl)phenyl)-1 H-pyrazole-3-carboxylate
To a solution of 2-(3-(3-(tert-butoxycarbonyl)-1 H-pyrazol-5-yl)phenyl)oxazole-5-carboxylic acid (Intermediate 1) (0.47 g, 1.32 mmol) and dicyclopropylmethanamine hydrochloride (0.215 g, 1.455 mmol) in DMF (10 mL) were added DIPEA (0.513 g, 3.97 mmol) and HATU (0.553 g, 1.455 mmol) at 0° C. The reaction mixture was stirred overnight. The mixture was quenched with sat. NaHCO₃solution and extracted with EtOAc. The organic layer was washed with 1 M HCl, water, and brine. The organic extracts were dried over Na2SO4 and then purified by FCC (0-100% EtOAc/Heptane) to give 0.47 g (79%) of tert-butyl 5-(3-(5-((dicyclopropylmethyl)carbamoyl)oxazol-2-yl)phenyl)-1 H-pyrazole-3-carboxylate.
1H NMR (400 MHz, DMSO-d6) δ 14.04 (d, J=10.7 Hz, 1H), 8.71 (dd, J=19.3, 8.7 Hz, 1H), 8.58 (s, 1H), 8.19-7.97 (m, 2H), 7.92 (d, J=13.0 Hz, 1H), 7.67 (dt, J=23.6, 7.8 Hz, 1H), 7.32 (s, 1H), 2.92 (q, J=8.6 Hz, 1H), 1.56 (d, J=7.6 Hz, 9H), 1.16 (dt, J=14.6, 7.2 Hz, 2H), 0.54 (dt, J=8.4, 4.6 Hz, 2H), 0.38 (tq, J=10.9, 5.2 Hz, 4H), 0.31-0.20 (m, 2H).
LCMS: Rt 1.57 min, MS m/z [M+H]+; 449.3, RXNMON_Acidic_NonPolar
Step 2: 3-(3-(5-((dicyclopropylmethyl)carbamoyl)oxazol-2-yl)phenyl)-1-(2-hydroxy-2-methylpropyl)-1 H-pyrazole-5-carboxylic acid
To a solution of tert-butyl 5-(3-(5-((dicyclopropylmethyl)carbamoyl)oxazol-2-yl)phenyl)-1 H-pyrazole-3-carboxylate (200 mg, 0.445 mmol) in DMF (0.6 mL) were added Cs2CO3 (291 mg, 0.892 mmol) and 2,2-dimethyloxirane (96 mg, 1.338 mmol). The mixture was heated to 100° C. for 3 h. The reaction mixture was diluted with water and acidified (pH 2) with 1 M HCl solution. The mixture was extracted with EtOAc (×2). The extracts were dried over Na2SO4 and concentrated in vacuo. The resulting 3-(3-(5-((dicyclopropylmethyl)carbamoyl)oxazol-2-yl)phenyl)-1-(2-hydroxy-2-methylpropyl)-1 H-pyrazole-5-carboxylic acid was used crude for the next step.
LCMS Rt: 1.34 min; MS m/z 465.2 [M+H]+ RXNMON Acidic_NonPolar
1H NMR (400 MHz, DMSO-d6) δ 13.49 (s, 1H), 8.73 (d, J=8.7 Hz, 1H), 8.57 (d, J=8.0 Hz, 1H), 8.12 (d, J=8.0 Hz, 1H), 8.05 (d, J=7.8 Hz, 1H), 7.93 (s, 1H), 7.65 (t, J=7.8 Hz, 1H), 7.40 (s, 1H), 4.59 (s, 2H), 2.91 (q, J=8.6 Hz, 1H), 1.87 (d, J=18.8 Hz, 1H), 1.20-1.13 (m, 2H), 1.11 (s, 6H), 0.60-0.49 (m, 2H), 0.42-0.34 (m, 4H), 0.30-0.20 (m, 2H).
Step 3: (S)-ethyl 2-(3-(3-(5-((dicyclopropylmethyl)carbamoyl)oxazol-2-yl)phenyl)-1-(2-hydroxy-2-methylpropyl)-1 H-pyrazole-5-carboxamido)-3-methylbutanoate
To a solution of 3-(3-(5-((dicyclopropylmethyl)carbamoyl)oxazol-2-yl)phenyl)-1-(2-hydroxy-2-methylpropyl)-1H-pyrazole-5-carboxylic acid (48 mg, 0.103 mmol) in DMF were added (S)-ethyl 2-amino-3-methylbutanoate hydrochloride (20.6 mg, 0.114 mmol), N-ethyl-N-isopropylpropan-2-amine (40 mg, 0.31 mmol), and HATU (43 mg, 0.114 mmol) at 0 0C. The mixture was stirred overnight. The mixture was quenched with sat. NaHCO₃and extracted with 10% MeOH in CH2Cl2 (×2). The extracts were dried over Na2SO4 and concentrated in vacuo. The crude material was purified by FCC (0-100% EtOAc/Heptane) to give 32 mg (52% yield) of (S)-ethyl 2-(3-(3-(5-((dicyclopropylmethyl)carbamoyl)oxazol-2-yl)phenyl)-1-(2-hydroxy-2-methylpropyl)-1 H-pyrazole-5-carboxamido)-3-methylbutanoate.
LCMS Rt: 1.60 min; MS m/z 592.3 [M+H]+ RXNMON_Acidic_NonPolar 1H NMR (400 MHz, Methanol-d4) δ 8.65 (t, J=1.5 Hz, 1H), 8.15 (dt, J=7.8, 1.3 Hz, 1H), 8.05 (dt, J=7.8, 1.2 Hz, 1H), 7.84 (s, 1H), 7.61 (t, J=7.8 Hz, 1H), 7.29 (s, 1H), 4.57 (dd, J=34.8, 13.7 Hz, 2H), 4.49 (s, 1H), 4.23 (qd, J=7.1, 2.4 Hz, 2H), 2.99 (t, J=8.6 Hz, 1H), 2.37-2.21 (m, J=6.8 Hz, 1H), 1.34-1.28 (m, 6H), 1.27 (s, 3H), 1.22-1.12 (m, 2H), 1.05 (dd, J=6.8, 3.5 Hz, 6H), 0.61 (tdd, J=7.9, 5.0, 3.4 Hz, 2H), 0.48 (ddd, J=8.1, 3.9, 2.8 Hz, 2H), 0.37 (dtt, J=10.6, 5.3, 2.7 Hz, 4H).
Examples 10.1 and 10.2 were prepared by a similar method to that of Example 10.0 by replacing with the appropriate amines and halo-alkyl species.

### Example 10.1: N-((R)-1-Cyclopropylethyl)-2-(3-(5-(((R)-1-Cyclopropylethyl)Carbamoyl)-1-(2-Hydroxyethyl)-1H-Pyrazol-3-yl)Phenyl)Oxazole-5-Carboxamide

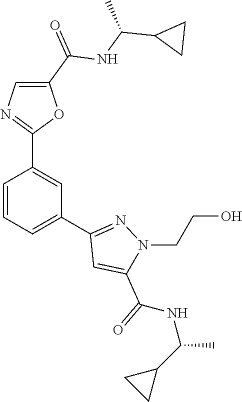 [SMILES: C[C@@H](NC(=O)c1cnc(-c2cccc(-c3cc(C(=O)N[C@H](C)C4CC4)n(CCO)n3)c2)o1)C1CC1]

LCMS Rt: 0.64 min; MS m/z 478.2 [M+H]+ RXNMON_Acidic_NonPolar
1H NMR (400 MHz, DMSO-d₆) δ ppm 8.63 (d, J=8.3 Hz, 1H) 8.49 (t, J=1.5 Hz, 1H) 8.47 (d, J=8.3 Hz, 1H) 8.10 (dt, J=8.0, 1.3 Hz, 1H) 7.98 (dt, J=8.1, 1.3 Hz, 1H) 7.92 (s, 1H) 7.66 (t, J=7.8 Hz, 1H) 7.41 (s, 1H) 4.90 (br s, 1H) 4.61 (t, J=6.1 Hz, 2H) 3.76 (t, J=6.0 Hz, 2H) 3.38-3.54 (m, 2H) 1.26 (d, J=6.7 Hz, 3H) 1.23 (d, J=6.7 Hz, 3H) 0.92-1.06 (m, 2H) 0.45-0.54 (m, 2H) 0.38-0.46 (m, 2H) 0.29-0.37 (m, 2H) 0.24 (dq, J=11.9, 4.5 Hz, 2H) 0.00-0.00 (m, 1H).

### Example 10.2: N-((R)-1-Cyclopropylethyl)-2-(3-(5-(((S)-1-Cyclopropylethyl)Carbamoyl)-1-(2-Hydroxyethyl)-1H-Pyrazol-3-Yl)Phenyl)Oxazole-5-Carboxamide

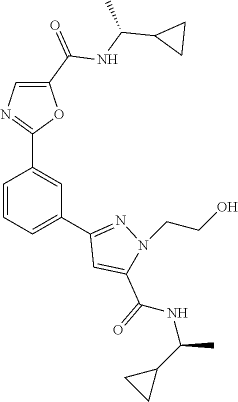 [SMILES: C[C@H](NC(=O)c1cc(-c2cccc(-c3ncc(C(=O)N[C@H](C)C4CC4)o3)c2)nn1CCO)C1CC1]

LCMS Rt: 1.08 min; MS m/z 478.2 [M+H]+ RXNMON_Acidic
1H NMR (400 MHz, DMSO-d₆) δ ppm 8.63 (d, J=8.3 Hz, 1H) 8.49 (t, J=1.5 Hz, 1H) 8.47 (d, J=8.3 Hz, 1H) 8.10 (dt, J=8.0, 1.3 Hz, 1H) 7.98 (dt, J=8.1, 1.3 Hz, 1H) 7.92 (s, 1H) 7.66 (t, J=7.8 Hz, 1H) 7.41 (s, 1H) 4.78-5.04 (m, 1H) 4.61 (s, 2H) 3.76 (t, J=6.0 Hz, 2H) 3.45 (td, J=8.5, 6.8 Hz, 2H) 1.25 (app. dd, J=10.9, 6.7 Hz, 6H) 0.94-1.07 (m, 2H) 0.46-0.56 (m, 2H) 0.38-0.45 (m, 2H) 0.29-0.36 (m, 2H) 0.20-0.28 (m, 2H).
Example 11.0 of the present invention may be prepared according to Scheme 17.

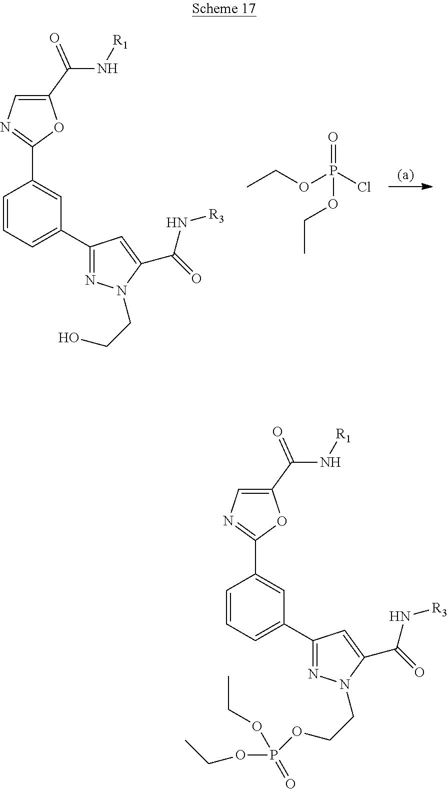

Step (a) involves phosphorylation of a free hydroxyl group in a solvent such as THF or DCM with a base such as DMAP, DIPEA or TEA.

### Example 11.0: (S)-Ethyl 2-(1-(2-((Diethoxyphosphoryl)Oxy)Ethyl)-3-(3-(5-(Pentan-3-Ylcarbamoyl)Oxazol-2-Yl)Phenyl)-1H-Pyrazole-5-Carboxamido)-3-Methylbutanoate

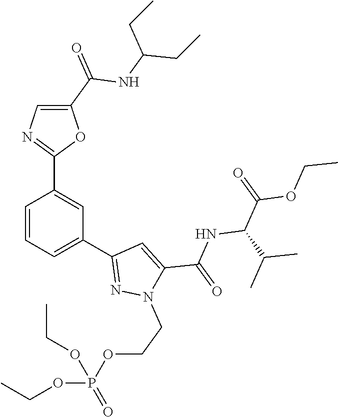 [SMILES: CCOC(=O)[C@@H](NC(=O)c1cc(-c2cccc(-c3ncc(C(=O)NC(CC)CC)o3)c2)nn1CCOP(=O)(OCC)OCC)C(C)C]

Step 1: (S)-ethyl 2-(1-(2-((diethoxyphosphoryl)oxy)ethyl)-3-(3-(5-(pentan-3-ylcarbamoyl)oxazol-2-yl)phenyl)-1 H-pyrazole-5-carboxamido)-3-methylbutanoate
A stirred solution of (S)-ethyl 2-(1-(2-hydroxyethyl)-3-(3-(5-(pentan-3-ylcarbamoyl)oxazol-2-yl)phenyl)-1 H-pyrazole-5-carboxamido)-3-methylbutanoate (Example 5.0), 23 mg, 0.043 mmol), TEA (14.9 μL, 0.107 mmol), and DMAP (catalytic amount) in THF (0.43 mL) was cooled in an ice-bath. To this was added diethyl phosphorochloridate (10 μL, 0.069 mmol) and the RM was allowed to stir at room temperature for 96 h. A further 2.5 Eq TEA (14.9 μL, 0.107 mmol), and 1.62 Eq diethyl phosphorochloridate (10 PL, 0.069 mmol) were added and stirring continued for 5 hours. The RM was diluted with DCM (5 mL) and washed with water (1 mL).
The organic phase was separated and purified by prep HPLC Method 2 (formic acid modifier) to give 14.4 mg, (50%) of (S)-ethyl 2-(1-(2-((diethoxyphosphoryl)oxy)ethyl)-3-(3-(5-(pentan-3-ylcarbamoyl)oxazol-2-yl)phenyl)-1 H-pyrazole-5-carboxamido)-3-methylbutanoate.
LCMS Rt: 1.27 min; MS m/z 676.3 [M+H]+ RXNMON_Acidic
1H NMR (400 MHz, DMSO-d₆) δ 8.71 (d, J=7.9 Hz, 1H), 8.51 (t, J=1.5 Hz, 1H), 8.30 (d, J=8.9 Hz, 1H), 8.12 (dt, J=7.8, 1.2 Hz, 1H), 8.02-7.98 (m, 1H), 7.93 (s, 1H), 7.72 (s, 1H), 7.68 (t, J=7.8 Hz, 1H), 4.83 (t, J=5.0 Hz, 2H), 4.39-4.28 (m, 3H), 4.21-4.09 (m, 2H), 3.94-3.85 (m, 4H), 3.82-3.73 (m, 1H), 2.25-2.15 (m, 1H), 1.63-1.42 (m, 4H), 1.22 (t, J=7.1 Hz, 3H), 1.13 (t, J=7.1 Hz, 6H), 0.99 (dd, J=14.9, 6.8 Hz, 6H), 0.88 (t, J=7.4 Hz, 6H).
Example 12 of the present invention may be prepared according to Scheme 18.

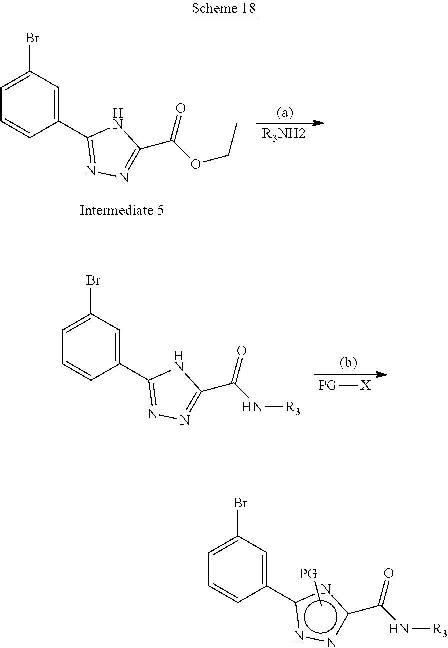

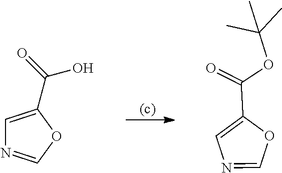

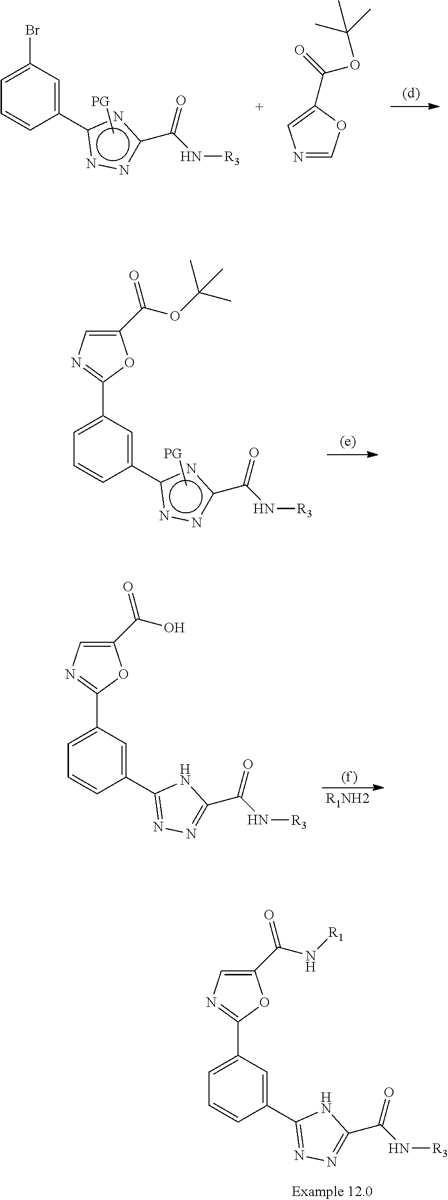

Step (a) involves reaction of an amine(R₃NH2) with Intermediate 5 in a suitable solvent such as THE with a suitable base such as 2,3,4,6,7,8-hexahydro-1 H-pyrimido[1,2-a]pyrimidine to give an amide.
Step (b) involves protection of triazole nitrogen with suitable protective group such as benzyl or SEM-CI by use of appropriate alkyl halide in the presence of a base such as NaH, NEt3, DIPEA, Cs2CO3.
Step (c) involves formation of tert-butyl ester from carboxylic acid by reaction with di-tert-butyl dicarbonate in the presence of a base such as DIPEA or TEA with DMAP is a solvent such as THE or acetonitrile.
Step (d) involves C-H insertion reaction of oxazole to bromophenyltriazole in a suitable solvent such as DME, DMA, DMF, THE or toluene in the presence of a suitable palladium catalyst such as Pd(OAc)2 or Pd2(dba)3 and ligand such as Xphos, Sphos, cy-JohnPhos, CatacXium A, or RuPhos or by using commercially available pre-formed palladium ligand adduct catalysts such as Xphos-Pd-G1, G2 or G3, RuPhos-Pd -G1,G2, G3 in the presence of pivalic acid and suitable base such as Cs2CO3 with heating under inert atmosphere.
Step (e) involves removal of acid labile protective group with liberation of oxazole carboxylic acid from tert-butyl ester by treatment with an acid such as HCl or TFA in a solvent such as DCM or dioxane. Alternatively, if the protective group is a benzyl, it may be removed by treatment with hydrogen in the presence of Pd(0) on carbon black in a solvent such as methanol, ethanol or THE with subsequent acid treatment to produce the free carboxylic acid.
Step (f) involves reaction of an amine(R₁NH2) with free acid in a suitable solvent such as DMF or ethyl acetate with a suitable base such as diisopropylethylamine or triethylamine and an amide coupling reagent such as T3P or pyBOP.

### Example 12.0: N-((S)-1-Cyclopropylethyl)-2-(3-(5-(((S)-1-Cyclopropylethyl)Carbamoyl)-4H-1,2,4-Triazol-3-Yl)Phenyl)Oxazole-5-Carboxamide

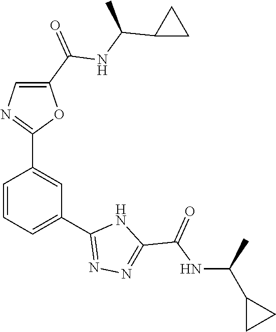 [SMILES: C[C@H](NC(=O)c1nnc(-c2cccc(-c3ncc(C(=O)N[C@@H](C)C4CC4)o3)c2)[nH]1)C1CC1]

Step 1: Tert-butyl oxazole-5-carboxylate
To a solution of oxazole-5-carboxylic acid (5. g, 44.2 mmol) in acetonitrile (100 mL) with DMAP (0.540 g, 4.42 mmol) and NEt3 (12.33 mL, 88 mmol) was added di-tert-butyl dicarbonate (20.53 mL, 88 mmol). The RM was stirred at room temp for 21 h. The RM was concentrated and purified by FCC (0-50% EtOAc/heptane) to afford 6.1 g (71.8%) of tert-butyl oxazole-5-carboxylate as a colorless oil.
LCMS Rt: 1.11 min; MS m/z 170.2 [M+H]+ RXNMON_Acidic

¹H NMR (400 MHz, METHANOL-d₄) δ ppm 8.29-8.39 (m, 1H) 7.73 (s, 1H) 1.58 (s, 9H).

Step 2: (S)-5-(3-bromophenyl)-N-(1-cyclopropylethyl)-4H-1,2,4-triazole-3-carboxamide
In a 20 mL microwave vial was placed Ethyl 5-(3-bromophenyl)-4H-1,2,4-triazole-3-carboxylate, (Intermediate 5) (3.54 g, 11.95 mmol) with (S)-1-cyclopropylethanamine (3.5 mL, 32.8 mmol) in THE (12 mL) with 1,3,4,6,7,8-hexahydro-2H-pyrimido[1,2-a]pyrimidine (0.333 g, 2.391 mmol). The RM was heated by microwave at 140° C. for 1 h. The RM was concentrated and purified by FCC (0-10% MeOH/DCM) to afford 3.7 g (92%) of (S)-5-(3-bromophenyl)-N-(1-cyclopropylethyl)-4H-1,2,4-triazole-3-carboxamide as a white foam.
LCMS Rt: 1.39 min; MS m/z 336.9 [M+H]+ RXNMON_Acidic

¹H NMR (400 MHz, METHANOL-d₄) Q ppm 8.27 (t, J=1.71 Hz, 1H) 8.04 (d, J=7.82 Hz, 1H) 7.64 (d, J=7.82 Hz, 1H) 7.43 (t, J=7.89 Hz, 1H) 3.45-3.50 (m, 1H) 1.35 (d, J=6.72 Hz, 3H) 1.29 (br. s., 2H) 1.02-1.12 (m, 1H) 0.54-0.61 (m, 1H) 0.46-0.53 (m, 1H) 0.36-0.43 (m, 1H) 0.25-0.33 (m, 1H)

Step 3: (S)-5-(3-bromophenyl)-N-(1-cyclopropylethyl)-1-((2-(trimethylsilyl)ethoxy)methyl)-1 H-1,2,4-triazole-3-carboxamide or (S)-3-(3-bromophenyl)-N-(1-cyclopropylethyl)-1-((2-(trimethylsilyl)ethoxy)methyl)-1 H-1,2,4-triazole-5-carboxamide
To a solution of (S)-5-(3-bromophenyl)-N-(1-cyclopropylethyl)-1 H-1,2,4-triazole-3-carboxamide (1.15 g, 3.43 mmol) in THE (30 mL) was added SEMCI (0.852 mL, 4.80 mmol) and NEt3 (0.956 mL, 6.86 mmol). The RM was stirred at room temperature for 2 h. Water and EtOAc were added, the organic layer was separated and washed with brine, dried over Na₂SO₄ and concentrated. The crude material was purified by FCC (0-50% EtOAc/heptane) to afford 1.3 g (81%) of one of the two possible regioisomers (S)-5-(3-bromophenyl)-N-(1-cyclopropylethyl)-1-((2-(trimethylsilyl)ethoxy)methyl)-1 H-1,2,4-triazole-3-carboxamide or (S)-3-(3-bromophenyl)-N-(1-cyclopropylethyl)-1-((2-(trimethylsilyl)ethoxy)methyl)-1H-1,2,4-triazole-5-carboxamide as a colorless oil.

LCMS Rt: 1.82 min; MS m/z 427.9 [M-CH3]+ RXNMON_Acidic

¹H NMR (400 MHz, DICHLOROMETHANE-d₂) δ ppm 8.33 (t, J=1.8 Hz, 1H) 8.10 (dt, J=7.8, 1.3 Hz, 1H) 7.59 (ddd, J=8.0, 2.1, 1.1 Hz, 1H) 7.50 (br d, J=7.8 Hz, 1H) 7.38 (t, J=7.7 Hz, 1H) 6.01 (s, 2H) 3.69-3.78 (m, 2H) 3.47-3.58 (m, 1H) 1.36 (d, J=6.6 Hz, 3H) 0.99-1.06 (m, 1H) 0.93-0.98 (m, 2H) 0.58-0.65 (m, 1H) 0.49-0.58 (m, 1H) 0.43 (td, J=9.5, 5.3 Hz, 1H) 0.29-0.37 (m, 1H) 0.00 (s, 9H).

Step 4: (S)-tert-butyl 2-(3-(3-((1-cyclopropylethyl)carbamoyl)-1-((2-(trimethylsilyl)ethoxy)methyl)-1 H-1,2,4-triazol-5-yl)phenyl)oxazole-5-carboxylate or tert-butyl (S)-2-(3-(5-((1-cyclopropylethyl)carbamoyl)-1-((2-(trimethylsilyl)ethoxy)methyl)-1 H-1,2,4-triazol-3-yl)phenyl)oxazole-5-carboxylate
A suspension of (S)-5-(3-bromophenyl)-N-(1-cyclopropylethyl)-1-((2-(trimethylsilyl)ethoxy)methyl)-1 H-1,2,4-triazole-3-carboxamide (3.4 g, 7.30 mmol), tert-butyl oxazole-5-carboxylate (1.643 g, 8.25 mmol), and cesium carbonate (5.95 g, 18.26 mmol) in toluene (12 mL) was degassed and placed under nitrogen. X-Phos-Pd-G3 (CAS#1445085-55-1) (0.618 g, 0.730 mmol) and pivalic acid (0.424 mL, 3.65 mmol) were added to the mixture and the reaction was heated at 105° C. for 18 h. The resulting suspension was filtered washing with EtOAc. The filtrate was concentrated and purified by FCC (0-40% EtOAc/heptane) to afford 2.75 g (64.6%) of (S)-tert-butyl 2-(3-(3-((1-cyclopropylethyl)carbamoyl)-1-((2-(trimethylsilyl)ethoxy)methyl)-1 H-1,2,4-triazol-5-yl)phenyl)oxazole-5-carboxylate or tert-butyl (S)-2-(3-(5-((1-cyclopropylethyl)carbamoyl)-1-((2-(trimethylsilyl)ethoxy)methyl)-1 H-1,2,4-triazol-3-yl)phenyl)oxazole-5-carboxylate as a white solid.

LCMS Rt: 1.85 min; MS m/z 554.2 [M+H]+ RXNMON_Acidic

¹H NMR (400 MHz, DMSO-d₆) δ ppm 9.13 (d, J=8.80 Hz, 1H) 8.83 (t, J=1.53 Hz, 1H) 8.35 (dt, J=8.04, 1.24 Hz, 1H) 8.16-8.24 (m, 1H) 8.12 (s, 1H) 7.82 (t, J=7.82 Hz, 1H) 6.02 (s, 2H) 3.74 (t, J=7.95 Hz, 2H) 3.46 (td, J=8.68, 6.85 Hz, 1H) 1.64 (s, 9H) 1.35 (d, J=6.72 Hz, 2 H) 1.16-1.25 (m, 1H) 0.93 (t, J=7.95 Hz, 2H) 0.52-0.62 (m, 1H) 0.42-0.51 (m, 1H) 0.26-0.38 (m, 2H) 0.00 (s, 9H)

Step 3: (S)-2-(3-(3-((1-cyclopropylethyl)carbamoyl)-1 H-1,2,4-triazol-5-yl)phenyl)oxazole-5-carboxylic acid
To a solution of (S)-tert-butyl 2-(3-(3-((1-cyclopropylethyl)carbamoyl)-1-((2-(trimethylsilyl)ethoxy)methyl)-1 H-1,2,4-triazol-5-yl)phenyl)oxazole-5-carboxylate and tert-butyl (S)-2-(3-(5-((1-cyclopropylethyl)carbamoyl)-1-((2-(trimethylsilyl)ethoxy)methyl)-1H-1,2,4-triazol-3-yl)phenyl)oxazole-5-carboxylate (2.75 g, 4.97 mmol) in DCM (30 mL) was added TFA (10 mL, 130 mmol). The RM was stirred for 48 h and concentrated. EtOAc and water were added with stirring and the organic phase was separated and washed with water (3×). Concentration of the organic phase gave 2.16 g of (S)-2-(3-(3-((1-cyclopropylethyl)carbamoyl)-1 H-1,2,4-triazol-5-yl)phenyl)oxazole-5-carboxylic acid as a beige solid which was used crude for the next step.
LCMS Rt: 1.13 min; MS m/z 368.1 [M+H]+ RXNMON_Acidic
Step 6: N-((S)-1-cyclopropylethyl)-2-(3-(5-(((S)-1-cyclopropylethyl)carbamoyl)-4H-1,2,4-triazol-3-yl)phenyl)oxazole-5-carboxamide
To a suspension of (S)-2-(3-(5-((1-cyclopropylethyl)carbamoyl)-4H-1,2,4-triazol-3-yl)phenyl)oxazole-5-carboxylic acid (210 mg, 0.372 mmol) in EtOAc (3 mL) was added (S)-1-cyclopropylethanamine (0.069 mL, 0.743 mmol) and NEt3 (0.259 mL, 1.858 mmol) followed by T3P (50% in ETOAc) (0.285 mL, 0.483 mmol). The RM was sonicated then stirred at room temperature for 18 h. The RM was diluted with water, EtOAc and 10% citric acid. The organic phase was washed sequentially with water and brine, dried over Na₂SO₄ and concentrated. The crude material was purified by FCC (0-6% MeOH in DCM) to afford 78 mg of N-((S)-1-cyclopropylethyl)-2-(3-(5-(((S)-1-cyclopropylethyl)carbamoyl)-4H-1,2,4-triazol-3-yl)phenyl)oxazole-5-carboxamide as a white flaky solid.
LCMS Rt: 1.34 min; MS m/z 435.1 [M+H]+ RXNMON_Acidic 1H NMR (400 MHz, DMSO-d₆) δ ppm 15.13 (br s, 1H) 8.89 (br s, 1H) 8.75-8.86 (m, 1H) 8.66 (d, J=8.4 Hz, 1H) 8.18-8.32 (m, 2H) 7.90-7.98 (m, 1H) 7.63-7.79 (m, 1H) 3.35-3.53 (m, 2H) 1.24-1.32 (m, 5H) 1.07-1.18 (m, 1H) 0.97-1.07 (m, 1H) 0.45-0.54 (m, 2H) 0.38-0.45 (m, 1H) 0.19-0.34 (m, 4H).
Examples 12.1 to 12.17 were prepared by a method similar to that of Example 12.0 by replacing with appropriate commercially available amines.

### Example 12.1:N-((R)-1-Cyclopropylethyl)-2-(3-(5-(((S)-1-Cyclopropylethyl)Carbamoyl)-4H-1,2,4-Triazol-3-Yl)Phenyl)Oxazole-5-Carboxamide

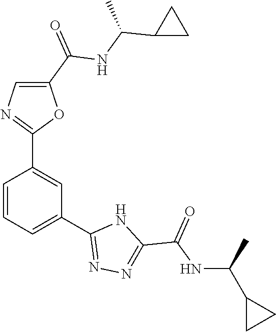 [SMILES: C[C@H](NC(=O)c1nnc(-c2cccc(-c3ncc(C(=O)N[C@H](C)C4CC4)o3)c2)[nH]1)C1CC1]

LCMS Rt: 1.34 min; MS m/z 435.1 [M+H]+ RXNMON_Acidic
1H NMR (400 MHz, DMSO-d₆) δ ppm 8.85-8.97 (m, 1H) 8.75-8.84 (m, 1H) 8.60-8.71 (m, 1H) 8.16-8.29 (m, 2H) 7.93 (s, 1H) 7.66-7.79 (m, 1H) 3.35-3.49 (m, 2H) 1.23-1.31 (m, 6H) 1.15 (br dd, J=11.7, 7.7 Hz, 1H) 0.96-1.07 (m, 1H) 0.45-0.55 (m, 2H) 0.37-0.45 (m, 2H) 0.27-0.36 (m, 2H) 0.20-0.27 (m, 2H).

### Example 12.2:(S)-Ethyl 2-(2-(3-(5-(((S)-1-Cyclopropylethyl)Carbamoyl)-4H-1,2,4-Triazol-3-Yl)Phenyl)Oxazole-5-Carboxamido)-3-Methylbutanoate

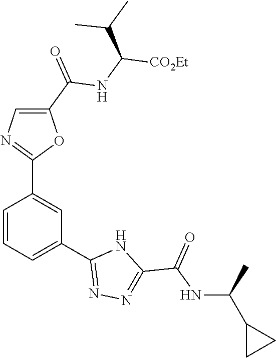 [SMILES: CCOC(=O)[C@@H](NC(=O)c1cnc(-c2cccc(-c3nnc(C(=O)N[C@@H](C)C4CC4)[nH]3)c2)o1)C(C)C]

LCMS Rt: 1.42 min; MS m/z 495.1 [M+H]+ RXNMON_Acidic
1H NMR (400 MHz, METHANOL-d₄) δ ppm 8.87-8.90 (m, 1H) 8.24-8.30 (m, 2H) 7.94 (s, 1H) 7.69 (t, J=7.83 Hz, 1H) 4.50 (d, J=6.97 Hz, 1H) 4.19-4.28 (m, 2H) 3.48-3.55 (m, 1H) 2.26-2.36 (m, 1H) 1.36 (d, J=6.72 Hz, 3H) 1.30 (t, J=7.15 Hz, 4H) 1.06 (dd, J=8.68, 6.85 Hz, 7H) 0.55-0.63 (m, 1H) 0.47-0.54 (m, 1H) 0.38-0.45 (m, 1H) 0.27-0.34 (m, 1 H)

### Example 12.3:(S)-Methyl 2-(2-(3-(5-(((S)-1-Cyclopropylethyl)Carbamoyl)-4H-1,2,4-Triazol-3-Yl)Phenyl)Oxazole-5-Carboxamido)-3-Methylbutanoate

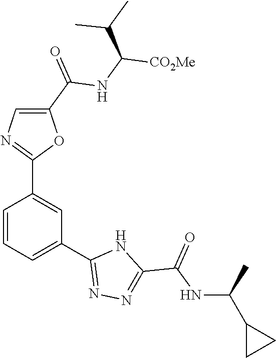 [SMILES: COC(=O)[C@@H](NC(=O)c1cnc(-c2cccc(-c3nnc(C(=O)N[C@@H](C)C4CC4)[nH]3)c2)o1)C(C)C]

LCMS Rt: 1.34 min; MS m/z 481.0 [M+H]+ RXNMON_Acidic
1H NMR (400 MHz, METHANOL-d₄) δ ppm 8.89 (s, 1H) 8.28 (t, J=7.33 Hz, 2H) 7.94 (s, 1H) 7.70 (t, J=7.71 Hz, 1H) 4.53 (d, J=7.07 Hz, 1H) 3.77 (s, 3H) 3.72 (s, 1H) 3.49-3.54 (m, 1H) 2.26-2.36 (m, 1H) 1.36 (d, J=6.69 Hz, 3H) 1.02-1.11 (m, 7H) 0.89-0.97 (m, 2H) 0.54-0.63 (m, 1H) 0.47-0.53 (m, 1H) 0.38-0.46 (m, 1H) 0.26-0.35 (m, 1H)

### Example 12.4:(S)-Tert-Butyl 2-(2-(3-(5-(((S)-1-Cyclopropylethyl)Carbamoyl)-4H-1,2,4-Triazol-3-Yl)Phenyl)Oxazole-5-Carboxamido)-3-Methylbutanoate

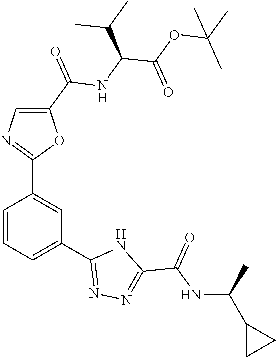 [SMILES: CC(C)[C@H](NC(=O)c1cnc(-c2cccc(-c3nnc(C(=O)N[C@@H](C)C4CC4)[nH]3)c2)o1)C(=O)OC(C)(C)C]

LCMS Rt: 1.54 min; MS m/z 523.1 [M+H]+ RXNMON_Acidic

¹H NMR (400 MHz, METHANOL-d₄) δ ppm 8.89 (s, 1H) 8.28 (t, J=8.93 Hz, 2H) 7.95 (s, 1H) 7.70 (t, J=7.89 Hz, 1H) 4.39 (d, J=6.72 Hz, 1H) 3.48-3.55 (m, 1H) 2.22-2.34 (m, 1H) 1.51 (s, 9H) 1.37 (d, J=6.60 Hz, 3H) 1.10-1.13 (m, 1H) 1.06 (dd, J=6.79, 4.95 Hz, 6H) 0.91-0.97 (m, 1H) 0.56-0.63 (m, 1H) 0.47-0.54 (m, 1H) 0.37-0.45 (m, 1H) 0.26-0.35 (m, 1H)

### Example 12.5:(S)-2-(3-(5-((1-Cyclopropylethyl)Carbamoyl)-4H-1,2,4-Triazol-3-Yl)Phenyl)-N-(Dicyclopropylmethyl)Oxazole-5-Carboxamide

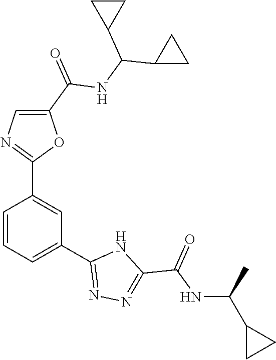 [SMILES: C[C@H](NC(=O)c1nnc(-c2cccc(-c3ncc(C(=O)NC(C4CC4)C4CC4)o3)c2)[nH]1)C1CC1]

LCMS Rt: 1.39 min; MS m/z 461.1 [M+H]+ RXNMON_Acidic
1H NMR (400 MHz, METHANOL-d₄) δ ppm 8.90 (s, 1H) 8.30 (d, J=7.82 Hz, 1H) 8.26 (d, J=7.82 Hz, 1H) 7.85 (s, 1H) 7.70 (t, J=7.89 Hz, 1H) 3.48-3.55 (m, 1H) 3.01 (t, J=8.62 Hz, 1H) 1.37 (d, J=6.72 Hz, 3H) 1.13-1.23 (m, 2H) 1.03-1.12 (m, 1H) 0.56-0.65 (m, 3H) 0.35-0.54 (m, 8H) 0.27-0.34 (m, 1H)

### Example 12.6:(S)-Ethyl 2-(2-(3-(5-((Dicyclopropylmethyl)Carbamoyl)-4H-1,2,4-Triazol-3-Yl)Phenyl)Oxazole-5-Carboxamido)-3-Methylbutanoate

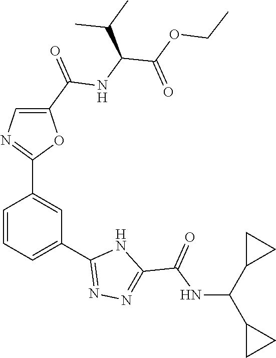 [SMILES: CCOC(=O)[C@@H](NC(=O)c1cnc(-c2cccc(-c3nnc(C(=O)NC(C4CC4)C4CC4)[nH]3)c2)o1)C(C)C]

LCMS Rt: 0.91 min; MS m/z 521.2 [M+H]+ RXNMON_Acidic_NonPolar
1H NMR (400 MHz, METHANOL-d₄) δ ppm 8.90 (s, 1H) 8.28 (br t, J=8.1 Hz, 2H) 7.95 (s, 1H) 7.71 (t, J=7.8 Hz, 1H) 4.50 (d, J=7.0 Hz, 1H) 4.24 (qd, J=7.1, 3.4 Hz, 2H) 3.05 (t, J=8.4 Hz, 1H) 2.24-2.37 (m, 1H) 1.27-1.34 (m, 6H) 1.13-1.22 (m, 2H) 1.06 (dd, J=8.4, 6.8 Hz, 6H) 0.56-0.65 (m, 2H) 0.38-0.51 (m, 6H).

### Example 12.7:(S)-N-(1-Cyclopropylethyl)-2-(3-(5-((Dicyclopropylmethyl)Carbamoyl)-4H-1,2,4-Triazol-3-Yl)Phenyl)Oxazole-5-Carboxamide

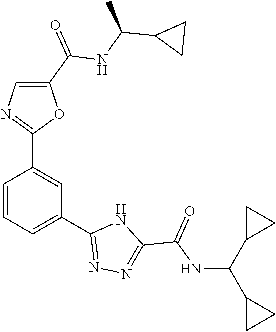 [SMILES: C[C@H](NC(=O)c1cnc(-c2cccc(-c3nnc(C(=O)NC(C4CC4)C4CC4)[nH]3)c2)o1)C1CC1]

LCMS Rt: 0.76 min; MS m/z 461.2 [M+H]+ RXNMON_Acidic_NonPolar
1H NMR (400 MHz, METHANOL-d₄) δ ppm 8.90 (s, 1H) 8.22-8.34 (m, 2H) 7.84 (s, 1H) 7.70 (t, J=7.8 Hz, 1H) 3.48-3.53 (m, 1H) 3.05 (t, J=8.4 Hz, 1H) 1.36 (d, J=6.7 Hz, 3H) 1.14-1.26 (m, 3H) 1.02-1.12 (m, 1H) 0.55-0.66 (m, 3H) 0.43-0.54 (m, 3H) 0.38-0.42 (m, 5H) 0.26-0.33 (m, 1H).

### Example 12.8:N-(Dicyclopropylmethyl)-2-(3-(5-((Dicyclopropylmethyl)Carbamoyl)-4H-1,2,4-Triazol-3-Yl)Phenyl)Oxazole-5-Carboxamide

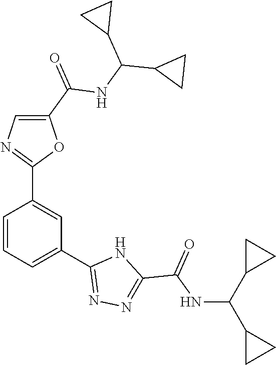 [SMILES: O=C(NC(C1CC1)C1CC1)c1nnc(-c2cccc(-c3ncc(C(=O)NC(C4CC4)C4CC4)o3)c2)[nH]1]

LCMS Rt: 0.88 min; MS m/z 487.2 [M+H]+ RXNMON_Acidic_NonPolar

### 1H NMR (400 MHz, METHANOL-d₄) δ ppm 8.90 (s, 1H) 8.24-8.31 (m, 2H) 7.85 (s, 1H) 7.68-7.73 (m, 1H) 3.06 (t, J=8.4 Hz, 1H) 3.00 (t, J=8.6 Hz, 1H) 1.13-1.23 (m, 4H) 0.57-0.65 (m, 4H) 0.43-0.51 (m, 4H) 0.32-0.43 (m, 8H).

### Example 12.9:(S)-Methyl 2-(2-(3-(5-((Dicyclopropylmethyl)Carbamoyl)-4H-1,2,4-Triazol-3-Yl)Phenyl)Oxazole-5-Carboxamido)-3-Methylbutanoate

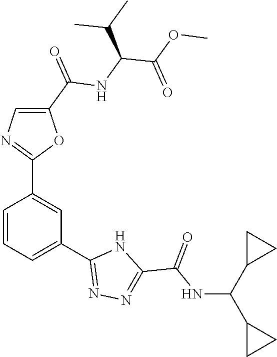 [SMILES: COC(=O)[C@@H](NC(=O)c1cnc(-c2cccc(-c3nnc(C(=O)NC(C4CC4)C4CC4)[nH]3)c2)o1)C(C)C]

LCMS Rt: 1.42 min; MS m/z 507.2 [M+H]+ RXNMON_Acidic

¹H NMR (400 MHz, METHANOL-d₄) δ ppm 8.90 (br s, 1H) 8.24-8.35 (m, 2H) 7.95 (s, 1H) 7.71 (br t, J=7.8 Hz, 1H) 4.53 (d, J=7.1 Hz, 1H) 3.77 (s, 3H) 3.05 (t, J=8.5 Hz, 1H) 2.31 (dq, J=13.7, 6.8 Hz, 1H) 1.14-1.23 (m, 2H) 1.05 (dd, J=9.8, 6.8 Hz, 6H) 0.58-0.64 (m, 2H) 0.38-0.50 (m, 6H).

### Example 12.10:(S)-Methyl 2-(2-(3-(5-(((S)-1-Cyclopropylethyl)Carbamoyl)-4H-1,2,4-Triazol-3-Yl)Phenyl)Oxazole-5-Carboxamido)-3,3-Dimethylbutanoate

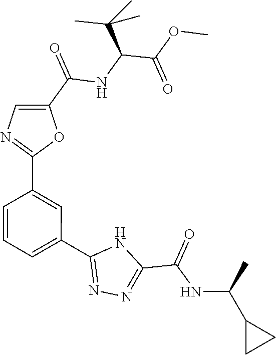 [SMILES: COC(=O)[C@@H](NC(=O)c1cnc(-c2cccc(-c3nnc(C(=O)N[C@@H](C)C4CC4)[nH]3)c2)o1)C(C)(C)C]

LCMS Rt: 1.43 min; MS m/z 495.1 [M+H]+ RXNMON_Acidic
1H NMR (400 MHz, METHANOL-d4) δ ppm 8.89 (br s, 1H) 8.28 (br t, J=7.4 Hz, 2H) 7.99 (s, 1H) 7.71 (br t, J=7.8 Hz, 1H) 4.64 (s, 1H) 3.77 (s, 3H) 3.51 (br d, J=8.4 Hz, 1H) 1.32-1.45 (m, 3H) 1.11 (s, 9H) 0.92-1.00 (m, 1H) 0.55-0.64 (m, 1H) 0.55-0.64 (m, 1H) 0.51 (br dd, J=8.0, 4.5 Hz, 1H) 0.38-0.46 (m, 1H) 0.24-0.36 (m, 1H).

### Example 12.11:(2-(3-(5-(((S)-1-Cyclopropylethyl)Carbamoyl)-4H-1,2,4-Triazol-3-Yl)Phenyl)-N-((S)-3-Methylbutan-2-Yl)Oxazole-5-Carboxamide

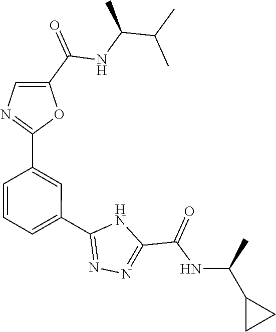 [SMILES: CC(C)[C@H](C)NC(=O)c1cnc(-c2cccc(-c3nnc(C(=O)N[C@@H](C)C4CC4)[nH]3)c2)o1]

LCMS Rt: 1.38 min; MS m/z 437.1 [M+H]+ RXNMON_Acidic
1H NMR (400 MHz, METHANOL-d₄) δ ppm 8.88 (s, 1H) 8.21-8.33 (m, 2H) 7.85 (s, 1H) 7.70 (t, J=7.9 Hz, 1H) 3.95 (quin, J=6.9 Hz, 1H) 3.49-3.56 (m, 1H) 1.77-1.92 (m, 1H) 1.37 (d, J=6.7 Hz, 3H) 1.25 (d, J=6.8 Hz, 3H) 1.03-1.15 (m, 1H) 1.00 (dd, J=6.8, 2.3 Hz, 6H) 0.95 (dd, J=6.7, 4.9 Hz, 1H) 0.54-0.63 (m, 1H) 0.47-0.54 (m, 1H) 0.41 (dt, J=9.7, 4.6 Hz, 1H) 0.26-0.33 (m, 1H).

### Example 12.12:((S)-Isopropyl 2-(2-(3-(5-(((S)-1-Cyclopropylethyl)Carbamoyl)-4H-1,2,4-Triazol-3-yl)Phenyl)Oxazole-5-Carboxamido)-3-Methylbutanoate

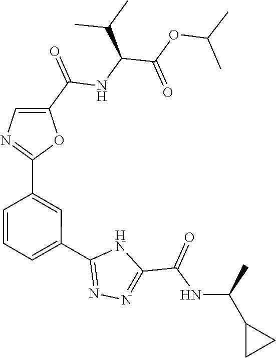 [SMILES: CC(C)OC(=O)[C@@H](NC(=O)c1cnc(-c2cccc(-c3nnc(C(=O)N[C@@H](C)C4CC4)[nH]3)c2)o1)C(C)C]

LCMS Rt: 1.49 min; MS m/z 509.2 [M+H]+ RXNMON_Acidic
1H NMR (400 MHz, METHANOL-d₄) δ ppm 8.89 (s, 1H) 8.28 (br t, J=9.2 Hz, 2H) 7.95 (s, 1H) 7.70 (t, J=7.8 Hz, 1H) 5.07 (quin, J=6.2 Hz, 1H) 4.45 (d, J=7.0 Hz, 1H) 3.50 (dd, J=8.7, 6.8 Hz, 1H) 2.23-2.36 (m, 1H) 1.37 (d, J=6.7 Hz, 3H) 1.29 (dd, J=6.2, 4.0 Hz, 7H) 1.09-1.13 (m, 1H) 1.06 (t, J=7.2 Hz, 7H) 0.54-0.63 (m, 1H) 0.46-0.54 (m, 1H) 0.36-0.46 (m, 1H) 0.31 (dt, J=9.5, 4.6 Hz, 1H).

### Example 12.13:((S)-Methyl 2-(2-(3-(5-(((S)-1-Cyclopropylethyl)Carbamoyl)-4H-1,2,4-Triazol-3-Yl)Phenyl)Oxazole-5-Carboxamido)-4-Methylpentanoate

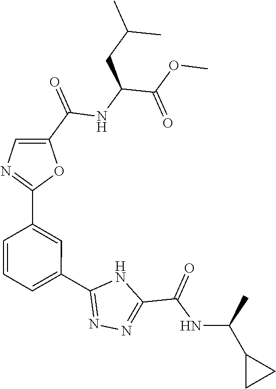 [SMILES: COC(=O)[C@H](CC(C)C)NC(=O)c1cnc(-c2cccc(-c3nnc(C(=O)N[C@@H](C)C4CC4)[nH]3)c2)o1]

LCMS Rt: 1.41 min; MS m/z 495.1 [M+H]+ RXNMON_Acidic

¹H NMR (400 MHz, METHANOL-d₄) δ ppm 8.89 (s, 1H) 8.28 (t, J=8.31 Hz, 2H) 7.91 (s, 1H) 7.70 (t, J=7.83 Hz, 1H) 4.70-4.75 (m, 1H) 3.75 (s, 3H) 3.49-3.55 (m, 1H) 1.81-1.89 (m, 1H) 1.72-1.80 (m, 2H) 1.37 (d, J=6.72 Hz, 3H) 1.05-1.13 (m, 1H) 1.00 (dd, J=11.74, 6.11 Hz, 7H) 0.55-0.63 (m, 1H) 0.47-0.55 (m, 1H) 0.37-0.45 (m, 1H) 0.26-0.34 (m, 1 H)

### Example 12.14:((2S)-Ethyl 2-(2-(3-(3-((1-Cyclopropyl-2,2,2-Trifluoroethyl)Carbamoyl)-1H-1,2,4-Triazol-5-yl)Phenyl)Oxazole-5-Carboxamido)-3-Methylbutanoate

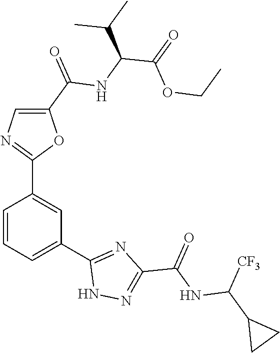 [SMILES: CCOC(=O)[C@@H](NC(=O)c1cnc(-c2cccc(-c3nc(C(=O)NC(C4CC4)C(F)(F)F)n[nH]3)c2)o1)C(C)C]

LCMS Rt: 1.48 min; MS m/z 549.3 [M+H]+ RXNMON_Acidic

¹H NMR (400 MHz, Methanol-d4) δ 8.86 (s, 1H), 8.31-8.23 (m, 2H), 7.94 (s, 1H), 7.69 (t, J=7.8 Hz, 1H), 4.51 (d, J=6.9 Hz, 1H), 4.24 (qd, J=7.1, 3.7 Hz, 2H), 4.09 (dd, J=9.8, 7.5 Hz, 1H), 2.31 (h, J=6.8 Hz, 1H), 1.43-1.34 (m, 1H), 1.30 (t, J=7.1 Hz, 3H), 1.06 (dd, J=8.5, 6.8 Hz, 6H), 0.80 (ddd, J=8.2, 6.1, 3.9 Hz, 1H), 0.64 (dt, J=9.5, 4.6 Hz, 2H), 0.46-0.38 (m, 1H).

### Example 12.15:((S)-N-([1,1′-Bi(Cyclopropan)]-1-Yi)-2-(3-(5-((1-Cyclopropylethyl)Carbamoyl)-4H-1,2,4-Triazol-3-Yl)Phenyl)Oxazole-5-Carboxamide

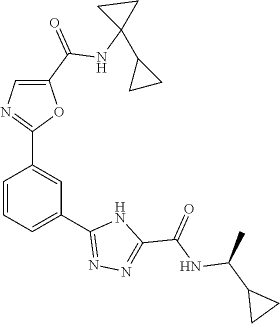 [SMILES: C[C@H](NC(=O)c1nnc(-c2cccc(-c3ncc(C(=O)NC4(C5CC5)CC4)o3)c2)[nH]1)C1CC1]

LCMS Rt: 1.32 min; MS m/z 447.1 [M+H]+ RXNMON_Acidic
1H NMR (400 MHz, METHANOL-d₄) δ ppm 8.85-8.89 (m, 1H) 8.23-8.29 (m, 2H) 7.82 (s, 1H) 7.69 (t, J=7.82 Hz, 1H) 3.48-3.54 (m, 1H) 2.58 (d, J=14.43 Hz, 1H) 2.41 (d, J=14.55 Hz, 1H) 1.44-1.54 (m, 1H) 1.37 (d, J=6.72 Hz, 3H) 1.21-1.32 (m, 1H) 1.04-1.15 (m, 1H) 0.79-0.85 (m, 1H) 0.27-0.74 (m, 10H) 0.14-0.21 (m, 1H)

### Example 12.1816:(S)-N-(Adamantan-1-Yl)-2-(3-(5-((1-Cyclopropylethyl)Carbamoyl)-4H-1,2,4-Triazol-3-Yl)Phenyl)Oxazole-5-Carboxamide

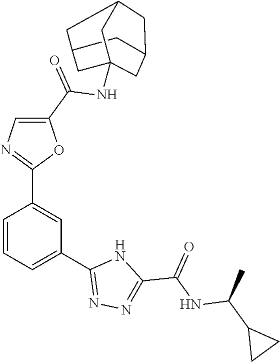 [SMILES: C[C@H](NC(=O)c1nnc(-c2cccc(-c3ncc(C(=O)NC45CC6CC(CC(C6)C4)C5)o3)c2)[nH]1)C1CC1]

LCMS Rt: 1.61 min; MS m/z 501.1 [M+H]+ RXNMON_Acidic
1H NMR (400 MHz, METHANOL-d₄) δ ppm 8.85-8.88 (m, 1H) 8.22-8.29 (m, 2H) 7.81 (s, 1H) 7.64-7.72 (m, 1H) 3.48-3.53 (m, 1H) 2.19-2.23 (m, 6H) 2.10-2.16 (m, 3H) 1.76-1.80 (m, 6H) 1.37 (d, J=6.85 Hz, 3H) 1.03-1.12 (m, 1H) 0.56-0.63 (m, 1H) 0.48-0.55 (m, 1H) 0.38-0.44 (m, 1H) 0.23-0.36 (m, 1H).

### Example 12.17:(S)-Methyl 3-Cyclohexyl-2-(2-(3-(5-(((S)-1-Cyclopropylethyl)Carbamoyl)-4H-1,2,4-Triazol-3-Yl)Phenyl)Oxazole-5-Carboxamido)Propanoate

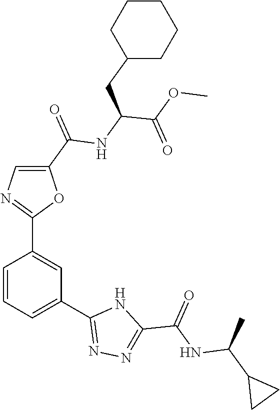 [SMILES: COC(=O)[C@H](CC1CCCCC1)NC(=O)c1cnc(-c2cccc(-c3nnc(C(=O)N[C@@H](C)C4CC4)[nH]3)c2)o1]

LCMS Rt: 1.56 min; MS m/z 535.2 [M+H]+ RXNMON_Acidic
1H NMR (400 MHz, METHANOL-d₄) δ ppm 8.89 (s, 1H) 8.28 (t, J=8.38 Hz, 2H) 7.91 (s, 1H) 7.70 (t, J=7.89 Hz, 1H) 4.72-4.78 (m, 1H) 3.75 (s, 3H) 3.48-3.54 (m, 1H) 1.63-1.87 (m, 8H) 1.36 (d, J=6.72 Hz, 3H) 1.21-1.33 (m, 5H) 1.02-1.11 (m, 2H) 0.57-0.63 (m, 1H) 0.48-0.55 (m, 1H) 0.38-0.46 (m, 1H) 0.26-0.35 (m, 1H)
Example 13 of the present invention may be prepared according to Scheme 19.

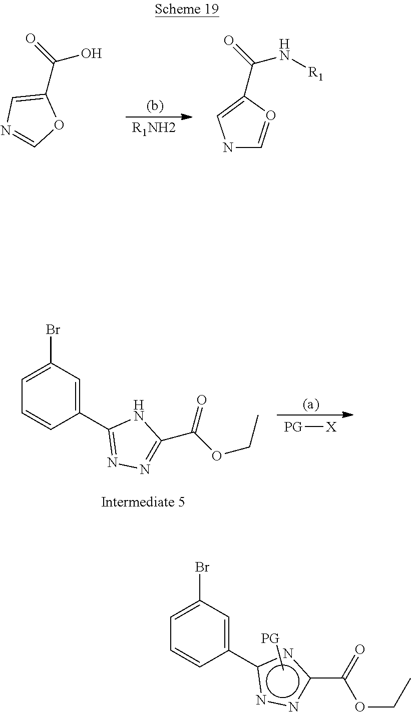

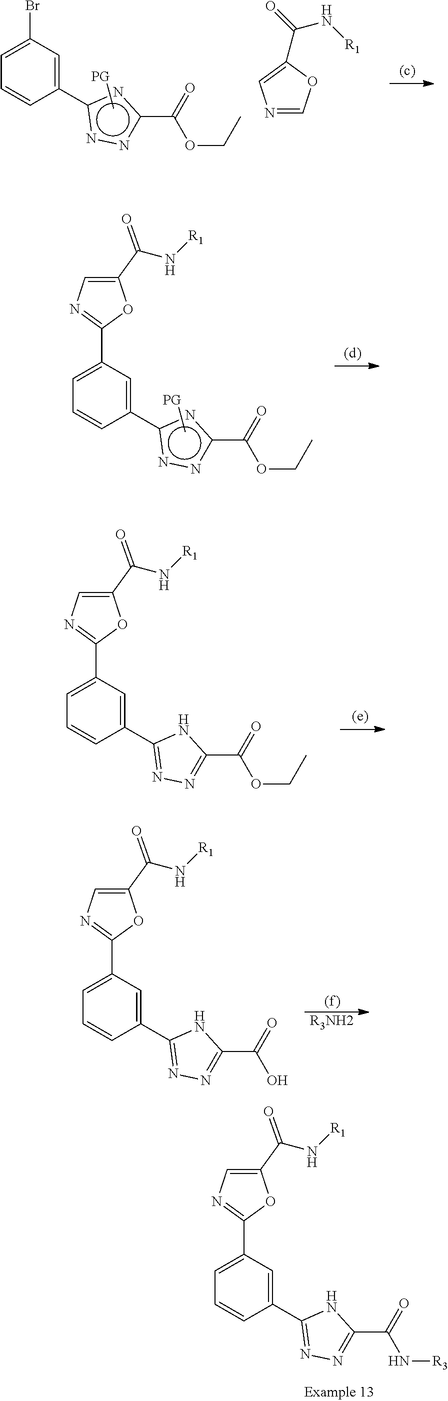

Step (a) involves protection of triazole nitrogen of ethyl 5-(3-bromophenyl)-4H-1,2,4-triazole-3-carboxylate, Intermediate 5, with suitable protective group such as benzyl or SEM-CI by use of appropriate alkyl halide in the presence of a base such as NaH, NEt3, DIPEA, or Cs2CO3.
Step (b) involves reaction of an amine(R₁NH2) with carboxylic acid in a suitable solvent such as DMF or ethyl acetate with a suitable base such as diisopropylethylamine or triethylamine and an amide coupling reagent such as T3P, HATU, TBTU, or pyBOP.
Step (c) involves C-H insertion reaction of oxazole to bromophenyltriazole in a suitable solvent such as DME, DMA, DMF, THE or toluene in the presence of a suitable palladium catalyst such as Pd(OAc)2 or Pd2(dba)3 and ligand such as Xphos, Sphos, cy-JohnPhos, CatacXium A, or RuPhos or by using commercially available pre-formed palladium ligand adduct catalysts such as Xphos-Pd-G1, G2 or G3, RuPhos-Pd -G1,G2, G3 in the presence of pivalic acid and suitable base such as Cs2CO3 with heating under inert atmosphere.
Step (d) involves removal of acid labile protective group by treatment with an acid such as HCl or TFA in a solvent such as DCM or dioxane or alternatively, if the protective group is a benzyl, it may be removed by treatment with hydrogen in the presence of Pd(0) on carbon black in a solvent such as methanol, ethanol or THF.
Step (e) involves conversion of the ester to a carboxylic acid using a suitable base such as NaOH, KOH or KOTMS in a solvent such as THF, methanol or water.
Step (f) involves reaction of an amine(R₃NH2) with free acid in a suitable solvent such as DMF or ethyl acetate with a suitable base such as diisopropylethylamine or triethylamine and an amide coupling reagent such as T3P or pyBOP.
Alternately, the C-H coupling can be run without the use of a protective group on the triazole nitrogen; however the yields may be compromised.
Alternately, the ethyl ester on the triazole may be directly converted to amide using amine R₃NH2 in a suitable solvent such as THF with a base such as 2,3,4,6,7,8-hexahydro-1 H-pyrimido[1,2-a]pyrimidine.
Alternately, the order in which the reactions to remove the protective group and synthesize the triazole amide formation may be switched.

### Example 13.0:N-((S)-1-Cyclopropylethyl)-2-(3-(5-(((R)-1-Cyclopropylethyl)Carbamoyl)-4H-1,2,4-Triazol-3-Yl)Phenyl)Oxazole-5-Carboxamide

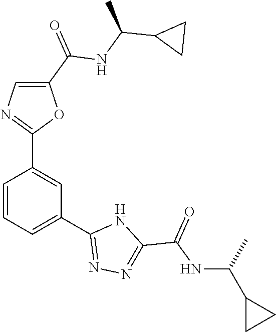 [SMILES: C[C@H](NC(=O)c1cnc(-c2cccc(-c3nnc(C(=O)N[C@H](C)C4CC4)[nH]3)c2)o1)C1CC1]

Step 1: Ethyl 1-benzyl-5-(3-bromophenyl)-1H-1,2,4-triazole-3-carboxylate, Ethyl 1-benzyl-3-(3-bromophenyl)-1 H-1,2,4-triazole-5-carboxylate
To a solution of ethyl 5-(3-bromophenyl)-4H-1,2,4-triazole-3-carboxylate, (Intermediate 5) (4.14 g, 13.98 mmol) in 100 mL THF was added NEt3 (3.90 mL, 28.0 mmol) followed by benzyl bromide (1.995 mL, 16.78 mmol). The RM was stirred 48h at room temp. An additional 1.5 mL of benzyl bromide and 2 mL more of NEt3 were added and the mixture was stirred an additional 24h at room temp and then stirred at 50° C. for 24h. The RM was diluted with water and EtOAc. The organic phase was washed with water, then brine, dried over Na2SO4, and concentrated. The crude material was purified by FCC (0-100% EtOAc in heptane) to provide 4.08 g of a mixture of benzylated regioisomers.

LCMS Rt: 1.34 min; MS m/z 387.9 [M+H]+ RXNMON_Acidic NonPolar

Step 2: (S)-N-(1-cyclopropylethyl)oxazole-5-carboxamide
To a solution of oxazole-5-carboxylic acid (3.65 g, 32.3 mmol) in 50 mL of DMF were added (S)-1-cyclopropylethanamine (3.14 mL, 33.9 mmol), NEt3 (13.50 mL, 97 mmol) and HATU (13.50 g, 35.5 mmol). The RM was stirred for 96h and then diluted with EtOAc. The RM was washed 3×with water, then with brine and then dried over Na2SO4. The crude material waas purified by FCC (0-10% MeOH in DCM) to give 3.03 g, (52%) of (S)-N-(1-cyclopropylethyl)oxazole-5-carboxamide as a brown solid.
LCMS Rt: 0.82 min; MS m/z 181.2 [M+H]+ RXNMON_Acidic

### 1H NMR (400 MHz, DMSO-d₆) δ ppm 8.46-8.56 (m, 2H) 7.75 (s, 1H) 3.33-3.44 (m, 1H) 1.20 (d, J=6.6 Hz, 3H) 0.89-1.07 (m, 1H) 0.42-0.52 (m, 1H) 0.34-0.42 (m, 1H) 0.23-0.30 (m, 1H) 0.15-0.23 (m, 1H)

Step 3: (S)-ethyl 1-benzyl-3-(3-(5-((1-cyclopropylethyl)carbamoyl)oxazol-2-yl)phenyl)-1H-1,2,4-triazole-5-carboxylate, (S)-methyl 1-benzyl-3-(3-(5-((1-cyclopropylethyl)carbamoyl)oxazol-2-yl)phenyl)-1 H-1,2,4-triazole-5-carboxylate
Under nitrogen (S)-N-(1-cyclopropylethyl)oxazole-5-carboxamide (2.094 g, 11.62 mmol), ethyl 1-benzyl-3-(3-bromophenyl)-1 H-1,2,4-triazole-5-carboxylate (4.08 g, 10.56 mmol), pivalic acid (0.490 mL, 4.23 mmol), Cs2CO3 (8.60 g, 26.4 mmol) were combined in 20 mL of Toluene. X-Phos-Pd-G3 (CAS#1445085-55-1) (0.536 g, 0.634 mmol) was added and the RM was heated to 105° C. for 20h. The RM was allowed to cool to room temp and then filtered through celite, washing through with EtOAc and MeOH. The crude mixture was concentrated and then purified by FCC (20-70% EtOAc in heptanes) to give a mixture of ethyl and methyl esters (some transesterification occurred during the filtration with methanol) as well as the benzyl protective group regioisomer mixture which was carried forward without further purification..
LCMS Rt: 1.57 min; MS m/z 472.1 [M+H]+ RXNMON_Acidic_NonPolar (methyl ester)
LCMS Rt: 1.64 min; MS m/z 486.1 [M+H]+ RXNMON_Acidic_NonPolar (ethyl ester)
Step 4: (S)-methyl 3-(3-(5-((1-cyclopropylethyl)carbamoyl)oxazol-2-yl)phenyl)-1 H-1,2,4-triazole-5-carboxylate
A solution of (S)-methyl 1-benzyl-3-(3-(5-((1-cyclopropylethyl)carbamoyl)oxazol-2-yl)phenyl)-1 H-1,2,4-triazole-5-carboxylate (2 g, 4.24 mmol) in 80 mL Methanol with Hydrochloric Acid, 37% (Volume: 0.1 mL) was stirred vigorously with Pd-C(10%, wet) (0.451 g, 0.424 mmol) under a balloon of H2 gas for 96 h. The RM was diluted with EtOAc and solid sodium bicarbonate was added. The mixture was filtered through celite and purified by FCC (0-10% MeOH in DCM) to give 1.34 g (83%) of (S)-methyl 3-(3-(5-((1-cyclopropylethyl)carbamoyl)oxazol-2-yl)phenyl)-1 H-1,2,4-triazole-5-carboxylate.
LCMS Rt: 1.16 min; MS m/z 382.0 [M+H]+ RXNMON_Acidic
1H NMR (400 MHz, METHANOL-d₄) δ ppm 8.87 (s, 1H) 8.31 (d, J=7.9 Hz, 1H) 8.24 (br d, J=7.9 Hz, 1H) 7.84 (s, 1H) 7.71 (t, J=7.9 Hz, 1H) 4.02 (s, 3H) 3.47-3.54 (m, 1H) 1.36 (d, J=6.7 Hz, 3H) 1.07 (br d, J=8.8 Hz, 1H) 0.56-0.64 (m, 1H) 0.47-0.55 (m, 1H) 0.39 (dt, J=9.8, 4.6 Hz, 1H) 0.25-0.34 (m, 1H).
Step 5: (S)-3-(3-(5-((1-cyclopropylethyl)carbamoyl)oxazol-2-yl)phenyl)-1 H-1,2,4-triazole-5-carboxylic acid
To a solution of (S)-methyl 3-(3-(5-((1-cyclopropylethyl)carbamoyl)oxazol-2-yl)phenyl)-1 H-1,2,4-triazole-5-carboxylate (1.34 g, 3.51 mmol) in 30 mL of THE (Volume: 30 mL) at room temp was added KOTMS (0.541 g, 4.22 mmol). The RM was stirred for 20 h. The RM was concentrated by rotary evaporator and then dried under vacuum to constant mass to give a quantitative yield of potassium (S)-3-(3-(5-((1-cyclopropylethyl)carbamoyl)oxazol-2-yl)phenyl)-1 H-1,2,4-triazole-5-carboxylate as a free flowing yellow solid which was used crude for the subsequent reaction.
LCMS Rt: 1.06 min; MS m/z 368.0 [M+H]+ RXNMON_Acidic
Step 6: N-((S)-1-cyclopropylethyl)-2-(3-(5-(((R)-1-cyclopropylethyl)carbamoyl)-4H-1,2,4-triazol-3-yl)phenyl)oxazole-5-carboxamide
To a suspension of potassium (S)-3-(3-(5-((1-cyclopropylethyl)carbamoyl)oxazol-2-yl)phenyl)-1 H-1,2,4-triazole-5-carboxylate (120 mg, 0.242 mmol) in 2 mL EtOAc was added (R)-1-cyclopropylethanamine (41.2 mg, 0.484 mmol), NEt3 (0.135 mL, 0.968 mmol), and T3P (50% in EtOAc) (0.214 mL, 0.363 mmol). The RM was stirred at room temperature for 8 days. The RM was diluted with water, 10% citric acid and DCM. The organic phase was concentrated and then purified by FCC (0-10% MeOH in DCM) to give 79 mg (72%) of N-((S)-1-cyclopropylethyl)-2-(3-(5-(((R)-1-cyclopropylethyl)carbamoyl)-4H-1,2,4-triazol-3-yl)phenyl)oxazole-5-carboxamide.
LCMS Rt: 1.34 min; MS m/z 435.2 [M+H]+ RXNMON_Acidic 1H NMR (400 MHz, METHANOL-d₄) δ ppm 8.89 (s, 1H) 8.28 (br dd, J=13.6, 7.7 Hz, 2H) 7.84 (s, 1H) 7.70 (t, J=7.9 Hz, 1H) 3.48-3.54 (m, 2H) 1.33-1.41 (m, 6H) 0.98-1.13 (m, 3H) 0.55-0.64 (m, 2H) 0.46-0.55 (m, 2H) 0.36-0.45 (m, 3H) 0.30 (tt, J=9.3, 4.6 Hz, 2H) 0.14-0.24 (m, 1H).
Examples 13.1 to 13.7: were prepared by a method similar to that of Example 13.0 by replacing with appropriate commercially available amines.

### Example 13.1:(R)-2-(3-(5-((1-Cyclopropylethyl)Carbamoyl)-4H-1,2,4-Triazol-3-Yl)Phenyl)-N-(Dicyclopropylmethyl)Oxazole-5-Carboxamide

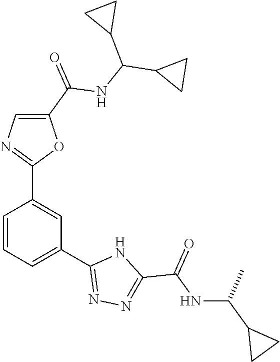 [SMILES: C[C@@H](NC(=O)c1nnc(-c2cccc(-c3ncc(C(=O)NC(C4CC4)C4CC4)o3)c2)[nH]1)C1CC1]

LCMS Rt: 1.41 min; MS m/z 461.1 [M+H]+ RXNMON_Acidic

¹H NMR (400 MHz, METHANOL-d₄) δ ppm 8.85-8.94 (m, 1H) 8.28-8.34 (m, 1H) 8.21-8.28 (m, 1H) 7.85 (s, 1H) 7.66-7.75 (m, 1H) 3.48-3.57 (m, 1H) 3.01 (t, J=8.6 Hz, 1H) 1.37 (d, J=6.6 Hz, 3H) 1.12-1.23 (m, 3H) 1.03-1.12 (m, 1H) 0.56-0.66 (m, 3H) 0.34-0.56 (m, 9H) 0.24-0.33 (m, 1H).

### Example 13.2:N-((R)-1-Cyclopropylethyl)-2-(3-(3-(((R)-1-Cyclopropylethyl)Carbamoyl)-1H-1,2,4-Triazol-5-Yl)Phenyl)Oxazole-5-Carboxamide

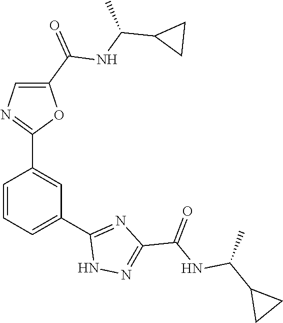 [SMILES: C[C@@H](NC(=O)c1n[nH]c(-c2cccc(-c3ncc(C(=O)N[C@H](C)C4CC4)o3)c2)n1)C1CC1]

LCMS Rt: 0.96 min; MS m/z 435.4 [M+H]+ RXNMON_Acidic
1H NMR (400 MHz, DMSO-d₆) δ ppm 15.13 (br s, 1H) 8.83-8.97 (br. s, 1H) 8.81 (t, J=1.53 Hz, 1H) 8.66 (d, J=8.31 Hz, 1H) 8.21-8.28 (m, 2H) 7.94 (s, 1H) 7.74 (br t, J=7.70 Hz, 1H) 3.35-3.50 (m, 2H) 1.22-1.31 (m, 6H) 1.09-1.17 (m, 1H) 0.98-1.05 (m, 1H) 0.46-0.53 (m, 2H) 0.37-0.44 (m, 2H) 0.28-0.34 (m, 2H) 0.20-0.27 (m, 2H).

### Example 13.3:N-(Pentan-3-Yl)-2-(3-(5-(Pentan-3-Ylcarbamoyl)-1 H-1,2,4-Triazol-3-Yl)Phenyl)Oxazole-5-Carboxamide

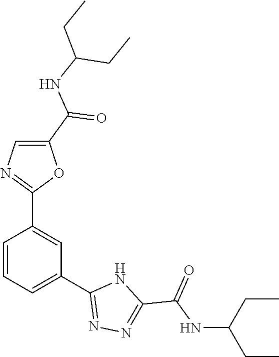 [SMILES: CCC(CC)NC(=O)c1nnc(-c2cccc(-c3ncc(C(=O)NC(CC)CC)o3)c2)[nH]1]

LCMS Rt: 1.35 mins MS m/z 439.4 [M+H]+ 2minLowpv03

¹H NMR (400 MHz, DMSO-d₆) δ ppm 15.11 (br s, 1H) 8.81 (s, 1H) 8.34 (d, J=8.80 Hz, 1H) 8.20-8.29 (m, 2H) 7.95 (s, 1H) 7.74 (br t, J=7.82 Hz, 1H) 3.79 (dt, J=8.68, 4.22 Hz, 2H) 1.52-1.65 (m, 6H) 1.50 (br d, J=8.31 Hz, 2H) 0.88 (q, J=3.42 Hz, 12H)

### Example 13.4:(S)-N-(1-Cyclopropylethyl)-2-(3-(5-((4,4-Difluorocyclohexyl)Carbamoyl)-4H-1,2,4-Triazol-3-Yl)Phenyl)Oxazole-5-Carboxamide

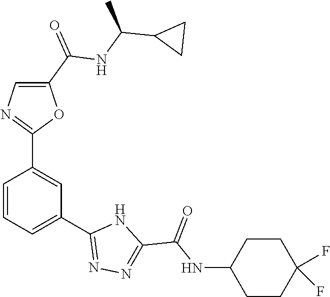 [SMILES: C[C@H](NC(=O)c1cnc(-c2cccc(-c3nnc(C(=O)NC4CCC(F)(F)CC4)[nH]3)c2)o1)C1CC1]

LCMS Rt: 1.33 min; MS m/z 485.3 [M+H]+ RXNMON_Acidic
1H NMR (400 MHz, METHANOL-d₄) δ ppm 8.88 (s, 1H) 8.29 (br d, J=7.7 Hz, 1H) 8.25 (br d, J=7.5 Hz, 1H) 7.84 (d, J=0.6 Hz, 1H) 7.70 (t, J=7.8 Hz, 1H) 5.20 (br d, J=16.1 Hz, 1H) 4.16-4.25 (m, 1H) 4.08 (br t, J=10.5 Hz, 1H) 3.49-3.54 (m, 1H) 2.39-2.50 (m, 1H) 2.34 (br d, J=4.2 Hz, 1H) 2.19-2.32 (m, 1H) 2.09-2.19 (m, 1H) 2.01-2.09 (m, 2H) 1.89-1.99 (m, 2H) 1.77-1.87 (m, 1H) 1.36 (d, J=6.7 Hz, 3H) 1.00-1.12 (m, 1H) 0.55-0.64 (m, 1H) 0.46-0.55 (m, 1H) 0.36-0.44 (m, 1H) 0.24-0.34 (m, 1H).

### Example 13.5:2-(3-(5-((Cyclohexylmethyl)Carbamoyl)-4H-1,2,4-Triazol-3-Yl)Phenyl)-N-(Pentan-3-Yl)Oxazole-5-Carboxamide

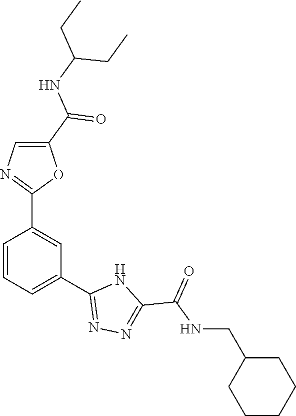 [SMILES: CCC(CC)NC(=O)c1cnc(-c2cccc(-c3nnc(C(=O)NCC4CCCCC4)[nH]3)c2)o1]

LCMS Rt: 1.44 min; MS m/z 465.4 [M+H]+ 2minLowpHv01

### Example 13.6:(S)-Ethyl 2-(5-(3-(5-(((S)-1-Cyclopropylethyl)Carbamoyl)Oxazol-2-Yl)Phenyl)-4H-1,2,4-Triazole-3-Carboxamido)-3-Methylbutanoate

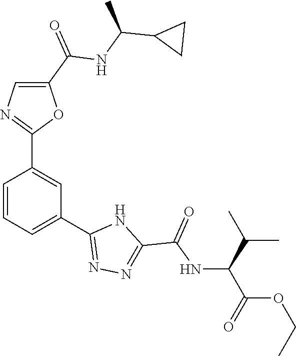 [SMILES: CCOC(=O)[C@@H](NC(=O)c1nnc(-c2cccc(-c3ncc(C(=O)N[C@@H](C)C4CC4)o3)c2)[nH]1)C(C)C]

LCMS Rt: 1.34 mins MS m/z 495.4 [M+H]+ 2minLowpv03

¹H NMR (400 MHz, CHLOROFORM-d) δ ppm 8.87 (s, 1H) 8.33 (br d, J=7.82 Hz, 1H) 8.21 (br d, J=7.82 Hz, 1H) 7.89 (s, 1H) 7.85 (br d, J=9.05 Hz, 1H) 7.64 (t, J=7.83 Hz, 1H) 6.56 (br d, J=7.34 Hz, 1H) 4.81 (dd, J=9.05, 4.89 Hz, 1H) 4.30 (br dd, J=7.09, 3.18 Hz, 2H) 3.56-3.69 (m, 1H) 2.32-2.47 (m, 1H) 1.39 (d, J=6.60 Hz, 3H) 1.35 (t, J=7.09 Hz, 3H) 1.08 (t, J=6.11 Hz, 6H) 0.99-1.04 (m, 1H) 0.53-0.65 (m, 2H) 0.48 (dq, J=9.54, 4.81 Hz, 1H) 0.35 (dq, J=9.23, 4.50 Hz, 1H)

### Example 13.7:(S)-Methyl 2-(3-(3-(5-((Dicyclopropylmethyl)Carbamoyl)Oxazol-2-Yl)Phenyl)-1H-1,2,4-Triazole-5-Carboxamido)-3-Methylbutanoate

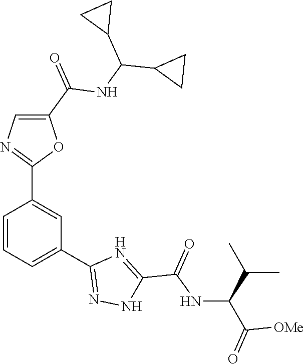

LCMS Rt: 1.43 min; MS m/z 507.2 [M+H]+ RXNMON_Acidic
1H NMR (400 MHz, DMSO-d₆) δ ppm 8.79 (s, 1H) 8.75 (d, J=8.7 Hz, 1H) 8.24 (d, J=7.8 Hz, 1H) 8.15 (br s, 1H) 7.92 (s, 1H) 7.66 (br s, 1H) 4.40 (dd, J=8.4, 6.7 Hz, 1H) 3.69 (s, 3H) 2.92 (q, J=8.6 Hz, 1H) 2.24 (br d, J=6.5 Hz, 1H) 1.07-1.18 (m, 3H) 0.96 (t, J=6.2 Hz, 6H) 0.79-0.88 (m, 1H) 0.48-0.58 (m, 2H) 0.32-0.44 (m, 4H) 0.22-0.30 (m, 2H).
Example 14 of the present invention may be prepared according to Scheme 20.

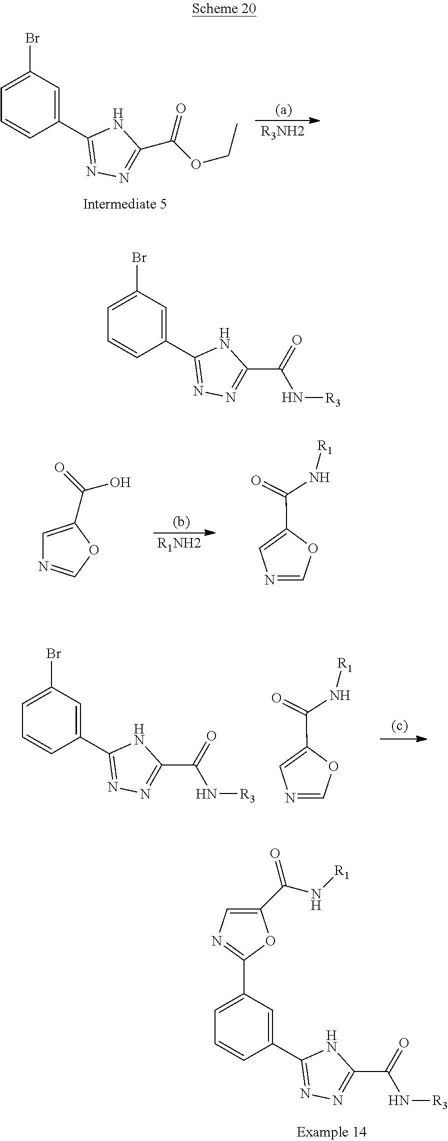

Step (a) involves reaction of an amine(R₃NH2) with ethyl 5-(3-bromophenyl)-4H-1,2,4-triazole-3-carboxylate, Intermediate 5, in a suitable solvent such as THE with a suitable base such as 2,3,4,6,7,8-hexahydro-1 H-pyrimido[1,2-a]pyrimidine to give an amide.
Step (b) involves reaction of an amine(R₁NH2) with free acid in a suitable solvent such as DMF or ethyl acetate with a suitable base such as diisopropylethylamine or triethylamine and an amide coupling reagent such as HATU, TBTU, T3P or pyBOP.
Step (c) involves C-H insertion reaction of oxazole to bromophenyltriazole in a suitable solvent such as DME, DMA, DMF, THE or toluene in the presence of a suitable palladium catalyst such as Pd(OAc)2 or Pd2(dba)3 and ligand such as Xphos, Sphos, cy-JohnPhos, CatacXium A, or RuPhos or by using commercially available pre-formed palladium ligand adduct catalysts such as Xphos-Pd-G1, G2 or G3, RuPhos-Pd -G1,G2, G3 in the presence of pivalic acid and/or Cul and suitable base such as Cs2CO3 or K2CO3 with heating under inert atmosphere.

### Example 14.0: (S)-2-(3-(5-((1-Cyclopropylethyl)Carbamoyl)-4H-1,2,4-Triazol-3-Yl)Phenyl)-N-(Pentan-3-Yl)Oxazole-5-Carboxamide

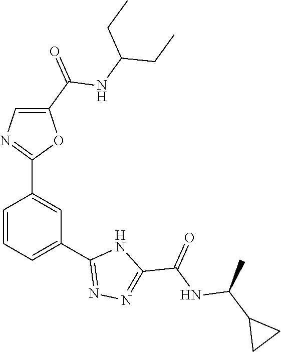 [SMILES: CCC(CC)NC(=O)c1cnc(-c2cccc(-c3nnc(C(=O)N[C@@H](C)C4CC4)[nH]3)c2)o1]

Step 1: (S)-5-(3-bromophenyl)-N-(1-cyclopropylethyl)-4H-1,2,4-triazole-3-carboxamide
In a 20 mL microwave vial was placed Ethyl 5-(3-bromophenyl)-4H-1,2,4-triazole-3-carboxylate, (Intermediate 5) (3.54 g, 11.95 mmol) with (S)-1-cyclopropylethanamine (3.5 mL, 32.8 mmol) in THE (12 mL) with 1,3,4,6,7,8-hexahydro-2H-pyrimido[1,2-a]pyrimidine (0.333 g, 2.391 mmol). The RM was heated by microwave at 140° C. for 1 h. The RM was concentrated and purified by FCC (0-10% MeOH/DCM) to afford 3.7 g (92%) of (S)-5-(3-bromophenyl)-N-(1-cyclopropylethyl)-4H-1,2,4-triazole-3-carboxamide as a white foam.
LCMS Rt: 1.39 min; MS m/z 336.9 [M+H]+ RXNMON_Acidic

¹H NMR (400 MHz, METHANOL-d₄) Q ppm 8.27 (t, J=1.71 Hz, 1H) 8.04 (d, J=7.82 Hz, 1H) 7.64 (d, J=7.82 Hz, 1H) 7.43 (t, J=7.89 Hz, 1H) 3.45-3.50 (m, 1H) 1.35 (d, J=6.72 Hz, 3H) 1.29 (br. s., 2H) 1.02-1.12 (m, 1H) 0.54-0.61 (m, 1H) 0.46-0.53 (m, 1H) 0.36-0.43 (m, 1H) 0.25-0.33 (m, 1H)

Step 2: N-(pentan-3-yl)oxazole-5-carboxamide
A solution of oxazole-5-carboxylic acid (3 g, 26.5 mmol) in dry DMF (30 ml), was treated with triethylamine (8.88 mL, 63.7 mmol), HATU (12.11 g, 31.8 mmol) and then pentan-3-amine (6.18 mL, 53.1 mmol). The reaction was diluted with water and EtOAc and the aqueous was extracted twice with 4:1 EtOAc:heptane. The organics were combined, washed with water (3×) and brine (1×) and then dried over Na2SO4. The crude material was purified by FCC (0-100% EtOAc in heptane) to give 0.8 g of N-(pentan-3-yl)oxazole-5-carboxamide as a yellow crystalline solid.

¹H NMR (400 MHz, CHLOROFORM-d) d=7.91 (s, 1H), 7.73 (s, 1H), 5.99-5.90 (m, 1H), 4.05-3.94 (m, 1H), 1.75-1.62 (m, 2H), 1.54-1.44 (m, 2H), 0.97 (t, J=7.5 Hz, 6H).

Step 3: (S)-2-(3-(5-((1-cyclopropylethyl)carbamoyl)-4H-1,2,4-triazol-3-yl)phenyl)-N-(pentan-3-yl)oxazole-5-carboxamide (S)-5-(3-bromophenyl)-N-(1-cyclopropylethyl)-4H-1,2,4-triazole-3-carboxamide (60 mg, 0.179 mmol), N-(pentan-3-yl)oxazole-5-carboxamide (45.7 mg, 0.251 mmol), Cul (40.9 mg, 0.215 mmol), and K2CO3 (49.5 mg, 0.358 mmol) were suspended in 1 mL DMF. Pd acetate (8.04 mg, 0.036 mmol) was added and the reaction was heated by microwave for 30 min at 150° C. EtOAc and sat'd NH4CI were added and the organic phase was washed with water, then brine and dried over Na2SO4. The crude material was purified by prep HPLC Method 2 (Low pH) to give 4 mg of (S)-2-(3-(5-((1-cyclopropylethyl)carbamoyl)-4H-1,2,4-triazol-3-yl)phenyl)-N-(pentan-3-yl)oxazole-5-carboxamide.
LCMS Rt: 1.39 min; MS m/z 437.4 [M+H]+ RXNMON_Acidic
1H NMR (400 MHz, METHANOL-d₄) δ ppm 8.88 (br. s., 1H) 8.22-8.31 (m, 2H) 7.85 (s, 1H) 7.65-7.73 (m, 1H) 3.85-3.96 (m, 1H) 1.64-1.74 (m, 2H) 1.53-1.63 (m, 2H) 1.36 (d, J=6.72 Hz, 3H) 1.29 (s, 4H) 0.94-1.00 (m, 6H) 0.82-0.91 (m, 1H) 0.55-0.62 (m, 1H) 0.47-0.54 (m, 1H) 0.38-0.45 (m, 1H) 0.27-0.33 (m, 1H) Example 15 of the present invention may be prepared according to Scheme 21.

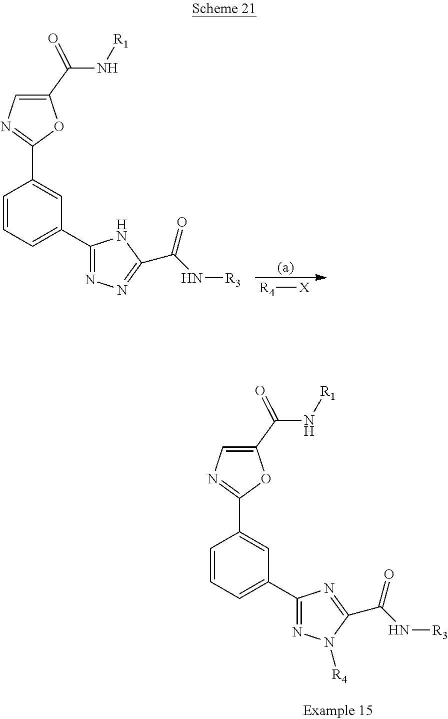

Step (a) involves alkylation of triazole nitrogen with appropriate alkyl halide in the presence of a base such as LiHMDS, or NaH followed by separation of the desired isomer by chromatography.

### Example 15.0: N-(Pentan-3-Yl)-2-(3-(5-(Pentan-3-Ylcarbamoyl)-1-(2-(Piperidin-1-Yl)Ethyl)-1H-1,2,4-Triazol-3-Yl)Phenyl)Oxazole-5-Carboxamide

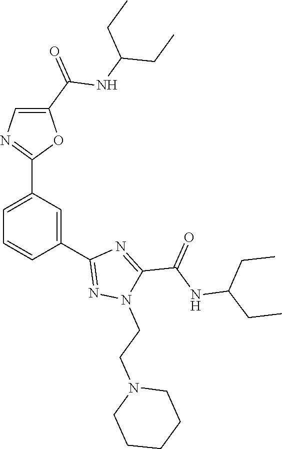 [SMILES: CCC(CC)NC(=O)c1cnc(-c2cccc(-c3nc(C(=O)NC(CC)CC)n(CCN4CCCCC4)n3)c2)o1]

A solution of N-(pentan-3-yl)-2-(3-(5-(pentan-3-ylcarbamoyl)-1 H-1,2,4-triazol-3-yl)phenyl)oxazole-5-carboxamide (Example 13.3) (400 mg, 0.912 mmol) in DMF (3 mL) was cooled to O ° C. under nitrogen. 1 M LiHMDS (2.189 mL, 2.189 mmol) was added dropwise followed by a solution of 1-(2-bromoethyl)piperidine (299 mg, 1.095 mmol) in DMF (1 mL).
The RM was allowed to warm to RT then was stirred at RT for 18 h. The RM was poured into ice water (50 mL) and the resulting ppt collected by filtration. The tan solid was dissolved in DCM (50 mL), washed with brine (20 mL) and passed through a phase separation cartridge and then concentrated under reduced pressure to give 470 mg of a yellow foam. The crude material was purified by FCC (0-100% EtOAc/iso-hexane) to afford N-(pentan-3-yl)-2-(3-(5-(pentan-3-ylcarbamoyl)-1-(2-(piperidin-1-yl)ethyl)-1 H-1,2,4-triazol-3-yl)phenyl)oxazole-5-carboxamide (270 mg, 51.2% yield) as a white foam.
LCMS Rt: 3.58 mins; MS m/z 550.5 [M+H]+; 8minLowpHv01

### 1H NMR (AVW14528): NMR1 (400 MHz, DMSO-d₆) δ 8.77 (t, 1H), 8.61 (d, 1H), 8.32 (d, 1H), 8.24 (tt, 2H), 7.95 (s, 1H), 7.73 (t, 1H), 4.78 (t, 2H), 3.84-3.73 (m, 2H), 2.74 (t, 2H), 2.41-2.31 (m, 4H), 1.66-1.28 (m, 14H), 0.90 (t, 6H), 0.88 (t, 6H).

Examples 15.1 to Triazole tether 15.1211 were prepared by a similar method to that of Example 15.0 by replacement of 1-(2-bromoethyl)piperidine with the appropriate bromide and replacement of N-(pentan-3-yl)-2-(3-(5-(pentan-3-ylcarbamoyl)-1 H-1,2,4-triazol-3-yl)phenyl)oxazole-5-carboxamide with the appropriate triazole.

### Example 15.1: 2-(3-(1-(2-Methoxyethyl)-5-(Pentan-3-Ylcarbamoyl)-1H-1,2,4-Triazol-3-Yl)Phenyl)-N-(Pentan-3-Yl)Oxazole-5-Carboxamide

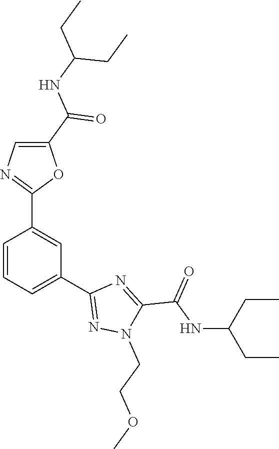 [SMILES: CCC(CC)NC(=O)c1cnc(-c2cccc(-c3nc(C(=O)NC(CC)CC)n(CCOC)n3)c2)o1]

LCMS Rt: 1.53 mins MS m/z 497.5 [M+H]+ 2minLowpHv03

¹H NMR (400 MHz, CHLOROFORM-d) δ ppm 8.82-8.86 (m, 1H) 8.27-8.31 (m, 1H) 8.16 (dd, J=7.83, 1.26 Hz, 1H) 7.84 (s, 1H) 7.60 (t, J=7.83 Hz, 1H) 7.24-7.29 (m, 1H) 6.11 (br d, J=9.35 Hz, 1H) 4.98 (t, J=5.56 Hz, 2H) 4.01-4.11 (m, 1H) 3.92-4.00 (m, 1H) 3.90 (t, J=5.56 Hz, 2H) 3.37 (s, 3H) 1.66-1.80 (m, 4H) 1.58 (dt, J=14.34, 7.36 Hz, 4H) 1.01 (td, J=7.33, 3.03 Hz, 12H)

### Example 15.2: Ethyl 2-(5-(Pentan-3-Ylcarbamoyl)-3-(3-(5-(Pentan-3-Ylcarbamoyl)Oxazol-2-Yl)Phenyl)-1 H-1,2,4-Triazol-1-Yl)Acetate

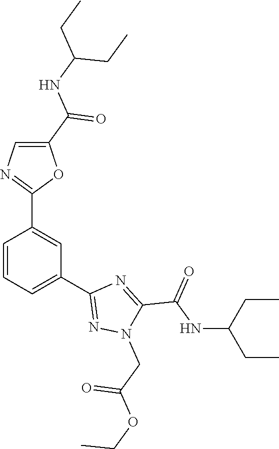 [SMILES: CCOC(=O)Cn1nc(-c2cccc(-c3ncc(C(=O)NC(CC)CC)o3)c2)nc1C(=O)NC(CC)CC]

LCMS Rt:1.57 mins MS m/z 525.5 [M+H]+ 2minLowpHv03

¹H NMR (400 MHz, CHLOROFORM-d) δ ppm 8.82 (s, 1H) 8.27 (br d, J=7.82 Hz, 1H) 8.16 (br d, J=7.82 Hz, 1H) 7.84 (s, 1H) 7.60 (t, J=7.83 Hz, 1H) 7.20 (br d, J=9.54 Hz, 1H) 6.09 (br d, J=9.05 Hz, 1H) 5.52 (s, 2H) 4.28 (q, J=7.09 Hz, 2H) 3.99-4.13 (m, 1H) 3.87-3.98 (m, 1H) 1.66-1.78 (m, 4H) 1.57 (dt, J=14.31, 7.27 Hz, 4H) 1.31 (t, J=7.09 Hz, 3H) 1.00 (q, J=7.34 Hz, 12H)

### Example 15.3: 2-(3-(4-(2-(2-Methoxyethoxy)Ethyl)-5-(Pentan-3-Ylcarbamoyl)-1H-1,2,4-Triazol-3-Yl)Phenyl)-N-(Pentan-3-Yl)Oxazole-5-Carboxamide

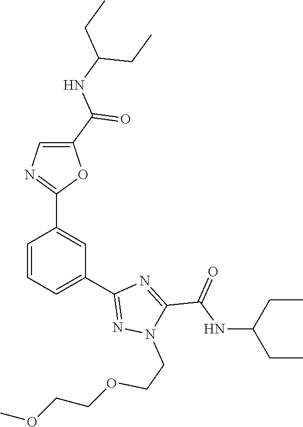 [SMILES: CCC(CC)NC(=O)c1cnc(-c2cccc(-c3nc(C(=O)NC(CC)CC)n(CCOCCOC)n3)c2)o1]

LCMS Rt: 1.70 MS m/z 581.8 [M+H]+ 2minLowpHv03

¹H NMR (400 MHz, CHLOROFORM-d) δ ppm 8.83 (s, 1H) 8.28 (br d, J=7.82 Hz, 1H) 8.16 (br d, J=7.58 Hz, 1H) 7.84 (s, 1H) 7.60 (t, J=7.82 Hz, 1H) 7.25-7.30 (m, 1H) 6.10 (br d, J=9.29 Hz, 1H) 4.96-5.02 (m, 2H) 4.00-4.11 (m, 3H) 3.89-3.99 (m, 1H) 3.64-3.69 (m, 2H) 3.51 (t, J=4.52 Hz, 2H) 3.33 (s, 3H) 1.67-1.80 (m, 4H) 1.58 (dt, J=14.31, 7.27 Hz, 4H) 1.01 (td, J=7.21, 3.67 Hz, 12H)

### Example 15.4: 2-(3-(1-(2-(Dimethylamino)Ethyl)-5-(Pentan-3-Ylcarbamoyl)-1H-1,2,4-Triazol-3-Yl)Phenyl)-N-(Pentan-3-Yl)Oxazole-5-Carboxamide

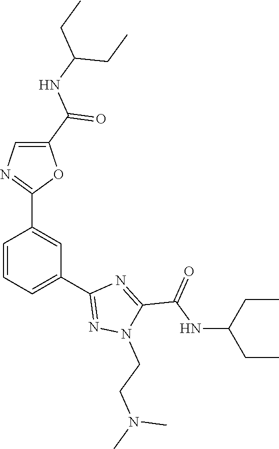 [SMILES: CCC(CC)NC(=O)c1cnc(-c2cccc(-c3nc(C(=O)NC(CC)CC)n(CCN(C)C)n3)c2)o1]

LCMS Rt: 1.16 mins MS m/z 510.6 [M+H]+ 2minLowpHv03

### Example 15.5: 2-(3-(4-(2-Methoxyethyl)-5-(Pentan-3-Ylcarbamoyl)-4H-1,2,4-Triazol-3-Yl)Phenyl)-N-(Pentan-3-Yl)Oxazole-5-Carboxamide

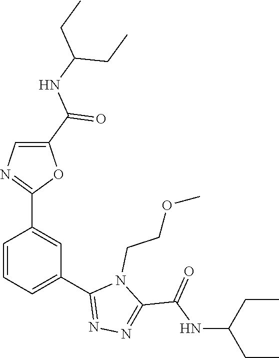 [SMILES: CCC(CC)NC(=O)c1cnc(-c2cccc(-c3nnc(C(=O)NC(CC)CC)n3CCOC)c2)o1]

LCMS Rt: 1.34 mins MS m/z 497.5 [M+H]+ 2minLowpHv03

¹H NMR (400 MHz, CHLOROFORM-d) δ ppm 8.59 (s, 1H) 8.30 (br d, J=8.31 Hz, 1H) 8.00 (br d, J=7.83 Hz, 1H) 7.84 (s, 1H) 7.68 (t, J=7.82 Hz, 1H) 7.00 (br d, J=9.54 Hz, 1H) 6.16 (br d, J=8.80 Hz, 1H) 4.43 (br t, J=4.65 Hz, 2H) 4.05 (br dd, J=13.45, 7.83 Hz, 2H) 3.97 (br t, J=4.77 Hz, 2H) 3.35 (s, 3H) 1.63-1.81 (m, 4H) 1.47-1.62 (m, 4H) 0.99 (br t, J=7.09 Hz, 12H)

### Example 15.6: 2-(3-(1-(2-(Benzylamino)Ethyl)-5-(Pentan-3-Ylcarbamoyl)-1H-1,2,4-Triazol-3-Yl)Phenyl)-N-(Pentan-3-Yl)Oxazole-5-Carboxamide

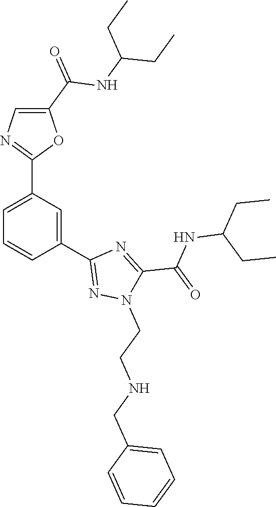 [SMILES: CCC(CC)NC(=O)c1cnc(-c2cccc(-c3nc(C(=O)NC(CC)CC)n(CCNCc4ccccc4)n3)c2)o1]

LCMS Rt: 1.15 mins, MS 572.5 [M+H]+ 2minLowpHv03

¹H NMR (400 MHz, METHANOL-d₄) δ ppm 8.92 (t, J=1.52 Hz, 1H) 8.24-8.33 (m, 2H) 7.87 (s, 1H) 7.67 (t, J=7.83 Hz, 1H) 7.25-7.34 (m, 4H) 7.22 (br d, J=6.82 Hz, 1H) 4.88-4.92 (m, 2H) 3.84-4.00 (m, 2H) 3.81 (s, 2H) 3.16 (t, J=6.06 Hz, 2H) 1.65-1.78 (m, 4H) 1.52-1.65 (m, 4H) 0.99 (td, J=7.45, 1.77 Hz, 12H)

### Example 15.7: 2-(3-(1-(2-(Isoindolin-2-Yl)Ethyl)-5-(Pentan-3-Ylcarbamoyl)-1H-1,2,4-Triazol-3-Yl)Phenyl)-N-(Pentan-3-Yl)Oxazole-5-Carboxamide

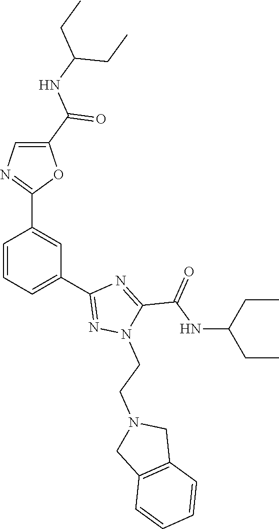 [SMILES: CCC(CC)NC(=O)c1cnc(-c2cccc(-c3nc(C(=O)NC(CC)CC)n(CCN4Cc5ccccc5C4)n3)c2)o1]

LCMS Rt: 1.13 mins MS m/z 584.7 [M+H]+ 2minLowpHv03

¹H NMR (400 MHz, CHLOROFORM-d) δ ppm 8.83 (s, 1H) 8.28 (br d, J=7.58 Hz, 1H) 8.16 (br d, J=7.83 Hz, 1H) 8.10 (br s, 1H) 7.85 (s, 1H) 7.61 (br t, J=7.70 Hz, 1H) 7.24-7.31 (m, 1H) 6.20 (br d, J=9.05 Hz, 1H) 5.71 (br s, 1H) 5.05 (br t, J=6.24 Hz, 2H) 4.19 (s, 4H) 4.00-4.10 (m, 1H) 3.91-3.99 (m, 1H) 3.43 (br t, J=6.24 Hz, 2H) 1.71 (dt, J=13.51, 6.82 Hz, 4H) 1.57 (dt, J=13.69, 6.85 Hz, 4H) 0.99 (dt, J=14.86, 7.37 Hz, 12H)

### Example 15.8: Ethyl 4-(5-(Pentan-3-Ylcarbamoyl)-3-(3-(5-(Pentan-3-Ylcarbamoyl)Oxazol-2-Yl)Phenyl)-1H-1,2,4-Triazol-1-Yl)Butanoate

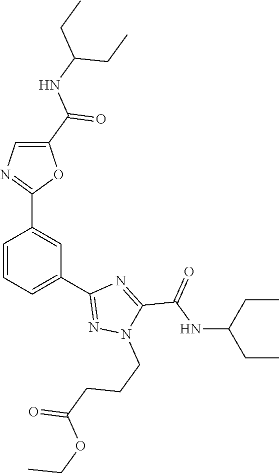 [SMILES: CCOC(=O)CCCn1nc(-c2cccc(-c3ncc(C(=O)NC(CC)CC)o3)c2)nc1C(=O)NC(CC)CC]

LCMS Rt: 1.662 mins MS m/z 553.7 [M+H]+ 2minLowpHv03

¹H NMR (400 MHz, CHLOROFORM-d) δ ppm 8.83 (s, 1H) 8.27 (br d, J=8.07 Hz, 1H) 8.16 (br d, J=7.83 Hz, 1H) 7.84 (s, 1H) 7.61 (t, J=7.70 Hz, 1H) 7.24 (br d, J=9.29 Hz, 1H) 6.08 (br d, J=9.05 Hz, 1H) 4.84 (t, J=6.72 Hz, 2H) 4.13 (q, J=7.17 Hz, 2H) 4.06 (br d, J=8.31 Hz, 1H) 3.88-3.98 (m, 1H) 2.41-2.49 (m, 2H) 2.26-2.37 (m, 2H) 1.67-1.79 (m, 4H) 1.52-1.65 (m, 4H) 1.25 (t, J=7.21 Hz, 3H) 1.01 (td, J=7.34, 2.69 Hz, 12H)

### Example 15.9: 2-(3-(1-(2-(2-(2-Methoxyethoxy)Ethoxy)Ethyl)-5-(Pentan-3-Ylcarbamoyl)-1H-1,2,4-Triazol-3-Yl)Phenyl)-N-(Pentan-3-Yl)Oxazole-5-Carboxamide

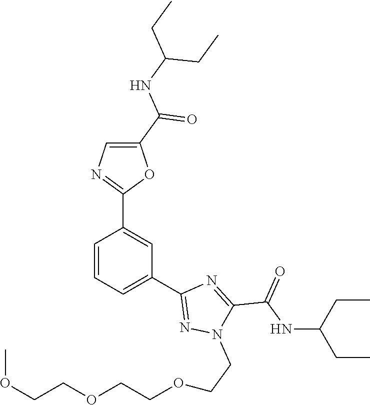 [SMILES: CCC(CC)NC(=O)c1cnc(-c2cccc(-c3nc(C(=O)NC(CC)CC)n(CCOCCOCCOC)n3)c2)o1]

LCMS Rt: 1.50 mins MS m/z 585.7 [M+H]+ 2minLowpHv03

¹H NMR (400 MHz, CHLOROFORM-d) δ ppm 8.82 (s, 1H) 8.28 (br d, J=7.82 Hz, 1H) 8.15 (br d, J=7.82 Hz, 1H) 7.84 (s, 1H) 7.60 (s, 1H) 7.24 (br d, J=9.29 Hz, 1H) 6.08 (br d, J=9.05 Hz, 1H) 4.97 (br t, J=5.50 Hz, 2H) 4.04-4.10 (m, 1H) 4.01 (br t, J=5.62 Hz, 2H) 3.89-3.97 (m, 1H) 3.65-3.74 (m, 2H) 3.55-3.64 (m, 4H) 3.45-3.51 (m, 2H) 3.34 (s, 3H) 1.66-1.78 (m, 4H) 1.58 (dt, J=14.37, 7.37 Hz, 4H) 1.01 (td, J=7.34, 4.16 Hz, 12H)

### Example 15.10: Tert-Butyl 2-(5-(Pentan-3-Ylcarbamoyl)-3-(3-(5-(Pentan-3-Ylcarbamoyl)Oxazol-2-Yl)Phenyl)-1H-1,2,4-Triazol-1-Yl)Acetate

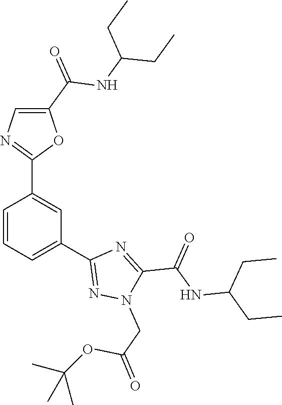 [SMILES: CCC(CC)NC(=O)c1cnc(-c2cccc(-c3nc(C(=O)NC(CC)CC)n(CC(=O)OC(C)(C)C)n3)c2)o1]

LCMS Rt: 1.69 mins MS m/z 497.6 [M+H des tert-butyl group]

¹H NMR (400 MHz, CHLOROFORM-d) δ ppm 8.43 (s, 1H) 8.31 (br d, J=7.82 Hz, 1H) 7.81-7.86 (m, 2H) 7.69 (s, 1H) 6.95 (br d, J=9.54 Hz, 1H) 6.21 (br d, J=9.05 Hz, 1H) 5.02 (s, 2H) 4.38 (s, 3H) 3.98-4.11 (m, 2H) 2.00 (br s, 2H) 1.71 (dq, J=14.27, 7.14 Hz, 4H) 1.54-1.63 (m, 4H) 1.45 (s, 9H) 1.00 (br s, 12H)

### Example 15.11: Ethyl 6-(5-(Pentan-3-Ylcarbamoyl)-3-(3-(5-(Pentan-3-Ylcarbamoyl-Carbamoyl)Oxazol-2-Yl)Phenyl)-1H-1,2,4-Triazol-1-Yl)Hexanoate

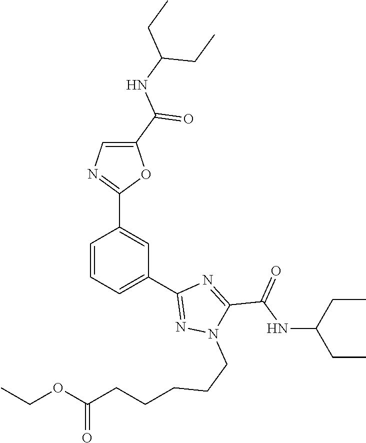 [SMILES: CCOC(=O)CCCCCn1nc(-c2cccc(-c3ncc(C(=O)NC(CC)CC)o3)c2)nc1C(=O)NC(CC)CC]

LCMS Rt: 1.70 MS m/z 581.8 [M+H]+ 2minLowpHv03

¹H NMR (400 MHz, CHLOROFORM-d) δ ppm 8.83 (s, 1H) 8.28 (br d, J=7.82 Hz, 1H) 8.16 (br d, J=7.82 Hz, 1H) 7.85 (s, 1H) 7.61 (s, 1H) 7.24-7.28 (m, 1H) 6.09 (br d, J=9.54 Hz, 1H) 4.77 (br t, J=7.21 Hz, 2H) 4.12 (d, J=7.34 Hz, 2H) 4.01-4.07 (m, 1H) 3.89-3.98 (m, 1H) 2.33 (t, J=7.34 Hz, 2H) 1.95-2.06 (m, 2H) 1.66-1.80 (m, 8H) 1.58 (br dd, J=13.20, 5.87 Hz, 5H) 1.46 (br d, J=7.09 Hz, 2H) 1.25 (t, J=7.21 Hz, 3H) 1.01 (td, J=7.21, 3.18 Hz, 12H)

Example 16 of the present invention may be prepared according to Scheme 22.

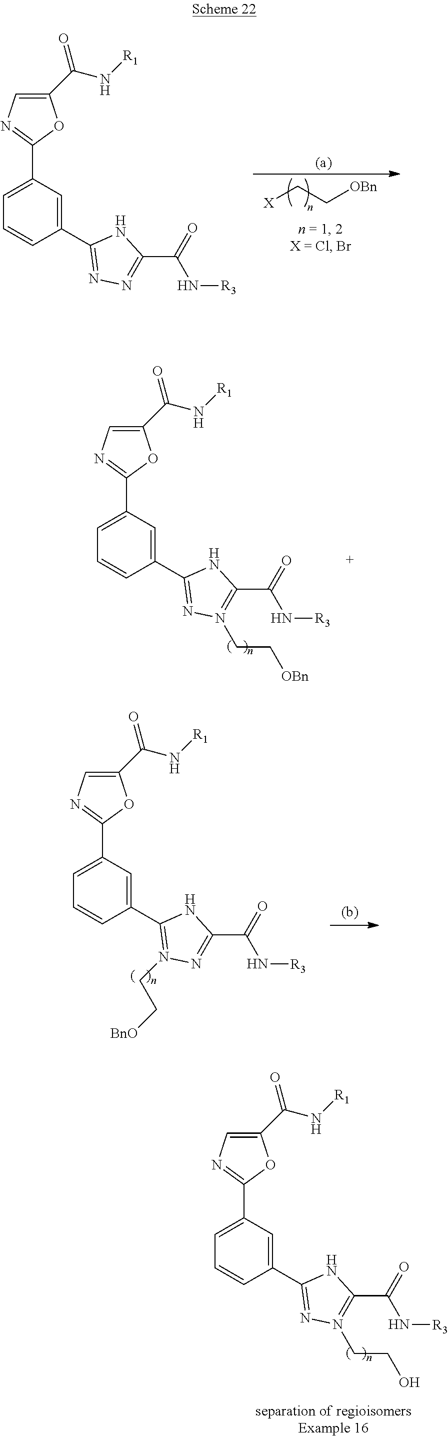

Step (a) involves alkylation of triazole nitrogen with haloalkylbenzyl ether to give varying chain lengths in the presence of a base such as LiHMDS, NaH, Cs2CO3, NEt3, Na2CO3 or K2C03 in a solvent such as THE or DMF to give a mixture of inseparable regioisomeric products.
Step (b) involves hydrogenation to liberate the alcohol of the tether from the benzyl protective group using a suitable palladium catalyst such as Pd (0) on carbon black in a suitable solvent such as methanol, ethanol followed by separation of regioisomers by chromatography to obtain the desired regioisomer.

### Example 16.0: 2-(3-(1-(2-Hydroxyethyl)-5-(Pentan-3-Ylcarbamoyl)-1H-1,2,4-Triazol-3-Yl)Phenyl)-N-(Pentan-3-Yl)Oxazole-5-Carboxamide

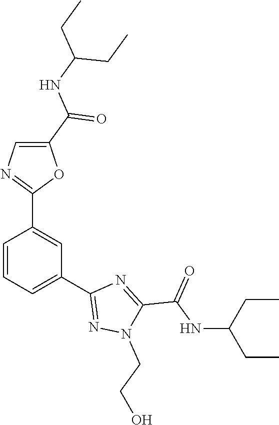 [SMILES: CCC(CC)NC(=O)c1cnc(-c2cccc(-c3nc(C(=O)NC(CC)CC)n(CCO)n3)c2)o1]

Step 1: 2-(3-(1-(2-(benzyloxy)ethyl)-5-(pentan-3-ylcarbamoyl)-1 H-1,2,4-triazol-3-yl)phenyl)-N-(pentan-3-yl)oxazole-5-carboxamide N-(pentan-3-yl)-2-(3-(5-(pentan-3-ylcarbamoyl)-1 H-1,2,4-triazol-3-yl)phenyl)oxazole-5-carboxamide, (200 mg, 0.456 mmol) was dissolved in DMF (2 ml) and cooled in an icebath. 1 M LiHMDS in THE (1 ml, 1.003 mmol) was added dropwise and the RM was stirred for 15 minutes. ((2-bromoethoxy)methyl)benzene (159ul, 1.003 mmol) was then added and the RM was allowed to warm to RT and stirred for 18 h. An additional 2.2 eq LHMDS (1 ml, 1.003 mmol) and 2.2 eq ((2-bromoethoxy)methyl)benzene (159ul, 1.003 mmol) were added, and the RM was stirred 18 h more before direct purification by prep HPLC to give 82 mg (30.2%) of 2-(3-(1-(2-(benzyloxy)ethyl)-5-(pentan-3-ylcarbamoyl)-1 H-1,2,4-triazol-3-yl)phenyl)-N-(pentan-3-yl)oxazole-5-carboxamide.
LCMS Rt: 1.68 mins MS m/z 573.7 [M+H]+ 2minLowpHv03
Step 2: 2-(3-(1-(2-hydroxyethyl)-5-(pentan-3-ylcarbamoyl)-1 H-pyrazol-3-yl)phenyl)-N-(pentan-3-yl)oxazole-5-carboxamide 2-(3-(1-(2-(benzyloxy)ethyl)-5-(pentan-3-ylcarbamoyl)-1 H-1,2,4-triazol-3-yl)phenyl)-N-(pentan-3-yl)oxazole-5-carboxamide (65 mg, 0.143 mmol) was dissolved in EtOH (14.3 ml). EtOAc (5 ml) was added and the RM was hydrogenated using the H-cube apparatus, using a 10% Pd on C cat cart, at 70° C., atmospheric pressure. The eluant was concentrated and then purified by FCC (10-90% EtOAc/iso-hexane) to give 52 mg (71.5%) of 2-(3-(1-(2-hydroxyethyl)-5-(pentan-3-ylcarbamoyl)-1 H-pyrazol-3-yl)phenyl)-N-(pentan-3-yl)oxazole-5-carboxamide.
LCMS Rt: 1.37 mins MS m/z 483.5 [M+H]+ 2minLowpH_v3

¹H NMR (400 MHz, CHLOROFORM-d) δ ppm 8.81 (s, 1H) 8.26 (br d, J=8.07 Hz, 1H) 8.15 (br d, J=7.82 Hz, 1H) 7.83 (s, 1H) 7.60 (t, J=7.82 Hz, 1H) 7.29-7.33 (m, 1H) 6.05 (br d, J=9.29 Hz, 1H) 4.95 (br t, J=4.89 Hz, 2H) 4.11 (br t, J=5.01 Hz, 2H) 4.00-4.07 (m, 1H) 3.89-3.98 (m, 1H) 1.67-1.79 (m, 4H) 1.57 (dq, J=14.46, 7.41 Hz, 4H) 0.97-1.05 (m, 12 H)

Examples 16.1 and 16.2 were prepared by a similar method to that of Example 16.0 by replacement of N-(pentan-3-yl)-2-(3-(5-(pentan-3-ylcarbamoyl)-1 H-1,2,4-triazol-3-yl)phenyl)oxazole-5-carboxamide with the appropriate triazole and replacement of the ((2-bromoethoxy)methyl)benzene with an appropriate halobenzyl ether.

### Example 16.1: N-((S)-1-Cyclopropylethyl)-2-(3-(5-(((S)-1-Cyclopropylethyl)Carbamoyl)-1-(2-Hydroxyethyl)-1H-1,2,4-Triazol-3-Yl)Phenyl)Oxazole-5-Carboxamide

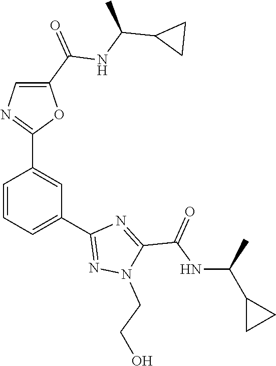 [SMILES: C[C@H](NC(=O)c1cnc(-c2cccc(-c3nc(C(=O)N[C@@H](C)C4CC4)n(CCO)n3)c2)o1)C1CC1]

LCMS Rt: 1.30 mins MS m/z 479.4 [M+H]+ 2minLowpHv03

¹H NMR (400 MHz, CHLOROFORM-d) δ ppm 8.73 (t, J=1.59 Hz, 1H) 8.14-8.20 (m, 1H) 8.07 (dt, J=8.07, 1.34 Hz, 1H) 7.74 (s, 1H) 7.48-7.55 (m, 2H) 6.31 (br d, J=8.31 Hz, 1H) 4.84 (dd, J=5.75, 4.03 Hz, 2H) 4.02 (t, J=4.89 Hz, 2H) 3.52 (br dd, J=8.31, 1.71 Hz, 1H) 3.38-3.49 (m, 1H) 1.29 (d, J=6.60 Hz, 6H) 0.87-0.98 (m, 2H) 0.49-0.58 (m, 2H) 0.43-0.49 (m, 2H) 0.32-0.42 (m, 2H) 0.20-0.30 (m, 2H)

### Example 16.2: N-((S)-1-Cyclopropylethyl)-2-(3-(5-(((S)-1-Cyclopropylethyl)Carbamoyl)-1-(3-Hydroxypropyl)-1H-1,2,4-Triazol-3-Yl)Phenyl)Oxazole-5-Carboxamide

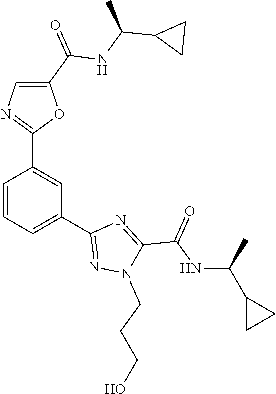 [SMILES: C[C@H](NC(=O)c1cnc(-c2cccc(-c3nc(C(=O)N[C@@H](C)C4CC4)n(CCCO)n3)c2)o1)C1CC1]

LCMS Rt: 1.32 mins MS m/z 493.4 [M+H]+ 2minLowpHv03

¹H NMR (400 MHz, CHLOROFORM-d) δ ppm 8.74 (t, J=1.47 Hz, 1H) 8.17 (dd, J=7.95, 1.59 Hz, 1H) 8.05-8.11 (m, 1H) 7.74 (s, 1H) 7.48-7.57 (m, 2H) 6.30 (br d, J=8.31 Hz, 1H) 4.80 (td, J=6.11, 1.47 Hz, 2H) 3.40-3.59 (m, 4H) 2.04-2.12 (m, 2H) 1.29 (dd, J=6.72, 3.06 Hz, 6H) 0.87-0.97 (m, 2H) 0.50-0.58 (m, 2H) 0.43-0.50 (m, 2H) 0.33-0.42 (m, 2H) 0.20-0.30 (m, 2H)

### Example 17.0:2-(3-(2-(((S)-1-Cyclopropylethyl)Carbamoyl)-1-(3,3,3-Trifluoro-2-Hydroxypropyl)-1H-Imidazol-4-Yl)Phenyl)-N-(Pentan-3-Yl)Oxazole-5-Carboxamide

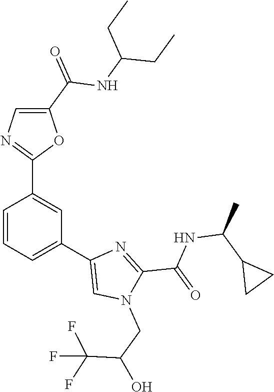 [SMILES: CCC(CC)NC(=O)c1cnc(-c2cccc(-c3cn(CC(O)C(F)(F)F)c(C(=O)N[C@@H](C)C4CC4)n3)c2)o1]

Step 1: Ethyl 4-bromo-1-((2-(trimethylsilyl)ethoxy)methyl)-1 H-imidazole-2-carboxylate, Ethyl 5-bromo-1-((2-(trimethylsilyl)ethoxy)methyl)-1 H-imidazole-2-carboxylate A solution of ethyl 4-bromo-1H-imidazole-2-carboxylate (180 mg, 0.867 mmol) and TEA (302 μL, 2.169 mmol) in THE (4.34 mL) was cooled in an ice-bath. SEM-CI (184 μL, 1.041 mmol) was added and the RM left to stir at ice-bath temperature for 20 minutes before allowing to warm to room temperature and leaving to stir for 18 h. The RM was diluted with water (40 mL) and extracted with EtOAc (40 mL). The organic phase was separated, dried over MgSO₄, and filtered. The filtrate was concentrated and purified by FCC (5-35% EtOAc/heptane), to afford
108 mg (35%) of ethyl 5-bromo-1-((2-(trimethylsilyl)ethoxy)methyl)-1 H-imidazole-2-carboxylate and 68 mg (22%) of ethyl 4-bromo-1-((2-(trimethylsilyl)ethoxy)methyl)-1 H-imidazole-2-carboxylate.
ethyl 4-bromo-1-((2-(trimethylsilyl)ethoxy)methyl)-1 H-imidazole-2-carboxylate LCMS Rt: 1.29 min; MS m/z 351.0 [M+H]+ RXNMON_Acidic 1H NMR (400 MHz, DMSO-d₆) δ 7.30 (s, 1H), 5.73 (s, 2H), 4.32 (q, J=7.1 Hz, 2H), 3.58-3.51 (m, 2H), 1.30 (t, J=7.1 Hz, 3H), 0.86-0.80 (m, 2H), -0.07 (s, 9H).
Step 2: Ethyl 4-(3-chlorophenyl)-1-((2-(trimethylsilyl)ethoxy)methyl)-1H-imidazole-2-carboxylate
Nitrogen was bubbled through a stirred suspension of (3-chlorophenyl)boronic acid (46 mg, 0.292 mmol) and ethyl 4-bromo-1-((2-(trimethylsilyl)ethoxy)methyl)-1 H-imidazole-2-carboxylate (68 mg, 0.195 mmol) in dioxane (0.78 mL). To this was added Pd(PPh₃)₄(23 mg, 0.019 mmol) followed by a solution of Na₂CO₃ (62 mg, 0.584 mmol) in water (0.2 mL). The RM was sealed and heated at 100° C. for 45 minutes under microwave irradiation. The RM was diluted with EtOAc (40 mL) and washed with water (20 mL). The organic phase was separated, dried over MgSO₄, and filtered. The filtrate was concentrated and purified by FCC (0-20% EtOAc/heptane) to afford 25 mg (33%) of ethyl 4-(3-chlorophenyl)-1-((2-(trimethylsilyl)ethoxy)methyl)-1 H-imidazole-2-carboxylate.
LCMS Rt: 1.48 min; MS m/z 381.1/383.0 [M+H]+ RXNMON_Acidic

### 1H NMR (400 MHz, DMSO-d₆) δ 7.73-7.69 (m, 1H), 7.61-7.53 (m, 3H), 7.38 (s, 1H), 5.69 (s, 2H), 4.35 (q, J=7.1 Hz, 2H), 3.50-3.43 (m, 2H), 1.33 (t, J=7.1 Hz, 3H), 0.83-0.76 (m, 2H), -0.08 (s, 9H).

Step 3: Ethyl 4-(3-(5-(pentan-3-ylcarbamoyl)oxazol-2-yl)phenyl)-1-((2-(trimethylsilyl)ethoxy)methyl)-1 H-imidazole-2-carboxylate
Pivalic acid (3 mg, 0.026 mmol), RuPhos-Pd-G1 TBME adduct (4 mg, 0.005 mmol), ethyl 4-(3-chlorophenyl)-1-((2-(trimethylsilyl)ethoxy)methyl)-1H-imidazole-2-carboxylate (25 mg, 0.066 mmol), N-(pentan-3-yl)oxazole-5-carboxamide (Intermediate 6), 24 mg, 0.131 mmol) and K₂CO₃ (27 mg, 0.197 mmol)] were combined under nitrogen with toluene (0.328 mL) and heated at 110° C. for 16 hours. The RM was diluted with EtOAc (30 mL) and washed with water (15 mL). The organic phase was separated, dried over MgSO₄, filtered and concentrated. The crude material was purified by prep HPLC Method 1 (basic) to afford 13 mg (37.6%) of ethyl 4-(3-(5-(pentan-3-ylcarbamoyl)oxazol-2-yl)phenyl)-1-((2-(trimethylsilyl)ethoxy)methyl)-1 H-imidazole-2-carboxylate.
LCMS Rt: 1.40 min; MS m/z 527.2 [M+H]+ RXNMON_Acidic

¹H NMR (400 MHz, DMSO-d₆) δ 8.31 (t, J=1.5 Hz, 1H), 8.28-8.20 (m, 2H), 7.91 (s, 1H), 7.80 (dt, J=7.8, 1.4 Hz, 1H), 7.74 (t, J=7.7 Hz, 1H), 7.42 (s, 1H), 5.67 (s, 2H), 4.36 (q, J=7.1 Hz, 2H), 3.83-3.72 (m, 1H), 3.49-3.42 (m, 2H), 1.64-1.54 (m, 2H), 1.53-1.43 (m, 2H), 1.34 (t, J=7.1 Hz, 3H), 0.90-0.80 (m, 8H), -0.11 (s, 9H).

Step 4: 4-(3-(5-(pentan-3-ylcarbamoyl)oxazol-2-yl)phenyl)-1-((2-(trimethylsilyl)ethoxy)methyl)-1 H-imidazole-2-carboxylic acid 1 M NaOH (30 μL, 0.03 mmol) was added to a stirred solution of ethyl 4-(3-(5-(pentan-3-ylcarbamoyl)oxazol-2-yl)phenyl)-1-((2-(trimethylsilyl)ethoxy)methyl)-1 H-imidazole-2-carboxylate (13 mg, 0.025 mmol) in EtOH (123 μL). After 1h the RM was concentrated to give 4-(3-(5-(pentan-3-ylcarbamoyl)oxazol-2-yl)phenyl)-1-((2-(trimethylsilyl)ethoxy)methyl)-1 H-imidazole-2-carboxylic acid which was used crude for the next step.
LCMS Rt: 1.06 min; MS m/z 499.2 [M+H]+ RXNMON_Acidic
Step 5: (S)-2-(3-(2-((1-cyclopropylethyl)carbamoyl)-1-((2-(trimethylsilyl)ethoxy)methyl)-1 H-imidazol-4-yl)phenyl)-N-(pentan-3-yl)oxazole-5-carboxamide
HATU (14 mg, 0.038 mmol) was added to a stirred solution of crude 4-(3-(5-(pentan-3-ylcarbamoyl)oxazol-2-yl)phenyl)-1-((2-(trimethylsilyl)ethoxy)methyl)-1 H-imidazole-2-carboxylic acid (12 mg, 0.025 mmol), TEA (9 μL, 0.063 mmol), and (S)-1-cyclopropylethanamine (4 mg, 0.05 mmol) in DMF (0.25 mL). After 2 the RM was diluted with diethyl ether (20 mL) and washed with water (10 mL). The organic phase was separated, dried over MgSO₄, filtered and concentrated to afford (S)-2-(3-(2-((1-cyclopropylethyl)carbamoyl)-1-((2-(trimethylsilyl)ethoxy)methyl)-1 H-imidazol-4-yl)phenyl)-N-(pentan-3-yl)oxazole-5-carboxamide which was used crude for the next step.
LCMS Rt: 1.55 min; MS m/z 566.3 [M+H]+ RXNMON_Acidic
1H NMR (400 MHz, DMSO-d₆) δ 8.51 (d, J=8.7 Hz, 1H), 8.31 (t, J=1.5 Hz, 1H), 8.25 (d, J=8.9 Hz, 1H), 8.20 (dt, J=7.8, 1.3 Hz, 1H), 7.90 (s, 1H), 7.81 (dt, J=7.7, 1.2 Hz, 1H), 7.71 (t, J=7.8 Hz, 1H), 7.33 (s, 1H), 5.84 (d, J=1.7 Hz, 2H), 3.84-3.72 (m, 1H), 3.54-3.45 (m, 2H), 3.45-3.36 (m, 1H), 1.64-1.42 (m, 4H), 1.25 (d, J=6.7 Hz, 3H), 1.14-1.05 (m, 1H), 0.89-0.80 (m, 8H), 0.50-0.42 (m, 1H), 0.41-0.33 (m, 1H), 0.32-0.25 (m, 1H), 0.25-0.18 (m, 1H), -0.11 (s, 9H).
Step 6: (S)-2-(3-(2-((1-cyclopropylethyl)carbamoyl)-1 H-imidazol-4-yl)phenyl)-N-(pentan-3-yl)oxazole-5-carboxamide
TFA (197 μL, 2.56 mmol) was added to a stirred solution of (S)-2-(3-(2-((1-cyclopropylethyl)carbamoyl)-1-((2-(trimethylsilyl)ethoxy)methyl)-1 H-imidazol-4-yl)phenyl)-N-(pentan-3-yl)oxazole-5-carboxamide (14 mg, 0.025 mmol) in DCM (0.64 mL) and the RM left to stir at room temperature for 72 h. The RM was concentrated and purified by prep HPLC Method 1 (basic) to afford (S)-2-(3-(2-((1-cyclopropylethyl)carbamoyl)-1H-imidazol-4-yl)phenyl)-N-(pentan-3-yl)oxazole-5-carboxamide.
LCMS Rt: 1.12 min; MS m/z 436.3 [M+H]+ RXNMON_Acidic
1H NMR (400 MHz, DMSO-d₆) δ 13.19 (s, 1H), 8.65-8.60 (m, 1H), 8.35 (d, J=8.8 Hz, 1H), 8.26 (d, J=8.9 Hz, 1H), 8.08-7.98 (m, 2H), 7.94-7.89 (m, 2H), 7.59 (t, J=7.8 Hz, 1H), 3.85-3.72 (m, 1H), 3.45-3.35 (m, 1H), 1.66-1.54 (m, 2H), 1.54-1.41 (m, 2H), 1.28 (d, J=6.7 Hz, 3H), 1.20-1.10 (m, 1H), 0.88 (t, J=7.4 Hz, 6H), 0.54-0.44 (m, 1H), 0.44-0.36 (m, 1H), 0.34-0.20 (m, 2H).
Step 7: 2-(3-(2-(((S)-1-cyclopropylethyl)carbamoyl)-1-(3,3,3-trifluoro-2-hydroxypropyl)-1H-imidazol-4-yl)phenyl)-N-(pentan-3-yl)oxazole-5-carboxamide
Nitrogen was bubbled through a stirred solution of (S)-2-(3-(2-((1-cyclopropylethyl)carbamoyl)-1 H-imidazol-4-yl)phenyl)-N-(pentan-3-yl)oxazole-5-carboxamide (25 mg, 0.057 mmol) and 3-bromo-1,1,1-trifluoropropan-2-ol (15 μL, 0.144 mmol) in DMF (0.574 mL). Na₂CO₃ (30 mg, 0.287 mmol) was added and the RM sealed and heated at 120° C. under microwave irradiation for an hour. The RM was diluted with EtOAc (30 mL) and washed with water (15 mL). The organic phase was separated, dried over MgSO₄, filtered and concentrated. The crude material was purified by prep HPLC Method 1 (basic) to afford 15.5 mg (48%) of 2-(3-(2-(((S)-1-cyclopropylethyl)carbamoyl)-1-(3,3,3-trifluoro-2-hydroxypropyl)-1 H-imidazol-4-yl)phenyl)-N-(pentan-3-yl)oxazole-5-carboxamide.

LCMS Rt: 1.29 min; MS m/z 5483 [M+H]+ RXNMON_Acidic

1H NMR (400 MHz, DMSO-d₆) δ 8.58 (t, J=1.5 Hz, 1H), 8.45 (d, J=7.9 Hz, 1H), 8.26 (d, J=8.8 Hz, 1H), 8.06-8.00 (m, 3H), 7.93 (s, 1H), 7.62 (t, J=7.8 Hz, 1H), 6.69 (s, 1H), 4.89 (ddd, J=13.2, 7.1, 2.8 Hz, 1H), 4.49 (s, 1H), 4.44-4.35 (m, 1H), 3.84-3.73 (m, 1H), 3.45-3.35 (m, 1H), 1.65-1.43 (m, 4H), 1.28 (dd, J=6.7, 2.6 Hz, 3H), 1.20-1.09 (m, 1H), 0.88 (t, J=7.4 Hz, 6H), 0.53-0.36 (m, 2H), 0.34-0.20 (m, 2H).

### Example 18.0: 2,2′-(1,3-Phenylene)Bis(N-(Pentan-3-Yl)Oxazole-5-Carboxamide)

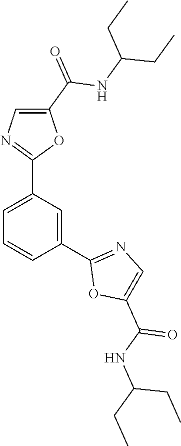 [SMILES: CCC(CC)NC(=O)c1cnc(-c2cccc(-c3ncc(C(=O)NC(CC)CC)o3)c2)o1]

To a solution of N-(pentan-3-yl)oxazole-5-carboxamide (Intermediate 6) (717 mg, 3.93 mmol) in NMP (Volume: 30 mL) was added 1,3-dibromobenzene (717 mg, 3.93 mmol) followed by Pd(OAc)₂ (44.2 mg, 0.197 mmol) cesium carbonate (3846 mg, 11.80 mmol) and cyclic-Johnphos (138 mg, 0.393 mmol). The RM was heated to 100° C. and stirred for 2 h.
The RM was diluted with EtOAc and washed with sat. NH₄Cl and brine. The organic phase was dried by filtration through a phase separator and concentrated. The crude material were purified by Prep HPLC Method 1, High pH 40-80%) to give 10.4 mg (0.57%) of 2,2′-(1,3-phenylene)bis(N-(pentan-3-yl)oxazole-5-carboxamide).
LCMS Rt: 1.23 min; MS m/z 439.4 [M+H]+ 2minLowpHv03

¹H NMR (400 MHz, DMSO-d₆) δ ppm 8.28-8.33 (m, 2H) 8.14 (dt, J=7.95, 1.16 Hz, 1H) 7.89 (s, 1H) 7.78-7.83 (m, 1H) 7.56 (t, J=7.95 Hz, 1H) 3.72-3.85 (m, 1H) 1.58 (br dd, J=7.46, 5.26 Hz, 2H) 1.43-1.54 (m, 2H) 0.88 (t, J=7.34 Hz, 6H)

### Automated Patch Clamping Assay to Measure TMEM16A Activity in Whole-Cell

### Cell Line Maintenance and Preparation

Chinese hamster ovary cells (CHO) cells expressing abc isoform of human TMEM16A channel (Ref) were grown in vented cell-culture flasks (Corning) containing cell culture medium (1×F-12 Ham, 10% FBS, 1% Penicillin-streptomycin, 5 μg/mL Blasticidin S HCl, 400 μg/mL Zeocin), maintained at 37° C. in an incubator with 5% C02, 95% 02 and 100% humidity. The cells were passaged every 2-3 days at 80-90% confluence by aspirating the cell culture medium, washing twice with 10 mL D-PBS then incubating with 4 mL Trypsin-EDTA for no longer than 5 minutes. Trypsin was neutralized with the addition of 32 mL growth medium to the cell suspension, the cells were counted using a viable-cell counter (Vi-CELL; Beckmann-Coulter) and new flasks were seeded at a cell density of 0.01, 0.02, or 0.05×106 cells per cm2 for 3, 2, or 1 day(s) growth, respectively. Cell suspension was diluted in the new 175 cm2 flask with 50 mL of growth medium. Doxycycline was used to induce the expression of TMEM16A channel protein and was added directly to the culture flask 18-24 hours in advance of assay at a final concentration of 1 μg/mL. Induced cells were washed with D-PBS and detached from their culture flasks by adding 10 mL of Detachin and incubating for 10 minutes. Once detached, 5 mL of QPatch assay medium (1×CHO-S-SFM II (Sigma), 25 mM HEPES) was added and the resultant cell suspension counted using the Vi-CELL viable cell counter. The optimal cell density for this QPatch assay is 2-5×1 06 cells/mL; re-adjusted as necessary with QPatch assay medium.

### Automated Patch Clamping Recording

The ion channel activity of TMEM16A (abc) expressed in CHO cells was assed using an automated patch clamp system (Qpatch, Sophion). These systems use planar patch-clamp technology to enable high-resistance, continuous whole-cell recordings from multiple cells in parallel, whilst maintaining each individual cell as an isolated experiment. First, each well was primed with intracellular and extracellular solutions described below. Cells were spun down, washed and then resuspended using extracellular solution.
Intracellular solution: 130 mM N-methyl-D-glucamine, 10 mM EGTA, 20 mM CaCl2, 1 mM MgCl2, 10 mM HEPES 10, 10 mM BAPTA, 99 mM Sucrose, 2 mM Mg-ATP, pH 7.3, 320 mOsm
Extracellular solution: 130 mM N-methyl-D-glucamine, 10 mM EGTA, 20 mM CaCl2, 1 mM MgCl2, 10 mM HEPES 10, 10 mM BAPTA, 99 mM Sucrose, 2 mM Mg-ATP, pH 7.3, 320 mOsm.
Resuspended cells were then added to each well and suction was applied from the intracellular side in order to position a cell on the chip aperture, form a high resistance (GO) seal and achieve a whole-cell recording configuration.
Following successful whole cell access, cell membranes were maintained at a voltage of −70 mV until the voltage protocol is applied. From this voltage, membranes were depolarized to +70 mV for 1500 ms, and then hyperpolarized to −70 mV before being depolarized again from −90 to +70 mV in a continuous ramp waveform. At the end of the ramp, the membrane voltage is stepped back to −70 mV until the next waveform was applied. Compounds were dissolved at 10 mM in 100% DMSO and then diluted into extracellular solution (0.3% DMSO final) to the desired final concentration.
The assay lower and upper limit were defined as follow, average amplitude (nA) of the last 3 sweeps in the vehicle addition of each cell was considered the basal current (lower limit); the average maximum current response in 3 cells for a reference potentiator at maximal concentration (upper limit).
Current values for each compound concentration were then plotted against time. EC50 curves are fitted to concentration-response data using curve-fitting functions (Hill fit) within the QPatch software. Curve fitting is constrained between the lowest concentration (vehicle-only addition) and the highest current value measured within the concentration range.

Compound % TMEM16A max activation was calculated as follow: Compound % TMEM16A max activation=(maximum current at the highest dose-lower limit)/(upper limit-lower limit)*100

TABLE
Calculated EC50 and % TMEM16A max
activation of tested compounds

| Example | TMEM16A QPATCH assay | TMEM16A QPATCH assay |
|---|---|---|
| number | EC50 V3 EC50 | EC50 V3 Assay Window |
| 1.0 | 0.04 | 79 |
| 1.1 | 0.02 | 90 |
| 1.1 | 0.01 | 100 |
| 1.2 | 0.80 | 83 |
| 1.3 | 0.56 | 82 |
| 1.4 | 0.91 | 103 |
| 1.5 | 0.06 | 98 |
| 1.6 | 0.13 | 61 |
| 1.7 | 0.09 | 69 |
| 1.8 | 0.17 | 54 |
| 1.9 | 0.03 | 146 |
| 1.11 | 0.27 | 165 |
| 1.12 | 0.02 | 87 |
| 1.13 | 0.40 | 112 |
| 1.14 | 0.68 | 111 |
| 1.15 | 0.41 | 100 |
| 1.16 | 0.83 | 100 |
| 1.17 | 0.58 | 94 |
| 1.18 | 0.18 | 39 |
| 1.19 | 0.13 | 69 |
| 1.20 | 0.29 | 112 |
| 1.21 | 0.93 | 92 |
| 1.22 | 0.03 | 119 |
| 1.24 | 0.18 | 83 |
| 1.25 | 0.26 | 83 |
| 1.26 | 0.64 | 91 |
| 1.27 | 0.92 | 86 |
| 1.28 | 0.61 | 84 |
| 1.29 | 0.01 | 113 |
| 1.30 | 0.87 | 62 |
| 1.31 | 0.77 | 65 |
| 1.32 | 0.88 | 77 |
| 1.33 | 0.72 | 66 |
| 1.34 | 0.57 | 72 |
| 1.35 | 0.07 | 90 |
| 1.36 | 1.10 | 102 |
| 1.37 | 0.20 | 81 |
| 1.38 | 0.90 | 61 |
| 1.39 | 0.88 | 66 |
| 1.40 | 0.16 | 93 |
| 1.41 | 0.95 | 75 |
| 1.42 | 0.75 | 72 |
| 1.43 | 0.43 | 70 |
| 1.44 | 0.51 | 69 |
| 1.45 | 0.71 | 75 |
| 1.46 | 0.99 | 19 |
| 1.47 | 0.15 | 75 |
| 1.48 | 0.07 | 59 |
| 1.49 | 0.40 | 70 |
| 1.50 | 0.45 | 115 |
| 1.51 | 0.98 | 64 |
| 2.0 | 0.14 | 87 |
| 2.1 | 0.03 | 83 |
| 2.2 | 0.032 | 112 |
| 2.3 (i) | 0.03 | 86 |
| 2.3 (ii) | 0.08 | 97 |
| 2.4 | 0.02 | 99 |
| 2.5 | 0.01 | 32 |
| 3.0 (i) | 0.49 | 92 |
| 3.0 (ii) | 0.95 | 79 |
| 3.1 | 0.30 | 140 |
| 3.2 | 0.03 | 90 |
| 3.3 | 0.18 | 133 |
| 3.4 (i) | 0.71 | 90 |
| 3.4 (ii) | 0.26 | 115 |
| 3.4 (iii) | 0.55 | 73 |
| 3.4 (iv) | 0.38 | 73 |
| 3.5 | 0.04 | 124 |
| 3.6 | 0.17 | 122 |
| 3.7 | 0.02 | 118 |
| 3.8 | 0.05 | 99 |
| 3.9 | 0.50 | 96 |
| 3.10 | 0.09 | 95 |
| 3.11 | 0.59 | 91 |
| 3.12 | 0.27 | 76 |
| 3.13 | 0.07 | 70 |
| 3.14 | 0.49 | 43 |
| 3.15 | 0.20 | 72 |
| 3.16 | 0.14 | 67 |
| 3.17 | 0.17 | 66 |
| 3.18 | 0.03 | 66 |
| 3.19 | 0.27 | 69 |
| 3.20 | 0.45 | 82 |
| 3.21 | 0.19 | 74 |
| 3.22 | 0.14 | 60 |
| 3.23 | 0.13 | 79 |
| 3.24 | 0.04 | 76 |
| 3.25 | 0.04 | 90 |
| 3.26 | 0.24 | 109 |
| 3.27 | 0.01 | 87 |
| 3.28 | 0.35 | 84 |
| 3.29 | 0.04 | 94 |
| 3.30 | 0.75 | 90 |
| 3.31 | 0.18 | 87 |
| 3.32 | 0.11 | 55 |
| 3.33 | 0.84 | 61 |
| 3.34 | 0.22 | 77 |
| 3.35 | 0.12 | 69 |
| 3.36 | 0.19 | 97 |
| 3.37 | 0.20 | 109 |
| 3.38 | 0.15 | 100 |
| 3.39 | 0.15 | 98 |
| 3.40 | 0.07 | 96 |
| 3.41 | 0.13 | 89 |
| 3.42 | 0.69 | 86 |
| 3.43 | 0.13 | 84 |
| 3.44 | 0.23 | 82 |
| 3.45 | 0.55 | 81 |
| 3.46 | 0.80 | 80 |
| 3.47 | 0.94 | 77 |
| 3.48 | 0.12 | 74 |
| 3.49 | 0.17 | 72 |
| 3.50 | 0.35 | 70 |
| 3.51 | 0.11 | 68 |
| 3.52 | 0.03 | 95 |
| 3.53 | 0.72 | 109 |
| 3.54 | 0.06 | 109 |
| 3.55 | 0.02 | 69 |
| 3.56 | 0.09 | 78 |
| 3.57 | 0.65 | 111 |
| 3.58 | 0.27 | 77 |
| 3.59 | 0.06 | 106 |
| 3.60 | 0.16 | 150 |
| 3.61 | 0.13 | 123 |
| 3.62 | 0.96 | 70 |
| 3.63 | 0.86 | 72 |
| 3.64 | 0.15 | 72 |
| 3.65 | 0.78 | 28 |
| 3.66 | 0.14 | 80 |
| 3.67 | 0.79 | 68 |
| 3.68 | 0.16 | 88 |
| 3.69 | 0.26 | 76 |
| 4.0 | 0.33 | 106 |
| 4.1 | 0.027 | 81 |
| 4.2 | 0.68 | 125 |
| 4.3 | 0.09 | 167 |
| 4.4 | 0.13 | 172 |
| 4.5 | 0.12 | 154 |
| 5.0 | 0.04 | 110 |
| 5.1 | 0.32 | 125 |
| 5.2 | 0.84 | 201 |
| 6.0 | 0.31 | 90 |
| 6.1 | 0.12 | 220 |
| 6.2 | 0.05 | 131 |
| 6.3 | 0.25 | 73 |
| 6.4 | 0.03 | 109 |
| 7.0 | 0.44 | 175 |
| 8.0 | 0.20 | 106 |
| 8.1 (i) | 0.01 | 135 |
| 8.1 (ii) | 0.02 | 124 |
| 8.2 | 0.45 | 119 |
| 9.0 | 0.15 | 137 |
| 9.1 | 0.08 | 155 |
| 9.2 (i) | 0.13 | 115 |
| 9.2 (ii) | 0.04 | 83 |
| 9.3 | 0.40 | 143 |
| 9.4 | 0.42 | 105 |
| 9.5 | 0.24 | 104 |
| 9.6 | 0.03 | 100 |
| 9.7 | 0.09 | 85 |
| 9.8 | 0.18 | 88 |
| 10.0 | 0.04 | 116 |
| 10.1 | 0.32 | 101 |
| 10.2 | 0.68 | 108 |
| 11.0 | 0.61 | 128 |
| 12.0 | 0.30 | 63 |
| 12.1 | 0.27 | 80 |
| 12.2 | 0.05 | 112 |
| 12.3 | 0.22 | 92 |
| 12.4 | 0.07 | 119 |
| 12.5 | 0.03 | 92 |
| 12.6 | 0.02 | 142 |
| 12.7 | 0.02 | 91 |
| 12.8 | 0.02 | 119 |
| 12.9 | 0.02 | 134 |
| 12.10 | 0.19 | 92 |
| 12.11 | 0.15 | 80 |
| 12.12 | 0.19 | 92 |
| 12.13 | 0.29 | 99 |
| 12.14 | 0.08 | 97 |
| 12.15 | 0.39 | 72 |
| 12.16 | 0.42 | 85 |
| 12.17 | 0.47 | 77 |
| 13.0 | 0.31 | 74 |
| 13.1 | 0.11 | 88 |
| 13.2 | 0.65 | 77 |
| 13.3 | 0.05 | 95 |
| 13.4 | 0.94 | 80 |
| 13.5 | 0.03 | 88 |
| 13.6 | 0.37 | 97 |
| 13.7 | 0.47 | 121 |
| 14.0 | 0.58 | 177 |
| 15.0 | 0.42 | 161 |
| 15.1 | 0.24 | 155 |
| 15.2 | 0.08 | 80 |
| 15.3 | 0.13 | 139 |
| 15.4 | 1.0 | 61 |
| 15.5 | 0.44 | 83 |
| 15.6 | 0.33 | 154 |
| 15.7 | 0.32 | 103 |
| 15.8 | 0.09 | 95 |
| 15.9 | 1.0 | 95 |
| 15.10 | 0.08 | 73 |
| 15.11 | 0.44 | 88 |
| 16.0 | 0.13 | 177 |
| 16.1 | 0.46 | 102 |
| 16.2 | 0.29 | 140 |
| 17.0 | 0.79 | 186 |
| 18.0 | 0.14 | 97 |

As indicated by the test results described hereinbefore, compounds of the present invention may be useful for treating diseases, conditions and disorders through the modulation of TMEM16A function; consequently, the compounds of the present invention (including the compositions and processes used therein) may be used in the manufacture of a medicament for the therapeutic applications described herein. Hence, another Embodiment of the present invention is a pharmaceutical composition comprising a compound of the present invention either alone or in combination with at least one additional therapeutic agent, or a pharmaceutically acceptable salt, hydrate, or co-crystal thereof, and a pharmaceutically acceptable diluent or carrier.

1. A compound of formula (1): 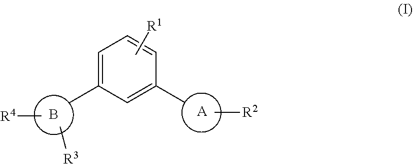
wherein:
Ring A is a 5 membered heteroaryl ring containing 2 heteroatoms independently selected from N and 0;
Ring B is a 5 membered heteroaryl ring containing 2 or 3 heteroatoms each independently selected from N, S and O, wherein at least one of said heteroatoms is N or ring B is a 6 membered heteroaryl containing 1 or 2 heteroatoms selected from N;
R¹ is hydrogen or halogen;
R² is selected from the group consisting of: 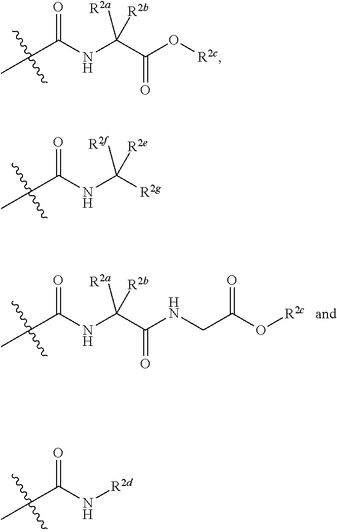
wherein
R^{2a} is H, (C₁-C₄)alkyl or phenyl, wherein said (C₁-C₄)alkyl is optionally substituted with halogen, (C₃-C₆)cycloalkyl, phenyl, —O—(C₁-C₄)alkyl or —S—(C₁-C₄)alkyl;
R^{2b} is H, (C₁-C₄)alkyl or R^{2b} taken together with R^{2a} forms a (C₃-C₅)cycloalkyl ring;
R^{2c} is (C₁-C₄)alkyl, (C₂-C₄)alkenyl or benzyl;
R^{2d} is (C,-C₄)alkyl, (C₃-C₆)cycloalkyl, adamantyl, a 5 or 6 membered heteroaryl wherein said heteroaryl contains 1 or 2 heteroatoms independently selected from N and O, or phenyl; wherein said phenyl is optionally substituted with 1 or 2 substituents independently selected from (C₁-C₄)alkyl, halo-(C₁-C₄)alkyl and nitrile;
R^{2e} is H, (C₁-C₄)alkyl or (C₃-C₆)cycloalkyl ring;
R^{2f} is H, (C₁-C₄)alkyl or (C₃-C₅)cycloalkyl ring optionally substituted with (C₁-C₄)alkyl or R^{2c} taken together with R²¹ forms a (C₃-C₆)cycloalkyl ring;
R^{2g} is H, (C₁-C₄)alkyl, a fused moiety selected from benzo[d][1,3]dioxole and indolin-2-one, where said fused moiety is optionally substituted with halogen or (C₁-C₄)alkyl, (C₃—Cs)heterocycloalkyl containing 1 or 2 heteroatoms selected from N and O, -(C₀-C₂)alkyl-phenyl wherein said phenyl is optionally substituted 1 or 2 groups independently selected from halogen and (C₁-C₄)alkyl;
R³ is H, (C₁-C₅)alkyl or a 4 to 6 membered saturated heterocycle containing O; wherein said (C₁-C₅)alkyl is optionally substituted with 1 to 3 groups independently selected from hydroxyl, (C₁-C₅)alkoxy, halogen, diethyl phosphate, —C(O)O(C₁-C₄)alkyl, NH-benzyl, O-benzyl, benzo[d][1,3]dioxole, isoindolinyl, —O—(C₂-C₄)alkyl-O—(C₁-C₄)alkyl, and a 4 to 6 membered saturated heterocycle containing 1 or 2 heteroatoms selected from N and O wherein said heterocycle is optionally substituted with 1 or 2 groups selected from (C₁-C₄)alkyl, and -C(O)NH(CHR⁵)C(O)O—(C₁-C₄)alkyl;
R⁴ is selected from the group consisting of: 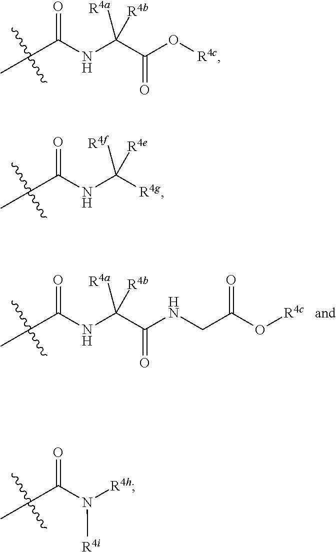
where
R^{4a} is H, (C₁-C₄)alkyl or phenyl, wherein said (C₁-C₄)alkyl is optionally substituted with 1 to 3 halogens, (C₃-C₆)cycloalkyl, phenyl, —O—(C₁-C₄)alkyl or —S—(C₁-C₄)alkyl;
R^{4b} is H or (C₁-C₄)alkyl or R^{4b} taken together with R^{4e} a to form a (C₃-C₅)cycloalkyl ring;
R^{4c} is (C₁-C₄)alkyl, (C₂-C₄)alkenyl or benzyl;
R^{4e} e is H, (C₁-C₄)alkyl, (C₁-C₄)alkoxy or (C₃-C₆)cycloalkyl ring;
R^{4f} is H, (C₁-C₄)alkyl or (C₃-C₅)cycloalkyl ring optionally substituted with nitrile or (C₁-C₄)akyl or R^{4e} taken together with R^{4f} to form a (C₃-C₆)cycloalkyl ring;
R^{4g} is H, (C₁-C₄)alkyl, a fused moiety selected from benzo[d][1,3]dioxole and indolin-2-one, where said fused moiety is optionally substituted with halogen or (C₁-C₄)alkyl, (C₃—Cs)heterocycloalkyl containing 1 or 2 heteroatoms selected from N and O, -(C₀-C₂)alkyl-phenyl wherein said phenyl is optionally substituted with 1 or 2 halogens;
R^{4h} is (C₁-C₄)alkyl, (C₃-C₅)cycloalkyl optionally substituted with 1 or 2 halogens, adamantyl, 5 or 6 membered heteroaryl wherein said heteroaryl contains 1 or 2 heteroatoms independently selected from N and O, phenyl; wherein said phenyl is optionally substituted with 1 or 2 substituents independently selected from (C₁-C₄)alkyl, (C₁-C₅)alkoxy, halo-(C₁-C₄)alkyl, halo-(C₁-C₄)alkoxy and nitrile;
R⁴ⁱ is H or R⁴ⁱ taken together with R^{4h} forms a (C₃-C₅)heterocycloalkyl ring optionally substituted with 1 or 2 substituents independently selected from (C₁-C₄)alkyl, (C₁-C₄)alkoxy and —C(O)O(C₁-C₄)alkyl; and
R⁵ is H or (C₁-C₄)alkyl, wherein said (C₁-C₄)alkyl is optionally substituted with (C₃—Cs)cycloalkyl, phenyl, —O—(C₁-C₄)alkyl or —S—(C₁-C₄)alkyl;
or a pharmaceutically acceptable salt, hydrate, or co-crystal thereof.

2. The compound of claim 1 of formula (Ia): 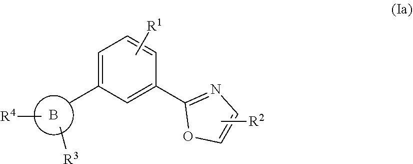
wherein:
Ring B is selected from the group consisting of the following wherein * indicates the point of attachment 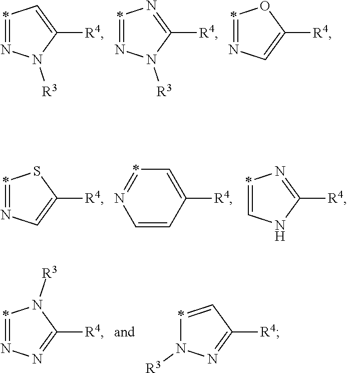
or a pharmaceutically acceptable salt, hydrate, or co-crystal thereof.

3. The compound of claim 1 of formula (Ia): 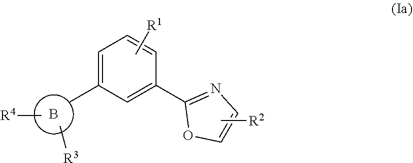
wherein:
Ring B is selected from the group consisting of the following wherein * indicates the point of attachment: 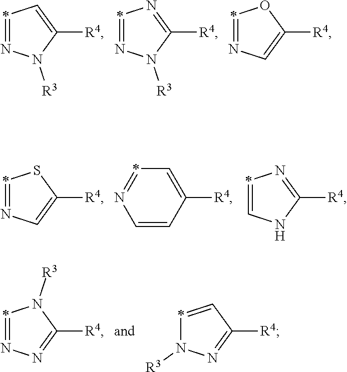
R³ is selected from the group consisting of the following wherein * indicates the point of attachment: *H, 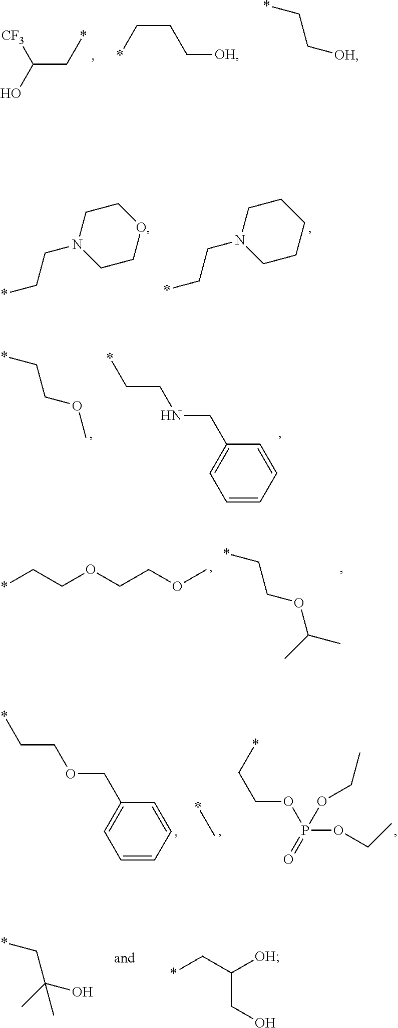
or a pharmaceutically acceptable salt, hydrate, or co-crystal thereof.

4. The compound according to claim 1 wherein:
R¹ is hydrogen;
or a pharmaceutically acceptable salt, hydrate, or co-crystal thereof.

5. The compound of claim 1 of formula (IIa): 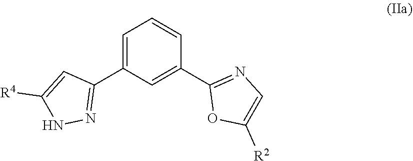
or a pharmaceutically acceptable salt, hydrate, or co-crystal thereof.

6. The compound of claim 1 of formula (IIb): 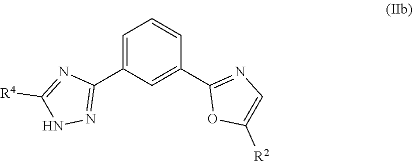
or a pharmaceutically acceptable salt, hydrate, or co-crystal thereof.

7. The compound of claim 1 of formula (IIc): 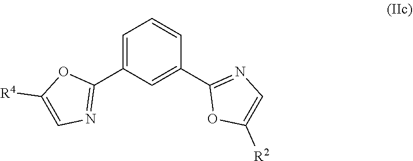
or a pharmaceutically acceptable salt, hydrate, or co-crystal thereof.

8. The compound of claim 1 of formula (lid): 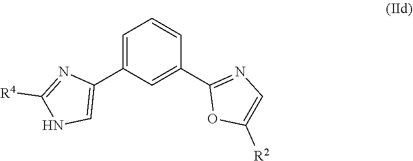
or a pharmaceutically acceptable salt, hydrate, or co-crystal thereof.

9. The compound of claim 1, wherein:
R¹ is H;
R² is selected from the group consisting of: 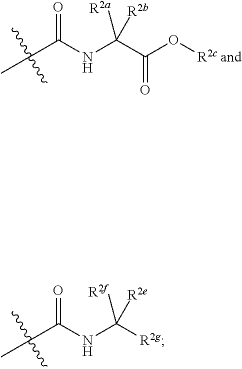
where
R^{2a} is H, (C₁-C₄)alkyl or phenyl, wherein said (C₁-C₄)alkyl is optionally substituted with halogen, (C₃-C₆)cycloalkyl, phenyl, —O—(C₁-C₄)alkyl or —S—(C₁-C₄)alkyl;
R^{2b} is H, (C₁-C₄)alkyl or R^{2b} taken together with R^{2a} forms a (C₃-C₅)cycloalkyl ring;
R^{2c} is (C₁-C₄)alkyl, (C₂-C₄)alkenyl or benzyl;
R^{2e} is H, (C₁-C₄)alkyl or (C₃-C₆)cycloalkyl ring;
R^{2f} is H, (C₁-C₄)alkyl or (C₃-C₅)cycloalkyl ring optionally substituted with (C₁-C₄)alkyl or R^{2c} taken together with R²¹ forms a (C₃-C₆)cycloalkyl ring;
R^{2g} is H, (C₁-C₄)alkyl, (C₃-C₅)heterocycloalkyl containing 1 or 2 heteroatoms selected from N and O, -(C₀-C₂)alkyl-phenyl wherein said phenyl is optionally substituted 1 or 2 groups independently selected from halogen and (C₁-C₄)alkyl;
R³ is H;
R⁴ is selected from the group consisting of: 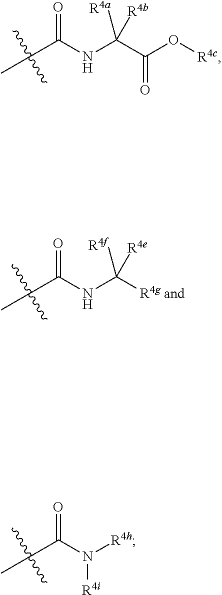
where
R^{4a} a is H, (C₁-C₄)alkyl, phenyl, wherein said (C₁-C₄)alkyl is optionally substituted with 1 to 3 halogens, (C₃-C₆)cycloalkyl, phenyl, —O—(C₁-C₄)alkyl or —S—(C₁-C₄)alkyl;
R^{4b} is H or (C₁-C₄)alkyl or R^{4b} taken together with R^{4a} to form a (C₃-C₅)cycloalkyl ring;
R^{4c} is (C₁-C₄)alkyl, (C₂-C₄)alkenyl and benzyl;
R^{4e} e is H, (C₁-C₄)alkyl, (C₁-C₄)alkoxy or (C₃-C₆)cycloalkyl ring;
R^{4f} is H, (C₁-C₄)alkyl or (C₃-C₅)cycloalkyl ring optionally substituted with nitrile or (C₁-C₄)akyl or R^{4e} taken together with R^{4e} f to form a (C₃-C₆)cycloalkyl ring;
R^{4g} is H, (C₁-C₄)alkyl, (C₃-C₅)heterocycloalkyl containing 1 or 2 heteroatoms selected from N and O, -(C₀-C₂)alkyl-phenyl wherein said phenyl is optionally substituted with 1 or 2 halogens;
R^{4h} is (C₁-C₄)alkyl, (C₃-C₅)cycloalkyl optionally substituted with 1 or 2 halogens, adamantyl, a 5 or 6 membered heteroaryl wherein said heteroaryl contains 1 or 2 heteroatoms independently selected from N and O, or phenyl; wherein said phenyl is optionally substituted with 1 or 2 substituents independently selected from (C₁-C₄)alkyl, (C₁-C₅)alkoxy, halo-(C₁-C₄)alkyl, halo-(C₁-C₄)alkoxy and nitrile; and
R⁴ⁱ is H or R⁴ⁱ taken together with R^{4h} to form a (C₃-C₅)heterocycloalkyl ring optionally substituted with 1 or 2 substituents independently selected from (C₁-C₄)alkyl, (C₁-C₄)alkoxy and —C(O)O(C₁-C₄)alkyl;
or a pharmaceutically acceptable salt, hydrate, or co-crystal thereof.

10. The compound of claim 1 wherein:
R² is selected from the group consisting of: 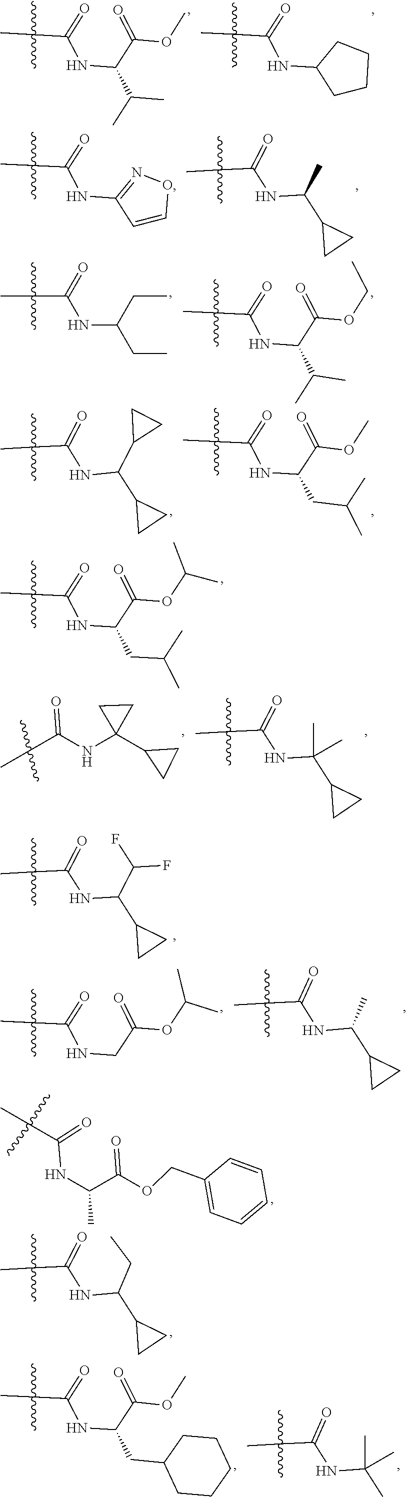 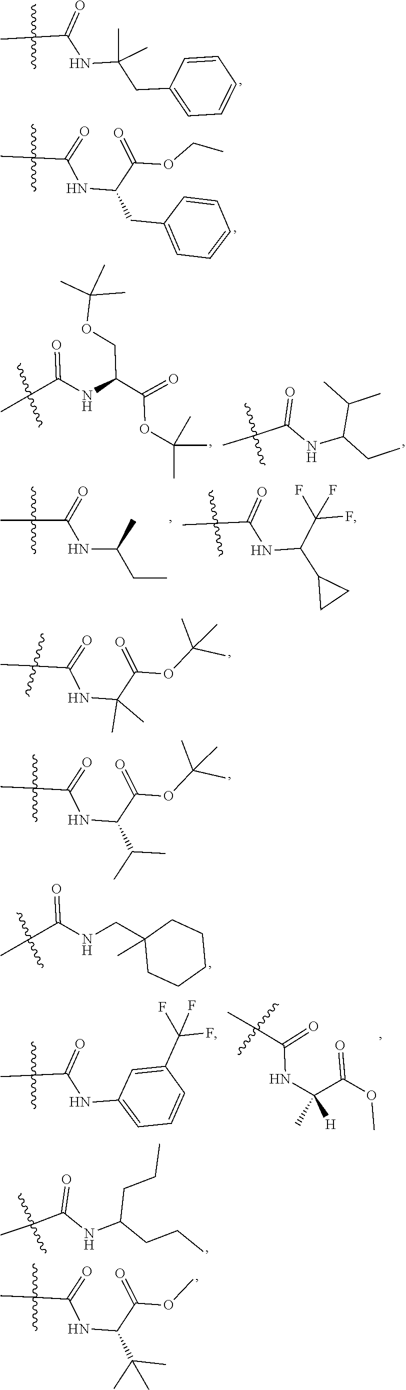 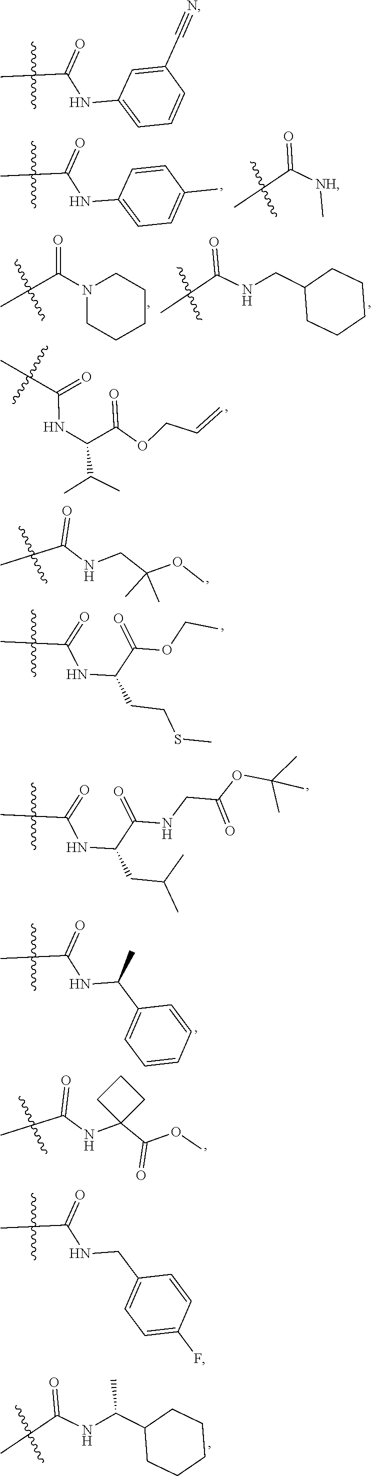 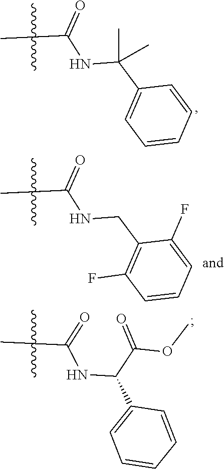
or a pharmaceutically acceptable salt, hydrate, or co-crystal thereof.

11. The compound of claim 1 wherein:
R⁴ is selected from the group consisting of: 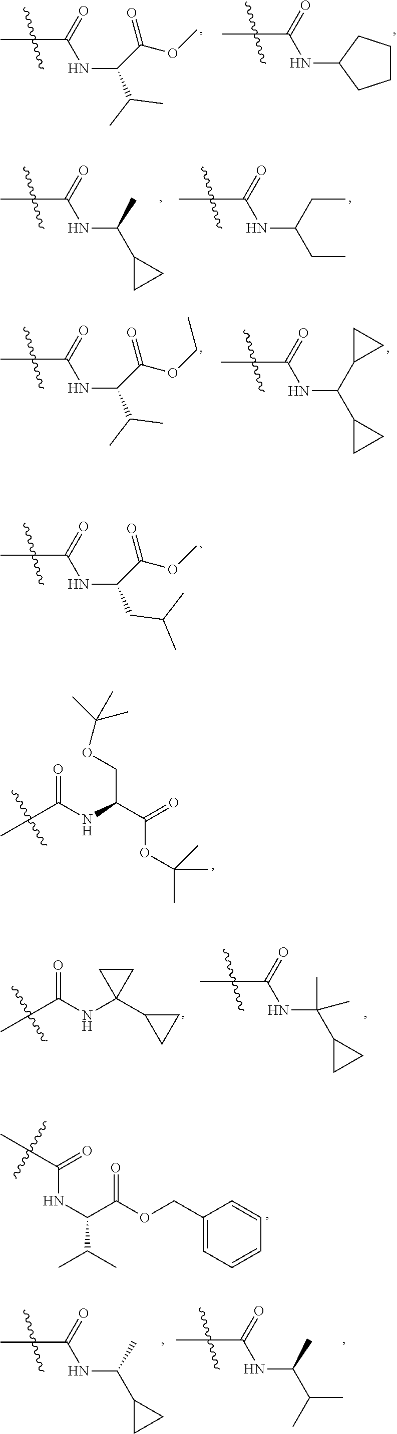 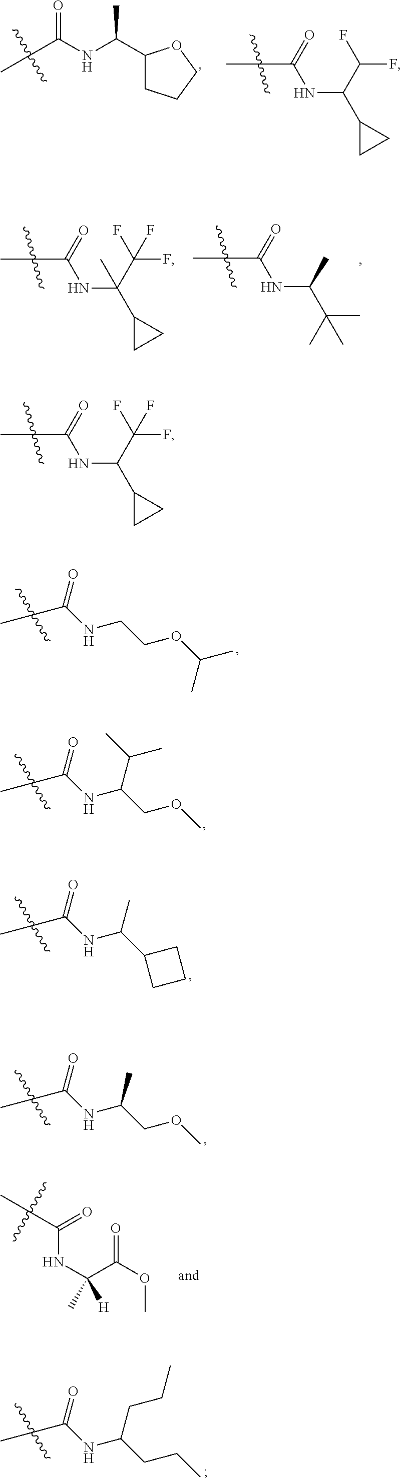
or a pharmaceutically acceptable salt, hydrate, or co-crystal thereof.

12. The compound of claim 1 of formula (IIa): 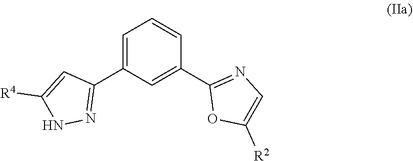
wherein R² is selected from the group consisting of: 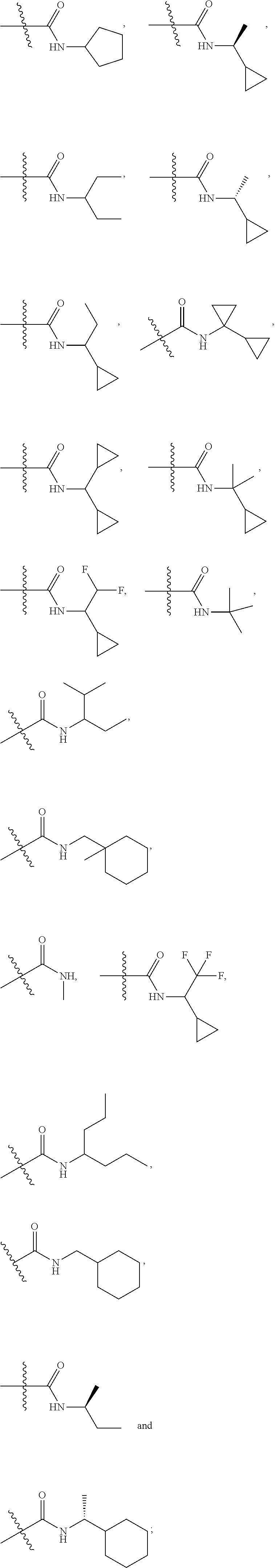
R⁴ is selected from the group consisting of: 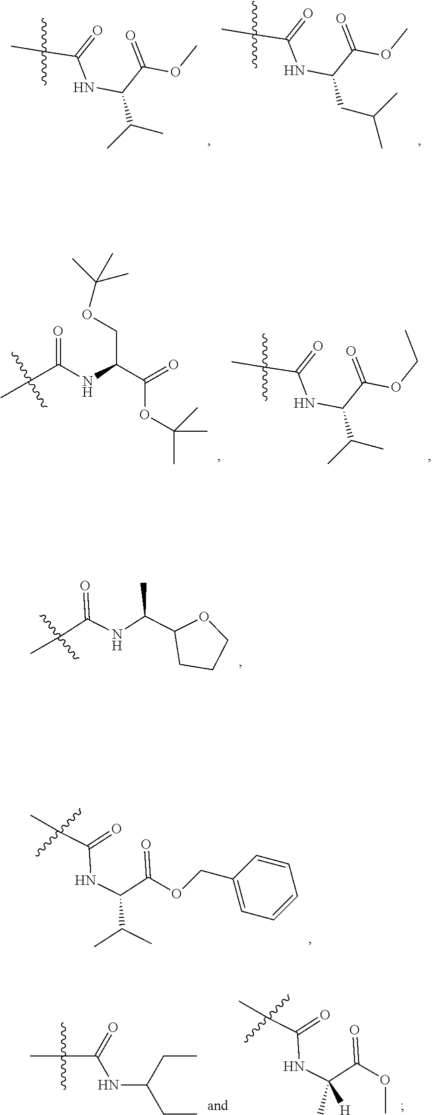
or a pharmaceutically acceptable salt, hydrate, or co-crystal thereof.

13. The compound according to claim 1 of formula (IIb): 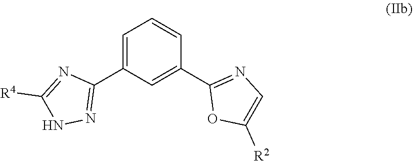
wherein R² is selected from the group consisting of: 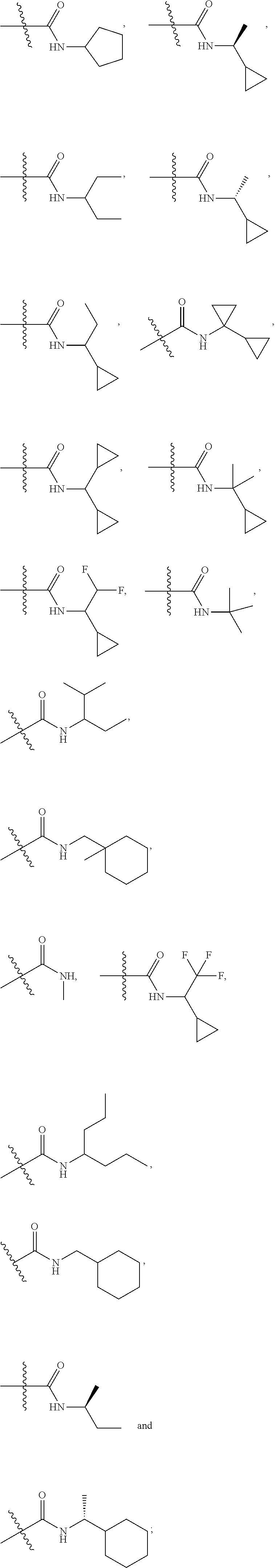
R⁴ is selected from the group consisting of: 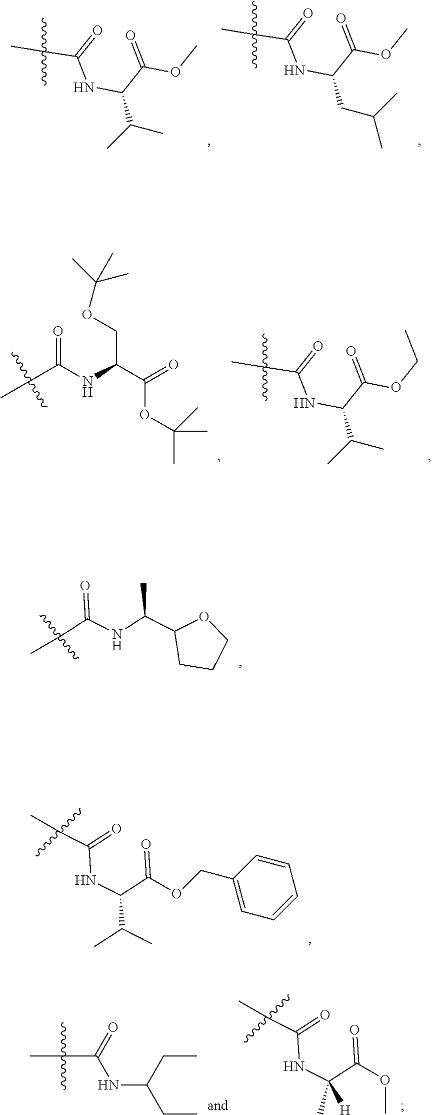
or a pharmaceutically acceptable salt, hydrate, or co-crystal thereof.

14. The compound according to claim 1 of formula (IIc): 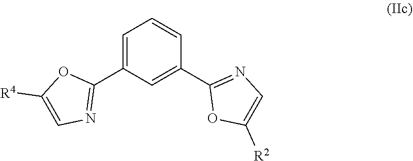
wherein R² is selected from the group consisting of: 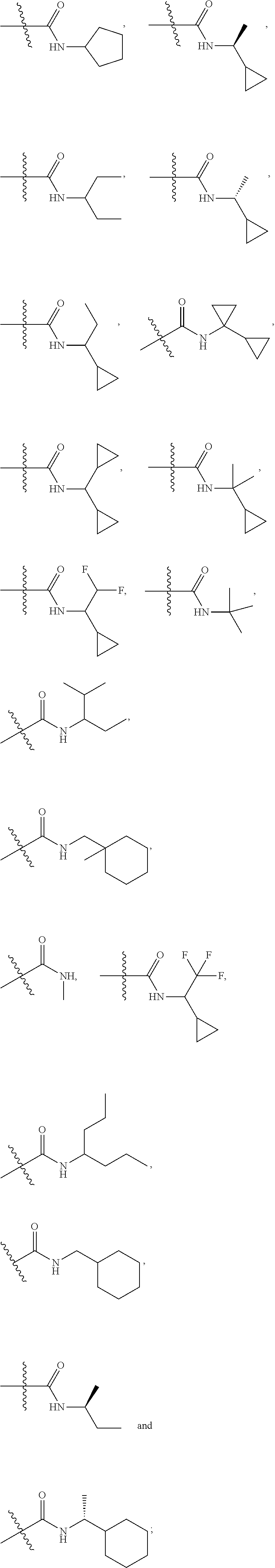
R⁴ is selected from the group consisting of: 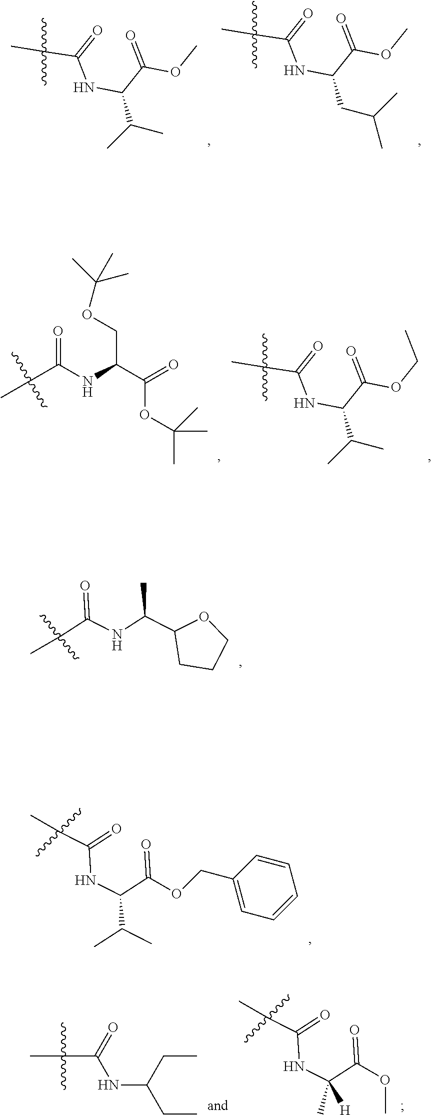
or a pharmaceutically acceptable salt, hydrate, or co-crystal thereof.

15. The compound according to claim 1 of formula (lid): 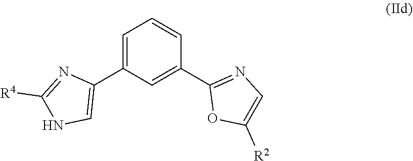
wherein R² is selected from the group consisting of: 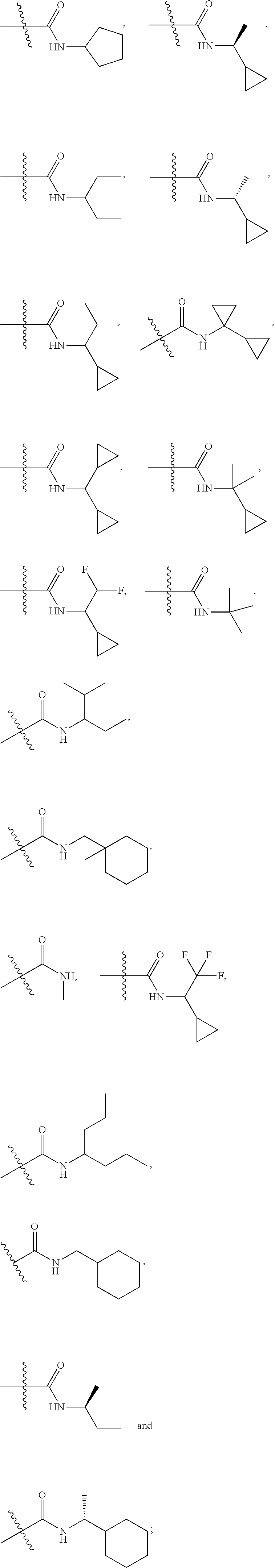
R⁴ is selected from the group consisting of: 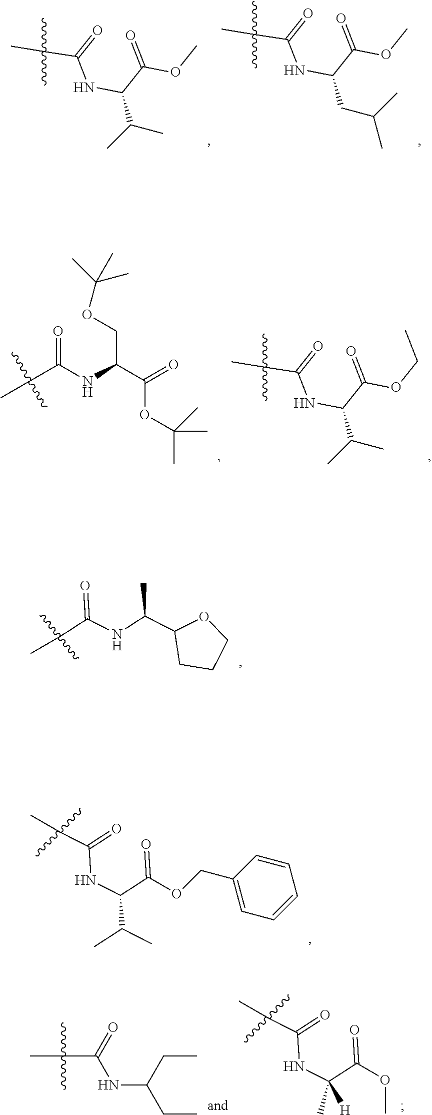
or a pharmaceutically acceptable salt hydrate or co-crystal thereof.

16. The compound according to claim 1 wherein R² is selected from the group consisting of: 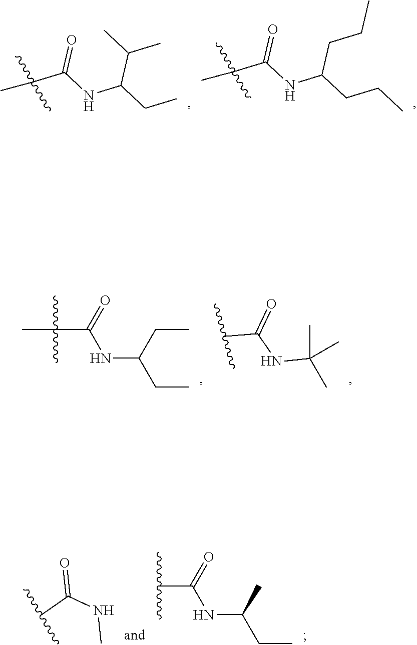
R⁴ is selected from the group consisting of: 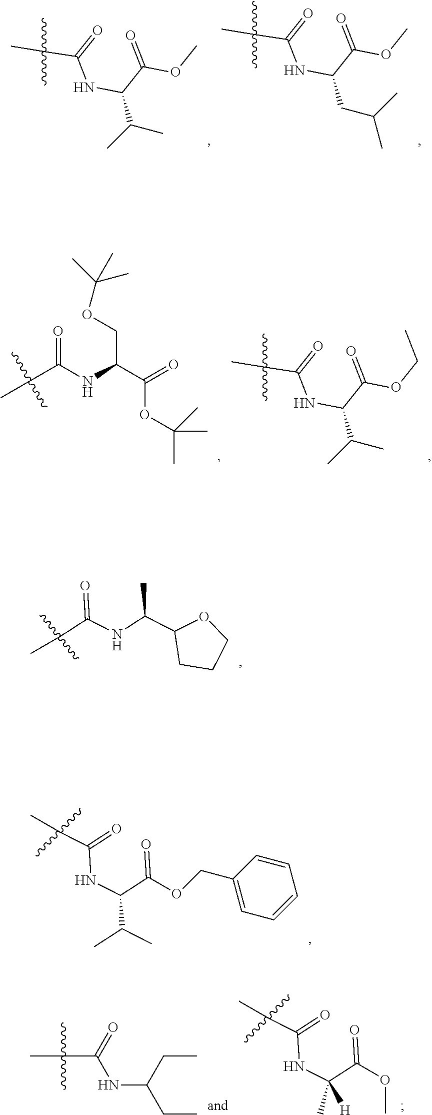
or a pharmaceutically acceptable salt, hydrate, or co-crystal thereof.

17. The compound of claim 1 selected from the group consisting of:
methyl (2-(3-(5-((dicyclopropylmethyl)carbamoyl)-1-(3,3,3-trifluoro-2-hydroxypropyl)-1 H-pyrazol-3-yl)phenyl)oxazole-5-carbonyl)-L-valinate;
N-cyclopentyl-2-(3-(5-(cyclopentylcarbamoyl)-1-(3-hydroxypropyl)-1 H-pyrazol-3-yl)phenyl)oxazole-5-carboxamide;
2-(3-(2-(((S)-1-cyclopropylethyl)carbamoyl)-1-(3,3,3-trifluoro-2-hydroxypropyl)-1 H-imidazol-4-yl)phenyl)-N-(pentan-3-yl)oxazole-5-carboxamide;
2-(3-(1-(2-hydroxyethyl)-5-(pentan-3-ylcarbamoyl)-1 H-1,2,4-triazol-3-yl)phenyl)-N-(pentan-3-yl)oxazole-5-carboxamide;
(S)-2-(3-(5-((1-cyclopropylethyl)carbamoyl)-4H-1,2,4-triazol-3-yl)phenyl)-N-(pentan-3-yl)oxazole-5-carboxamide;
ethyl (1-(2-morpholinoethyl)-3-(3-(5-(pentan-3-ylcarbamoyl)oxazol-2-yl)phenyl)-1 H-pyrazole-5-carbonyl)-L-valinate;
ethyl (2-(3-(5-((dicyclopropylmethyl)carbamoyl)-1-(2-hydroxyethyl)-1 H-pyrazol-3-yl)phenyl)oxazole-5-carbonyl)-L-valinate;
methyl (2-(3-(5-((dicyclopropylmethyl)carbamoyl)-1-(2-hydroxyethyl)-1H-pyrazol-3-yl)phenyl)oxazole-5-carbonyl)-L-valinate;
N-(2-methylpentan-3-yl)-2-(3-(3-(pentan-3-ylcarbamoyl)-1 H-pyrazol-5-yl)phenyl)oxazole-5-carboxamide;
N-(pentan-3-yl)-2-(3-(5-(pentan-3-ylcarbamoyl)-1-(2-(piperidin-1-yl)ethyl)-1H-1,2,4-triazol-3-yl)phenyl)oxazole-5-carboxamide;
methyl (2-(3-(5-(((S)-1-cyclopropylethyl)carbamoyl)-1-(3,3,3-trifluoro-2-hydroxypropyl)-1 H-pyrazol-3-yl)phenyl)oxazole-5-carbonyl)-L-valinate;
2-(3-(5-(((S)-1-cyclopropylethyl)carbamoyl)-1-(3,3,3-trifluoro-2-hydroxypropyl)-1 H-pyrazol-3-yl)phenyl)-N-(pentan-3-yl)oxazole-5-carboxamide;
2-(3-(1-(2-methoxyethyl)-5-(pentan-3-ylcarbamoyl)-1 H-1,2,4-triazol-3-yl)phenyl)-N-(pentan-3-yl)oxazole-5-carboxamide;
ethyl (2-(3-(5-((dicyclopropylmethyl)carbamoyl)-1-(3-hydroxypropyl)-1 H-pyrazol-3-yl)phenyl)oxazole-5-carbonyl)-L-valinate;
2-(3-(1-(2-(benzylamino)ethyl)-5-(pentan-3-ylcarbamoyl)-1H-1,2,4-triazol-3-yl)phenyl)-N-(pentan-3-yl)oxazole-5-carboxamide;
ethyl (5-(3-(5-(pentan-3-ylcarbamoyl)oxazol-2-yl)phenyl)-1 H-pyrazole-3-carbonyl)-L-leucinate;
N-(pentan-3-yl)-2-(3-(3-((1-(tetrahydrofuran-2-yl)ethyl)carbamoyl)-1 H-pyrazol-5-yl)phenyl)oxazole-5-carboxamide;
tert-butyl (2-(3-(3-(pentan-3-ylcarbamoyl)-1H-pyrazol-5-yl)phenyl)oxazole-5-carbonyl)-L-valinate;
(S)-2-(3-(5-((1-cyclopropylethyl)carbamoyl)-1-(oxetan-3-yl)-1 H-pyrazol-3-yl)phenyl)-N-(pentan-3-yl)oxazole-5-carboxamide;
ethyl (2-(3-(5-((dicyclopropylmethyl)carbamoyl)-4H-1,2,4-triazol-3-yl)phenyl)oxazole-5-carbonyl)-L-valinate;
2-(3-(3-((1-cyanopropyl)carbamoyl)-1 H-pyrazol-5-yl)phenyl)-N-(pentan-3-yl)oxazole-5-carboxamide;
2-(3-(3-((1-cyclopropyl-2-methoxyethyl)carbamoyl)-1 H-pyrazol-5-yl)phenyl)-N-(pentan-3-yl)oxazole-5-carboxamide;
N-((S)-1-cyclopropylethyl)-2-(3-(5-(((S)-1-cyclopropylethyl)carbamoyl)-1-(3-hydroxypropyl)-1H-1,2,4-triazol-3-yl)phenyl)oxazole-5-carboxamide;
2-(3-(1-(2-(2-methoxyethoxy)ethyl)-5-(pentan-3-ylcarbamoyl)-1H-1,2,4-triazol-3-yl)phenyl)-N-(pentan-3-yl)oxazole-5-carboxamide;
(S)-2-(3-(5-((1-cyclopropylethyl)carbamoyl)-1-(2-isopropoxyethyl)-1 H-pyrazol-3-yl)phenyl)-N-(pentan-3-yl)oxazole-5-carboxamide;
methyl (2-(3-(5-((dicyclopropylmethyl)carbamoyl)-4H-1,2,4-triazol-3-yl)phenyl)oxazole-5-carbonyl)-L-valinate;
methyl (2-(3-(5-(((S)-1-cyclopropylethyl)carbamoyl)-1-(3,3,3-trifluoro-2-hydroxypropyl)-1 H-pyrazol-3-yl)phenyl)oxazole-5-carbonyl)-L-valinate;
benzyl (5-(3-(5-(pentan-3-ylcarbamoyl)oxazol-2-yl)phenyl)-1 H-pyrazole-3-carbonyl)-L-valinate;
ethyl (1-(2-(benzyloxy)ethyl)-3-(3-(5-(pentan-3-ylcarbamoyl)oxazol-2-yl)phenyl)-1 H-pyrazole-5-carbonyl)-L-valinate;
(S)-2-(3-(5-((1-cyclopropylethyl)carbamoyl)-1-methyl-1 H-pyrazol-3-yl)phenyl)-N-(pentan-3-yl)oxazole-5-carboxamide;
(S)-2-(3-(2-((1-cyclopropylethyl)carbamoyl)-1 H-imidazol-4-yl)phenyl)-N-(pentan-3-yl)oxazole-5-carboxamide;
ethyl (1-(2-((diethoxyphosphoryl)oxy)ethyl)-3-(3-(5-(pentan-3-ylcarbamoyl)oxazol-2-yl)phenyl)-1 H-pyrazole-5-carbonyl)-L-valinate;
(R)-2-(3-(5-((1-cyclopropylethyl)carbamoyl)-1-(2-hydroxyethyl)-1 H-pyrazol-3-yl)phenyl)-N-(pentan-3-yl)oxazole-5-carboxamide;
ethyl (2-(3-(5-(((S)-1-cyclopropylethyl)carbamoyl)-1-(2-hydroxyethyl)-1H-pyrazol-3-yl)phenyl)oxazole-5-carbonyl)-L-valinate;
methyl (2-(3-(5-(((S)-1-cyclopropylethyl)carbamoyl)-1-(3,3,3-trifluoro-2-hydroxypropyl)-1 H-pyrazol-3-yl)phenyl)oxazole-5-carbonyl)-L-valinate;
2-(3-(3-((cyclobutylmethyl)carbamoyl)-1 H-pyrazol-5-yl)phenyl)-N-(pentan-3-yl)oxazole-5-carboxamide;
tert-butyl O-(tert-butyl)-N-(5-(3-(5-(pentan-3-ylcarbamoyl)oxazol-2-yl)phenyl)-1 H-pyrazole-3-carbonyl)-L-serinate;
2-(3-(3-([1,1′-bi(cyclopropan)]-1-ylcarbamoyl)-1 H-pyrazol-5-yl)phenyl)-N-(pentan-3-yl)oxazole-5-carboxamide;
methyl (3-(3-(5-((dicyclopropylmethyl)carbamoyl)oxazol-2-yl)phenyl)-1 H-1,2,4-triazole-5-carbonyl)-L-valinate;
N-(pentan-3-yl)-2-(3-(4-(pentan-3-ylcarbamoyl)pyridin-2-yl)phenyl)oxazole-5-carboxamide;
N-(dicyclopropylmethyl)-2-(3-(5-((dicyclopropylmethyl)carbamoyl)-4H-1,2,4-triazol-3-yl)phenyl)oxazole-5-carboxamide;
N-((R)-1-cyclopropylethyl)-2-(3-(5-(((S)-1-cyclopropylethyl)carbamoyl)-1-(3,3,3-trifluoro-2-hydroxypropyl)-1H-pyrazol-3-yl)phenyl)oxazole-5-carboxamide
tert-butyl (2-(3-(5-(((S)-1-cyclopropylethyl)carbamoyl)-4H-1,2,4-triazol-3-yl)phenyl)oxazole-5-carbonyl)-L-valinate;
ethyl (2-(3-(5-((1,1,1-trifluorobutan-2-yl)carbamoyl)-1H-pyrazol-3-yl)phenyl)oxazole-5-carbonyl)-L-valinate;
2-(3-(3-((2-cyclopropylpropan-2-yl)carbamoyl)-1H-pyrazol-5-yl)phenyl)-N-(pentan-3-yl)oxazole-5-carboxamide;
2-(3-(3-((1-cyanopropyl)carbamoyl)-1 H-pyrazol-5-yl)phenyl)-N-(pentan-3-yl)oxazole-5-carboxamide;
ethyl (2-(3-(5-((1-cyclopropyl-2,2,2-trifluoroethyl)carbamoyl)-1 H-pyrazol-3-yl)phenyl)oxazole-5-carbonyl)-L-valinate;
ethyl (3-(3-(5-((dicyclopropylmethyl)carbamoyl)oxazol-2-yl)phenyl)-1-(2-hydroxy-2-methylpropyl)-1 H-pyrazole-5-carbonyl)-L-valinate;
2-(3-(3-((cyclopropyl(tetrahydrofuran-2-yl)methyl)carbamoyl)-1 H-pyrazol-5-yl)phenyl)-N-(pentan-3-yl)oxazole-5-carboxamide;
tert-butyl O-(tert-butyl)-N-(2-(3-(3-(pentan-3-ylcarbamoyl)-1 H-pyrazol-5-yl)phenyl)oxazole-5-carbonyl)-L-serinate;
2-(3-(5-(((S)-1-cyclopropylethyl)carbamoyl)-1-(3,3,3-trifluoro-2-hydroxypropyl)-1 H-pyrazol-3-yl)phenyl)-N-(dicyclopropylmethyl)oxazole-5-carboxamide;
methyl (2-(3-(5-((dicyclopropylmethyl)carbamoyl)-1 H-pyrazol-3-yl)phenyl)oxazole-5-carbonyl)-L-valinate;
ethyl (2-(3-(5-(((S)-1-cyclopropylethyl)carbamoyl)-4H-1,2,4-triazol-3-yl)phenyl)oxazole-5-carbonyl)-L-valinate;
N-(pentan-3-yl)-2-(3-(5-(pentan-3-ylcarbamoyl)-1 H-pyrazol-3-yl)phenyl)oxazole-5-carboxamide;
ethyl (2-(3-(3-(((S)-1-ethoxy-3-methyl-1-oxobutan-2-yl)carbamoyl)-1 H-pyrazol-5-yl)phenyl)oxazole-5-carbonyl)-L-valinate;
2-(3-(1-(2-hydroxyethyl)-5-(pentan-3-ylcarbamoyl)-1 H-pyrazol-3-yl)phenyl)-N-(2-methylpentan-3-yl)oxazole-5-carboxamide;
N-(tert-butyl)-2-(3-(3-(pentan-3-ylcarbamoyl)-1 H-pyrazol-5-yl)phenyl)oxazole-5-carboxamide;
ethyl (2-(3-(3-(pentan-3-ylcarbamoyl)-1 H-pyrazol-5-yl)phenyl)oxazole-5-carbonyl)-L-methioninate;
tert-butyl (5-(3-(5-(pentan-3-ylcarbamoyl)oxazol-2-yl)phenyl)-1 H-pyrazole-3-carbonyl)-L-leucylglycinate;
ethyl (1-(2-hydroxyethyl)-3-(3-(5-(pentan-3-ylcarbamoyl)oxazol-2-yl)phenyl)-1 H-pyrazole-5-carbonyl)-L-valinate;
(R)-2-(3-(3-((3-methylbutan-2-yl)carbamoyl)-1 H-pyrazol-5-yl)phenyl)-N-(pentan-3-yl)oxazole-5-carboxamide;
methyl (2-(3-(5-(((S)-1-methoxy-3-methyl-1-oxobutan-2-yl)carbamoyl)-1-(3,3,3-trifluoro-2-hydroxypropyl)-1 H-pyrazol-3-yl)phenyl)oxazole-5-carbonyl)-L-valinate;
N-(pentan-3-yl)-2-(3-(3-((2-phenylpropan-2-yl)carbamoyl)-1 H-pyrazol-5-yl)phenyl)oxazole-5-carboxamide;
2-(3-(3-((1-cyanopropyl)carbamoyl)-1 H-pyrazol-5-yl)phenyl)-N-(pentan-3-yl)oxazole-5-carboxamide;
2-(3-(3-(((R)-1-((2R,5R)-5-methyltetrahydrofuran-2-yl)propyl)carbamoyl)-1 H-pyrazol-5-yl)phenyl)-N-(pentan-3-yl)oxazole-5-carboxamide;
N-((R)-1-cyclopropylethyl)-2-(3-(5-(((S)-1-cyclopropylethyl)carbamoyl)-1-(2-hydroxyethyl)-1 H-pyrazol-3-yl)phenyl)oxazole-5-carboxamide;
ethyl (2-(3-(1-(2-(((S)-1-ethoxy-3-methyl-1-oxobutan-2-yl)amino)-2-oxoethyl)-5-(pentan-3-ylcarbamoyl)-1 H-pyrazol-3-yl)phenyl)oxazole-5-carbonyl)-L-valinate;
2-(3-(1-(2-hydroxyethyl)-5-(pentan-3-ylcarbamoyl)-1 H-pyrazol-3-yl)phenyl)-N-(p-tolyl)oxazole-5-carboxamide;
(S)-N-(1-cyclopropylethyl)-2-(3-(1-(2-hydroxyethyl)-5-(pentan-3-ylcarbamoyl)-1 H-pyrazol-3-yl)phenyl)oxazole-5-carboxamide;
ethyl (R)-2-(5-(3-(5-(pentan-3-ylcarbamoyl)oxazol-2-yl)phenyl)-1 H-pyrazole-3-carboxamido)-2-phenylacetate;
2-(3-(1-(2-(benzyloxy)ethyl)-5-(pentan-3-ylcarbamoyl)-1 H-1,2,4-triazol-3-yl)phenyl)-N-(pentan-3-yl)oxazole-5-carboxamide;
(S)-2-(3-(5-((1-cyclopropylethyl)carbamoyl)-1-(2-hydroxy-2-methylpropyl)-1 H-pyrazol-3-yl)phenyl)-N-(pentan-3-yl)oxazole-5-carboxamide;
methyl (2-(3-(5-((dicyclopropylmethyl)carbamoyl)-1-(2-hydroxy-2-methylpropyl)-1H-pyrazol-3-yl)phenyl)oxazole-5-carbonyl)-L-valinate;
ethyl (2-(3-(1-(4-(tert-butoxy)-4-oxobutyl)-5-(pentan-3-ylcarbamoyl)-1 H-pyrazol-3-yl)phenyl)oxazole-5-carbonyl)-L-valinate;
diethyl 2,2′-((2,2′-(1,3-phenylene)bis(oxazole-2,5-diyl-5-carbonyl))bis(azanediyl))(2S,2'S)-bis(3-methylbutanoate);
2-(3-(5-(((S)-1-cyclopropylethyl)carbamoyl)-1-(2-hydroxypropyl)-1 H-pyrazol-3-yl)phenyl)-N-(pentan-3-yl)oxazole-5-carboxamide;
2-(3-(5-(((S)-1-cyclopropylethyl)carbamoyl)-1-(2,3-dihydroxypropyl)-1 H-pyrazol-3-yl)phenyl)-N-(pentan-3-yl)oxazole-5-carboxamide;
2-(3-(1-(2-(isoindolin-2-yl)ethyl)-5-(pentan-3-ylcarbamoyl)-1 H-1,2,4-triazol-3-yl)phenyl)-N-(pentan-3-yl)oxazole-5-carboxamide;
methyl (S)-3-cyclohexyl-2-(2-(3-(3-((dicyclopropylmethyl)carbamoyl)-1 H-pyrazol-5-yl)phenyl)oxazole-5-carboxamido)propanoate;
N-((S)-1-cyclopropylethyl)-2-(3-(5-(((S)-1-cyclopropylethyl)carbamoyl)-1-(2-hydroxyethyl)-1 H-1,2,4-triazol-3-yl)phenyl)oxazole-5-carboxamide;
N-(pentan-3-yl)-2-(3-(3-(((1S)-1-(tetrahydrofuran-2-yl)ethyl)carbamoyl)-1 H-pyrazol-5-yl)phenyl)oxazole-5-carboxamide;
N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide;
N-(pentan-3-yl)-2-(3-(3-(((S)-1-((R)-tetrahydrofuran-2-yl)ethyl)carbamoyl)-1 H-pyrazol-5-yl)phenyl)oxazole-5-carboxamide
ethyl (2-(3-(1-(3-(tert-butoxy)-3-oxopropyl)-5-(pentan-3-ylcarbamoyl)-1 H-pyrazol-3-yl)phenyl)oxazole-5-carbonyl)-L-valinate;
ethyl (2-(3-(1-(3-(tert-butoxy)-3-oxopropyl)-5-(((S)-1-cyclopropylethyl)carbamoyl)-1 H-pyrazol-3-yl)phenyl)oxazole-5-carbonyl)-L-valinate;
N-((R)-1-cyclopropylethyl)-2-(3-(5-(((R)-1-cyclopropylethyl)carbamoyl)-1-(2-hydroxyethyl)-1 H-pyrazol-3-yl)phenyl)oxazole-5-carboxamide;
ethyl (2-(3-(3-(pentan-3-ylcarbamoyl)-1 H-pyrazol-5-yl)phenyl)oxazole-5-carbonyl)-L-valinate;
(S)-N-(pentan-3-yl)-2-(3-(3-((1-phenylethyl)carbamoyl)-1 H-pyrazol-5-yl)phenyl)oxazole-5-carboxamide;
methyl (S)-2-(2-(3-(3-(pentan-3-ylcarbamoyl)-1 H-pyrazol-5-yl)phenyl)oxazole-5-carboxamido)-2-phenylacetate;
tert-butyl (2-(3-(3-(pentan-3-ylcarbamoyl)-1 H-pyrazol-5-yl)phenyl)oxazole-5-carbonyl)-L-leucylglycinate;
ethyl (2-(3-(5-((dicyclopropylmethyl)carbamoyl)-1-(2-hydroxy-2-methylpropyl)-1 H-pyrazol-3-yl)phenyl)oxazole-5-carbonyl)-L-valinate;
2,2′-(2-methyl-1,3-phenylene)bis(N-(pentan-3-yl)oxazole-5-carboxamide);
methyl (2-(3-(5-(((S)-1-cyclopropylethyl)carbamoyl)-4H-1,2,4-triazol-3-yl)phenyl)oxazole-5-carbonyl)-L-leucinate;
ethyl (2-(3-(5-((1-cyclopropyl-2,2-difluoroethyl)carbamoyl)-1H-pyrazol-3-yl)phenyl)oxazole-5-carbonyl)-L-valinate;
2-(3-(3-((2-cyclopropyl-1,1,1-trifluoropropan-2-yl)carbamoyl)-1 H-pyrazol-5-yl)phenyl)-N-(pentan-3-yl)oxazole-5-carboxamide;
methyl (2-(3-(3-(((R)-1-cyclopropylethyl)carbamoyl)-1H-pyrazol-5-yl)phenyl)oxazole-5-carbonyl)-L-valinate;
2-(3-(3-((2-methyl-4-phenylbutan-2-yl)carbamoyl)-1 H-pyrazol-5-yl)phenyl)-N-(pentan-3-yl)oxazole-5-carboxamide;
ethyl (2-(3-(5-((1-cyclopropyl-2,2,2-trifluoroethyl)carbamoyl)-1 H-pyrazol-3-yl)phenyl)oxazole-5-carbonyl)-L-valinate;
N-(pentan-3-yl)-2-(3-(5-(pentan-3-ylcarbamoyl)-1-(2-(piperidin-1-yl)ethyl)-1 H-pyrazol-3-yl)phenyl)oxazole-5-carboxamide;
2,2′-(1,3-phenylene)bis(N-(pentan-3-yl)oxazole-5-carboxamide);
2-(3-(3-((1-methoxy-3-methylbutan-2-yl)carbamoyl)-1 H-pyrazol-5-yl)phenyl)-N-(pentan-3-yl)oxazole-5-carboxamide;
ethyl (5-(3-(5-(((S)-1-cyclopropylethyl)carbamoyl)oxazol-2-yl)phenyl)-4H-1,2,4-triazole-3-carbonyl)-L-valinate;
ethyl (2-(3-(3-((1-cyclopropyl-2,2,2-trifluoroethyl)carbamoyl)-1H-1,2,4-triazol-5-yl)phenyl)oxazole-5-carbonyl)-L-valinate;
2-(3-(3-((2-isopropoxyethyl)carbamoyl)-1 H-pyrazol-5-yl)phenyl)-N-(pentan-3-yl)oxazole-5-carboxamide;
2-(3-(3-(cyclohexylcarbamoyl)-1 H-pyrazol-5-yl)phenyl)-N-(pentan-3-yl)oxazole-5-carboxamide;
N-(pentan-3-yl)-2-(3-(5-(pentan-3-ylcarbamoyl)-4H-1,2,4-triazol-3-yl)phenyl)oxazole-5-carboxamide;
ethyl 4-(5-(pentan-3-ylcarbamoyl)-3-(3-(5-(pentan-3-ylcarbamoyl)oxazol-2-yl)phenyl)-1 H-1,2,4-triazol-1-yl)butanoate;
2-(3-(3-((1-cyclobutylethyl)carbamoyl)-1 H-pyrazol-5-yl)phenyl)-N-(pentan-3-yl)oxazole-5-carboxamide;
ethyl (5-(3-(5-(pentan-3-ylcarbamoyl)oxazol-2-yl)phenyl)-1 H-pyrazole-3-carbonyl)-L-valinate;
N-(4-fluorobenzyl)-2-(3-(3-(pentan-3-ylcarbamoyl)-1 H-pyrazol-5-yl)phenyl)oxazole-5-carboxamide;
2-(3-(3-((2-methylpentan-3-yl)carbamoyl)-1 H-pyrazol-5-yl)phenyl)-N-(pentan-3-yl)oxazole-5-carboxamide;
(R)-N-(1-cyclopropylethyl)-2-(3-(5-(pentan-3-ylcarbamoyl)-1 H-pyrazol-3-yl)phenyl)oxazole-5-carboxamide;
N-(pentan-3-yl)-2-(3-(3-(((1S)-1-(tetrahydrofuran-2-yl)ethyl)carbamoyl)-1 H-pyrazol-5-yl)phenyl)oxazole-5-carboxamide;
N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide;
N-(pentan-3-yl)-2-(3-(3-(((S)-1-((R)-tetrahydrofuran-2-yl)ethyl)carbamoyl)-1H-pyrazol-5-yl)phenyl)oxazole-5-carboxamide;
methyl (S)-2-(2-(3-(5-(((S)-1-cyclopropylethyl)carbamoyl)-4H-1,2,4-triazol-3-yl)phenyl)oxazole-5-carboxamido)-3,3-dimethylbutanoate;
(S)-2-(3-(5-((1-cyclopropylethyl)carbamoyl)-4H-1,2,4-triazol-3-yl)phenyl)-N-(dicyclopropylmethyl)oxazole-5-carboxamide;
methyl (2-(3-(5-(((S)-1-cyclopropylethyl)carbamoyl)-4H-1,2,4-triazol-3-yl)phenyl)oxazole-5-carbonyl)-L-valinate;
(S)-2-(3-(3-(pentan-3-ylcarbamoyl)-1 H-pyrazol-5-yl)phenyl)-N-(1-phenylethyl)oxazole-5-carboxamide;
isopropyl (2-(3-(5-(((S)-1-cyclopropylethyl)carbamoyl)-4H-1,2,4-triazol-3-yl)phenyl)oxazole-5-carbonyl)-L-valinate;
(S)-2-(3-(3-((1-methoxypropan-2-yl)carbamoyl)-1 H-pyrazol-5-yl)phenyl)-N-(pentan-3-yl)oxazole-5-carboxamide;
methyl (2-(3-(3-(pentan-3-ylcarbamoyl)-1 H-pyrazol-5-yl)phenyl)oxazole-5-carbonyl)-L-leucinate;
(S)-N-(1-cyclopropylethyl)-2-(3-(5-((dicyclopropylmethyl)carbamoyl)-4H-1,2,4-triazol-3-yl)phenyl)oxazole-5-carboxamide;
N-(1-cyclopropylethyl)-2-(3-(5-((1-cyclopropylethyl)carbamoyl)-1-(2-hydroxyethyl)-1 H-pyrazol-3-yl)phenyl)oxazole-5-carboxamide;
ethyl (2-(3-(1-(2-morpholinoethyl)-5-(pentan-3-ylcarbamoyl)-1 H-pyrazol-3-yl)phenyl)oxazole-5-carbonyl)-L-valinate;
(S)-2-(3-(3-((1-cyclopropylethyl)carbamoyl)-1 H-pyrazol-5-yl)phenyl)-N-(pentan-3-yl)oxazole-5-carboxamide;
(S)-2-(3-(5-((1-cyclopropylethyl)carbamoyl)-1 H-pyrazol-3-yl)phenyl)-N-(dicyclopropylmethyl)oxazole-5-carboxamide;
methyl 1-(5-(3-(5-(pentan-3-ylcarbamoyl)oxazol-2-yl)phenyl)-1 H-pyrazole-3-carbonyl)pyrrolidine-3-carboxylate;
N-(heptan-4-yl)-2-(3-(3-(pentan-3-ylcarbamoyl)-1 H-pyrazol-5-yl)phenyl)oxazole-5-carboxamide;
2-(3-(3-(heptan-4-ylcarbamoyl)-1 H-pyrazol-5-yl)phenyl)-N-(pentan-3-yl)oxazole-5-carboxamide;
2-(3-(3-((cyclopropyl(tetrahydrofuran-2-yl)methyl)carbamoyl)-1 H-pyrazol-5-yl)phenyl)-N-(pentan-3-yl)oxazole-5-carboxamide;
N-((S)-1-cyclopropylethyl)-2-(3-(5-(((S)-1-cyclopropylethyl)carbamoyl)-1-(2-hydroxyethyl)-1 H-pyrazol-3-yl)phenyl)oxazole-5-carboxamide;
methyl (5-(3-(5-(pentan-3-ylcarbamoyl)oxazol-2-yl)phenyl)-1 H-pyrazole-3-carbonyl)-L-valinate;
(S)-2-(3-(4-((1-cyclopropylethyl)carbamoyl)thiazol-2-yl)phenyl)-N-(pentan-3-yl)oxazole-5-carboxamide;
2-(3-(5-((cyclohexylmethyl)carbamoyl)-4H-1,2,4-triazol-3-yl)phenyl)-N-(pentan-3-yl)oxazole-5-carboxamide;
N-(2-methyl-4-phenylbutan-2-yl)-2-(3-(3-(pentan-3-ylcarbamoyl)-1 H-pyrazol-5-yl)phenyl)oxazole-5-carboxamide;
ethyl 6-(5-(pentan-3-ylcarbamoyl)-3-(3-(5-(pentan-3-ylcarbamoyl)oxazol-2-yl)phenyl)-1 H-1,2,4-triazol-1-yl)hexanoate;
(S)-2-(3-(5-((1-cyclopropylethyl)carbamoyl)-1-(2-hydroxyethyl)-1 H-pyrazol-3-yl)phenyl)-N-(pentan-3-yl)oxazole-5-carboxamide;
(R)-2-(3-(5-((1-cyclopropylethyl)carbamoyl)-4H-1,2,4-triazol-3-yl)phenyl)-N-(dicyclopropylmethyl)oxazole-5-carboxamide;
ethyl (2-(3-(5-(((R)-1-methoxy-3-methyl-1-oxobutan-2-yl)carbamoyl)-1H-pyrazol-3-yl)phenyl)oxazole-5-carbonyl)-L-valinate;
methyl (5-(3-(5-(pentan-3-ylcarbamoyl)oxazol-2-yl)phenyl)-1 H-pyrazole-3-carbonyl)-L-leucinate;
2-(3-(3-(2-isopropylpyrrolidine-1-carbonyl)-1H-pyrazol-5-yl)phenyl)-N-(pentan-3-yl)oxazole-5-carboxamide;
ethyl (2-(3-(5-((dicyclopropylmethyl)carbamoyl)-1H-pyrazol-3-yl)phenyl)oxazole-5-carbonyl)-L-valinate;
2-(3-(3-(pentan-3-ylcarbamoyl)-1H-pyrazol-5-yl)phenyl)-N-(3-(trifluoromethyl)phenyl)oxazole-5-carboxamide;
ethyl (2-(3-(3-(pentan-3-ylcarbamoyl)-1H-pyrazol-5-yl)phenyl)oxazole-5-carbonyl)-L-leucinate;
N-(3-cyanophenyl)-2-(3-(3-(pentan-3-ylcarbamoyl)-1 H-pyrazol-5-yl)phenyl)oxazole-5-carboxamide
2-(3-(1-(2-(benzyloxy)ethyl)-5-(pentan-3-ylcarbamoyl)-1 H-pyrazol-3-yl)phenyl)-N-(pentan-3-yl)oxazole-5-carboxamide;
ethyl (2-(3-(5-((1-cyclopropyl-2,2,2-trifluoroethyl)carbamoyl)-1 H-pyrazol-3-yl)phenyl)oxazole-5-carbonyl)-L-valinate;
methyl (5-(3-(5-(pentan-3-ylcarbamoyl)oxazol-2-yl)phenyl)-1 H-pyrazole-3-carbonyl)-L-alaninate;
2-(3-(1-(2-hydroxyethyl)-5-(pentan-3-ylcarbamoyl)-1 H-pyrazol-3-yl)phenyl)-N-(pentan-3-yl)oxazole-5-carboxamide;
ethyl (2-(3-(1-(4-(tert-butoxy)-4-oxobutyl)-5-(((S)-1-cyclopropylethyl)carbamoyl)-1 H-pyrazol-3-yl)phenyl)oxazole-5-carbonyl)-L-valinate;
(S)-N-(adamantan-1-yl)-2-(3-(5-((1-cyclopropylethyl)carbamoyl)-4H-1,2,4-triazol-3-yl)phenyl)oxazole-5-carboxamide;
ethyl (R)-2-(2-(3-(3-(pentan-3-ylcarbamoyl)-1 H-pyrazol-5-yl)phenyl)oxazole-5-carboxamido)-2-phenylacetate;
methyl (5-(3-(5-(pentan-3-ylcarbamoyl)oxazol-2-yl)phenyl)-1 H-pyrazole-3-carbonyl)phenylalaninate;
2-(3-(3-(tert-butylcarbamoyl)-1 H-pyrazol-5-yl)phenyl)-N-(pentan-3-yl)oxazole-5-carboxamide;
2-(3-(5-(((S)-1-cyclopropylethyl)carbamoyl)-1-(3,3,3-trifluoro-2-hydroxypropyl)-1 H-pyrazol-3-yl)phenyl)-N-(dicyclopropylmethyl)oxazole-5-carboxamide;
(S)-N-(1-cyclohexylethyl)-2-(3-(3-(pentan-3-ylcarbamoyl)-1 H-pyrazol-5-yl)phenyl)oxazole-5-carboxamide;
methyl N-(2-(3-(3-(((S)-1-cyclopropylethyl)carbamoyl)-1 H-pyrazol-5-yl)phenyl)oxazole-5-carbonyl)-S-methyl-D-cysteinate;
2-(3-(4-(2-methoxyethyl)-5-(pentan-3-ylcarbamoyl)-4H-1,2,4-triazol-3-yl)phenyl)-N-(pentan-3-yl)oxazole-5-carboxamide;
N-cyclopentyl-2-(3-(3-(pentan-3-ylcarbamoyl)-1 H-pyrazol-5-yl)phenyl)oxazole-5-carboxamide;
methyl (5-(3-(5-(((S)-1-ethoxy-3-methyl-1-oxobutan-2-yl)carbamoyl)oxazol-2-yl)phenyl)-1 H-pyrazole-3-carbonyl)-L-leucinate;
(R)-2-(3-(3-((1-cyclohexylethyl)carbamoyl)-1 H-pyrazol-5-yl)phenyl)-N-(pentan-3-yl)oxazole-5-carboxamide;
N-(3,5-dimethylphenyl)-2-(3-(3-(pentan-3-ylcarbamoyl)-1 H-pyrazol-5-yl)phenyl)oxazole-5-carboxamide;
(S)-2-(3-(4-((1-cyclopropylethyl)carbamoyl)-1 H-imidazol-2-yl)phenyl)-N-(pentan-3-yl)oxazole-5-carboxamide;
ethyl 3-methyl-1-(5-(3-(5-(pentan-3-ylcarbamoyl)oxazol-2-yl)phenyl)-1H-pyrazole-3-carbonyl)pyrrolidine-3-carboxylate;
ethyl (2-(3-(3-(((R)-1-cyclopropylethyl)carbamoyl)-1H-pyrazol-5-yl)phenyl)oxazole-5-carbonyl)-L-valinate;
tert-butyl (S)-2-(2-(3-(3-(pentan-3-ylcarbamoyl)-1 H-pyrazol-5-yl)phenyl)oxazole-5-carboxamido)-2-phenylacetate;
ethyl (2-(3-(1-(2-hydroxyethyl)-5-(pentan-3-ylcarbamoyl)-1 H-pyrazol-3-yl)phenyl)oxazole-5-carbonyl)-L-valinate;
2-(3-(3-((4-fluorobenzyl)carbamoyl)-1 H-pyrazol-5-yl)phenyl)-N-(pentan-3-yl)oxazole-5-carboxamide;
2-(3-(5-(((S)-1-cyclopropylethyl)carbamoyl)-1-((2,2-dimethyl-1,3-dioxolan-4-yl)methyl)-1H-pyrazol-3-yl)phenyl)-N-(pentan-3-yl)oxazole-5-carboxamide;
ethyl 2-(5-(pentan-3-ylcarbamoyl)-3-(3-(5-(pentan-3-ylcarbamoyl)oxazol-2-yl)phenyl)-1 H-1,2,4-triazol-1-yl)acetate;
2-(3-(5-(((S)-1-cyclopropylethyl)carbamoyl)-4H-1,2,4-triazol-3-yl)phenyl)-N-((S)-3-methylbutan-2-yl)oxazole-5-carboxamide;
methyl (5-(3-(5-(pentan-3-ylcarbamoyl)oxazol-2-yl)phenyl)-1 H-pyrazole-3-carbonyl)-D-methioninate;
N-((R)-1-cyclopropylethyl)-2-(3-(5-(((S)-1-cyclopropylethyl)carbamoyl)-4H-1,2,4-triazol-3-yl)phenyl)oxazole-5-carboxamide;
(S)-N-(1-cyclopropylethyl)-2-(3-(5-((4,4-difluorocyclohexyl)carbamoyl)-4H-1,2,4-triazol-3-yl)phenyl)oxazole-5-carboxamide;
ethyl (5-(3-(5-(pentan-3-ylcarbamoyl)oxazol-2-yl)phenyl)-1 H-pyrazole-3-carbonyl)-L-phenylalaninate;
ethyl (2-(3-(3-(((S)-1-cyclopropylethyl)carbamoyl)-1 H-pyrazol-5-yl)phenyl)oxazole-5-carbonyl)-L-valinate;
N-(pentan-3-yl)-2-(3-(3-(((1S)-1-(tetrahydrofuran-2-yl)ethyl)carbamoyl)-1 H-pyrazol-5-yl)phenyl)oxazole-5-carboxamide;
N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide;
N-(pentan-3-yl)-2-(3-(3-(((S)-1-((R)-tetrahydrofuran-2-yl)ethyl)carbamoyl)-1 H-pyrazol-5-yl)phenyl)oxazole-5-carboxamide;
diallyl 2,2′-((2,2′-(1,3-phenylene)bis(oxazole-2,5-diyl-5-carbonyl))bis(azanediyl))(2S,2'S)-bis(3-methylbutanoate);
2-(3-(3-((2-(tert-butylthio)ethyl)carbamoyl)-1 H-pyrazol-5-yl)phenyl)-N-(pentan-3-yl)oxazole-5-carboxamide;
(R)-2-(3-(5-((1-cyclopropylethyl)carbamoyl)-1 H-pyrazol-3-yl)phenyl)-N-(pentan-3-yl)oxazole-5-carboxamide;
2-(3-(3-(pentan-3-ylcarbamoyl)-1 H-pyrazol-5-yl)phenyl)-N-((tetrahydro-2H-pyran-2-yl)methyl)oxazole-5-carboxamide;
N-(pentan-3-yl)-2-(3-(4-(pentan-3-ylcarbamoyl)-1 H-imidazol-2-yl)phenyl)oxazole-5-carboxamide;
N-((R)-1-cyclopropylethyl)-2-(3-(3-(((R)-1-cyclopropylethyl)carbamoyl)-1H-1,2,4-triazol-5-yl)phenyl)oxazole-5-carboxamide;
isopropyl (2-(3-(3-(pentan-3-ylcarbamoyl)-1H-pyrazol-5-yl)phenyl)oxazole-5-carbonyl)glycinate;
methyl (S)-3-cyclohexyl-2-(2-(3-(5-(((S)-1-cyclopropylethyl)carbamoyl)-4H-1,2,4-triazol-3-yl)phenyl)oxazole-5-carboxamido)propanoate;
methyl (2-(3-(3-(((S)-1-methoxy-4-methyl-1-oxopentan-2-yl)carbamoyl)-1 H-pyrazol-5-yl)phenyl)oxazole-5-carbonyl)-L-leucinate;
2-(3-(3-((2,6-difluorobenzyl)carbamoyl)-1 H-pyrazol-5-yl)phenyl)-N-(pentan-3-yl)oxazole-5-carboxamide;
2-(3-(3-(4-methoxy-4-methylpiperidine-1-carbonyl)-1 H-pyrazol-5-yl)phenyl)-N-(pentan-3-yl)oxazole-5-carboxamide;
(S)-2-(3-(3-(sec-butylcarbamoyl)-1 H-pyrazol-5-yl)phenyl)-N-(pentan-3-yl)oxazole-5-carboxamide;
2-(3-(3-((2-methoxy-2-methylpropyl)carbamoyl)-1 H-pyrazol-5-yl)phenyl)-N-(pentan-3-yl)oxazole-5-carboxamide;
tert-butyl 2-methyl-2-(2-(3-(3-(pentan-3-ylcarbamoyl)-1 H-pyrazol-5-yl)phenyl)oxazole-5-carboxamido)propanoate;
methyl (2-(3-(3-(pentan-3-ylcarbamoyl)-1 H-pyrazol-5-yl)phenyl)oxazole-5-carbonyl)-L-valinate;
benzyl (2-(3-(3-(pentan-3-ylcarbamoyl)-1 H-pyrazol-5-yl)phenyl)oxazole-5-carbonyl)-L-alaninate;
tert-butyl (5-(3-(5-(pentan-3-ylcarbamoyl)oxazol-2-yl)phenyl)-1 H-pyrazole-3-carbonyl)-L-valinate;
methyl (R)-2-(5-(3-(5-(pentan-3-ylcarbamoyl)oxazol-2-yl)phenyl)-1 H-pyrazole-3-carboxamido)-2-phenylacetate;
(S)-N-(sec-butyl)-2-(3-(3-(pentan-3-ylcarbamoyl)-1 H-pyrazol-5-yl)phenyl)oxazole-5-carboxamide;
2-(3-(3-((3-isopropoxyphenyl)carbamoyl)-1 H-pyrazol-5-yl)phenyl)-N-(pentan-3-yl)oxazole-5-carboxamide;
N-((S)-1-cyclopropylethyl)-2-(3-(5-(((R)-1-cyclopropylethyl)carbamoyl)-4H-1,2,4-triazol-3-yl)phenyl)oxazole-5-carboxamide;
2-(3-(3-(cyclopentylcarbamoyl)-1 H-pyrazol-5-yl)phenyl)-N-(pentan-3-yl)oxazole-5-carboxamide;
N-((S)-1-cyclopropylethyl)-2-(3-(5-(((S)-1-cyclopropylethyl)carbamoyl)-1-(2-hydroxy-2-methylpropyl)-1 H-pyrazol-3-yl)phenyl)oxazole-5-carboxamide;
2-(3-(3-((cyclopropyl(tetrahydrofuran-2-yl)methyl)carbamoyl)-1 H-pyrazol-5-yl)phenyl)-N-(pentan-3-yl)oxazole-5-carboxamide;
tert-butyl 2-(5-(pentan-3-ylcarbamoyl)-3-(3-(5-(pentan-3-ylcarbamoyl)oxazol-2-yl)phenyl)-1 H-1,2,4-triazol-1-yl)acetate;
ethyl (5-(3-(5-(pentan-3-ylcarbamoyl)oxazol-2-yl)phenyl)-1 H-pyrazole-3-carbonyl)-L-alaninate;
2-(3-(3-(3,3-dimethylpiperidine-1-carbonyl)-1 H-pyrazol-5-yl)phenyl)-N-(pentan-3-yl)oxazole-5-carboxamide;
(S)-N-([1,1′-bi(cyclopropan)]-1-yl)-2-(3-(5-((1-cyclopropylethyl)carbamoyl)-4H-1,2,4-triazol-3-yl)phenyl)oxazole-5-carboxamide;
benzyl (5-(3-(5-(pentan-3-ylcarbamoyl)oxazol-2-yl)phenyl)-1 H-pyrazole-3-carbonyl)-L-alaninate;
(S)-2-(3-(3-((1-cyclohexylethyl)carbamoyl)-1 H-pyrazol-5-yl)phenyl)-N-(pentan-3-yl)oxazole-5-carboxamide;
N-((1-methylcyclohexyl)methyl)-2-(3-(3-(pentan-3-ylcarbamoyl)-1 H-pyrazol-5-yl)phenyl)oxazole-5-carboxamide;
(R)-N-(1-cyclohexylethyl)-2-(3-(3-(pentan-3-ylcarbamoyl)-1 H-pyrazol-5-yl)phenyl)oxazole-5-carboxamide;
methyl (5-(3-(5-(pentan-3-ylcarbamoyl)oxazol-2-yl)phenyl)-1 H-pyrazole-3-carbonyl)-L-phenylalaninate;
tert-butyl 1-(5-(3-(5-(pentan-3-ylcarbamoyl)oxazol-2-yl)phenyl)-1 H-pyrazole-3-carbonyl)pyrrolidine-3-carboxylate;
methyl (S)-1-(2-(3-(5-((1-cyclopropylethyl)carbamoyl)-1 H-pyrazol-3-yl)phenyl)oxazole-5-carboxamido)cyclobutane-1-carboxylate;
N-(2,6-difluorobenzyl)-2-(3-(3-(pentan-3-ylcarbamoyl)-1 H-pyrazol-5-yl)phenyl)oxazole-5-carboxamide;
2-(3-(3-(((1-methylcyclopropyl)methyl)carbamoyl)-1 H-pyrazol-5-yl)phenyl)-N-(pentan-3-yl)oxazole-5-carboxamide;
ethyl (2-(3-(5-(pentan-3-ylcarbamoyl)-1 H-pyrazol-3-yl)phenyl)oxazole-5-carbonyl)-D-valinate;
N-benzyl-2-(3-(3-(pentan-3-ylcarbamoyl)-1 H-pyrazol-5-yl)phenyl)oxazole-5-carboxamide;
methyl (2-(3-(3-(((S)-1-cyclopropylethyl)carbamoyl)-1 H-pyrazol-5-yl)phenyl)oxazole-5-carbonyl)-L-valinate;
(S)-2-(3-(3-((3,3-dimethylbutan-2-yl)carbamoyl)-1 H-pyrazol-5-yl)phenyl)-N-(pentan-3-yl)oxazole-5-carboxamide;
ethyl (2-(3-(1-(2-(tert-butoxy)-2-oxoethyl)-5-(pentan-3-ylcarbamoyl)-1 H-pyrazol-3-yl)phenyl)oxazole-5-carbonyl)-L-valinate;
ethyl (2-(3-(5-((1,1,1-trifluoropropan-2-yl)carbamoyl)-1H-pyrazol-3-yl)phenyl)oxazole-5-carbonyl)-L-valinate;
(R)-N-(3-methylbutan-2-yl)-2-(3-(3-(pentan-3-ylcarbamoyl)-1 H-pyrazol-5-yl)phenyl)oxazole-5-carboxamide;
2-(3-(3-(((1-morpholinocyclohexyl)methyl)carbamoyl)-1 H-pyrazol-5-yl)phenyl)-N-(pentan-3-yl)oxazole-5-carboxamide;
(R)-N-(pentan-3-yl)-2-(3-(3-((1-phenylethyl)carbamoyl)-1 H-pyrazol-5-yl)phenyl)oxazole-5-carboxamide;
N-(pentan-3-yl)-2-(3-(3-((3-(trifluoromethoxy)phenyl)carbamoyl)-1 H-pyrazol-5-yl)phenyl)oxazole-5-carboxamide;
2-(3-(3-(benzylcarbamoyl)-1 H-pyrazol-5-yl)phenyl)-N-(pentan-3-yl)oxazole-5-carboxamide;
2-(3-(5-(((S)-1-cyclopropylethyl)carbamoyl)-1 H-pyrazol-3-yl)phenyl)-N-(1-cyclopropylpropyl)oxazole-5-carboxamide;
ethyl (S)-3-cyclohexyl-2-(5-(3-(5-(pentan-3-ylcarbamoyl)oxazol-2-yl)phenyl)-1 H-pyrazole-3-carboxamido)propanoate;
2-(3-(3-((cyclohexylmethyl)carbamoyl)-1 H-pyrazol-5-yl)phenyl)-N-(pentan-3-yl)oxazole-5-carboxamide;
N-(3-chlorophenyl)-2-(3-(3-(pentan-3-ylcarbamoyl)-1 H-pyrazol-5-yl)phenyl)oxazole-5-carboxamide;
methyl (R)-2-(2-(3-(3-(pentan-3-ylcarbamoyl)-1 H-pyrazol-5-yl)phenyl)oxazole-5-carboxamido)-2-phenylacetate;
2,2′-(4-fluoro-1,3-phenylene)bis(N-(pentan-3-yl)oxazole-5-carboxamide);
N-(benzo[d][1,3]dioxol-5-ylmethyl)-2-(3-(3-(pentan-3-ylcarbamoyl)-1 H-pyrazol-5-yl)phenyl)oxazole-5-carboxamide;
ethyl 2-methyl-2-(2-(3-(3-(pentan-3-ylcarbamoyl)-1 H-pyrazol-5-yl)phenyl)oxazole-5-carboxamido)propanoate;
tert-butyl 2-(5-(pentan-3-ylcarbamoyl)-3-(3-(5-(pentan-3-ylcarbamoyl)oxazol-2-yl)phenyl)-1 H-1,2,4-triazol-1-yl)acetate;
N-((S)-1-cyclopropylethyl)-2-(3-(5-(((S)-1-cyclopropylethyl)carbamoyl)-4H-1,2,4-triazol-3-yl)phenyl)oxazole-5-carboxamide;
ethyl (S)-2-(2-(3-(3-(pentan-3-ylcarbamoyl)-1 H-pyrazol-5-yl)phenyl)oxazole-5-carboxamido)-2-phenylacetate;
N-(isoxazol-3-yl)-2-(3-(3-(pentan-3-ylcarbamoyl)-1 H-pyrazol-5-yl)phenyl)oxazole-5-carboxamide;
N-(1-cyclopropylethyl)-2-(3-(5-((1-cyclopropylethyl)carbamoyl)-1 H-pyrazol-3-yl)phenyl)oxazole-5-carboxamide;
(S)-N-(1-cyclopropylethyl)-2-(3-(3-(pentan-3-ylcarbamoyl)-1 H-pyrazol-5-yl)phenyl)oxazole-5-carboxamide;
N-(pentan-3-yl)-2-(3-(3-(piperidine-1-carbonyl)-1 H-pyrazol-5-yl)phenyl)oxazole-5-carboxamide;
ethyl (2-(3-(3-(pentan-3-ylcarbamoyl)-1 H-pyrazol-5-yl)phenyl)oxazole-5-carbonyl)-L-phenylalaninate; and
2-(3-(3-((benzo[d][1,3]dioxol-5-ylmethyl)carbamoyl)-1 H-pyrazol-5-yl)phenyl)-N-(pentan-3-yl)oxazole-5-carboxamide;
or a pharmaceutically acceptable salt, hydrate, or co-crystal thereof.

18. A pharmaceutical composition comprising a compound of claim 1 or a pharmaceutically acceptable salt, hydrate, or co-crystal thereof, and a pharmaceutically acceptable carrier, or diluent.

19. The pharmaceutical composition of claim 18, further comprising one or more additional pharmaceutical agent(s).

20. The pharmaceutical composition of claim 19, wherein the one or more additional pharmaceutical agent(s) is selected from a mucolytic agent(s), nebulized hypertonic saline, bronchodilator(s), an antibiotic(s), an anti-infective agent(s), a CFTR modulator(s), and an anti-inflammatory agent(s).

21. The pharmaceutical composition of claim 19, wherein the one or more additional pharmaceutical agent(s) is a CFTR modulator(s).

22. The pharmaceutical composition of claim 19, wherein the one or more additional pharmaceutical agent(s) is a CFTR corrector(s).

23. The pharmaceutical composition of claim 19, wherein the one or more additional pharmaceutical agent(s) is a CFTR potentiator(s).

24. The pharmaceutical composition of claim 19, wherein the one or more additional pharmaceutical agent(s) comprise a CFTR amplifier(s).

25. A method for treating a disease associated with impaired mucociliary clearance in a subject comprising administering to the subject a compound or a pharmaceutically acceptable salt, hydrate, or co-crystal thereof of claim 1.

26. The method of claim 25, wherein the disease associated with impaired mucociliary clearance is selected from cystic fibrosis, asthma, bronchiectasis, COPD, and chronic bronchitis.

27. The method of claim 26, wherein the disease associated with impaired mucociliary clearance is cystic fibrosis, or COPD.

28. The method of claim 26, wherein the disease associated with impaired mucociliary clearance is cystic fibrosis.

29. The method of claim 25, further comprising administering to the subject one or more additional pharmaceutical agent(s) prior to, concurrent with, or subsequent to said compound.

30. The method of claim 29, wherein the one or more additional pharmaceutical agent(s) is selected from a mucolytic agent(s), nebulized hypertonic saline, bronchodilator(s), an antibiotic(s), an anti-infective agent(s), a CFTR modulator(s), and an anti-inflammatory agent(s).

31. The method of claim 29, wherein the one or more additional pharmaceutical agent(s) is a CFTR modulator(s).

32. The method of claim 29, wherein the one or more additional pharmaceutical agent(s) is a CFTR potentiator(s).

33. The method of claim 29, wherein the one or more additional pharmaceutical agent(s) comprise a CFTR amplifier(s).

34. A monohydrate form of the free base of N-(pentan-3-yl)-2-(3-(3-(((S)-1-((S)-tetrahydrofuran-2-yl)ethyl)carbamoyl)-1H-pyrazol-5-yl)phenyl)oxazole-5-carboxamide wherein the monohydrate form has an X-ray powder diffraction pattern comprising a characteristic peak, in terms of 20, at about 24.6°.

35. The monohydrate form of claim 34, wherein the X-ray powder diffraction pattern further comprises one or more characteristic peaks, in terms of 2θ, selected from peaks at about 7.6°, about 12.0°, about 15.6°, about 16.6°, about 18.6°, about 18.9°, about 21.5°, and about 23.1°.

36. The monohydrate form of claim 34 having an X-ray powder diffraction pattern substantially as shown in FIG. 1A.

37. The monohydrate form of claim 34 having a differential scanning calorimetry thermogram showing an onset of an endotherm at about 104.6° C.

38. The monohydrate form of claim 34 having a differential scanning calorimetry thermogram substantially as shown in FIG. 1B.

39. The monohydrate form of claim 34 having a differential scanning calorimetry thermogram substantially as shown in FIG. 1C.

40. A metastable hydrate form of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide wherein the metastable hydrate form has an X-ray powder diffraction pattern comprising a characteristic peak, in terms of 2θ, at about 5.0°.

41. The metastable hydrate form of claim 40 where in the X-ray powder diffraction pattern further comprises one or more characteristic peaks, in terms of 2θ, selected from peaks at about 15.1°, about 16.3°, about 18.9°, about 19.1°, and about 20.6°.

42. The metastable hydrate form of claim 40 having an X-ray powder diffraction pattern substantially as shown in FIG. 2A.

43. The metastable hydrate form of claim 40 having a differential scanning calorimetry thermogram showing an onset of an endotherm at about 34.0° C. and a second onset of an endotherm at 159.0° C.

44. The metastable hydrate form of claim 40 having a differential scanning calorimetry thermogram substantially as shown in FIG. 2B.

45. An anhydrous form A of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide wherein the monohydrate form has an X-ray powder diffraction pattern comprising a characteristic peak, in terms of 2θ, at about 6.2°.

46. The anhydrous form A of claim 45, wherein the X-ray powder diffraction pattern further comprises one or more characteristic peaks, in terms of 2θ, selected from peaks at about 13.5°, about 16.5°, about 18.5°, about 18.9°, about 20.4°, and about 24.8°.

47. The anhydrous form A of claim 45 having an X-ray powder diffraction pattern substantially as shown in FIG. 3A.

48. The anhydrous form A of claim 45 having a differential scanning calorimetry thermogram showing an onset of an endotherm at about 191.6° C.

49. The anhydrous form A of claim 45 having a differential scanning calorimetry thermogram substantially as shown in FIG. 3B.

50. An anhydrous form B of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide wherein the monohydrate form has an X-ray powder diffraction pattern comprising a characteristic peak, in terms of 2θ, at about 5.1°.

51. The anhydrous form B of claim 50, wherein the X-ray powder diffraction pattern further comprises one or more characteristic peaks, in terms of 2θ, selected from peaks at about 8.5°, about 15.3°, about 17.6°, about 19.5°, and about 21.0°.

52. The anhydrous form A of claim 50 having an X-ray powder diffraction pattern substantially as shown in FIG. 4A.

53. The anhydrous form B of claim 50 having a differential scanning calorimetry thermogram showing an onset of an endotherm at about 159.2C.

54. The anhydrous form B of claim 50 having a differential scanning calorimetry thermogram substantially as shown in FIG. 4B.

55. An anhydrous form C of the free base of N-(pentan-3-yl)-2-(3-(3-(((S)-1-((S)-tetrahydrofuran-2-yl)ethyl)carbamoyl)-1 H-pyrazol-5-yl)phenyl)oxazole-5-carboxamide wherein the monohydrate form has an X-ray powder diffraction pattern comprising a characteristic peak, in terms of 2θ, at about 5.4°.

56. The anhydrous form C of claim 55, wherein the X-ray powder diffraction pattern further comprises one or more characteristic peaks, in terms of 2θ, selected from peaks at about 14.8°, about 15.1°, about 16.9°, about 18.5°, and about 19.6°.

57. The anhydrous form C of claim 55 having an X-ray powder diffraction pattern substantially as shown in FIG. 5A.

58. The anhydrous form C of claim 55 having a differential scanning calorimetry thermogram showing an onset of an endotherm at about 166.2C.

59. The anhydrous form C of claim 55 having a differential scanning calorimetry thermogram substantially as shown in FIG. 5B.

60. A solid form of N-(pentan-3-yl)-2-(3-(3-(((S)-1-((S)-tetrahydrofuran-2-yl)ethyl)carbamoyl)-1H-pyrazol-5-yl)phenyl)oxazole-5-carboxamide wherein the solid form has an X-ray powder diffraction pattern comprising a characteristic peak, in terms of 2θ, at about 24.6°.

61. The compound according to claim 1 wherein the compound of formula (1) is 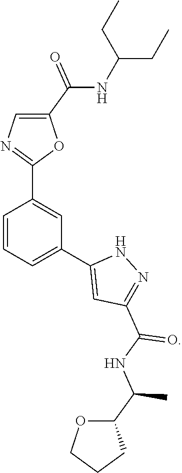 [SMILES: CCC(CC)NC(=O)c1cnc(-c2cccc(-c3cc(C(=O)N[C@@H](C)[C@@H]4CCCO4)n[nH]3)c2)o1]
